SEQUENCE LISTING (6 sequences)

SEQ ID NO: 1 - protein, 214 aa, Artificial sequence
  Pertuzumab light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, Artificial sequence
  Pertuzumab heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 219 aa, Artificial sequence
  Nimotuzumab light chain
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 453 aa, Artificial sequence
  Nimotuzumab heavy chain
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial sequence
  Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, Artificial sequence
  Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2018/0177890 A1
(43) Pub. Date: Jun. 28, 2018

(54) LIGAND-CYTOTOXIC DRUG CONJUGATE, PREPARATION METHOD THEREOF, AND USE THEREOF

(71) Applicants: Jiangsu Hengrui Medicine Co., Ltd., Lianyungang, Jiangsu (CN); Shanghai Hengrui Pharmaceutical Co., Ltd., Minhang District, Shanghai (CN)

(72) Inventors: Jianyan XU, Minhang District, Shanghai (CN); Ying ZHANG, Minhang District, Shanghai (CN); Bolei QU, Minhang District, Shanghai (CN); Fuyao ZHANG, Minhang District, Shanghai (CN); Xiuzhao YU, Minhang District, Shanghai (CN); Jindong LIANG, Minhang District, Shanghai (CN); Guiyang JIANG, Minhang District, Shanghai (CN); Lianshan ZHANG, Minhang District, Shanghai (CN); Ang LI, Minhang District, Shanghai (CN); Yali WANG, Minhang District, Shanghai (CN)

(21) Appl. No.: 15/549,710

(22) PCT Filed: Jan. 26, 2016
(86) PCT No.: PCT/CN2016/072129

§ 371 Date: Aug. 9, 2017

(30) Foreign Application Priority Data
Feb. 15, 2015 (CN) 201510083765.0

(51) Int. Cl. A61K 47/68; C07K 7/06; C07K 16/32; C07K 16/28; C07K 16/30; A61P 35/00

(52) U.S. Cl. CPC A61K 47/6889; C07K 7/06; A61K 47/6817; A61K 47/6857; A61K 47/6849; C07K 16/32; C07K 2317/76; A61K 47/6863; C07K 16/2863; C07K 16/3023; A61P 35/00; C07K 2317/56; C07K 2317/73; A61K 47/6855

(57) ABSTRACT

Provide are ligand-cytotoxic drug conjugates with a general formula of PC-L-Dr, a preparation method thereof, and uses of the ligand-cytotoxic drug conjugate and pharmaceutical compositions containing the same in preparing drugs for treating cancers by means of receptor regulation.

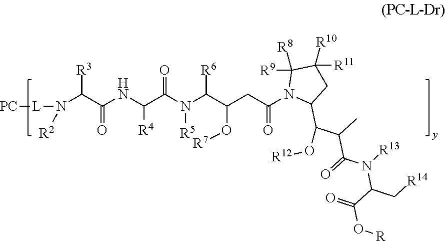

### CROSS-REFERENCE TO RELATED APPLICATION

This application is a Section 371 of International Application No. PCT/CN2016072129, filed Jan. 26, 2016, which was published in the Chinese language on Aug. 18, 2016 under International Publication No. WO 2016/127790 A1, and the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “Sequence Listing 688452_49US”, creation date of Aug. 7, 2017, and having a size of 18.0 KB. The sequence listing submitted via EFS-Web is part of the specification and is herein incorporated by reference in its entirety.

### FIELD OF THE INVENTION

The present disclosure relates to a novel kind of ligand-cytotoxic drug conjugate, and specifically relates to a ligand-cytotoxic drug conjugate, a preparation method thereof, a pharmaceutical composition comprising the same and use of the ligand-cytotoxic drug conjugate and the pharmaceutical composition.

### BACKGROUND OF THE INVENTION

Chemotherapy remains one of the most important anti-cancer treatments along with surgery, radiotherapy, and targeted therapy. Although there are many types of highly efficient cytotoxins, the poor ability of chemotherapy agents to distinguish between tumor and healthy cells limits their broader clinical applications due to toxic side effects. Therapeutic antibodies have emerged as an important class of biological anticancer agents because of their ability to specifically bind to tumor-associated antigens. While this important class of biologics can be used as single agents for the treatment of cancer, their therapeutica efficacy is often limited.

Antibody drug conjugates (ADCs) combine a monoclonal antibody or antibody fragment with a biologically active cytotoxin through a chemically stable linker, taking full advantage of the specificity of antibody binding to the surface antigens of normal cells and tumor cells and the high efficiency of the cytotoxin, while avoiding low efficacy of the antibody and the toxic side effects of the cytotoxin. Therefore, compared with traditional chemotherapy drugs, antibody drug conjugates can accurately recognize tumor cells and reduce damage to normal cells.

Earlier ADCs mainly use mouse monoclonal antibodies, and so some of the drugs were difficult to reach the target due to the human immune response. Secondly, the effector molecules on the early application such as doxorubicin exhibit low biological activity, limiting the efficiency of the first generation antibody drug conjugates. In addition, the source of the antibody, the way and the number of the linker connections were not optimized.

Kadcyla® (ado-trastuzumab emtansine, T-DM1) was approved by the U.S. FDA in February 2013 for the treatment of HER2-positive advanced or metastatic breast cancer patients resistant to trastuzumab (Trastuzumab, trade name: Herceptin®) and paclitaxel. Mylotarg® and Adcetris® are used in targeted therapy against blood tumors, whose tissue structure is relatively simple compared to that of solid tumors. Kadcyla® is the first ADC drug approved by the FDA for the treatment of solid tumors.

Kadcyla® links the highly active mitotic inhibitor DM1 to Roche's Trastuzumab with a stable thioether linker using ImmunoGen's technology, with an average drug to antibody ratio of about 3.5. Trastuzumab specifically binds to breast cancer cells in the patient. After endocytosis, DM1 is released, and the concentration of DM1 in the cells is sufficient to kill the cells due to the mitotic catastrophe. T-DM1 retains the antibody-dependent cell proliferation inhibition effect of Herceptin®, while increasing the potential effect of cytotoxic drugs. Because its toxin is released inside the tumor cells, its side effects do not increase as the curative effect increases.

Pertuzumab (also known as 2C4, trade name Perjeta®) is a recombinant humanized monoclonal antibody, which is the first monoclonal antibody known as the “HER dimerization inhibitor.” Pertuzumab blocks the dimerization of HER2 with other HER receptors by binding to HER2. Pertuzumab has been shown to inhibit tumor growth in prostate cancer with HER2 overexpression or low expression.

Unlike Trastuzumab (trade name Herceptin®), which inhibits downstream signaling pathways by binding to sites located in the HER2 extracellular domain of the proximal region IV sub-domain, Pertuzumab can effectively inhibit HER2 heterodimerization by binding to the II domain (dimerization Domain). Thus, Trastuzumab can only have a certain effect on patients with HER2 overexpressing cancer, especially on patients with breast cancer. However, Pertuzumab, which has the same target and endocytosis as Trastuzumab, but a different mechanism of action, may have a wider range of applications than those drugs blocking the HER2 signaling pathway, because Pertuzumab can cut off the signaling pathway mediated by ErbB family receptors upon inhibiting dimerization.

At present, there are mainly two ADC drug coupling methods: one method used in T-DM1 is the random conjugation between cytotoxic drugs and the free amino groups of antibodies; and the other method used in Adcetris® is the conjugation between cytotoxic drugs and the free thiols resulting from the reduction of the antibody hinge region. Both coupling methods result in a mixture with inconsistent drug loading. For instance, although the average drug loading of T-DM1 is 3.5, the distribution of drug loading ranges from 0 to 8. A low drug loading can affect the efficacy of the ADC, while a high drug loading might cause the ADC drug to be recognized and destroyed by the tissue macrophage system. This not only reduces the half-life of the ADC, but also increases the toxic side effects due to accumulation of the drug in the non-target tissue. In Adcetris®, a reducing agent was used to reduce the disulfide bond in the antibody hinge region, which might affect the stability of the antibody itself. Related ADCs are disclosed in International Patent Application Publications WO2007008603, WO2013173393, WO2005081711, WO2013173391, WO2013173392, WO2013173393 and WO2012010287. The present invention provides a novel ADC compound, which has a novel coupling method and a novel combination of toxin and antibody, and thus has more beneficial effects.

### SUMMARY OF THE INVENTION

In order to improve the ligand, especially the coupling effect between antibody and drug, the present invention provides a compound of formula (PC-L-Dr) comprising an improved linker or a pharmaceutically acceptable salt or solvate thereof:

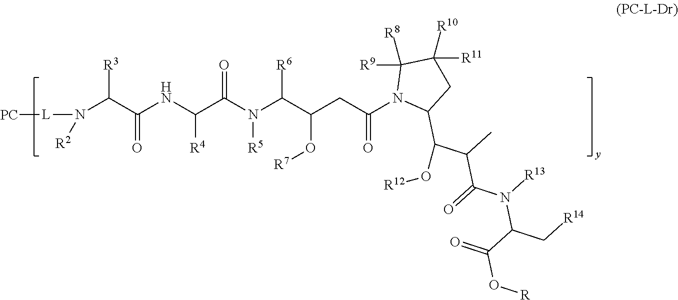

wherein:
each of R, R²-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are hydrogen;
or any two of R⁸-R¹¹ are attached to form a cycloalkyl, the other two being selected from the group consisting of hydrogen, alkyl and cycloalkyl;
each of R¹²-R¹³ is selected from the group consisting of hydrogen, alkyl and halogen; R¹⁴ is selected from the group consisting of aryl and heteroaryl, wherein the aryl or heteroaryl is optionally substituted with one or more groups selected from the group consisting of hydrogen, halogen, hydroxy, alkyl, alkoxy and cycloalkyl;
y is 1-8, preferably 2-5; y is a positive real number which can be an integer or a decimal number;
PC is a ligand; and L is a linker.

In a preferred embodiment of the present invention, a compound of formula (PC-L-Dr) or a pharmaceutically acceptable salt or solvate thereof, is a compound of formula (PC-L-D) or a pharmaceutically acceptable salt or solvate thereof:

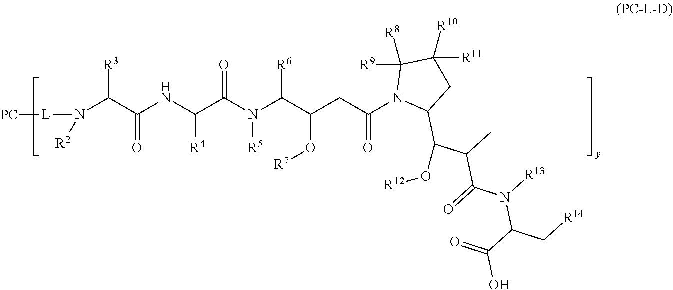

wherein, R²-R¹⁴ are as defined in formula (PC-L-Dr).

In another preferred embodiment of the present invention, a compound of formula (PC-L-Dr) or a pharmaceutically acceptable salt or solvate thereof, is a compound of formula (PC-L′-Dr) or a pharmaceutically acceptable salt or solvate thereof:

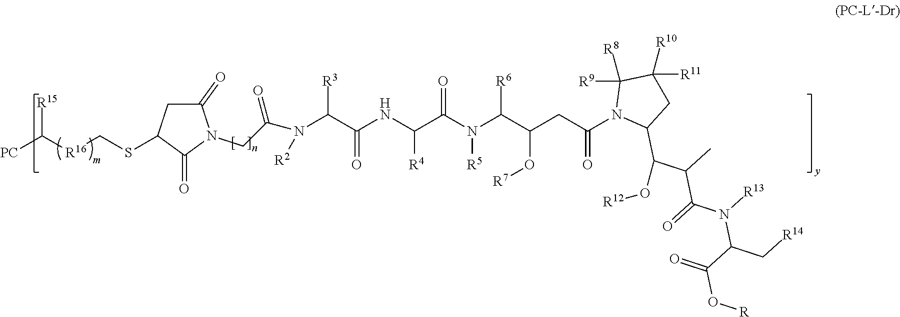

wherein:
R¹⁵ is selected from the group consisting of hydrogen, halogen, hydroxyl, cyano, alkyl, alkoxy and cycloalkyl;
R¹⁶ is selected from the group consisting of alkyl, cycloalkyl, alkoxy and heterocyclyl;
n is 2-6, preferably 2-5;
m is 0-5, preferably 1-3; and
PC, y, n, R, and R²-R¹⁴ are as defined in formula (PC-L-Dr).

In another preferred embodiment of the present invention, a compound of formula (PC-L-Dr) or a pharmaceutically acceptable salt or solvate thereof, is a compound of formula (PC-L′-D) or a pharmaceutically acceptable salt or solvate thereof:

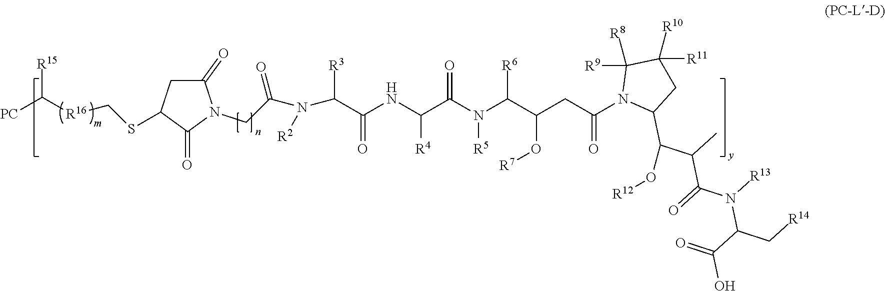

wherein:
R¹⁵, R¹⁶, and m are as defined in formula (PC-L′-Dr); and
PC, y, n, and R²-R¹⁴ are as defined in formula (PC-L-Dr).

In another preferred embodiment of the present invention, a compound of formula (PC-L′-D) or a pharmaceutically acceptable salt or solvate thereof, is a compound of formula (PC-L′-D1) or a pharmaceutically acceptable salt or solvate thereof:

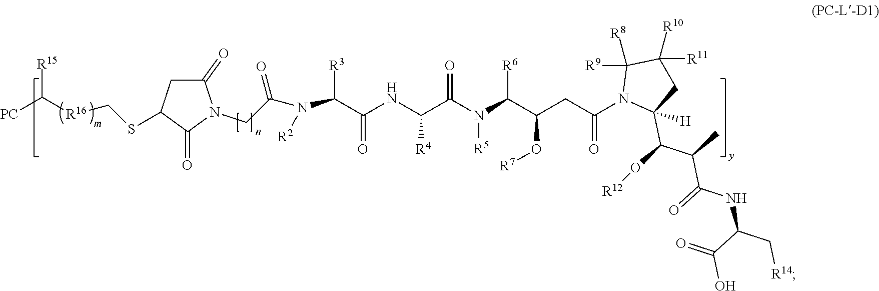

wherein PC, y, n, m, and R²-R¹⁶ are as defined in formula (PC-L′-D).

The ligand-cytotoxic drug conjugates of the present invention include, but are not limited to:

| | compound |
|---|---|
| | (D)/(L1-D) |
| Structure | example |
| 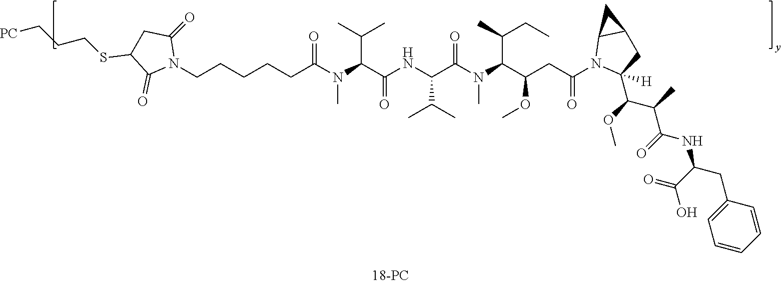 | 1/4 |
| 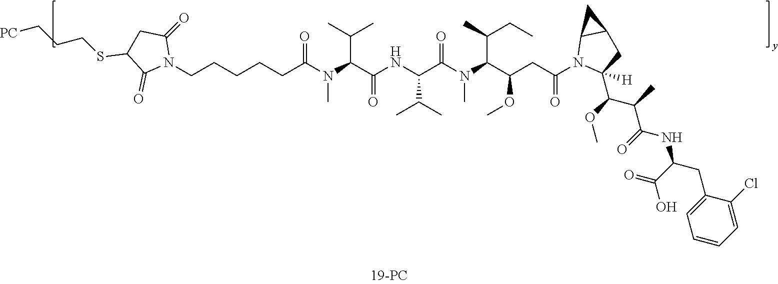 | 2/5 |
| 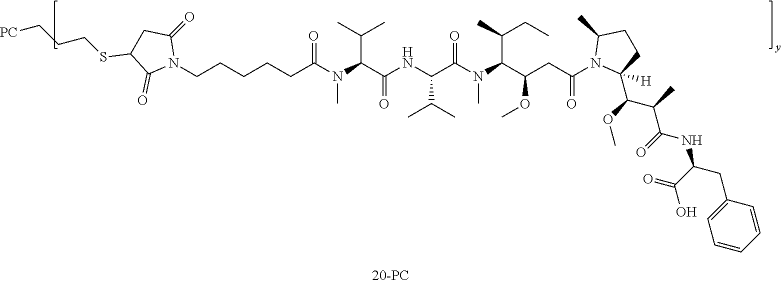 | 3/6 |
| 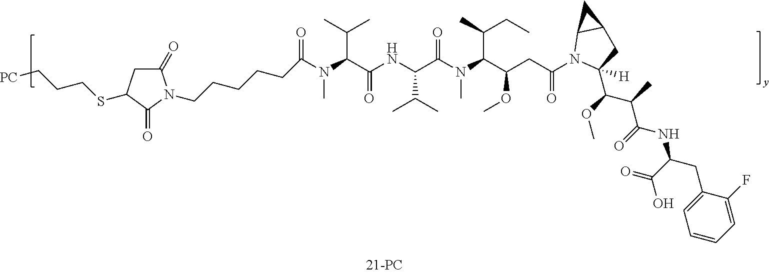 | 7/8 |
| 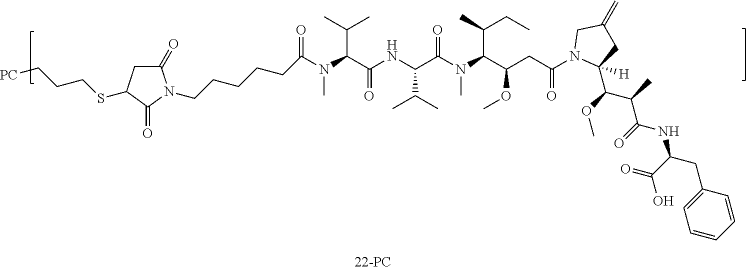 | 9/10 |
| 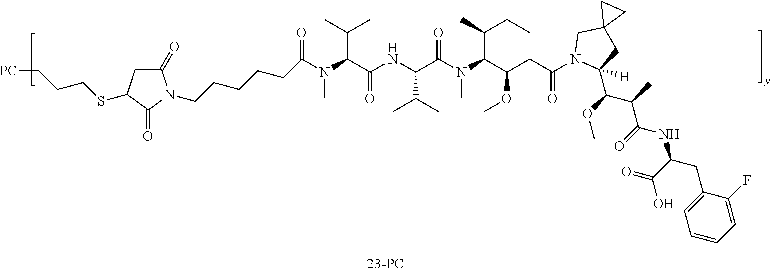 | 11/12 |
| 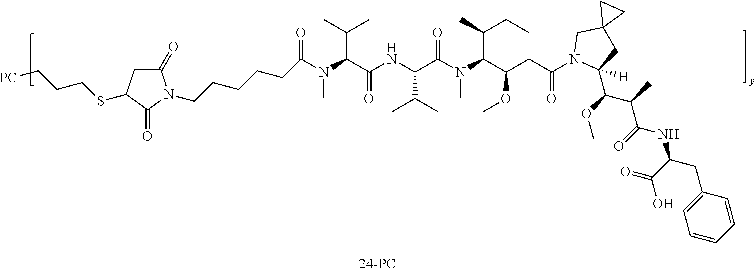 | 13/14 |
| 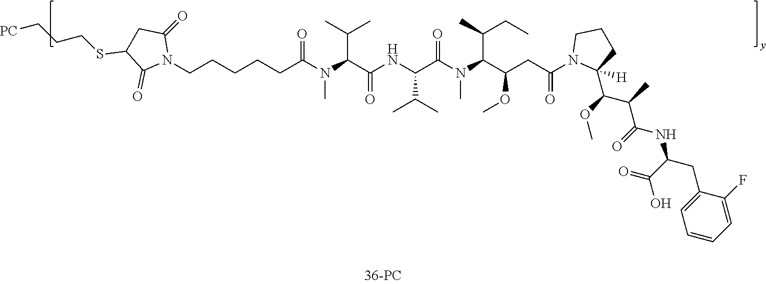 | 25/26 |
| 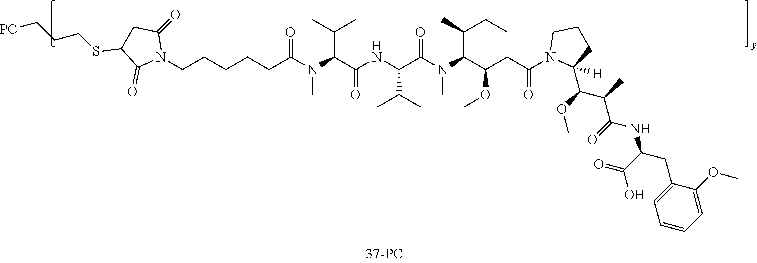 | 27/28 |
| 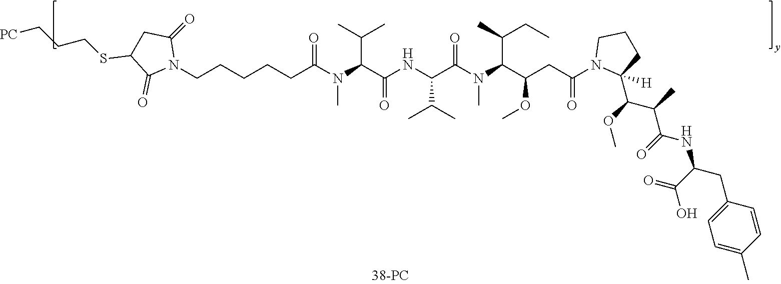 | 29/30 |
| 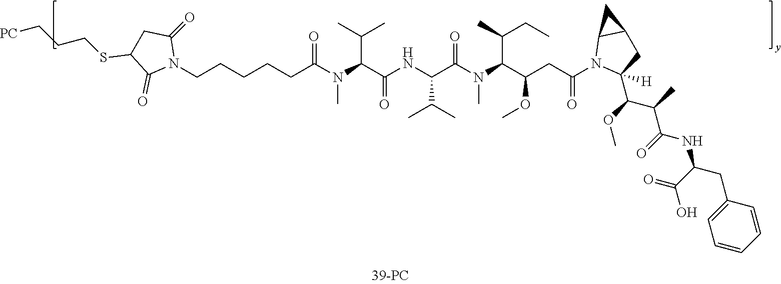 | 1/4 |
| 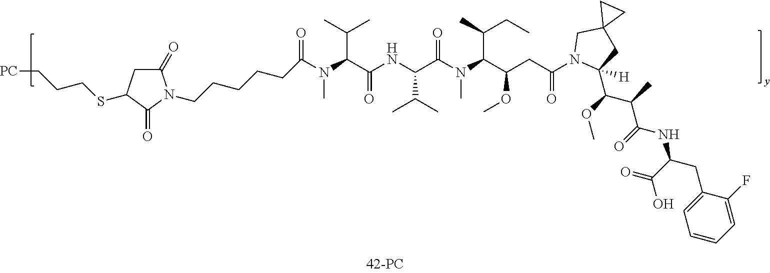 | 11/12 |
| 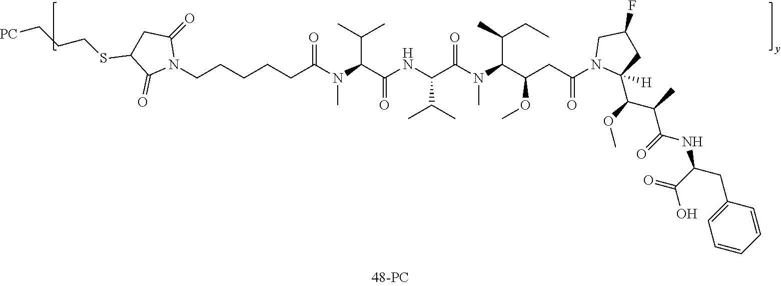 | 44/45 |
| 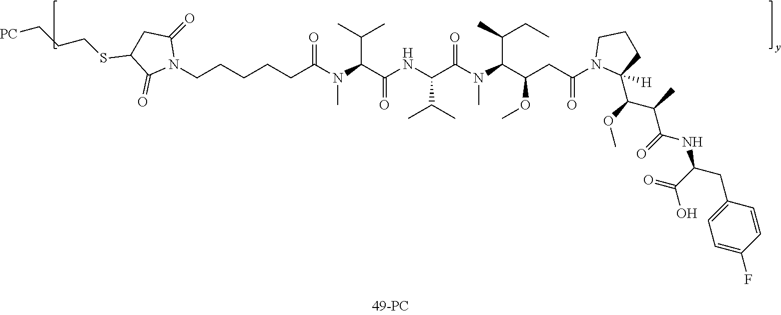 | 46/47 |

or a pharmaceutically acceptable salt or solvate thereof.

In another preferred embodiment of the present invention, a compound of formula (PC-L-Dr), wherein PC is an antibody, preferably selected from the group consisting of Pertuzumab, Nimotuzumab and Trastuzumab.

The typical ligand-cytotoxic drug conjugates of the present invention include, but are not limited to:

| | | com- |
|---|---|---|
| | | pound |
| | | (D)/ |
| | | (L1- |
| | | D) |
| Ex- | | Ex- |
| am- | | am |
| ple | Structure | ple |
| 18 | 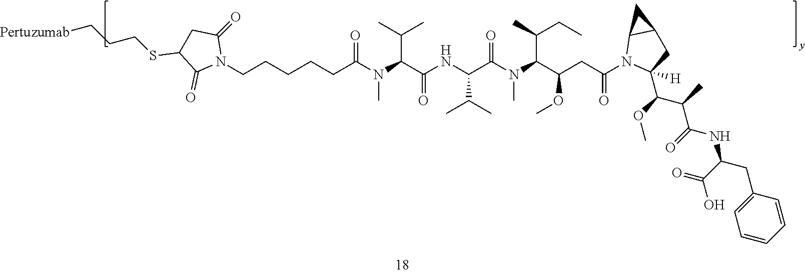 | 1/4 |
| 19 | 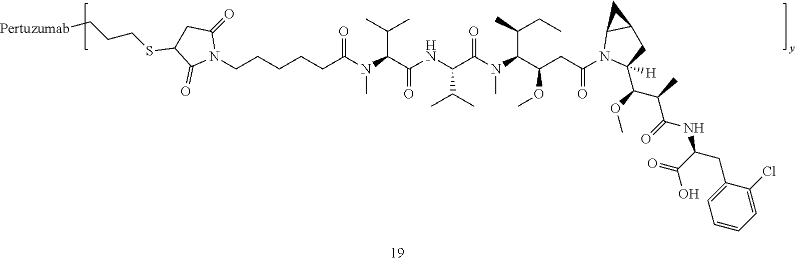 | 2/5 |
| 20 | 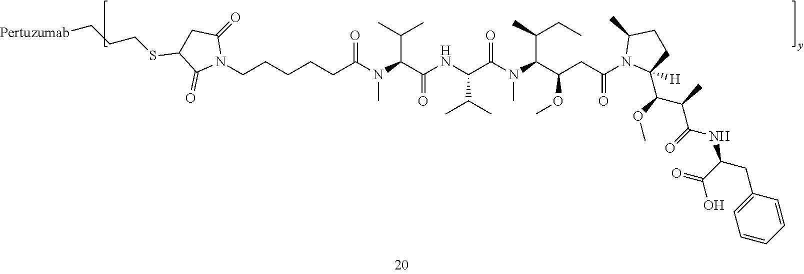 | 3/6 |
| 21 | 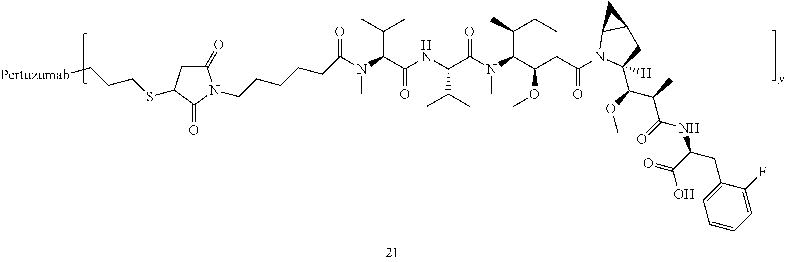 | 7/8 |
| 22 | 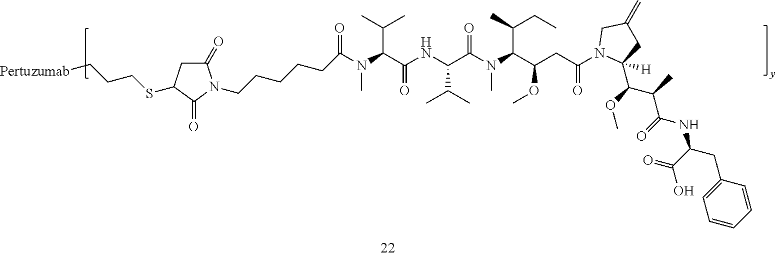 | 9/10 |
| 23 | 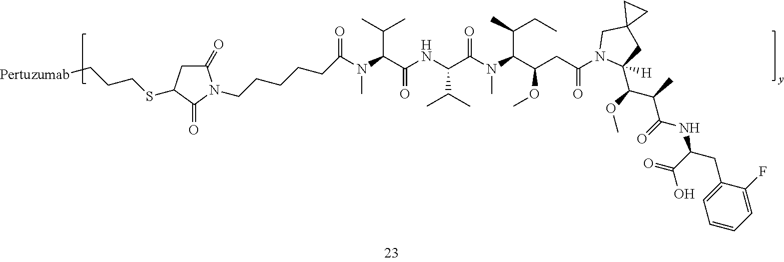 | 11/12 |
| 24 | 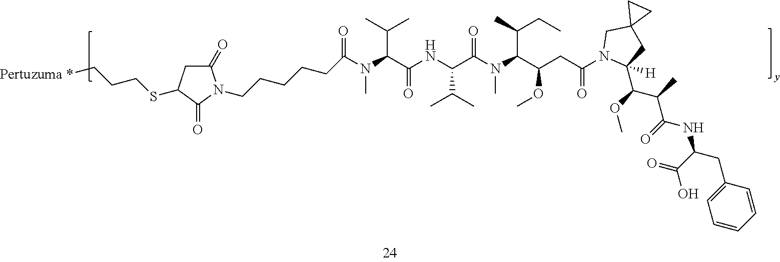 | 13/14 |
| 36 | 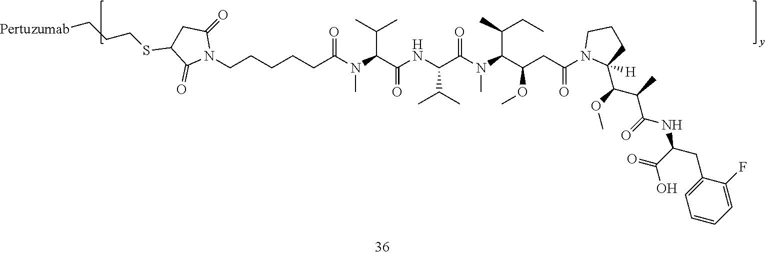 | 25/26 |
| 37 | 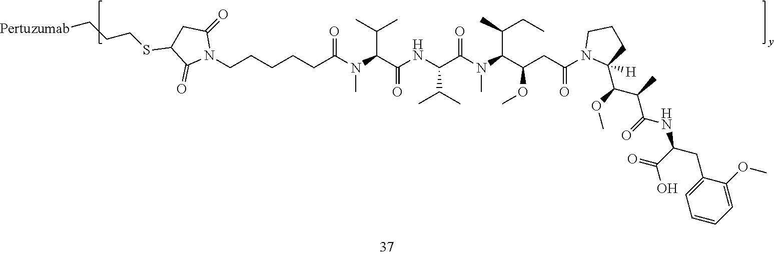 | 27/28 |
| 38 | 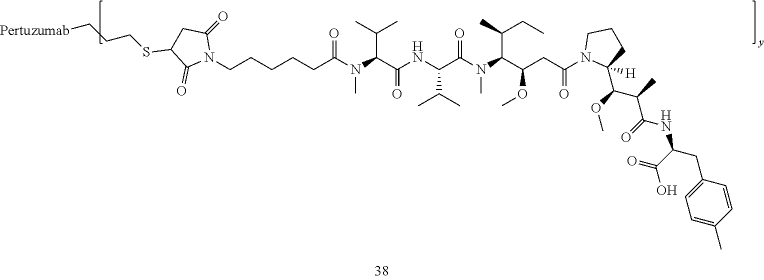 | 29/30 |
| 39 | 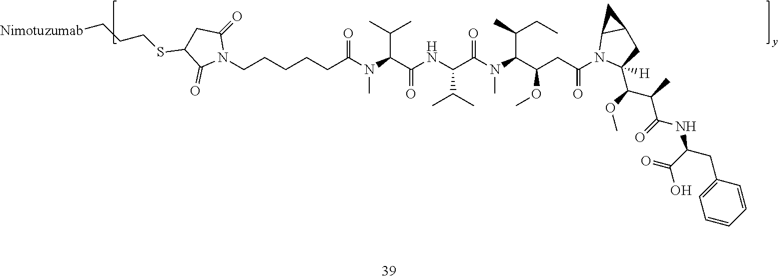 | 1/4 |
| 40 | 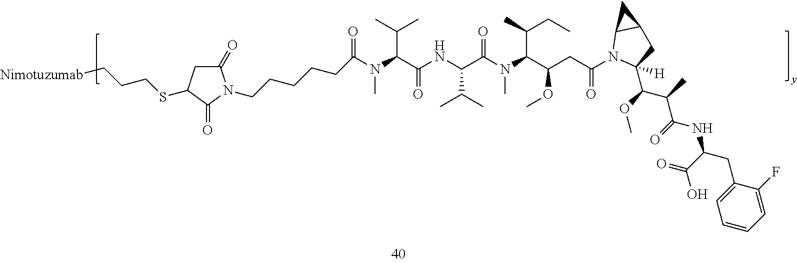 | 7/8 |
| 41 | 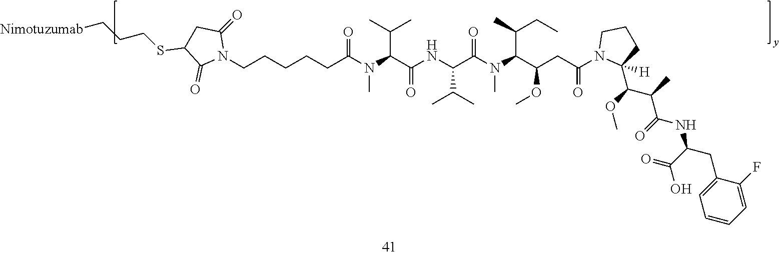 | 25/26 |
| 42 | 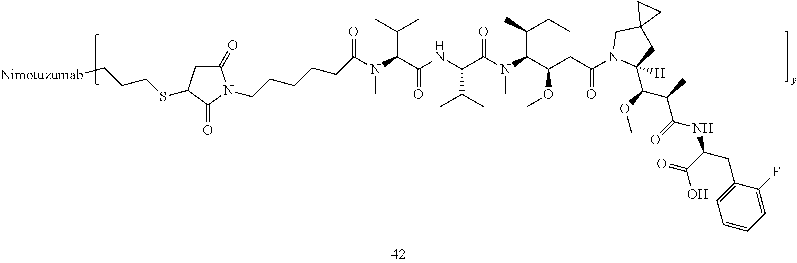 | 11/12 |
| 43 | 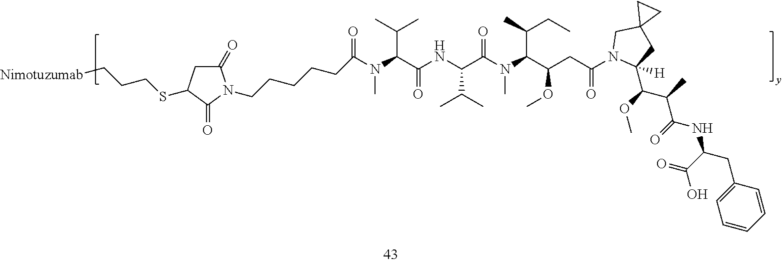 | 13/14 |
| 48 | 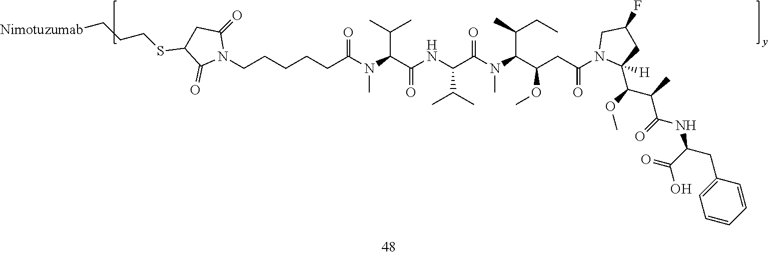 | 44/45 |
| 49 | 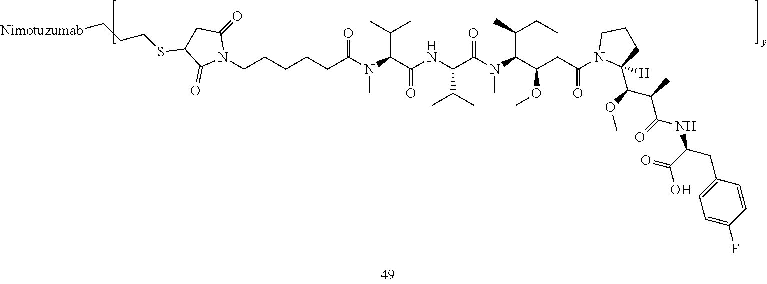 | 46/47 |
| 50 | 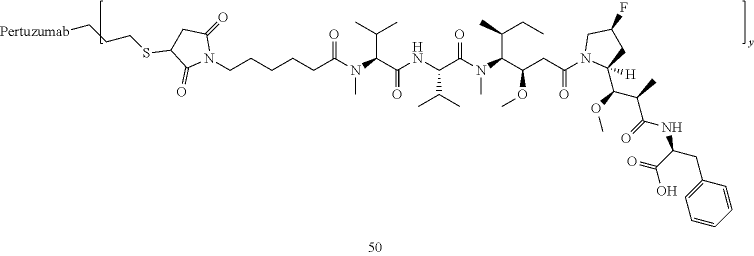 | 44/45 |
| 51 | 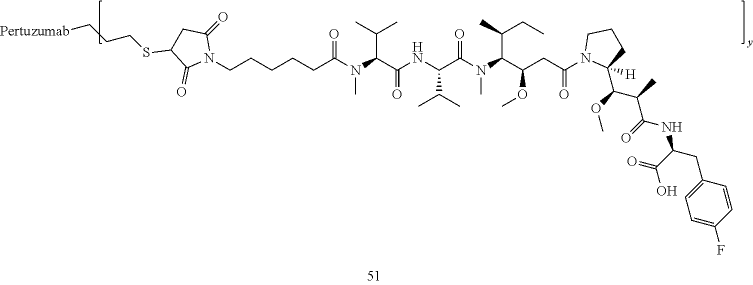 | 46/47 |
| 52 | 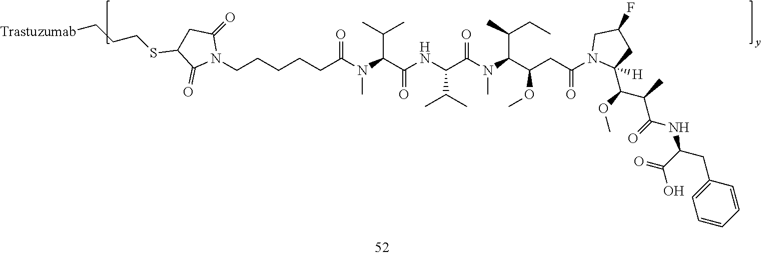 | 44/45 |
| 53 | 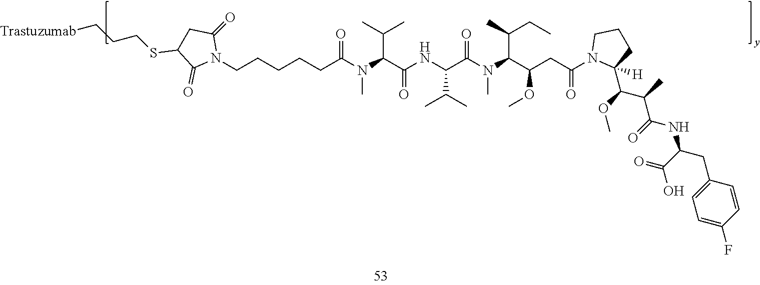 | 46/47 |

or a pharmaceutically acceptable salt or solvate thereof

Another aspect of this invention is directed to a compound of formula (Dr):

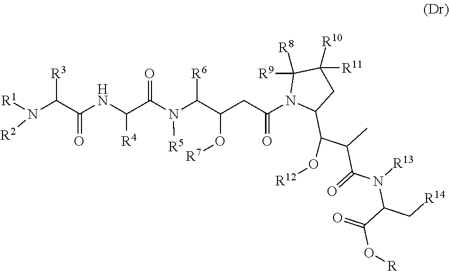

or a tautomer, mesomer, racemate, enantiomer, diastereomer, or mixture thereof, or a pharmaceutically acceptable salt thereof, which can be used as the intermediate in the preparation of a compound of formula (PC-L-Dr):
wherein:
each of R, R¹-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are hydrogen;
or any two of R⁸-R¹¹ are attached to form a cycloalkyl, and the other two are each selected from the group consisting of hydrogen, alkyl and cycloalkyl;
each of R¹²-R¹³ is selected from the group consisting of hydrogen, alkyl and halogen, R¹⁴ is selected from the group consisting of aryl and heteroaryl, wherein the aryl and heteroaryl are optionally further substituted with one or more groups selected from the group consisting of hydrogen, halogen, hydroxy, alkyl, alkoxy and cycloalkyl.

In another preferred embodiment of the present invention, a compound of formula (Dr), is a compound of formula (D) or a pharmaceutically acceptable salt or solvate thereof:

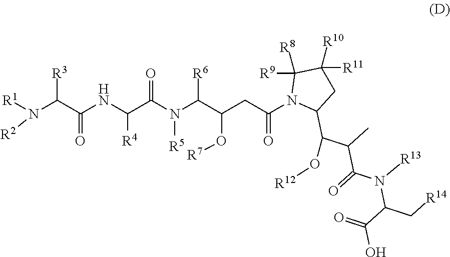

or a tautomer, mesomer, racemate, enantiomer, diastereomer, or mixture thereof, or a pharmaceutically acceptable salt thereof, wherein R¹-R¹⁴ are as defined in formula (Dr).

In another preferred embodiment of the present invention, a compound of formula (Dr) is a compound of formula (D1):

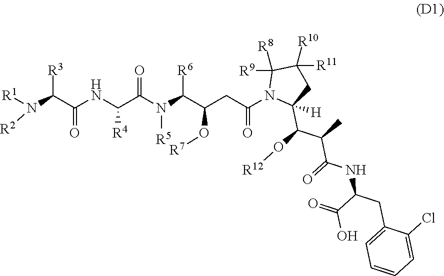

or a tautomer, mesomer, racemate, enantiomer, diastereomer, or mixture thereof, or a pharmaceutically acceptable salt thereof, wherein R¹-R¹⁴ are as defined in formula (D).

The typical compounds for the preparation of ligand-cytotoxic drug conjugate of the present invention include, but are not limited to:

| Example | Structure and Name |
|---|---|
| 1 | 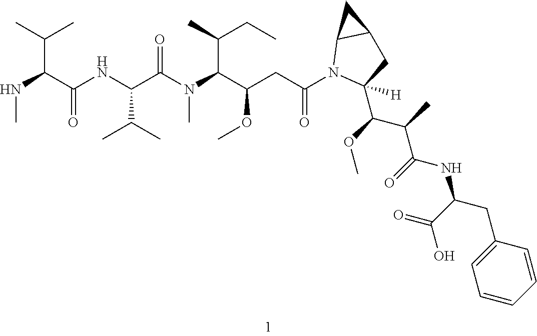 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] |
| | (S)-2-((2R,3R)-3-((1S,3S,5S)-2-((3R,4S,5S)-4-((S)-N,3-dimethyl-2-((S)-3- |
| | methyl-2-(methylamino)butanamido)butanamido)-3-methoxy-5-methylheptanoyl)-2-azabicyclo |
| | [3.1.0]hexan-3-yl)-3-methoxy-2-methylpropanamido)-3-phenylpropanoic acid |
| 2 | 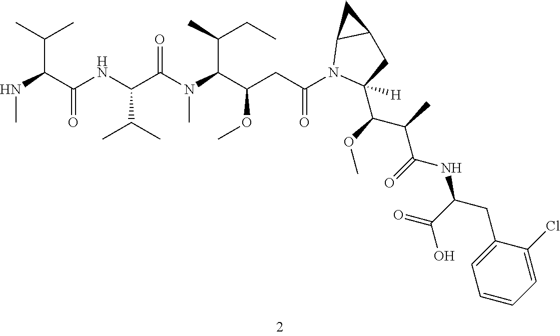 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@@H](NC)C(C)C)C(C)C] |
| | (S)-3-(2-chlorophenyl)-2-((2R,3R)-3-((1S,3S,5S)-2-((3R,4S,5S)-4-((S)-N,3- |
| | dimethyl-2-((S)-3-methyl-2-(methylamino)butanamido)butanamido)-3- |
| | methoxy-5-methylheptanoyl)-2-azabicyclo[3.1.0]hexan-3-yl)-3-methoxy- |
| | 2-methylpropanamido)propanoic acid |
| 3 | 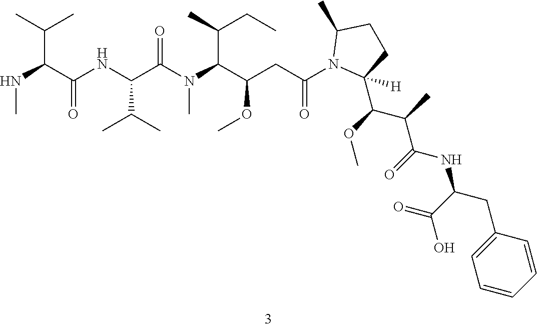 [SMILES: CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] |
| | (S)-2-((2R,3R)-3-((2S,5S)-1-((3R,4S,5S)-4-((S)-N,3-dimethyl-2-((S)-3- |
| | methyl-2-(methylamino)butanamido)butanamido)-3-methoxy-5-methyl- |
| | heptanoyl)-5-methylpyrrolidin-2-yl)-3-methoxy-2-methylpropanamido)-3-phenylpropanoic acid |
| 7 | 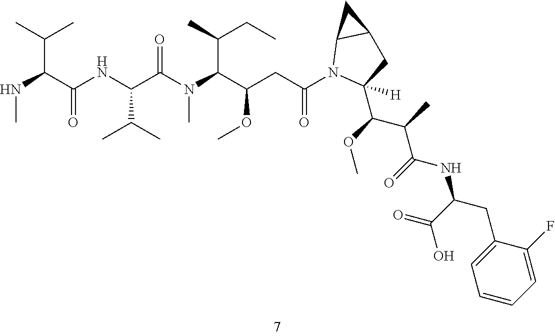 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C] |
| | (S)-2-((2R,3R)-3-((1S,3S,5S)-2-((3R,4S,5S)-4-((S)-N,3-dimethyl-2-((S)-3- |
| | methyl-2-(methylamino)butanamido)butanamido)-3-methoxy-5-methyl |
| | heptanoyl)-2-azabicyclo[3.1.0]hexan-3-yl)-3-methoxy-2-methylpropanamido)- |
| | 3-(2-fluorophenyl)propanoic acid |
| 9 | 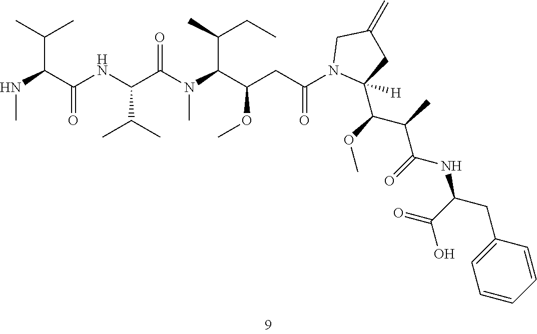 [SMILES: C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@@H](NC)C(C)C)C(C)C)C1] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl)- |
| | 4-methylenepyrrolidin-2-yl)-3-methoxy-2-methylpropanamido)-3-phenylpropanoic acid |
| 11 | 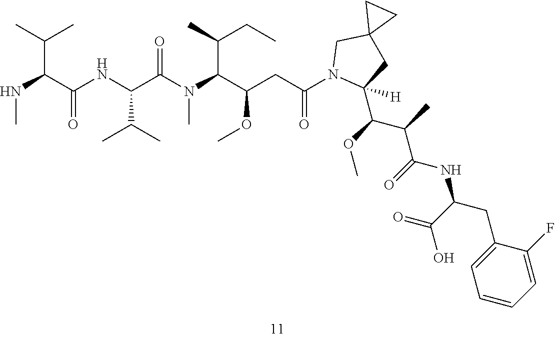 [SMILES: CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C] |
| | (S)-2-((2R,3R)-3-((S)-5-((3R,4S,5S)-4-((S)-N,3-dimethyl-2-((S)-3-methyl- |
| | 2-(methylamino)butanamido)butanamido)-3-methoxy-5-methylheptanoyl)-5-azaspiro[2.4] |
| | heptan-6-yl)-3-methoxy-2-methylpropanamido)-3-(2-fluorophenyl)propanoic acid |
| 13 | 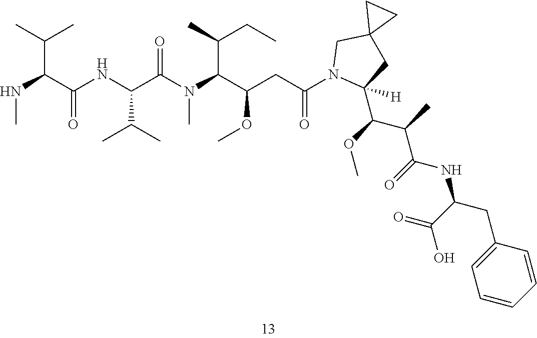 [SMILES: CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] |
| | (S)-2-((2R,3R)-3-((S)-5-((3R,4S,5S)-4-((S)-N,3-dimethyl-2-((S)-3-methyl- |
| | 2-(methylamino)butanamido)butanamido)-3-methoxy-5-methylheptanoyl)-5-azaspiro[2.4] |
| | heptan-6-yl)-3-methoxy-2-methylpropanamido)-3-phenylpropanoic acid |
| 15 | 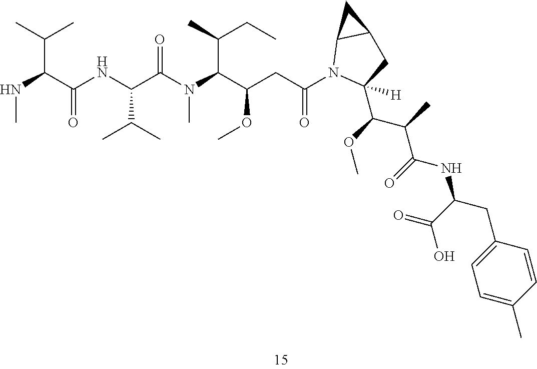 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccc(C)cc1)C(=O)O)OC)N(C)C(=O)[C@@H](NC(=O)[C@@H](NC)C(C)C)C(C)C] |
| | (S)-2-((2R,3R)-3-((1S,3S,5S)-2-((3R,4S,5S)-4-((S)-N,3-dimethyl-2-((S)-3- |
| | methyl-2-(methylamino)butanamido)butanamido)-3-methoxy-5-methylheptanoyl)-2- |
| | azabicyclo[3.1.0]hexan-3-yl)-3-methoxy-2-methylpropanamido)-3-(p-tolyl)propanoic acid |
| 16 | 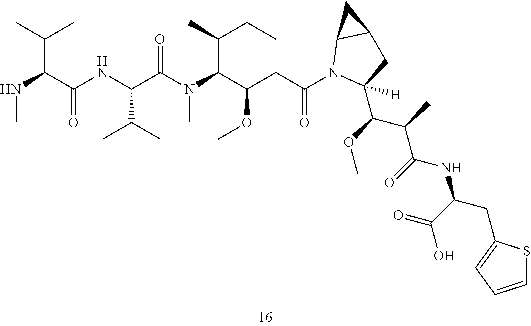 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccs1)C(=O)O)OC)N(C)C(=O)[C@@H](NC(=O)[C@@H](NC)C(C)C)C(C)C] |
| | (S)-2-((2R,3R)-3-((1S,3S,5S)-2-((3R,4S,5S)-4-((S)-N,3-dimethyl-2-((S)-3- |
| | methyl-2-(methylamino)butanamido)butanamido)-3-methoxy-5-methyl |
| | heptanoyl)-2-azabicyclo[3.1.0]hexan-3-yl)-3-methoxy-2-methyl |
| | propanamido)-3-(thiophen-2-yl)propanoic acid |
| 17 | 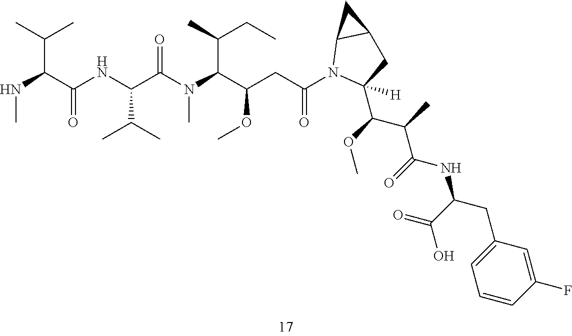 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccc(F)c1)C(=O)O)OC)N(C)C(=O)[C@@H](NC(=O)[C@@H](NC)C(C)C)C(C)C] |
| | (S)-2-((2R,3R)-3-((1S,3S,5S)-2-((3R,4S,5S)-4-((S)-N,3-dimethyl-2-((S)-3- |
| | methyl-2-(methylamino)butanamido)butanamido)-3-methoxy-5-methyl |
| | heptanoyl)-2-azabicyclo[3.1.0]hexan-3-yl)-3-methoxy-2-methylpropanamido)- |
| | 3-(3-fluorophenyl)propanoic acid |
| 25 | 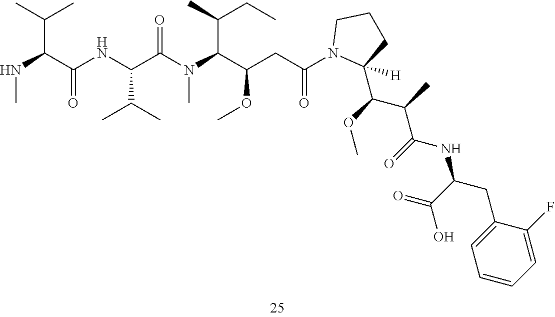 [SMILES: CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C] |
| | (S)-2-((2R,3R)-3-((S)-1-((3R,4R,5S)-4-((S)-N,3-dimethyl-2-((S)-3-methyl- |
| | 2-(methylamino)butanamido)butanamido)-3-methoxy-5-methylheptanoyl) |
| | pyrrolidin-2-yl)-3-methoxy-2-methylpropanamido)-3-(2-fluorophenyl)propanoic acid |
| 27 | 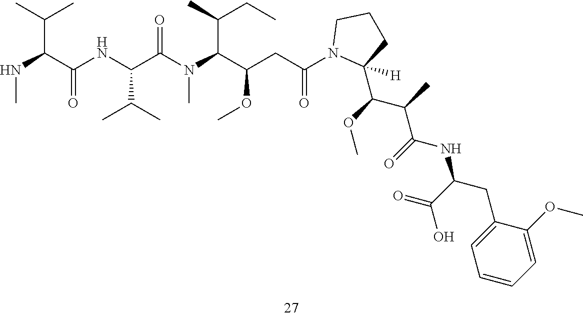 [SMILES: CC[C@H](C)[C@@H]([C@@H](CC(=O)N1CCC[C@H]1[C@H](OC)[C@@H](C)C(=O)N[C@@H](Cc1ccccc1OC)C(=O)O)OC)N(C)C(=O)[C@@H](NC(=O)[C@@H](NC)C(C)C)C(C)C] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl) |
| | pyrrolidin-2-yl)-3-methoxy-2-methylpropanamido)-3-(2-methoxyphenyl)propanoic acid |
| 29 | 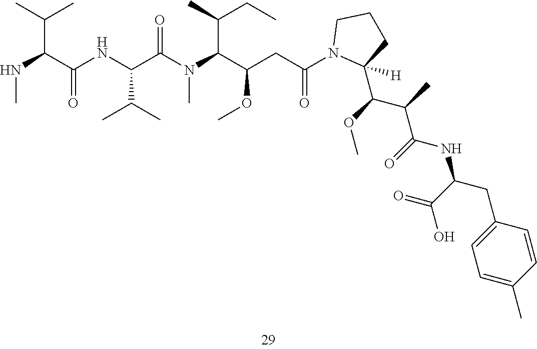 [SMILES: CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@@H](NC)C(C)C)C(C)C] |
| | (S)-2-((2R,3R)-3-((S)-1-((3R,4R,5S)-4-((S)-N,3-dimethyl-2-((S)-3- |
| | methyl-2-(methylamino)butanamido)butanamido)-3-methoxy-5-methyl- |
| | heptanoyl)pyrrolidin-2-yl)-3-methoxy-2-methylpropanamido)-3-(p-tolyl)propanoic acid |
| 31 | 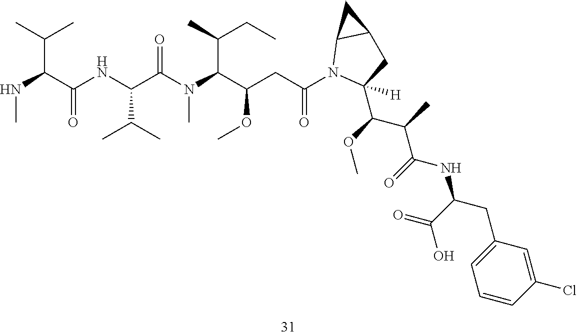 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccc(Cl)c1)C(=O)O)OC)N(C)C(=O)[C@@H](NC(=O)[C@@H](NC)C(C)C)C(C)C] |
| | (S)-3-(3-chlorophenyl)-2-((2R,3R)-3-((S)-1-((3R,4S,5S)-4-((S)-N,3- |
| | dimethyl-2-((S)-3-methyl-2-(methylamino)butanamido)butanamido)-3-methoxy-5- |
| | methylheptanoyl)pyrrolidin-2-yl)-3-methoxy-2-methylpropanamido)propanoic acid |
| 32 | 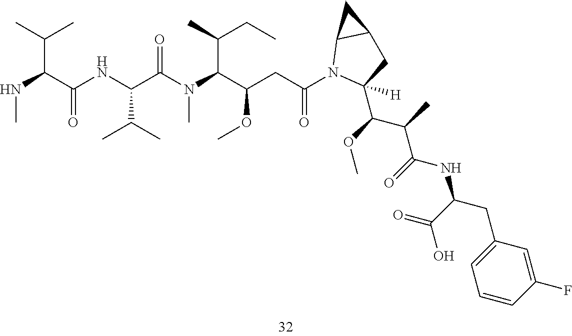 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccc(F)c1)C(=O)O)OC)N(C)C(=O)[C@@H](NC(=O)[C@@H](NC)C(C)C)C(C)C] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl)pyrrolidin- |
| | 2-yl)-3-methoxy-2-methylpropanamido)-3-(3-fluorophenyl)propanoic acid |
| 33 | 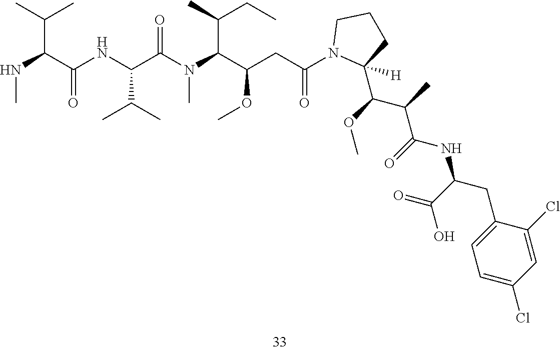 [SMILES: CC[C@H](C)[C@@H]([C@@H](CC(=O)N1CCC[C@H]1[C@H](OC)[C@@H](C)C(=O)N[C@@H](Cc1ccc(Cl)cc1Cl)C(=O)O)OC)N(C)C(=O)[C@@H](NC(=O)[C@@H](NC)C(C)C)C(C)C] |
| | (S)-3-(2,4-dichlorophenyl)-2-((2R,3R)-3-((S)-1-((3R,4S,5S)-4-((S)-N,3- |
| | dimethyl-2-((S)-3-methyl-2-(methylamino)butanamido)butanamido)-3-methoxy-5- |
| | methylheptanoyl)pyrrolidin-2-yl)-3-methoxy-2-methylpropanamido)propanoic acid |
| 34 | 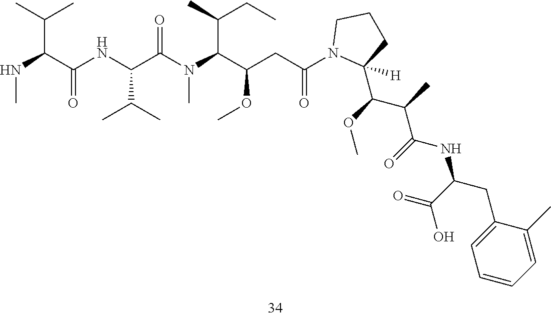 [SMILES: CC[C@H](C)[C@@H]([C@@H](CC(=O)N1CCC[C@H]1[C@H](OC)[C@@H](C)C(=O)N[C@@H](Cc1ccccc1C)C(=O)O)OC)N(C)C(=O)[C@@H](NC(=O)[C@@H](NC)C(C)C)C(C)C] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl) |
| | pyrrolidin-2-yl)-3-methoxy-2-methylpropanamido)-3-(o-tolyl)propanoic acid |
| 35 | 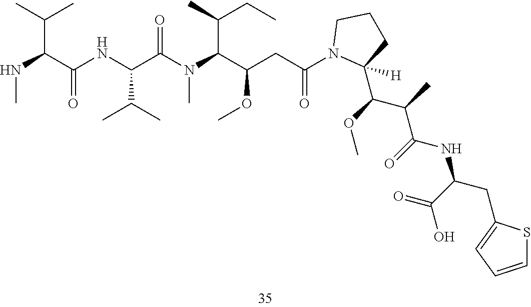 [SMILES: CC[C@H](C)[C@@H]([C@@H](CC(=O)N1CCC[C@H]1[C@H](OC)[C@@H](C)C(=O)N[C@@H](Cc1cccs1)C(=O)O)OC)N(C)C(=O)[C@@H](NC(=O)[C@@H](NC)C(C)C)C(C)C] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl) |
| | pyrrolidin-2-yl)-3-methoxy-2-methylpropanamido)-3-(thiophen-2-yl)propanoic acid |
| 44 | 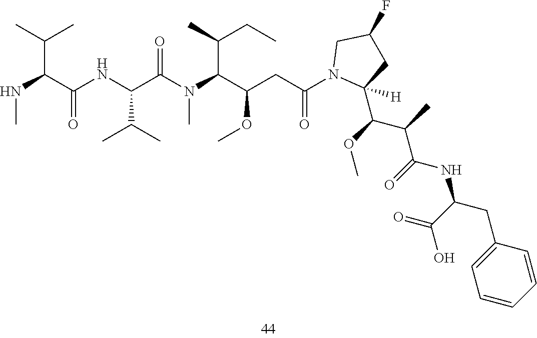 [SMILES: CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] |
| | (S)-2-((2R,3R)-3-((2S,4S)-1-((3R,4S,5S)-4-((S)-N,3-dimethyl-2-((S)-3- |
| | methyl-2-(methylamino)butanamido)butanamido)-3-methoxy-5-methylheptanoyl)-4- |
| | fluoropyrrolidin-2-yl)-3-methoxy-2-methylpropanamido)-3-phenylpropanoic acid |
| 46 | 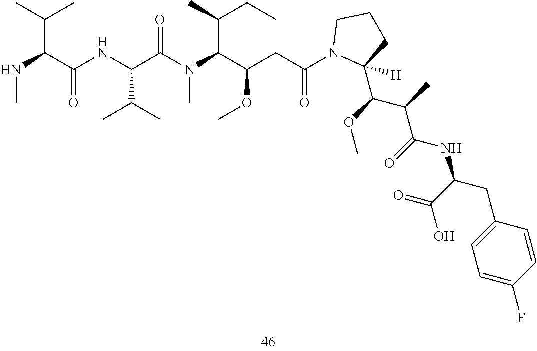 [SMILES: CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@@H](NC)C(C)C)C(C)C] |
| | (S)-2-((2R,3R)-3-((S)-1-((3R,4S,5S)-4-((S)-N,3-dimethyl-2-((S)-3-methyl- |
| | 2-(methylamino)butanamido)butanamido)-3-methoxy-5-methylheptanoyl)pyrrolidin- |
| | 2-yl)-3-methoxy-2-methylpropanamido)-3-(4-fluorophenyl)propanoic acid |

Another aspect of the present invention is directed to a compound of formula (L1-Dr):

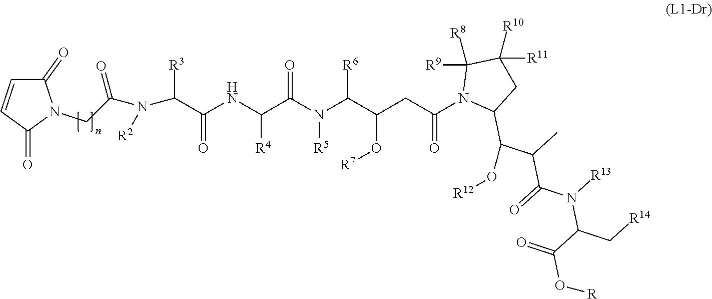

which can be used as an intermediate in the preparation of a compound of formula (PC-L′-D):
wherein:
n is 2-6, preferably 2-5;
each of R, and R¹-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are hydrogen;
or any two of R⁸-R¹¹ are attached to form a cycloalkyl, the other two are each selected from the group consisting of hydrogen, alkyl and cycloalkyl;
each of R¹²-R¹³ is selected from the group consisting of hydrogen, alkyl and halogen, R¹⁴ is selected from aryl and heteroaryl, wherein the aryl or heteroaryl are optionally further substituted with one or more groups selected from the group consisting of hydrogen, halogen, hydroxy, alkyl, alkoxy and cycloalkyl.

In another preferred embodiment of the present invention, a compound of formula (L₁-Dr) is a compound of formula (L₁-D):

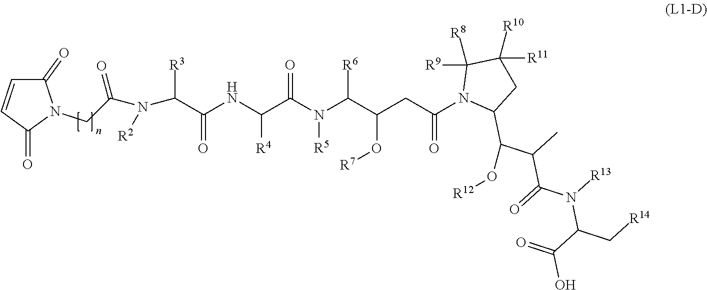

wherein n, and R²-R¹⁴ are as defined in formula (L₁-Dr).

In another preferred embodiment of the present invention, a compound of formula (L₁-D), is a compound of formula (L₁-D1):

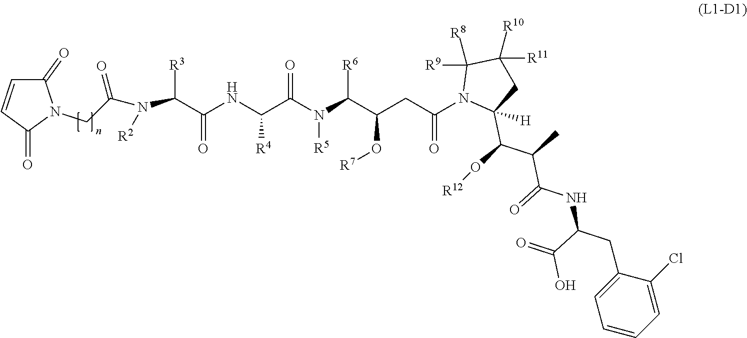

wherein n, and R¹-R¹² are as defined in formula (L₁-D).

Typical pro-drugs (with a linker) and intermediates for the preparation of a ligand-cytotoxic drug conjugate of the present invention include, but are not limited to:

| Exam- | |
|---|---|
| ple | Structure and Name |
| 4 | 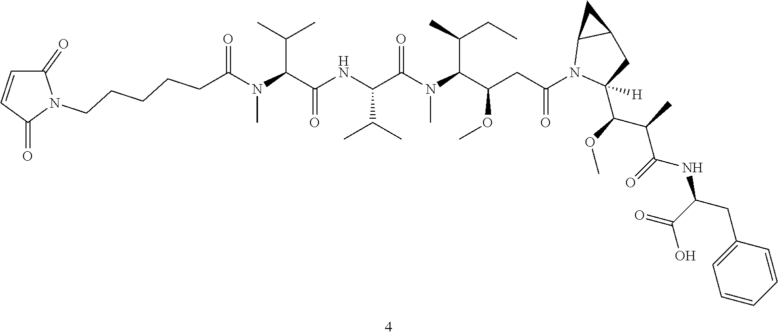 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)- |
| | 3-methylbutanamido)-N,3-dimethylbutanamido)-3-methoxy-5-methylheptanoyl)-2-azabicyclo[3.1.0]hexan-3-yl)-3-methoxy-2- |
| | methylpropanamido)-3-phenylpropanoic acid |
| 5 | 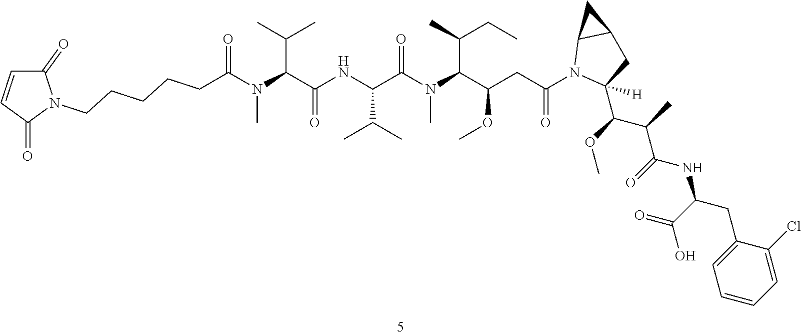 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@H](C(C)C)N(C)C(=O)CCCCCN1C(=O)C=CC1=O)C(C)C] |
| | (S)-3-(2-chlorophenyl)-2-((2R,3R)-3-((1S,3S,5S)-2-((3R,4S,5S)-4-((S)-2-((S)-2-(6-(2,5-dioxo-2,5-dihydro-1H-pyrrol- |
| | 1-yl)-N-methylhexanamido)-3-methylbutanamido)-N,3-dimethylbutanamido)-3-methoxy-5-methylheptanoyl)-2- |
| | azabicyclo[3.1.0]hexan-3-yl)-3-methoxy-2-methylpropanamido)propanoic acid |
| 6 | 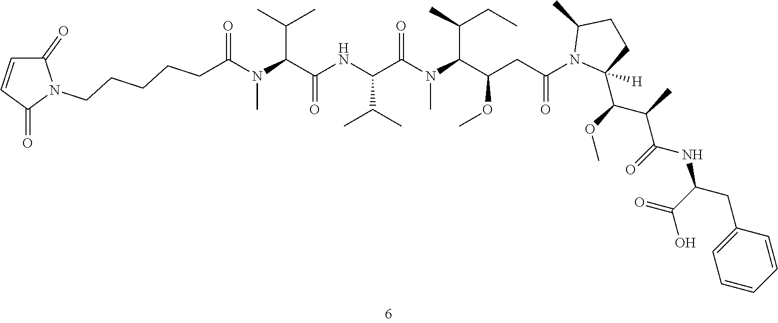 [SMILES: CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((2S,5S)-1-((3R,4S,5S)-4-((S)-2-((S)-2-(6-(2,5-dioxo-2,5-dihydro-1H-pyrrol-1-yl)-N-methylhexanamido)- |
| | 3-methylbutanamido)-N,3-dimethylbutanamido)-3-methoxy-5-methylheptanoyl)-5-methylpyrrolidin-2-yl)-3-methoxy-2- |
| | methylpropanamido)-3-phenylpropanoic acid |
| 8 | 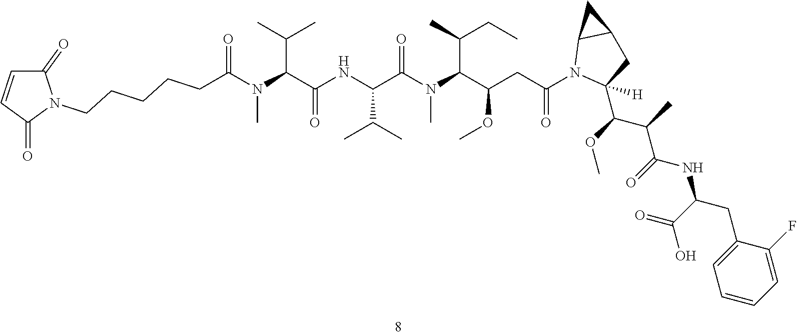 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)-2-azabicylo[3.1.0]hexan-3-yl)-3-methoxy-2- |
| | methylpropanamido)-3-(2-fluorophenyl)propanoic acid |
| 10 | 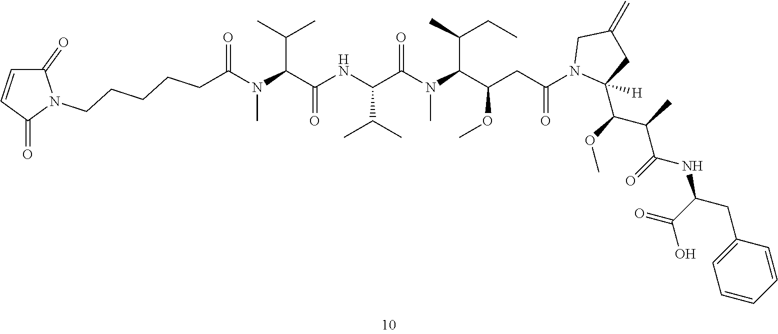 [SMILES: C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@H](C(C)C)N(C)C(=O)CCCCCN2C(=O)C=CC2=O)C(C)C)C1] |
| | (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)-4-methylenepyrrolidin-2-yl)-3- |
| | methoxy-2-methylpropanamido)-3-phenylpropanoic acid |
| 12 | 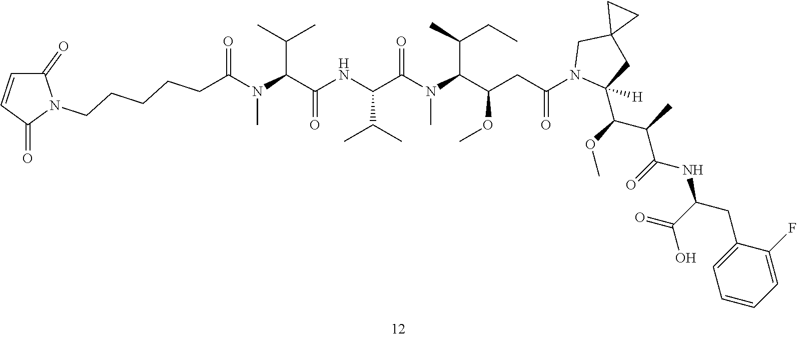 [SMILES: CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)-5-azaspiro[2.4]heptan-6-yl)-3-methoxy-2- |
| | methylpropanamido)-3-(2-fluorophenyl)propanoic acid |
| 14 | 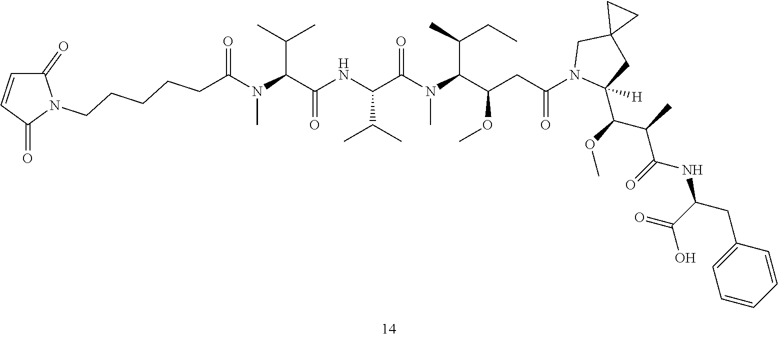 [SMILES: CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-5-((3R,4S,5S)-4-((S)-2-((S)-2-(6-(2,5-dihydro-1H-pyrrol-1-yl)-N-methylhexanamido)-3- |
| | methylbutanamido)-N,3-dimethylbutanamido)-3-methoxy-5-methylheptanoyl)-5-azaspiro[2.4]heptan- |
| | 6-yl)-3-methoxy-2-methylpropanamido)-3-phenylpropanoic acid |
| 45 | 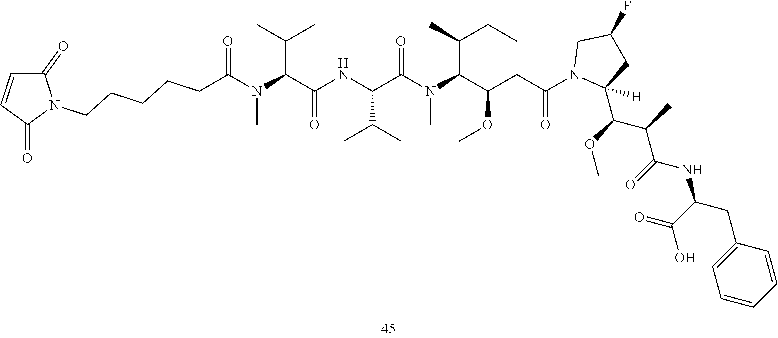 [SMILES: CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((2S,4S)-1-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)-4-fluoropyrrolidin-2-yl)- |
| | 3-methoxy-2-methylpropanamido)-3-phenylpropananoic acid |
| 26 | 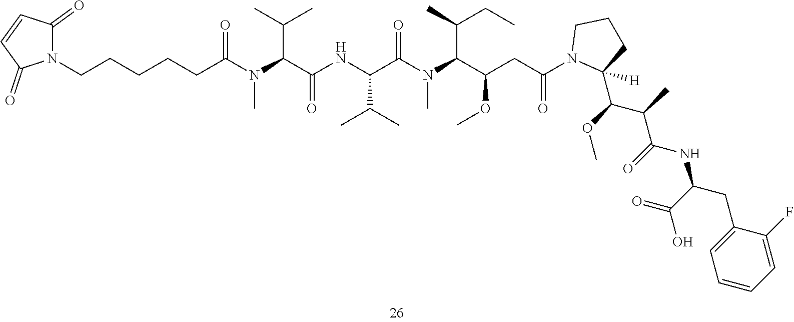 [SMILES: CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-1-((3R,4S,5S)-4-((S)-2-((S)-2-(6-(2,5-dioxo-2,5-dihydro-1H-pyrrol-1-yl)-N-methyhexanamido)-3- |
| | methylbutanamido)-N,3-dimethylbutanamido)-3-methoxy-5-methylheptanoyl)pyrrolidin-2-yl)-3-methoxy-2- |
| | methylpropanamido)-3-(2-fluorophenyl)propanoic acid |
| 28 | 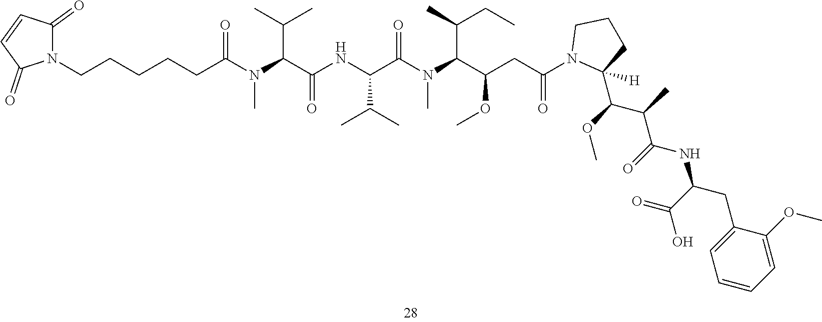 [SMILES: CC[C@H](C)[C@@H]([C@@H](CC(=O)N1CCC[C@H]1[C@H](OC)[C@@H](C)C(=O)N[C@@H](Cc1ccccc1OC)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)pyrrolidin-2-yl)-3-methoxy-2- |
| | methylpropanamido)-3-(2-methoxyphenyl)propanoic acid |
| 30 | 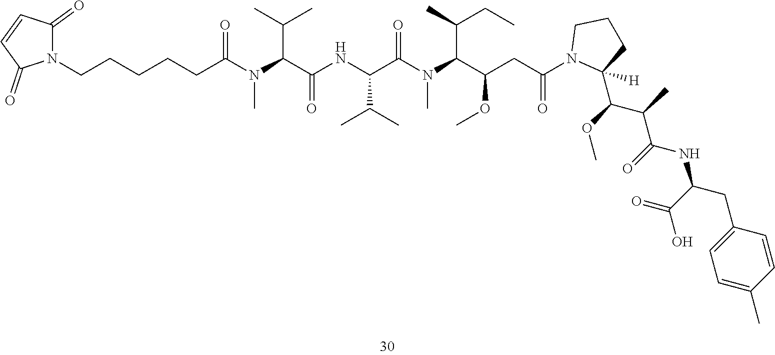 [SMILES: CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)pyrrolidin-2-yl)-3-methoxy-2- |
| | methylpropanamido)-3-(p-tolyl)propanoic acid |
| 47 | 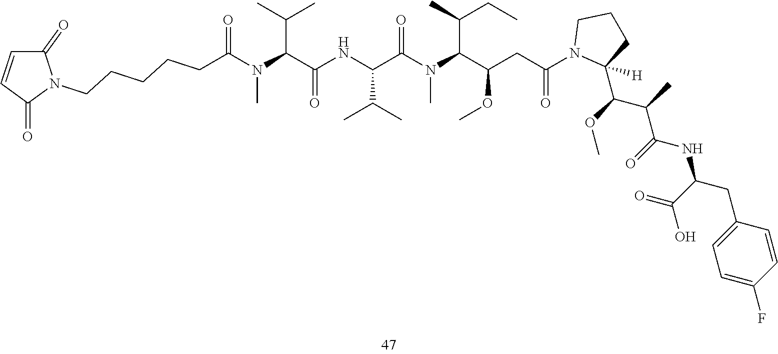 [SMILES: CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@H](C(C)C)N(C)C(=O)CCCCCN1C(=O)C=CC1=O)C(C)C] |
| | (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3- |
| | methylbutanamido)-N,3-dimethylbutanamido)-3-methoxy-5-methylheptanoyl)pyrrolidin-2-yl)-3-methoxy-2- |
| | methylpropanamido)-3-(4-fluorophenyl)propanoic acid |

Another aspect of this invention is directed to a compound of formula (PC-L₂):

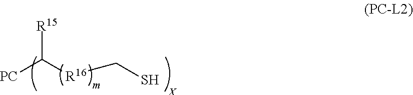

wherein
PC is a ligand, preferably an antibody, more preferably selected from the group consisting of Pertuzumab, Nimotuzumab and Trastuzumab;
R¹⁵ is selected from the group consisting of hydrogen, halogen, hydroxyl, cyano, alkyl, alkoxy and cycloalkyl;
R¹⁶ is selected from the group consisting of alkyl, cycloalkyl, alkoxy and heterocyclyl;
m is 0-5, preferably 1-3;
X is 0-5, preferably 1-3; and X is a positive real number, including decimals and integers.

In another preferred embodiment of the present invention, a compound of formula (PC-L₂) is selected the group consisting of:

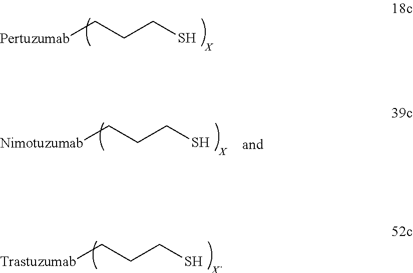

The present invention further relates to a process of preparing a compound of formula (PC-L₂) comprising the steps of:

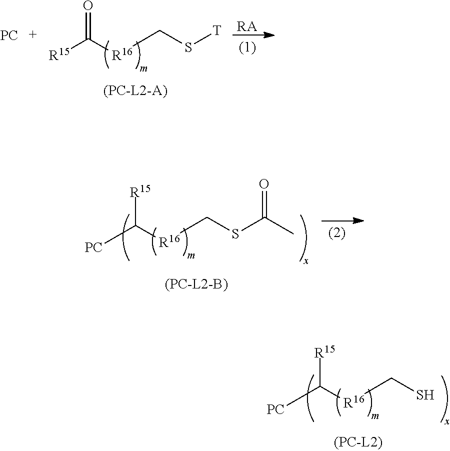

1) PC and a compound of formula (PC-L2-A) are reacted under a reducing agent RA to give a compound of formula (PC-L2-B); wherein RA is preferably sodium cyanoborohydride or sodium triacetoxyborohydride, more preferably sodium cyanoborohydride;
2) a compound of formula (PC-L2-B) is added with a deprotecting agent to remove the protecting group T of the thiol group to give a compound of formula (PC-L2),
wherein:
T is selected from the group consisting of H, t-butyl, acetyl, n-propionyl, isopropionyl, triphenylmethyl, methoxymethyl and 2-(trimethylsilyl) ethoxymethyl, preferably H or acetyl;
the compound of formula (PC-L2-A) is preferably S-(3-carbonylpropyl) thioacetate; PC, R¹⁵, R¹⁶, m and x are as defined in formula (PC-L2).

The present invention further relates to a process of preparing a compound of formula (PC-L′-D) comprising the steps of:

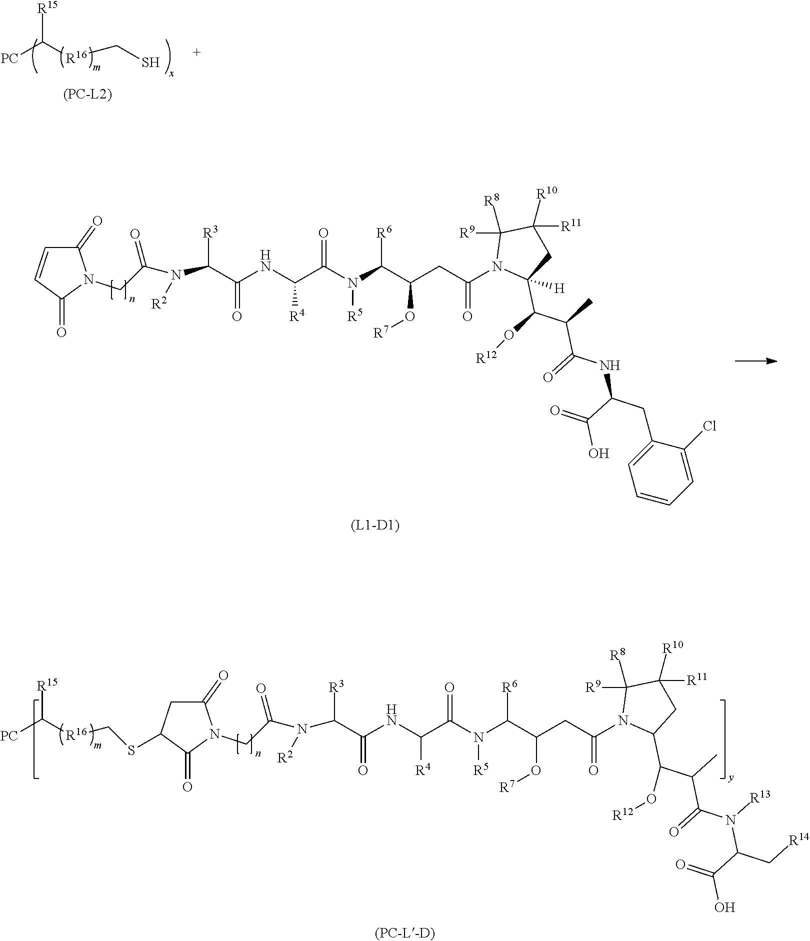

a compound of formula (PC-L2) is reacted with a compound of formula (L1-D1) to give a compound of formula (PC-L′-D);
wherein PC, m, n, y, and R²-R¹⁶ are as defined in formula (PC-L′-D).

The present invention further relates to a compound of formula (D-A a):

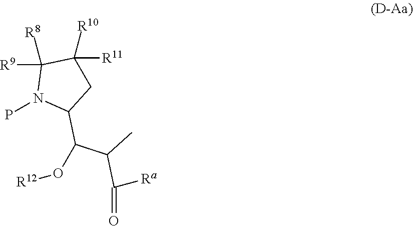

or a tautomer, mesomer, racemate, enantiomer, diastereomer, or mixture thereof, or a pharmaceutically acceptable salt thereof, which can be used as an intermediate for the synthesis of typical intermediate compounds of the ligand drug conjugate ADC of the present invention,
wherein:
any two of R⁸-R¹¹ are attached to form a cycloalkyl, the other two are each optionally selected from the group consisting of hydrogen, alkyl and cycloalkyl;
R¹² is selected from the group consisting of hydrogen, alkyl and halogen;
P is a hydrogen atom or a protecting group, and the protecting group is preferably Boc, Bn, or Cbz, most preferably Boc; and
R^{a} is selected from the group consisting of hydroxy, amino, alkoxy, cycloalkoxy and alkylamino.

The present invention further relates to a compound of formula (D-Aa), which is a compound of formula (D-A):

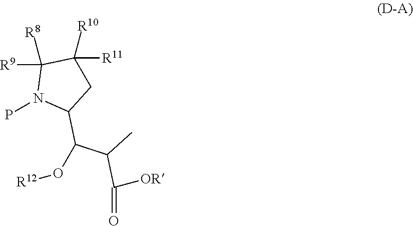

or a tautomer, mesomer, racemate, enantiomer, diastereomer, or mixture thereof, or a pharmaceutically acceptable salt thereof, which can be used as an intermediate for the synthesis of typical prodrug compounds of the ligand drug conjugate ADC of the present invention,
wherein:
any two of R⁸-R¹¹ and P are attached to form a cycloalkyl, the other two are each optionally selected from the group consisting of hydrogen, alkyl and cycloalkyl; and R′ is selected from the group consisting of hydrogen, alkyl and cycloalkyl.

The intermediates (D-Aa) or (D-A) of the typical prodrug compound of the ligand drug conjugate ADC of the present invention include, but are not limited to,

| Example | Structure and name |
|---|---|
| 1e | 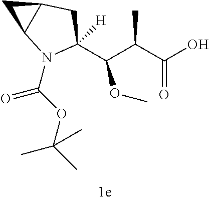 [SMILES: CO[C@H]([C@@H](C)C(=O)O)[C@@H]1C[C@@H]2C[C@@H]2N1C(=O)OC(C)(C)C] |
| | (2R,3R)-3-((1S,3S,5S)-2-(tert-butoxycarbonyl)-2-azabicyclo |
| | [3.1.0]hexan-3-yl)-3-methoxy-2-methylpropanoic acid |
| 3d | 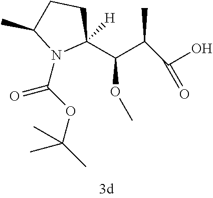 [SMILES: CO[C@H]([C@@H](C)C(=O)O)[C@@H]1CC[C@H](C)N1C(=O)OC(C)(C)C] |
| | (2R,3R)-3-((2S,5S)-1-(tert-butoxycarbonyl)-5- |
| | methylpyrrolidin-2-yl)-3-methoxy-2-methylpropanoic acid |
| 9e | 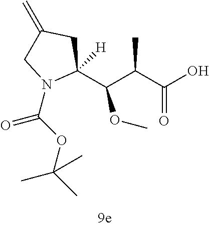 [SMILES: C=C1C[C@@H]([C@H](OC)[C@@H](C)C(=O)O)N(C(=O)OC(C)(C)C)C1] |
| | (2R,3R)-3-((S)-1-(tert-butoxycarbonyl)-4- |
| | methylenepyrrolidin-2-yl)-3-methoxy-2-methylpropanoic acid |
| 11e | 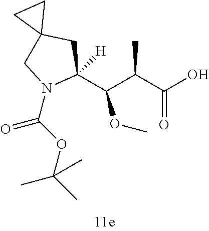 [SMILES: CO[C@H]([C@@H](C)C(=O)O)[C@@H]1CC2(CC2)CN1C(=O)OC(C)(C)C] |
| | (2R,3R)-3-((S)-5-(tert-butoxycarbonyl)-5-azaspiro |
| | [2.4]heptan-6-yl)-3-methoxy-2-methylpropanoic acid |
| 44e | 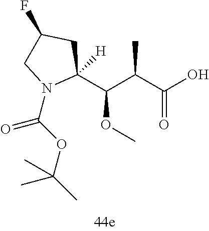 [SMILES: CO[C@H]([C@@H](C)C(=O)O)[C@@H]1C[C@H](F)CN1C(=O)OC(C)(C)C] |
| | (2R,3R)-3-((2S,4S)-1-(tert-butoxycarbonyl)-4- |
| | fluoropyrrolidin-2-yl)-3-methoxy-2-methylpropanoic acid |

The present invention further relates to a pharmaceutical composition comprising a therapeutically effective amount of the ligand-cytotoxic drug conjugate of formula (PC-L-Dr), formula (PC-L′-D), or other ligand-cytotoxic drug conjugates described above, or a compound of formula (Dr), formula (L1-Dr), formula (D), formula (L1-D), or other compounds described above, or a pharmaceutically acceptable salt or solvate thereof, and pharmaceutically acceptable carriers, diluents or excipients.

The present invention further relates to use of the ligand-cytotoxic drug conjugate of formula (PC-L-Dr), formula (PC-L′-D), or other ligand-cytotoxic drug conjugates described above, or a compound of formula (Dr), formula (L1-Dr), formula (D), formula (L1-D), or other compounds described above, or a pharmaceutically acceptable salt or solvate thereof, or a pharmaceutical composition comprising the same, in the preparation of a medicament for the treatment of cancer in a mammal, wherein the cancer is associated with HER2, HER3 or EGFR expression.

The present invention further relates to a method for treating cancer in a mammal comprising administering to the mammal a therapeutically effective amount of a ligand-cytotoxic drug conjugate of formula (PC-L-Dr), formula (PC-L′-D), or other ligand-cytotoxic drug conjugates described above, or a pharmaceutically acceptable salt or solvate thereof, or a compound of formula (Dr), formula (L1-Dr), formula (D), formula (L1-D), or other compounds described above, or a pharmaceutically acceptable salt or solvate thereof, or a pharmaceutical composition comprising the same; wherein the cancer is associated with HER2, HER3, or EGFR expression.

The present invention further relates to use of a ligand-cytotoxic drug conjugate of formula (PC-L-Dr), formula (PC-L′-D), or other ligand-cytotoxic drug conjugates described above, or a compound of formula (Dr), formula (L1-Dr), formula (D), or formula (L1-D), or other compounds described above, or a pharmaceutically acceptable salt or solvate thereof, or a pharmaceutical composition comprising the same, in the preparation of a medicament for the treatment of cancer in a mammal, wherein the mammal is a human, and the cancer is selected from the group consisting of breast cancer, ovarian cancer, stomach cancer, endometrial cancer, salivary gland cancer, lung cancer, colon cancer, renal cancer, colorectal cancer, thyroid cancer, pancreatic cancer, prostate cancer, bladder cancer, acute lymphocytic leukemia, acute myeloid leukemia, acute promyelocytic leukemia, chronic myelogenous leukemia, chronic lymphocytic leukemia, Hodgkin's lymphoma, non-Hodgkin's lymphoma and relapsed anaplastic large cell lymphoma, preferably breast cancer, Hodgkin's lymphoma or relapsed anaplastic large cell lymphoma; more preferably breast cancer associated with HER2 expression.

The present invention further relates to a method for treating cancer in a mammal, the method comprising administering to the mammal a therapeutically effective amount of a ligand-cytotoxic drug conjugate of formula (PC-L-Dr), formula (PC-L′-D), or other ligand-cytotoxic drug conjugates described above, or a compound of formula (Dr), formula (L1-Dr), formula (D), formula (L1-D), or other compounds described above, or a pharmaceutically acceptable salt or solvate thereof, or a pharmaceutical composition comprising the same; wherein the mammal is a human, and the cancer is selected from the group consisting of breast cancer, ovarian cancer, stomach cancer, endometrial cancer, salivary gland cancer, lung cancer, colon cancer, renal cancer, colorectal cancer, thyroid cancer, pancreatic cancer, prostate cancer, bladder cancer, acute lymphocytic leukemia, acute myeloid leukemia, acute promyelocytic leukemia, chronic myelogenous leukemia, chronic lymphocytic leukemia, Hodgkin's lymphoma, non-Hodgkin's lymphoma and relapsed anaplastic large cell lymphoma, preferably breast cancer, Hodgkin's lymphoma or relapsed anaplastic large cell lymphoma; more preferably HER2 over-expressing breast cancer of 2+ level or higher level, most preferably breast cancer associated with HER2 expression.

In the present invention, the free mercapto groups of the unit L link to the amino groups of the N-terminus and/or lysine residues of the antibody, thereby avoiding the need to reduce the antibody hinge region, and reducing the impact on the antibody structure. In addition, the introduced carbon-nitrogen bond structure is stable, and thus difficult to break down in body circulation. The drug loading can be distributed in the range of 0 to 5 by further controlling the reaction conditions.

### DETAILED DESCRIPTION OF THE INVENTION

Unless otherwise defined, all of the technical and scientific terms used herein are in accordance with the usual understanding of one of ordinary skill in the art to which this invention pertains. Although the present invention can be practiced or tested using any of the methods and materials similar or equivalent to those described herein, preferred methods and materials are described herein. The following terms are used to describe and protect the present invention in accordance with the following definitions.

When a trade name is used in the present invention, the applicant is intended to include a preparation of the product of the trade name, a generic drug and an active drug part of the product.

Unless otherwise stated, the terms used in the specification and claims have the meanings described below.

“Alkyl” refers to a saturated aliphatic hydrocarbon group including C₁-C₂₀ straight chain and branched chain groups. Preferably, an alkyl group is an alkyl having 1 to 12 carbon atoms, more preferably 1 to 10 carbon atoms, and most preferably an alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and isomers of branched chains thereof. More preferably, an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, and preferably the substituent group(s) is one or more groups independently selected from the group consisting of alkyl, alkenyl, alkynyl, alkyloxyl, alkylsulfo, alkylamino, halogen, thiol, hydroxy, nitro, cyano, cycloalkyl, heterocyclyl, aryl, heteroaryl, cycloalkoxy, heterocyclic alkoxy, cycloalkylthio, heterocyclic alkylthio, and oxo.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms. Unlimited examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl, and the like. Polycyclic cycloalkyl includes a cycloalkyl having a spiro ring, fused ring or bridged ring.

“Heterocyclyl” refers to a 3 to 20 membered saturated and/or partially unsaturated monocyclic or polycyclic hydrocarbon group having one or more heteroatoms selected from the group consisting of N, O, and S(O)ₘ (wherein m is an integer selected from 0 to 2) as ring atoms, but excluding —O—O—, —O—S— or —S—S— in the ring, and the remaining ring atoms being carbon atoms. Preferably, heterocyclyl has 3 to 12 atoms with 1 to 4 heteroatoms, more preferably 3 to 10 atoms. Unlimited examples of monocyclic heterocyclyl include, but are not limited to, pyrrolidinyl, piperidyl, piperazinyl, morpholinyl, thiomorpholinyl, homopiperazinyl and the like. Polycyclic heterocyclyl includes a heterocyclyl having a spiro ring, fused ring or bridged ring.

Said heterocyclyl can be fused to aryl, heteroaryl or cycloalkyl, wherein the ring bound to the parent structure is heterocyclyl. Representative examples include, but are not limited to:

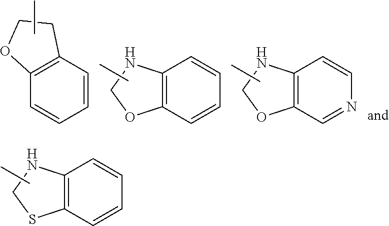

and the like.

The heterocyclyl can be optionally substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocylic alkoxy, cycloalkylthio, heterocylylthio, and oxo.

“Aryl” refers to a 6 to 14 membered full-carbon monocyclic ring or polycyclic fused ring (i.e. each ring in the system shares an adjacent pair of carbon atoms with another ring in the system) group having a completely conjugated pi-electron system; preferably 6 to 10 membered aryl, more preferably phenyl and naphthyl, and most preferably phenyl. The aryl can be fused to heteroaryl, heterocyclyl or cycloalkyl, wherein the ring bound to the parent structure is aryl. Representative examples include, but are not limited to:

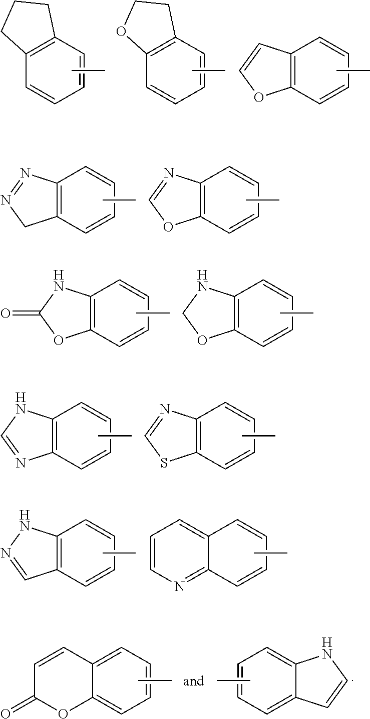

The aryl can be optionally substituted or unsubstituted. When substituted, the substituent group(s) is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocylic alkoxy, cycloalkylthio, and heterocyclylthio.

“Heteroaryl” refers to an aryl system having 1 to 4 heteroatoms selected from the group consisting of O, S and N, and having 5 to 14 ring atoms. Preferably, a heteroaryl is 5- to 10-membered, more preferably 5- or 6-membered, for example furyl, thienyl, pyridyl, pyrrolyl, N-alkyl pyrrolyl, pyrimidinyl, pyrazinyl, imidazolyl, tetrazolyl, etc. The heteroaryl can be fused with the ring of an aryl, heterocyclyl or cycloalkyl, wherein the ring bound to the parent structure is heteroaryl. Representative examples include, but are not limited to:

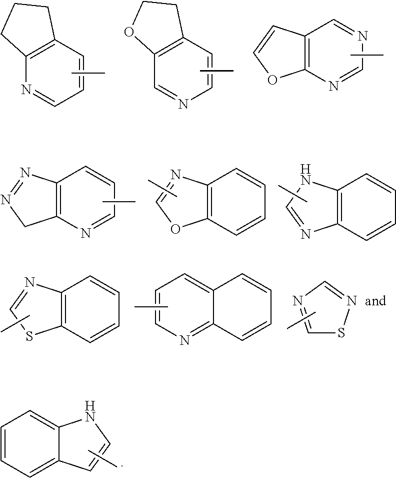

The heteroaryl group can be substituted or unsubstituted. When substituted, the substituent group(s) is preferably one or more groups independently selected from the group consisting of alkyl, alkenyl, alkynyl, alkoxy, alkylsulfo, alkylamino, halogen, thiol, hydroxy, nitro, cyano, cycloalkyl, heterocyclyl, aryl, heteroaryl, cycloalkoxy, heterocyclic alkoxy, cycloalkylthio, and heterocyclic alkylthio.

“Alkoxy” refers to an —O-(alkyl) or an —O-(unsubstituted cycloalkyl) group, wherein the alkyl or cycloalkyl is as defined above. Nonlimiting examples include methoxy, ethoxy, propoxy, butoxy, cyclopropyloxy, cyclobutyloxy, cyclopentyloxy, cyclohexyloxy, and the like. The alkoxy can be optionally substituted or unsubstituted. When substituted, the substituent is preferably one or more groups independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocylic alkoxy, cycloalkylthio, and heterocyclylthio.

“Alkylamino” refers to —N-(alkyl) and —N-(unsubstituted cycloalkyl), wherein the alkyl or cycloalkyl is as defined above. Nonlimiting examples of alkylamino groups include methylamino, ethylamino, propylamino, butylamino, cyclopropylamino, cyclobutylamino, cyclopentylamino, and cyclohexylamino. The alkylamino group can be optionally substituted or unsubstituted, and when substituted, the substituent is preferably one or more of the following groups independently selected from the group consisting of alkyl, alkenyl, alkynyl, alkoxy, alkylthio, alkylamino, halogen, mercapto, hydroxy, nitro, cyano, cycloalkyl, heterocycloalkyl, aryl, heteroaryl, cycloalkoxy, heterocycloalkoxy, cycloalkylthio, and heterocycloalkylthio.

The term “bond” refers to a covalent bond represented by “—”.

“Hydroxy” refers to an —OH group.

“Halogen” refers to fluorine, chlorine, bromine or iodine.

“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and such description includes the situation in which the event or circumstance may or may not occur. For example, “the heterocyclic group optionally substituted with an alkyl” means that an alkyl group can be, but need not be, present, and such description includes the situation of the heterocyclic group being substituted with an alkyl and the heterocyclic group being not substituted with an alkyl.

“Substituted” refers to one or more hydrogen atoms in a group, preferably up to 5, more preferably 1 to 3 hydrogen atoms, independently substituted with a corresponding numbers of substituents. It goes without saying that the substituents only exist in their possible chemical position. The person skilled in the art is able to determine whether the substitution is possible or impossible by experiments or theory without paying excessive efforts. For example, amino or hydroxy having a free hydrogen bound to a carbon atom having an unsaturated bond (such as olefinic) may be unstable.

A “pharmaceutical composition” refers to a mixture of one or more of the compounds according to the present invention or physiologically/pharmaceutically acceptable salts or prodrugs thereof and other chemical components, such as physiologically/pharmaceutically acceptable carriers and excipients. The purpose of a pharmaceutical composition is to facilitate administration of a compound to an organism and the absorption of the active ingredient, thus displaying biological activity.

“Pharmaceutically acceptable salts” refers to salts of ligand-cytotoxic drug conjugates of the invention that are safe and effective in mammals and have the desired biological activity. The antibody-drug conjugated compound of the present invention contains at least one amino group and thus can form a salt with an acid. Nonlimiting examples of pharmaceutically acceptable salts include hydrochloride, hydrobromide, hydroiodide, sulfate, bisulfate, citrate, acetate, succinate, ascorbate, oxalate, nitrate, sorbate, hydrogen phosphate, dihydrogen phosphate, salicylate, hydrogen citrate, tartrate, maleate, fumarate, formate, benzoate, mesylate, ethanesulfonate, benzenesulfonate, and p-toluenesulfonate.

“Solvate” refers to a pharmaceutically acceptable solvate resulting from coupling of a ligand-drug compound of the present invention with one or more solvent molecules. Unlimited examples of solvent molecules include water, ethanol, acetonitrile, isopropanol, DMSO, and ethyl acetate.

“Ligand” refers to a macromolecule compound capable of recognizing and binding to an antigen or receptor associated with a target cell. The role of the ligand is to deliver the drug to the target cell population that binds to the ligand. Such ligands include, but are not limited to, protein hormones, lectins, growth factors, antibodies, or other molecules that bind to cells. In an embodiment of the present invention, the ligand is expressed as PC, preferably an antibody, and the ligand can form a bond with the linker via a heteroatom on the ligand.

“Ligand drug conjugate” refers to a ligand linked to a biologically active cytotoxin by a chemically stable linker compound. In an embodiment of the present invention, the ligand is preferably an antibody, and the “ligand drug conjugate” is preferably an antibody drug conjugate (ADC), which refers to the connection of a monoclonal antibody or antibody fragment to a biologically active cytotoxin by a chemically stable linker compound.

“Antigen” or “receptor” can be identified by a ligand and bind to a target cell. The preferred ligands in the present invention are those cell surface antigens or receptors expressed on target cells and/or tissues of proliferative diseases, such as cancer. Nonlimiting examples of cell surface receptors include HER2, HER3, HER4, CD20, CD22, CD30, CD33, CD44, Lewis Y, CD56, CD105, VEGFR and GPNMB; and most preferably is selected from cell surface receptors of HER2 or EGFR. A specific preferred nonlimiting example is Trastuzumab, which specifically binds to the HER2 target; or Pertuzumab, which specifically binds to the HER2 target; or Nimotuzumab, which specifically binds to the EGFR target.

“Antibody” refers to any form of an antibody that exhibits the desired biological activity. Thus, it is used in its broadest sense, in particular, including, but not limited to full length antibodies, and antibody binding fragments or derivatives. Sources of antibodies include, but are not limited to, monoclonal antibodies, polyclonal antibodies, and genetically engineered antibodies (e.g., bispecific antibodies).

“Full length antibody” refers to an immunoglobulin molecule (e.g., IgM) comprising four polypeptide chains, i.e., two heavy chains and two light chains, which are cross-linked by disulfide bonds to form a polymer. Each heavy chain contains a heavy chain variable region (VH) and a heavy chain constant region, and the heavy chain constant region comprises three domains: CH1, CH2 and CH3. Each light chain comprises a light chain variable region (VL) and a light chain constant region, and the light chain constant region comprises one domain (CL1). The VH and VL regions can be further divided into hypervariable regions, the term of which is complementarity determining regions (CDRs), and the more conserved domains interspersed between the complementarity regions, which is called the framework region (FR).

“Antibody binding fragment or derivative” includes any polypeptide or glycoprotein that can specifically bind to an antigen to form a complex, which is naturally occurring or obtained enzymatically, synthetically, or by genetic engineering. It usually comprises at least part of the antigen-binding region or variable region (e.g., one or more CDRs) of the parent antibody and retains at least some of the binding specificity of the parent antibody. “Antibody binding fragments or derivatives” can be derived from antibodies, such as derived from the transformation of the whole length of the antibody by appropriate standard techniques including proteolytic or recombinant genetically engineered techniques (including manipulation and expression of DNA expressing antibody variable regions and partially constant regions). “Antibody binding fragment or derivative” includes, but I s not limited to: (i) Fab fragments; (ii) F(ab′)₂ fragments; (iii) Fd fragments; (iv) Fv fragments; (v) single chain Fv (scFv); (vi) dAb fragments; and (vii) the minimum recognition unit (e.g., an isolated complementarity determining region (CDR)) that mimics the amino acid residue of the hypervariable region of the antibody. Other engineered molecules, such as bivalent antibodies, trivalent antibodies, tetravalent antibodies and micro-antibodies, are also within the scope of “antibody-binding fragments or derivatives”.

“Fab fragment” consists of complete VH and CH1 functional regions of light and heavy chain. The heavy chain of the Fab molecule cannot form a disulfide bond with another heavy chain molecule.

“Fc” region contains two heavy chain fragments containing the CH1 and CH2 domains of the antibody. Two heavy chain fragments are held together by two or more disulfide bonds and hydrophobic effects through the CH3 domain.

“Fab′ fragment” contains a light chain and the VH and CH1 functional regions of the heavy chain, and also contains regions between the CH1 and CH2 domains, so that interchain disulfide bonds can be formed between the two heavy chains of the two Fab′ fragments to form F(ab′)2 molecules.

“F(ab′)2 fragment” contains two light chains and two heavy chains containing a partial constant region between the CH1 and CH2 domains, so that interchain disulfide bonds can be formed between the two heavy chains. Thus, a F(ab′)2 fragment is formed with two Fab′ fragments through the interchain disulfide bonds between the two heavy chains.

“Fv fragment” includes the variable region VH functional region of the light chain and/or the heavy chain.

“Fc region” corresponds to the CH2 and CH3 functional regions of IgG and has no antigen-binding activity, but is the interaction site between the antibody molecule and the effector molecule and cell.

“Hinge region” is used to link the Fab and Fc fragments of the antibody. In the present invention, it is used to link the bispecific fusion proteins to the Fc fragments.

The antibody of the present invention is preferably a specific antibody against a cell surface antigen on a target cell. Nonlimiting examples are as follows. Trastuzumab, a humanized anti-HER2 antibody for the treatment of breast cancer, is suitable for the treatment of metastatic breast cancer with HER2 overexpression. Pertuzumab, also known as 2C4, trade name of Perjeta®, is a recombinant humanized monoclonal antibody, and is the first monoclonal antibody known as the “HER dimerization inhibitor” that reduces tumor growth by binding HER2 to block the dimerization of HER2 with other HER receptors. Pertuzumab has been shown to inhibit tumor growth in prostate cancer patients with HER2 overexpression and low expression. Pertuzumab has been approved by the US FDA for the treatment of HER2-positive metastatic breast cancer. Nimotuzumab is a monoclonal antibody that targets the epidermal growth factor receptor (EGFR) and is a humanized monoclonal antibody that can be used to treat malignant tumors. EGFR is overexpressed in a variety of solid tumors, such as head and neck cancer, lung cancer, and colorectal cancer.

“Cytotoxic drug” refers to a chemical molecule that has a strong ability to destroy its normal growth in tumor cells. All principally cytotoxic drugs can kill tumor cells at a sufficiently high concentration, but because of the lack of specificity, it can cause normal cell apoptosis and cause serious side effects while killing tumor cells. In an embodiment of the present invention, the cytotoxic drug is expressed as D/D1.

“Linker” in the present invention is expressed as L. It is a chemical structural fragment or bond which is covalently linked to a ligand at one end and linked to a cytotoxic drug at another end. The structure of L in the present invention is as follows:

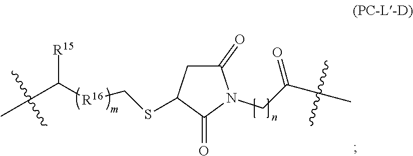

wherein R15, R16, m, and n are defined as formula (PC-L′-D).

“Drug loading” refers to the average number of cytotoxic drugs loaded on each ligand in formula (I), and can also be expressed as the ratio of the number of drug to the number of antibody. The drug loading can range from 1 to 8 cytotoxic drugs (D) per ligand (Pc). In an embodiment of the present invention, the drug loading is expressed as y, and the number of drug products per ADC molecule after coupling reaction can be determined by conventional methods such as UV/visible spectroscopy, mass spectrometry, ELISA test and HPLC characterization.

In the present invention, y can be limited by the number of connection sites. In an embodiment of the present invention, the cytotoxic drug is coupled to the N-terminal amino and/or the ε-amino of lysine residues of the ligand via a linker. Typically the number of drug molecules conjugated to the antibody in a coupling reaction will be less than the theoretical maximum.

The following nonlimiting methods can be used to control the loading of the ligand-cytotoxic drug conjugates:

(1) to control the molar ratio of the linking reagent to the monoclonal antibody,
(2) to control the reaction time and temperature,
(3) to select a different reaction reagent.

The preparation of conventional pharmaceutical compositions can be found in the Chinese Pharmacopoeia.

“Carrier” used in the medicament of the present invention refers to a system that can change the way a drug enters the human body and is distributed, the drug release rate, and delivery of the drug to the targeted organ. Drug carrier release and targeting systems can reduce drug degradation and loss, reduce side effects and improve bioavailability. For example, the polymer surfactants which can be used as carriers can be self-assembled to form various forms of aggregates due to their unique amphiphilic structure. Preferred examples include micelles, microemulsions, gels, liquid crystals, vesicles, and the like. These aggregates have the ability to encapsulate drug molecules, while having good permeability to the membrane, and can be used as an excellent drug carrier.

“Excipient” is an adjunct to a pharmaceutical formulation other than a main drug and can also be referred to as an adjuvant, such as adhesives, fillers, disintegrants or lubricants of tablets; ointments of semi-solid preparations; matrix parts of the cream; preservatives, antioxidants, flavoring agents, fragrances, co-solvents, emulsifiers, solubilizers, osmotic pressure regulators or colorants of liquid preparations, and the like.

“Diluent”, also known as a filler, is primarily intended to increase the weight and volume of the tablet. The addition of the diluent not only ensures a certain volume size, but also reduces the dose deviation of the main components, and improves the compression profile of the drug. When the tablet contains an oily component, the absorbent is added to the oily substance to keep the “dry” state to facilitate tablet formation, such as starch, lactose, inorganic salts of calcium, microcrystalline cellulose and the like.

The pharmaceutical composition can be in the form of a sterile injectable aqueous solution. The acceptable medium and solvents that can be employed are water, Ringer's solution and isotonic sodium chloride solution. The sterile injectable preparation can also be a sterile injectable oil-in-water microemulsion in which the active ingredient is dissolved in the oil phase. For example, the active ingredient can be first dissolved in a mixture of soybean oil and lecithin, and the oil solution can then be introduced into a mixture of water and glycerol to form a microemulsion. The injectable solution or microemulsion can be infused into an individual's bloodstream by local mass injection. Alternatively, it may be advantageous to administer the injectable solution or microemulsion in such a way of maintaining a constant circulating concentration of the present compound. In order to maintain such a constant concentration, a continuous intravenous delivery device can be utilized. An example of such device is a Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent, for example, a solution prepared in 1,3-butanediol. Moreover, sterile fixed oils can be used as a solvent or suspending medium. For this purpose, any blending fixed oils including synthetic mono- or di-glyceride can be employed. Moreover, fatty acids, such as oleic acid, can also be employed in the preparation of an injection.

“Reducing agent” is a substance that loses electrons in a redox reaction or has an electronic deviation. The reducing agent, in a broad sense, is an antioxidant, which has a reducing property and can be oxidized, and its product is called an oxidation product. In an embodiment of the present invention, the reducing agent is expressed as RA, and nonlimiting examples include H₂, C, CO, Fe, Zn, alkali metal (commonly used with Li, Na, K), other active metals (such as Mg, Al, Ca, La, etc.), SnCl₂, oxalic acid, KBH₄, NaBH₄, NaCNBH₃), (CH3COO)₃BHNa, LiAlH₄, hypophosphorous acid, sodium hypophosphite, and Na₂S₂O₃. The preferred reducing agent of the present invention is NaCNBH₃ or (CH3COO)₃BHNa.

“Mercapto protecting group” refers to a group which protects the mercapto group and can be removed at the end of the reaction, so that the reaction only occurs at an intended position while the mercapto group is not involved when a chemical molecule containing both mercapto groups and other groups is involved. In an embodiment of the present invention, the mercapto protecting group is expressed as T, and its nonlimiting examples include -tert-butyl, -acetyl, n-propionyl, -isopropanoyl, -triphenylmethyl, -methoxymethyl, and -2-(trimethylsilyl)ethoxymethyl. The preferred mercapto protecting group of the present invention is acetyl.

### Synthesis Method of the Present Invention

In order to accomplish the purpose of the synthesis of the present invention, the following synthetic scheme is adopted.

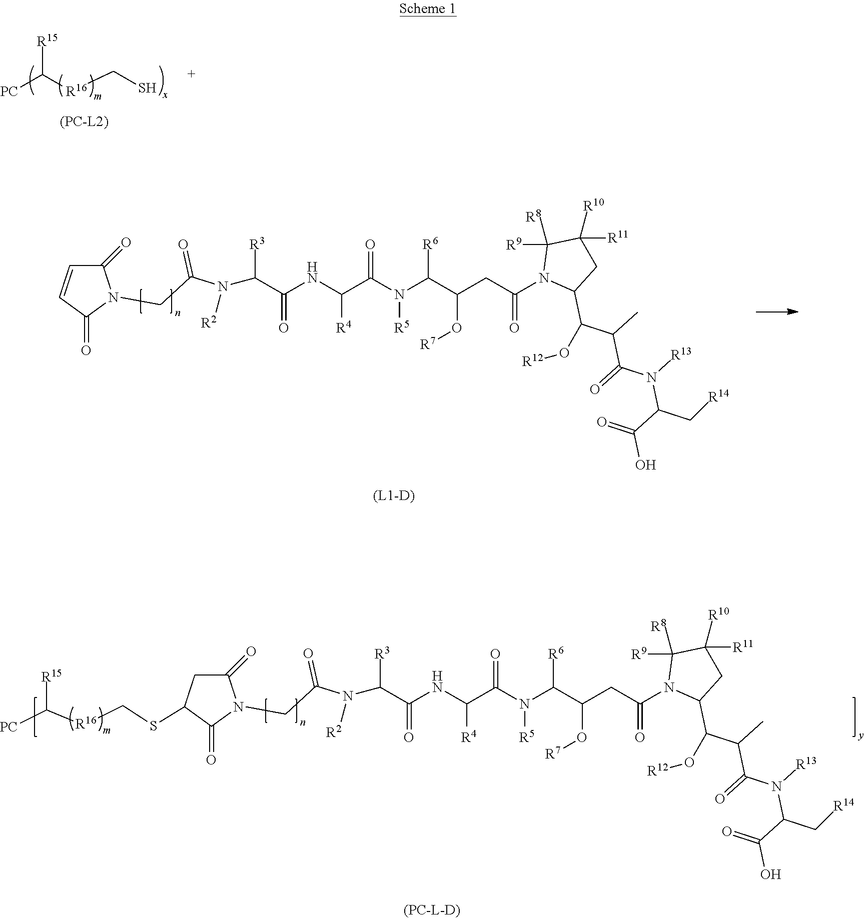

The process for preparing a compound of formula (PC-L-DR) according to the present invention comprises:

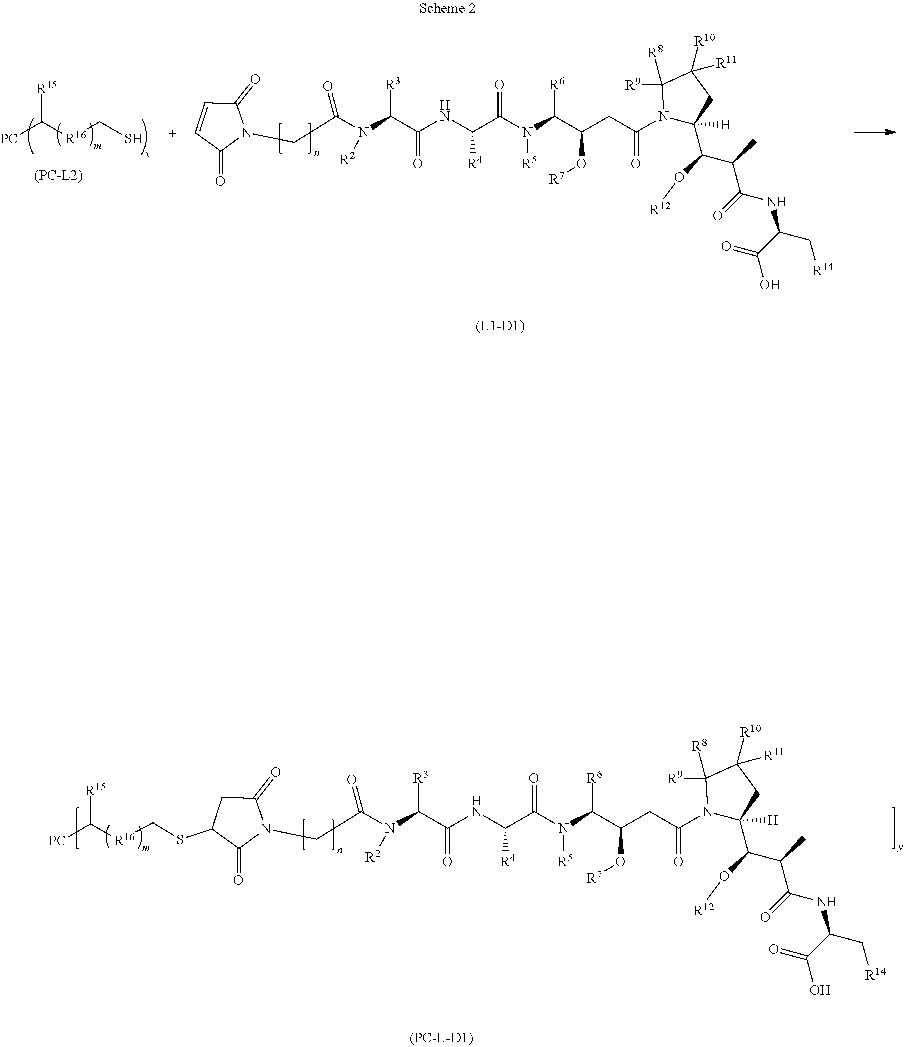

The process for preparing a compound of formula (PC-L-DR1) according to the present invention comprises:

A compound of formula (PC-L2) is reacted with a compound of formula (L1-D1) in an acetonitrile solution. A compound of formula (PC-L-DR) is obtained after desalting purification by the Sephadex G25 gel column;

wherein, PC, m, n, y, and R²-R¹⁶ are as defined in formula (PC-L-DR).

### DETAILED DESCRIPTION OF THE INVENTION

The present invention will be further described with the following examples, but the examples should not be considered as limiting the scope of the invention.

Conditions that are not specified in the examples are the common conditions in the art or the recommended conditions of the raw materials by the product manufacturer. For the reagents which are not indicated, it is the commercially available conventional reagent.

### EXAMPLES

The structure of the compound was identified by nuclear magnetic resonsance (NMR) and/or mass spectrometry (MS). NMR chemical shifts (8) are given in 10⁻⁶ (ppm). NMR was determined by Bruker AVANCE-400. The solvents were deuterated-dimethyl sulfoxide (DMSO-d₆), deuterated-chloroform (CDCl₃) and deuterated-methanol (CD₃OD) with tetramethylsilane (TMS) as an internal standard.

MS was determined by a FINNIGAN LCQAd (ESI) mass spectrometer (manufacturer: Thermo, type: Finnigan LCQ advantage MAX).

High performance liquid chromatography (HPLC) was determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column).

The average inhibition rate of kinase and IC₅₀ values were determined by a NovoStar ELISA (BMG Co., Germany).

Yantai Huanghai HSGF254 or Qingdao GF254 silica gel plate was used for thin-layer silica gel chromatography (TLC). The dimension of the silica gel plate used in TLC was 0.15 mm to 0.2 mm, and the dimension of the silica gel plate used in product purification was 0.4 mm to 0.5 mm.

Yantai Huanghai 200 to 300 mesh silica gel was used as the carrier for column chromatography.

The known raw materials of the present invention were prepared by conventional synthesis methods known in the art, or purchased from ABCR GmbH & Co. KG, Acros Organics, Aldrich Chemical Company, Accela ChemBio Inc., or Dari chemical Company, etc.

Unless otherwise stated, all of the reactions were carried out under nitrogen atmosphere or argon atmosphere.

The term “nitrogen atmosphere” or “argon atmosphere” means that a reaction flask was equipped with a 1 L nitrogen or argon balloon.

Unless otherwise stated, the solution used in the reactions refers to an aqueous solution.

Unless otherwise stated, the reaction temperature in the reactions refers to room temperature. Room temperature is the optimum reaction temperature which is in the range of 20° C. to 30° C.

Preparation of PBS buffer with pH=6.5 in the reaction: KH₂PO₄ 8.5 g, K₂HPO₄.3H₂O 8.56 g, NaCl 5.85 g, EDTA 1.5 g were set to 2 L in a bottle, and then subjected to ultrasonication to give the buffer.

Preparation of acetic acid/sodium acetate buffer with pH=4.5 in the reaction: 9 g of anhydrous sodium acetate were placed in a bottle, added with purified water, and set to 2 L, then sodium acetate 4.9 mL was added with stirring to give the buffer.

Preparation of phosphate buffer with pH=7.0 in the reaction: 39 mL of 0.2M NaH₂PO₄ was added to 61 mL of 0.2M Na₂HPO₄ with stirring to give a 0.2M buffer with pH=7.

The reaction process is monitored by thin layer chromatography (TLC), and the elution systems included: A: dichloromethane and methanol, B: n-hexane and ethyl acetate, C: petroleum ether and ethyl acetate, D: acetone. The ratio of the volume of the solvent was adjusted according to the polarity of the compounds.

The elution systems for purification of the compounds by column chromatography and thin layer chromatography included: A: dichloromethane and methanol, B: n-hexane and ethyl acetate, C: dichloromethane and acetone, D: ethyl acetate and dichloromethane, E: ethyl acetate and dichloromethane and n-hexane, F: ethyl acetate and dichloromethane and acetone. The ratio of the volume of the solvent was adjusted according to the polarity of the compounds, and sometimes a little alkaline reagent, such as triethylamine or acidic reagent, was added.

Some of the structures of the compounds of the present invention were determined by Q-TOF LC/MS. For Q-TOF LC/MS, Agilent 6530 Accurate-Mass Quadrupole-Time of Flight Mass Spectrometer and Agilent 1290-Infinity UHPLC (Agilent Poroshell 300SB-C8 5 μm, 2.1×75 mm Column) were used.

Known starting materials of the present invention were synthesized by adopting or using the methods known in the art, and the experimental methods in the following examples for which the specific conditions are not indicated were carried out according to conventional conditions or the conditions recommended by the product manufacturers. The experimental reagents for which the specific sources are not indicated are the conventional reagents generally purchased from market.

### 1. Preparation of Antibodies as Intermediates

The following antibodies were prepared according to conventional methods: for instance, vector construction, HEK293 cell transfection (Life Technologies Cat. No. 11625019), purification and expression.

### Antibody Sequences

(1) Pertuzumab, capable of specifically binding
to target HER2:
Sequence of light chain:
(SEQ ID NO: 1)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Sequence of heavy chain:
(SEQ ID NO: 2)
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(2) Nimotuzumab, capable of specifically binding
to target EGFR:
Sequence of light chain:
(SEQ ID NO: 3)
DIQMTQSPSSLSASVGDRVTITCRSSQNIVHSNGNTYLDWYQQTPGKAPK
LLIYKVSNRFSGVPSRFSGSGSGTDFTFTISSLQPEDIATYYCFQYSHVP
WTFGQGTKLQITRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Sequence of heavy chain:
(SEQ ID NO: 4)
QVQLQQSGAEVKKPGSSVKVSCKASGYTFTNYYIYWVRQAPGQGLEWIGG
INPTSGGSNFNEKFKTRVTITADESSTTAYMELSSLRSEDTAFYFCTRQG
LWFDSDGRGFDFWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK
(3) Trastuzumab, capable of specifically binding
to target HER2:
Sequence of light chain:
(SEQ ID NO: 5)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Sequence of heavy chain:
(SEQ ID NO: 6)
EVQLVESGGGLVQPGGSLRLSCAASGFNIKDTYIHWVRQAPGKGLEWVAR
TYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

2. Preparation of Drug, Drug Linker, and Ligand Drug Conjugate (ADC)

### Example 1

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoic acid

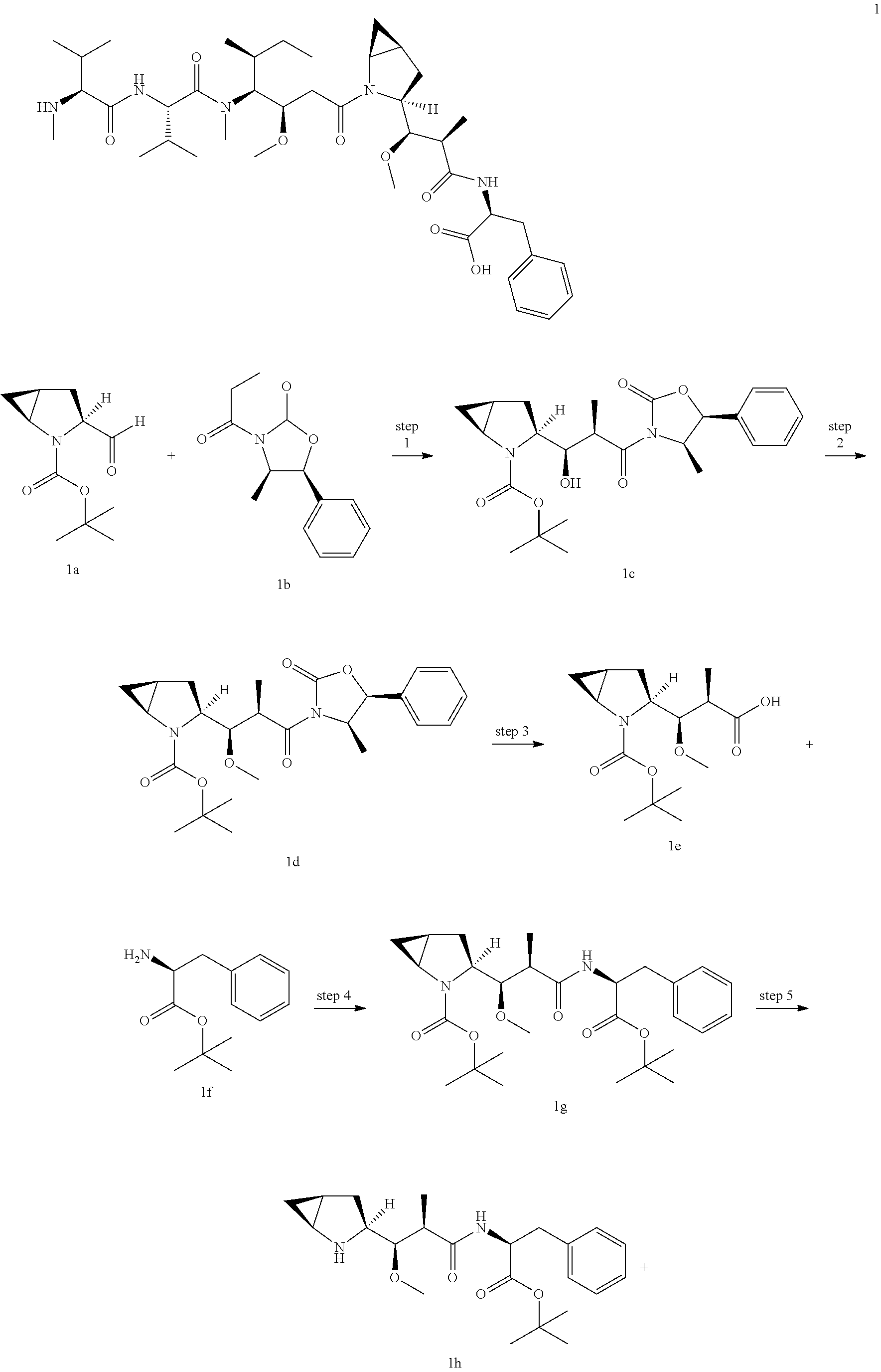

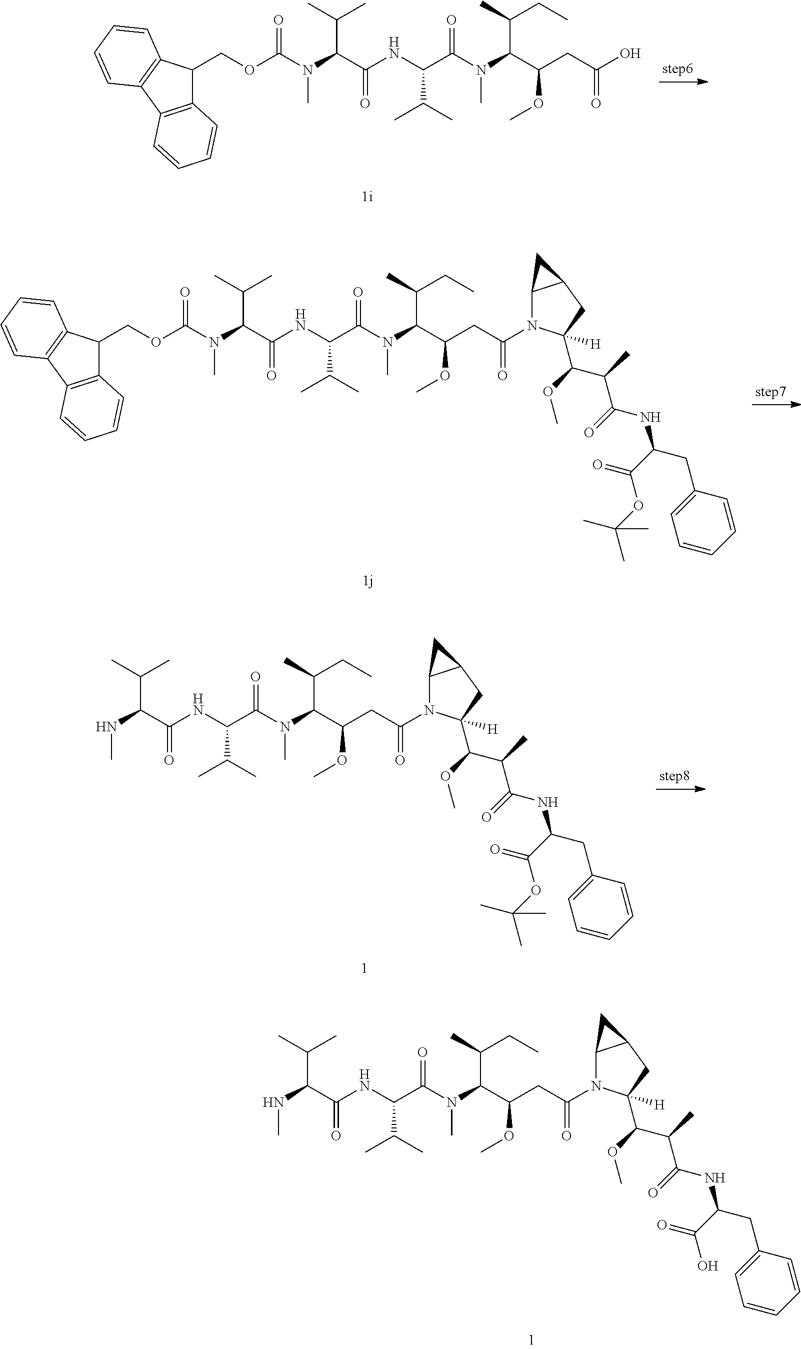

### Step 1

### (1S,3S,5S)-tert-butyl 3-((1R,2R)-1-hydroxy-2-methyl-3-((4R,5S)-4-methyl-2-oxo-5-phenyloxazolidin-3-yl)-3-oxopropyl)-2-azabicyclo[3 0.1.0]hexane-2-carboxylate

(4R,5S)-4-methyl-5-phenyl-3-propionyloxazolidin-2-one 1b (1.96 g, 9.26 mmol, prepared according to the known method of “Journal of the American Chemical Society, 2003, 125(50), 15512-15520”) was dissolved in 25 mL of dichloromethane under an argon atmosphere, and cooled to 0° C. The above reaction solution was added with trimethylamine (1.49 mL, 10.93 mmol) and dibutylboron trifluoromethanesulfonate (9.7 mL, 9.72 mmol) dropwise, and then stirred for 50 min at 0° C. The resulting mixture was cooled to −75° C. in a dry ice acetone bath, then added with a solution (7 mL) of (1S,3S,5S)-tert-butyl 3-formyl-2-azabicyclo[3.1.0]hexane-2-carboxylate 1a (2.16 g, 9.26 mmol, prepared according to the known method in “US20100249190”) in dichloromethane, and stirred for 1.5 hours at −75° C., then for 2 hours at 0° C., and then for 1 hour at room temperature. The reaction mixture was added with 36 mL of a mixture of phosphate buffer (pH=7.0) and methanol (V/V=1:3), then added with 36 mL of a mixed solution of methanol and hydrogen peroxide (30%) (V/V=2:1) at 0° C., and stirred for 1 hour at room temperature. The reaction mixture was concentrated under reduced pressure to remove the organic phase. The residues were added with a little water and extracted with ether (50 mL×3), washed sequentially with 5% sodium bicarbonate solution and 150 mL of saturated sodium chloride solution, dried over anhydrous sodium sulfate, filtered, and the filtrate was concentrated under reduced pressure. The residue was purified by silica gel column chromatography with eluent system B to give the title product of (1S,3S,5S)-tert-butyl 3-((1R,2R)-1-hydroxy-2-methyl-3-((4R,5S)-4-methyl-2-oxo-5-phenyloxazolidin-3-yl)-3-oxopropyl)-2-azabicyclo[3.1.0]hexane-2-carboxylate 1c (2.4 g, white foam solid), yield 58.5%.

MS m/z (ESI): 345.1 [M−100+1]

### Step 2

### (1S,3S,5S)-tert-butyl 3-((1R,2R)-1-methoxy-2-methyl-3-((4R,5S)-4-methyl-2-oxo-5-phenyloxazolidin-3-yl)-3-oxopropyl)-2-azabicyclo[3.1.0]hexane-2-carboxylate

(1S,3S,5S)-tert-butyl3-((1R,2R)-1-hydroxy-2-methyl-3-((4R,5 S)-4-methyl-2-oxo-5-phenyloxazolidin-3-yl)-3-oxopropyl)-2-azabicyclo[3.1.0]hexane-2-carboxylate 1c (1.4 g, 3.15 mmol) was dissolved in 20 mL dichloromethane, and added with 1.4 g crushed molecular sieves. The mixture was added with 1,8-bisdimethylaminonaphthalene (1.75 g, 8.19 mmol) and trimethyloxonium tetrafluoroboron (1.16 g, 7.87 mmol) at 0° C., under argon atmosphere. The reaction mixture was kept dark and stirred at room temperature for 40 hours. After the reaction was completed, the reaction mixture was filtered and the filter cake was washed with methylene chloride. The combined filtrate was washed with saturated ammonium chloride solution (50 mL×4) to remove the excess 1,8-bisdimethylaminonaphthalene and washed with saturated sodium chloride solution (120 mL), dried over anhydrous sodium sulfate, filtered, and the filtrate was concentrated under reduced pressure. The residues were purified by flash column chromatography using eluent system B to give the title product of (1S,3S,5S)-tert-butyl 3-((1R,2R)-1-methoxy-2-methyl-3-((4R,5S)-4-methyl-2-oxo-5-phenyloxazolidin-3-yl)-3-oxopropyl)-2-azabicyclo[3.1.0]hexane-2-carboxylate 1d (400 mg, white solid), yield 27.8%.

MS m/z (ESI): 459.4 [M+1]

### Step 3

### (2R,3R)-3-((1S,3S,5S)-2-(tert-butoxy carbonyl)-2-azabicyclo[3.1.0]hexan-3-yl)-3-methoxy-2-methylpropanoic acid

(1S,3S,5S)-tert-butyl 3-((1R,2R)-1-methoxy-2-methyl-3-((4R,5S)-4-methyl-2-oxo-5-phenyloxazolidin-3-yl)-3-oxopropyl)-2-azabicyclo[3.1.0]hexane-2-carboxylate 1d (400 mg, 0.87 mmol) was dissolved in 24 mL of tetrahydrofuran and cooled to 0° C. under argon atmosphere. 30% hydrogen peroxide (0.34 mL/0.38 g, 3.31 mmol) was added dropwise slowly, and then lithium hydroxide monohydrate (62 mg, 1.48 mmol) was added. The reaction mixture was allowed to react at room temperature for 20 hours. The reaction solution was added with sodium sulfite solid (440 mg, 3.48 mmol), and stirred at room temperature for 1 hour. Then 10 mL of water was added and the organic phase was concentrated under reduced pressure. The residues were extracted with dichloromethane (40 mL×2). The separated aqueous phase was added with 2N hydrochloric acid dropwise in an ice bath until the solution arrived at a pH of 3 to 4. Then the aqueous phase was extracted with ethyl acetate (25 mL×3), and the combined ethyl acetate phase was washed successively with water (50 mL) and saturated sodium chloride solution (50 mL), dried over anhydrous sodium sulfate, filtered and the filtrate was concentrated under reduced pressure to give the title product of (2R,3R)-3-((1S,3S,5S)-2-(tert-butoxycarbonyl)-2-azabicyclo[3.1.0]hexan-3-yl)-3-methoxy-2-methylpropanoic acid 1e (230 mg, colorless liquid), yield 88.0%.

MS m/z (ESI): 200.1 [M-100+1]

### Step 4

### (1S,3S,5S)-tert-butyl 3-((1R,2R)-3-((S)-1-tert-butoxy-1-oxo-3-phenylpropan-2-ylamino)-1-methoxy-2-methyl-3-oxopropyl)-2-azabicyclo[3.1.0]hexane-2-carboxylate

(2R,3R)-3-((1S,3S,5S)-2-(tert-butoxycarbonyl)-2-azabicyclo[3.1.0]hexan-3-yl)-3-methoxy-2-methylpropanoic acid 1e (100 mg, 0.334 mmol) was dissolved in 6 mL of a mixed solvent of dichloromethane and dimethylformamide (V/V=5:1), and added with (S)-tert-butyl 2-amino-3-phenylpropanoate if (73.9 mg, 0.334 mmol, prepared according to the known method in “Tetrahedron: Asymmetry, 2006, 17(4), 603-606”) and then N,N-diisopropylethylamine (0.29 mL, 67 mmol) and 2-(7-azobenzotriazole)-N,N,N′,N′-tetramethyluronium hexafluophosphate (152.3 mg, 0.40 mmol). The reaction mixture was stirred under an argon atmosphere at room temperature for 1 hour, and then added with 10 mL of water under stirring. The dichloromethane phase was washed with saturated sodium chloride solution (10 mL), dried over anhydrous sodium sulfate, filtered, and the filtrate was concentrated under reduced pressure. The residues were purified by silica gel column chromatography using eluent system B to give the title product of (1S,3S,5S)-tert-butyl 3-((1R,2R)-3-((S)-1-tert-butoxy-1-oxo-3-phenylpropan-2-ylamino)-1-methoxy-2-methyl-3-oxopropyl)-2-azabicyclo[3.1.0]hexane-2-carboxylate 1g (140 mg, colorless viscous liquid), yield 83.7%.

MS m/z (ESI): 503.3 [M+1]

### Step 5

### (S)-tert-butyl 2-((2R,3R)-3-((1S,3S,5S)-2-azabicyclo[3.1.0]hexan-3-yl)-3-methoxy-2-methylpropanamido)-3-phenylpropanoate (1S,3S,5S)-tert-butyl

3-((1R,2R)-3-((S)-1-tert-butoxy-1-oxo-3-phenylpropan-2-ylamino)-1-methoxy-2-methyl-3-oxopropyl)-2-azabicyclo[3.1.0]hexane-2-carboxylate 1g (140 mg, 0.28 mmol) was dissolved in 2 mL of dioxane, and then added with a 5.6 M solution of hydrogen chloride in dioxane (0.15 mL, 0.835 mmol). The reaction system was sealed and then stirred for 8 hours at room temperature and placed in a refrigerator for 12 hours at 0° C. After the reaction, 3 mL of dichloromethane, 3 mL of water, and 3 mL of saturated sodium bicarbonate solution were added successively and stirred for 10 minutes. The dichloromethane phase was washed with saturated sodium chloride solution (20 mL×2), dried over anhydrous sodium sulfate, filtered and the filtrate was concentrated under reduced pressure to obtain the title product of (S)-tert-butyl 2-((2R,3R)-3-((1S,3S,5S)-2-azabicyclo[3.1.0]hexan-3-yl)-3-methoxy-2-methylpropanamido)-3-phenylpropanoate 1h (86 mg, yellow solid), yield 76.7%.

MS m/z (ESI): 403.4 [M+1]

### Step 6

### (S)-tert-butyl 2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-phenylpropanoate

(S)-tert-butyl 2-((2R,3R)-3-((1S,3S,5S)-2-azabicyclo[3.1.0]hexan-3-yl)-3-methoxy-2-methylpropanamido)-3-phenylpropanoate 1h (86 mg, 0.213 mmol), (5S,8S,11S,12R)-11-((S)-sec-butyl)-1-(9H-fluoren-9-yl)-5,8-diisopropyl-12-methoxy-4,1 0-dimethyl-3,6,9-trioxo-2-oxa-4,7,10-triazatetradecan-14-oic acid 1i (136 mg, 0.213 mmol, prepared according to the known method “WO 2013072813”) was dissolved in 6 mL of a mixed solvent of dichloromethane and dimethylformamide (V/V=5:1), and then added with N,N-diisopropylethylamine (0.19 mL, 1.065 mmol) and 2-(7-azobenzotriazole)-N,N,N′,N′-tetramethyluronium hexafluophosphatehexafluorophosphate (97.4 mg, 0.256 mmol). The reaction mixture was stirred under an argon atmosphere at room temperature for 1 hour and then added with 20 mL of water. The dichloromethane layer was washed with saturated sodium chloride solution (20 mL), dried over anhydrous sodium sulfate, filtered and the filtrate was concentrated under reduced pressure. The residues were purified by silica gel column chromatography using eluent system B to give the title product (S)-tert-butyl 2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-phenylpropanoate 1j (120 mg, white foam solid), yield 54.9%.

MS m/z (ESI): 1023.1 [M+1]

### Step 7

### (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoate

(S)-tert-butyl 2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-phenylpropanoate 1j (120 mg, 0.117 mmol) was dissolved in 2 mL of dichloromethane and added with 2 mL of diethylamine. The reaction was stirred under an argon atmosphere at room temperature for 2 hours. The reaction solution was concentrated under reduced pressure to give the crude title product of (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoate 1k (124 mg, yellow liquid). The product was used in the next step without further purification.

MS m/z (ESI): 801.5 [M+1].

### Step 8

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoic acid

The crude product of (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoate 1k (90 mg, 0.112 mmol) was dissolved in 1 mL dioxane, and then added with a 5.6 M solution of hydrogen chloride in dioxane (3 mL). The reaction system was sealed and stirred at room temperature for 12 hours. The reaction mixture was concentrated under reduced pressure and the residues were purified by high performance liquid chromatography to obtain the title product of (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoic acid 1 (19 mg, white solid), yield 22.7%.

MS m/z (ESI): 744.6 [M+1]

¹H NMR (400 MHz, CD₃OD): δ 7.34-7.21 (m, 5H), 4.76-4.70 (m, 2H), 4.26-4.19 (m, 1H), 4.14-4.06 (m, 1H), 3.91-3.86 (m, 1H), 3.85-3.77 (m, 1H), 3.75-3.56 (m, 2H), 3.44-3.10 (m, 9H), 2.98-2.83 (m, 1H), 2.71-2.57 (m, 4H), 2.26-1.99 (m, 4H), 1.92-1.77 (m, 1H), 1.75-1.58 (m, 2H), 1.49-1.27 (m, 4H), 1.21-0.95 (m, 18H), 0.93-0.79 (m, 4H), 0.76-0.61 (m, 1H).

### Example 2

### (S)-3-(2-chlorophenyl)-2-((2R,3R)-3-((1S,3S,5S)-2-((3R,4S,5S)-4-((S)—N,3-dimethyl-2-4 S)-3-methyl-2-(methylamino)butanamido)butanamido)-3-methoxy-5-methylheptanoyl)-2-azabicyclo[3 0.1.0]hexan-3-yl)-3-methoxy-2-methylpropanamido)propanoic acid

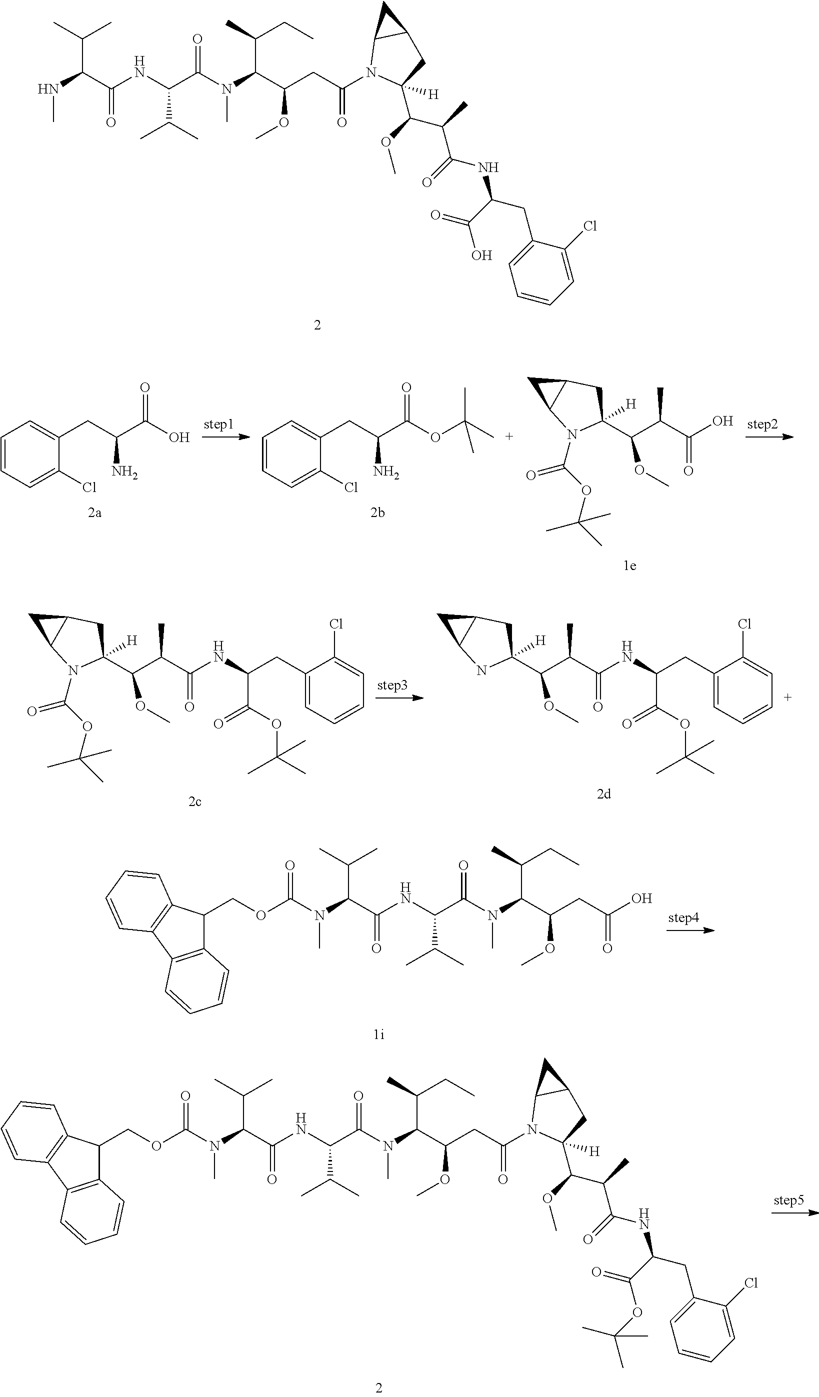

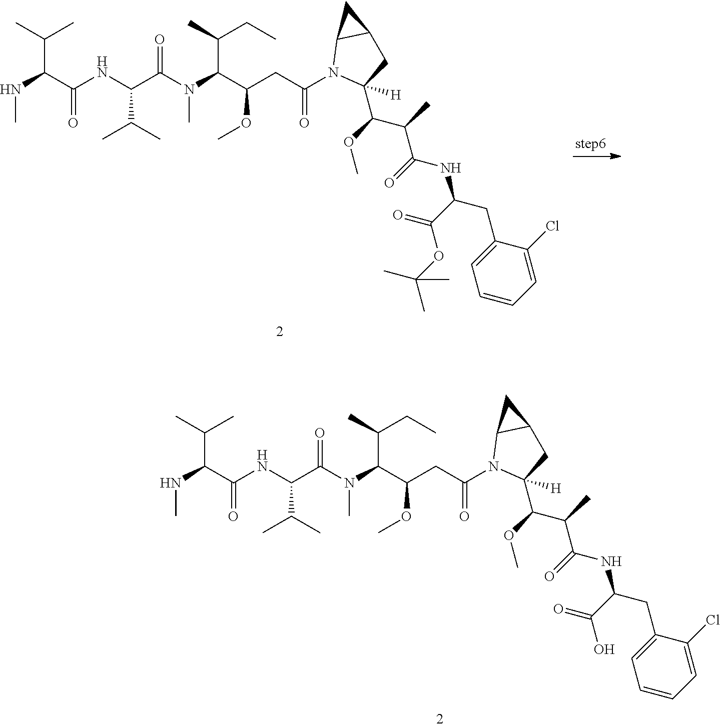 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@@H](NC)C(C)C)C(C)C]

### Step 1

### (S)-tert-butyl 2-amino-3-(2-chlorophenyl)propanoate

(S)-2-amino-3-(2-chlorophenyl)propanoic acid 2a (400 mg, 2.0 mmol, prepared according to the known method in “Journal of the American Chemical Society, 1940, 62, 565-8”) was dissolved in 10 mL of tert-butyl acetate, and added with perchloric acid (428 mg (70%), 3.00 mmol) and stirred at room temperature for 16 hours. The resulting mixture was added with 6 mL of water and the organic phase was washed with saturated sodium bicarbonate solution (3 mL). The aqueous phase was adjusted to pH=8 with saturated sodium bicarbonate solution and extracted with dichloromethane (5 mL×3). The organic phases were combined, washed successively with water (3 mL) and saturated sodium chloride solution (5 mL), dried over anhydrous sodium sulfate, filtered and the filtrate was concentrated under reduced pressure to give the crude title product of (S)-tert-butyl 2-amino-3-(2-chlorophenyl)propanoate 2b (400 mg, white solid). The product was used in the next step without further purification.

### Step 2

### (1S,3S,5S)-tert-butyl 3-((1R,2R)-3-((S)-1-tert-butoxy-3-(2-chlorophenyl)-1-oxopropan-2-ylamino)-1-methoxy-2-methyl-3-oxopropyl)-2-azabicyclo[3.1.0]hexane-2-carboxylate

(2R,3R)-3-((1S,3S,5S)-2-(tert-butoxy carbonyl)-2-azabicyclo[3.1.0]hexan-3-yl)-3-methoxy-2-methylpropanoic acid 1e (110 mg, 0.367 mmol) was dissolved in 6 mL of mixed solvent of dichloromethane and dimethylformamide (V/V=5:1), and then added with the crude product of (S)-tert-butyl 2-amino-3-(2-chlorophenyl)propanoate 2b (94 mg, 0.367 mmol), N,N-Diisopropylethylamine (0.32 mL, 1.835 mmol) and 2-(7-aza-1H-benzotriazole-1-yl)-N,N,N′,N′-tetramethyluronium hexafluorophosphate (168 mg, 0.44 mmol). The reaction mixture was stirred under argon atmosphere at room temperature for 1.5 hours and then added with 10 mL of water with stirring. The organic phase was washed with saturated sodium chloride solution (10 mL), dried over anhydrous sodium sulfate, filtered, and the filtrate was concentrated under reduced pressure. The residues were purified by silica gel column chromatography using eluent system B to give the title product of (1S,3S,5S)-tert-butyl 3-((1R,2R)-3-((S)-1-tert-butoxy-3-(2-chlorophenyl)-1-oxopropan-2-ylamino)-1-methoxy-2-methyl-3-oxopropyl)-2-azabicyclo[3.1.0]hexane-2-carboxylate 2c (112 mg, white foam solid), yield 56.8%.

MS m/z (ESI): 537.3 [M+1]

### Step 3

### (S)-tert-butyl 2-((2R,3R)-3-((1S,3S,5S)-2-azabicyclo[3.1.0]hexan-3-yl)-3-methoxy-2-methylpropanamido)-3-(2-chlorophenyl)propanoate

(1S,3S,5S)-tert-butyl 3-((1R,2R)-3-((S)-1-tert-butoxy-3-(2-chlorophenyl)-1-oxopropan-2-ylamino)-1-methoxy-2-methyl-3-oxopropyl)-2-azabicyclo[3.1.0]hexane-2-carboxylate 2c (110 mg, 0.205 mmol) was dissolved in 2 mL dioxane and then added with a 5.6 M solution of hydrogen chloride in dioxane (0.13 mL, 0.717 mmol). The reaction mixture was stirred under argon atmosphere at room temperature for 1 hour, and then placed in a refrigerator at 0° C. for 6 hours. Then, the reaction mixture was added with 5 mL of methylene chloride and 10 mL of saturated sodium bicarbonate solution and stirred for 10 minutes. The aqueous phase was extracted with 5 mL of dichloromethane. The combined methylene chloride phase was washed with saturated sodium chloride solution (20 mL), dried over anhydrous sodium sulfate, filtered, and the filtrate was concentrated under reduced pressure to give the crude title product of (S)-tert-butyl 2-((2R,3R)-3-((1S,3S,5S)-2-azabicyclo[3 0.1.0]hexan-3-yl)-3-methoxy-2-methylpropanamido)-3-(2-chlorophenyl)propanoate 2d (99 mg, yellow liquid). The product was used in the next step without further purification.

MS m/z (ESI): 437.2 [M+1]

### Step 4

### (S)-tert-butyl 2-((2R,3R)-3-((1S,3S,5S)-2-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-2-azabicyclo[3.1.0]hexan-3-yl)-3-methoxy-2-methylpropanamido)-3-(2-chlorophenyl)propanoat

The crude (S)-tert-butyl 2-((2R,3R)-3-((1S,3S,5S)-2-azabicyclo[3.1.0]hexan-3-yl)-3-methoxy-2-methylpropanamido)-3-(2-chlorophenyl)propanoate 2d (99 mg, 0.226 mmol), (5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid 1i (144.4 mg, 0.226 mmol) was dissolved in 6 mL of mixed solvent of dichloromethane and dimethylformamide (V/V=5:1), and then added with N,N-diisopropylethylamine (0.2 mL, 1.13 mmol) and 2-(7-azobenzotriazole)-N,N,N′,N′-tetramethyluronium hexafluorophosphate (103.3 mg, 0.271 mmol). The reaction mixture was stirred under argon atmosphere at room temperature for 1 hour and then added with 10 mL of water under stirring. The methylene chloride phase was washed with saturated sodium chloride solution (10 mL), dried over anhydrous sodium sulfate, filtrated, and the filtrate was concentrated under reduced pressure. The residues were purified by silica gel column chromatography eluting with eluent system B to give the title product of (S)-tert-butyl 2-((2R,3R)-3-((1S,3S,5S)-2-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-2-azabicyclo[3.1.0]hexan-3-yl)-3-methoxy-2-methylpropanamido)-3-(2-chlorophenyl)propanoat 2e (127 mg, white foam solid), yield 53.1%.

MS m/z (ESI): 1056.4 [M+1]

### Step 5

### (S)-tert-butyl 3-(2-chlorophenyl)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)propanoate

(S)-tert-butyl 2-((2R,3R)-3-((1S,3S,5S)-2-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-2-azabicyclo[3.1.0]hexan-3-yl)-3-methoxy-2-methylpropanamido)-3-(2-chlorophenyl)propanoat 2e (127 mg, 0.12 mmol) was dissolved in 2 mL of dichloromethane and then added with 2 mL of diethylamine. The reaction mixture was stirred under argon atmosphere at room temperature for 3 hours. Then the reaction solution was concentrated under reduced pressure to give the crude title product of (S)-tert-butyl 3-(2-chlorophenyl)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)propanoate 2f (130 mg, yellow sticky material). The product was used in the next step without further purification.

MS m/z (ESI): 834.5 [M+1]

### Step 6

### (S)-3-(2-chlorophenyl)-2-((2R,3R)-3-((1S,3S,5S)-2-((3R,4S,5S)-4-((S)—N,3-dimethyl-2-4 S)-3-methyl-2-(methylamino)butanamido)butanamido)-3-methoxy-5-methylheptanoyl)-2-azabicyclo[3 0.1.0]hexan-3-yl)-3-methoxy-2-methylpropanamido)propanoic acid

The crude product of (S)-tert-butyl 3-(2-chlorophenyl)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)propanoate 2f (100 mg, 0.12 mmol) was dissolved in 1 mL dioxane, and then added with 3 mL of a 5.6 M solution of hydrogen chloride in dioxane. The reaction mixture was stirred at room temperature for 12 hours under an argon atmosphere. Then the reaction solution was concentrated under reduced pressure, and the residues were purified by high performance liquid chromatography to give the title product of (S)-3-(2-chlorophenyl)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)propanoic acid 2 (35 mg, white solid), yield 35.7%.

MS m/z (ESI): 778.7 [M+1]

¹H NMR (400 MHz, CD₃OD): δ 7.41-7.32 (m, 2H), 7.31-7.16 (m, 2H), 4.80-4.65 (m, 2H), 4.22-4.13 (m, 1H), 4.10-4.03 (m, 1H), 3.98-3.91 (m, 1H), 3.87-3.82 (m, 1H), 3.73-3.63 (m, 2H), 3.47-3.12 (m, 9H), 3.09-3.01 (m, 1H), 2.67-2.57 (m, 4H), 2.24-2.11 (m, 3H), 2.09-1.98 (m, 1H), 1.89-1.67 (m, 3H), 1.51-1.25 (m, 4H), 1.20-0.93 (m, 18H), 0.92-0.79 (m, 4H), 0.75-0.66 (m, 1H).

### Example 3

### (S)-2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoic acid

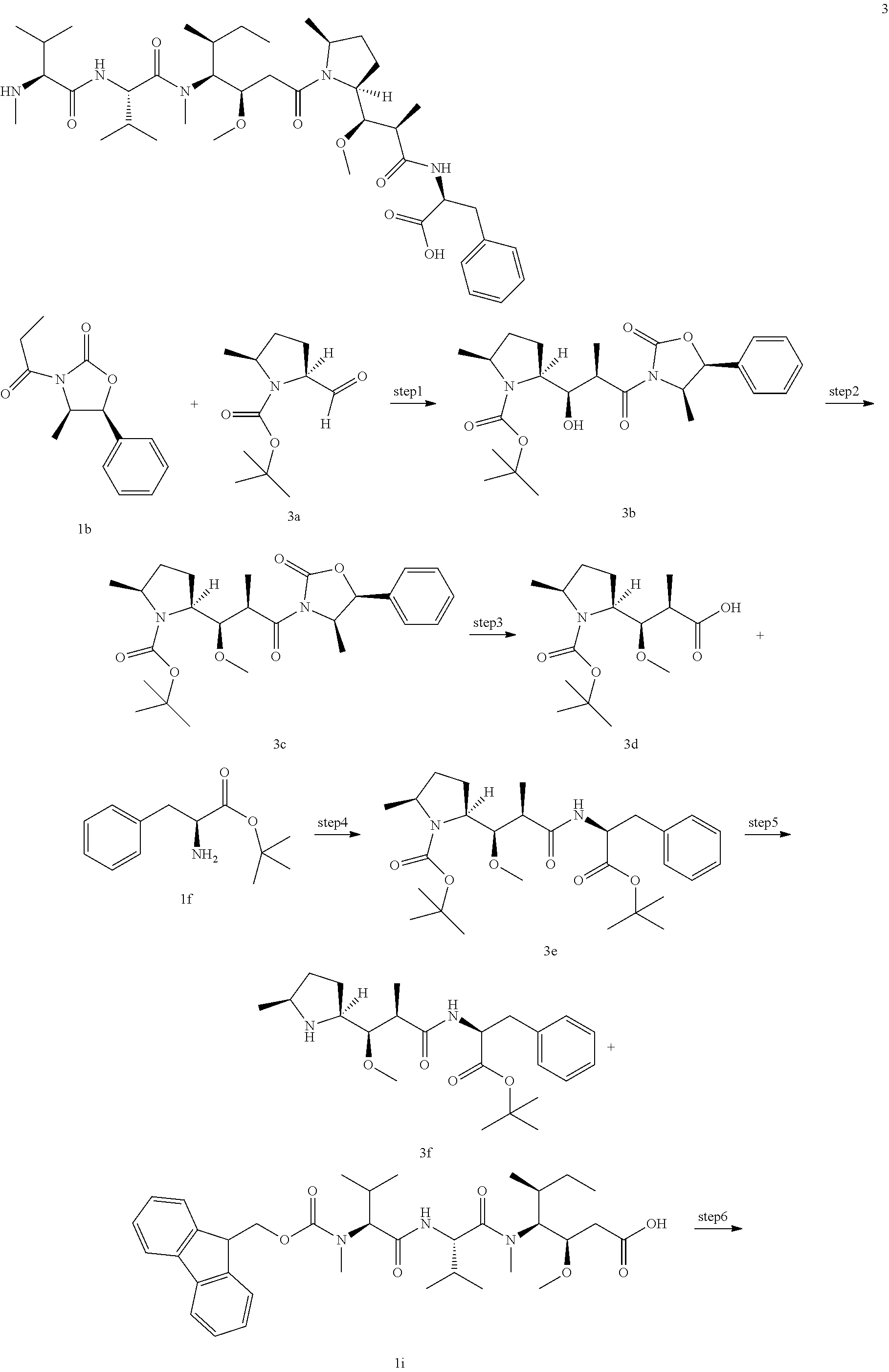

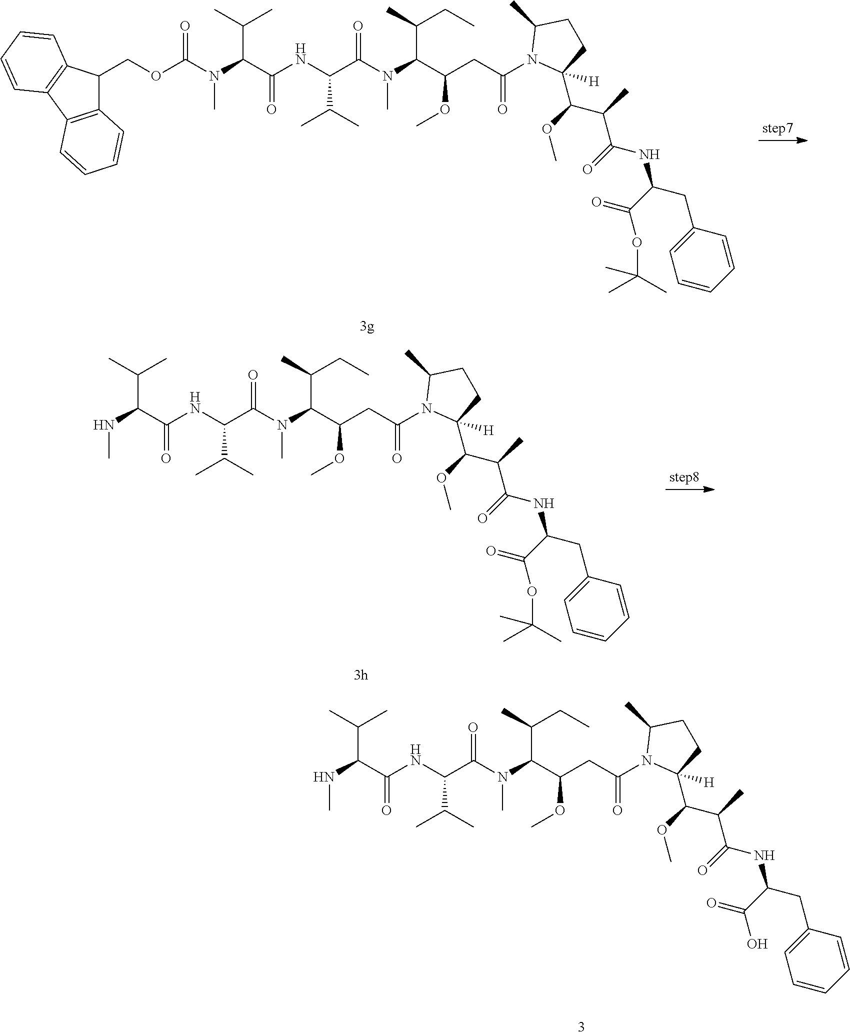 [SMILES: CC[C@H](C)[C@@H]([C@@H](CC(=O)N1[C@@H](C)CC[C@H]1[C@H](OC)[C@@H](C)C(=O)N[C@@H](Cc1ccccc1)C(=O)OC(C)(C)C)OC)N(C)C(=O)[C@@H](NC(=O)[C@H](C(C)C)N(C)C(=O)OCC1c2ccccc2-c2ccccc21)C(C)C ; CC[C@H](C)[C@@H]([C@@H](CC(=O)N1[C@@H](C)CC[C@H]1[C@H](OC)[C@@H](C)C(=O)N[C@@H](Cc1ccccc1)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

### Step 1

### (2S,5S)-tert-butyl 2-((1R,2R)-1-hydroxy-2-methyl-3-((4R,5S)-4-methyl-2-oxo-5-phenyloxazolidin-3-yl)-3-oxopropyl)-5-methylpyrrolidine-1-carboxylate

(4R,5S)-4-methyl-5-phenyl-3-propionyloxazolidin-2-one 1b (0.992 g, 4.2 mmol) was dissolved in 20 mL of dichloromethane, and cooled to 0° C. under argon atmosphere. The reaction mixture was added with triethylamine (0.69 mL, 4.96 mmol) and then dibutylboron trifluoromethanesulfonate (4.4 mL, 4.4 mmol) dropwise, and the mixture was stirred at 0° C. for 50 minutes. The reaction solution was cooled to −75° C. and added with a 5 mL solution of (2S,5S)-tert-butyl 2-formyl-5-methylpyrrolidine-1-carboxylate 3a (900 mg, 4.2 mmol, prepared according to the known method of “US 20120195857”) in methylene chloride, and then stirred at −75° C. for 1.5 hours, at 0° C. for 1.5 hours and at room temperature for 1 hour. Then, the reaction mixture was added with a 36 mL of mixture of phosphate buffer (pH=7.0) and methanol (V/V=1:3) at room temperature. The reaction mixture was cooled to 0° C., added with 36 mL of a mixture of methanol and hydrogen peroxide (30%) (V/V=2:1), and then stirred for 1 hour at room temperature. After completion of the reaction, the organic phase was concentrated under reduced pressure and 15 mL of water were added. The aqueous phase was extracted with ether (30 mL×3) and the combined ether phases were washed successively with 5% sodium bicarbonate solution, water, saturated sodium chloride solution (150 mL), dried over anhydrous sodium sulfate, filtered, and the filtrate was concentrated under reduced pressure. The residues were purified by silica gel column chromatography using eluent system B to give the title product of (2S,5S)-tert-butyl 2-((1R,2R)-1-hydroxy-2-methyl-3-((4R,5S)-4-methyl-2-oxo-5-phenyloxazolidin-3-yl)-3-oxopropyl)-5-methylpyrrolidine-1-carboxylate 3b (600 mg, white solid), yield 33%.

### Step 2

### (2S,5S)-tert-butyl 2-((1R,2R)-1-methoxy-2-methyl-3-((4R,5S)-4-methyl-2-oxo-5-phenyloxazolidin-3-yl)-3-oxopropyl)-5-methylpyrrolidine-1-carboxylate (2S,5S)-tert-butyl

2-((1R,2R)-1-hydroxy-2-methyl-3-((4R,5S)-4-methyl-2-oxo-5-phenyloxazolidin-3-yl)-3-oxopropyl)-5-methylpyrrolidine-1-carboxylate 3b (600 mg, 1.34 mmol) was dissolved in 15 mL of methylene chloride, and added with 1 g of crushed molecular sieves. The mixture was added with 1,8-bisdimethylaminonaphthalene (740 mg, 3.45 mmol) and trimethyloxonium tetrafluoroborate (500 mg, 3.38 mmol) at 0° C. under argon atmosphere. The reaction mixture was wrapped with tin foil and stirred at room temperature for 38 hours. After completion of the reaction, the resulting mixture was filtered and the filter cake was washed with dichloromethane. The filtrate was combined and the organic phase was washed with saturated ammonium chloride solution (20 mL×3) to remove excess 1,8-bis-dimethylaminonaphthalene, then washed with saturated sodium chloride solution and dried over anhydrous sodium sulfate, filtered, and the filtrate was concentrated under reduced pressure. The residues were purified by silica gel column chromatography using eluent system B to give the title product of (2S,5S)-tert-butyl 2-((1R,2R)-1-methoxy-2-methyl-3-((4R,5S)-4-methyl-2-oxo-5-phenyloxazolidin-3-yl)-3-oxopropyl)-5-methylpyrrolidine-1-carboxylate 3c (200 mg, white solid), yield 32%.

### Step 3

### (2R,3R)-3-((2S,5S)-1-(tert-butoxycarbonyl)-5-methylpyrrolidin-2-yl)-3-methoxy-2-methylpropanoic acid

(2S,5S)-tert-butyl 2-((1R,2R)-1-methoxy-2-methyl-3-((4R,5S)-4-methyl-2-oxo-5-phenyloxazolidin-3-yl)-3-oxopropyl)-5-methylpyrrolidine-1-carboxylate 3c (200 mg, 0.43 mmol) was dissolved in 22 mL of tetrahydrofuran and cooled to 0° C. under argon atmosphere. The mixture was then added dropwise slowly with hydrogen peroxide (186 mg, 1.6 mmol) and lithium hydroxide monohydrate (58 mg, 1.37 mmol). The reaction mixture was stirred at 0° C. for 10 minutes, and then the ice bath was removed and further stirred at room temperature for 44 hours. After completion of the reaction, the reaction solution was added with sodium sulfite solid (220 mg, 1.74 mmol) and stirred at room temperature for 1 hour, and then 15 mL of water was added. The organic phase was concentrated under reduced pressure and the residues were extracted with dichloromethane (20 mL×2). The aqueous phase was added dropwise with 2N hydrochloric acid until a pH of 3 to 4, then extracted with ethyl acetate (20 mL×3), and the ethyl acetate phase was washed successively with water and saturated sodium chloride solution, dried over anhydrous sodium sulfate, filtered and the filtrate was concentrated under reduced pressure to give the crude title product of (2R,3R)-3-((2S,5S)-1-(tert-butoxycarbonyl)-5-methylpyrrolidin-2-yl)-3-methoxy-2-methylpropanoic acid 3d (120 mg, white solid). The product was used in the next step without further purification.

### Step 4

### (2S,5S)-tert-butyl 2-((1R,2R)-3-((S)-1-tert-butoxy-1-oxo-3-phenylpropan-2-ylamino)-1-methoxy-2-methyl-3-oxopropyl)-5-methylpyrrolidine-1-carboxylate

The crude product of (2R,3R)-3-((2S,5S)-1-(tert-butoxycarbonyl)-5-methylpyrrolidin-2-yl)-3-methoxy-2-methylpropanoic acid 3d (106 mg, 0.35 mmol) was dissolved in 4.8 mL of a mixed solvent of dichloromethane and dimethylformamide (V/V=5:1), and then added with (S)-tert-butyl 2-amino-3-phenylpropionic acid if (80 mg, 0.36 mmol). The mixture was added with N,N-diisopropylethylamine (0.30 mL, 1.74 mmol) and 2-(7-azobenzotriazole)-N,N,N′,N′-tetramethyluronium hexafluorophosphate (160 mg, 0.42 mmol) under an argon atmosphere, and then stirred for 2 hours at room temperature. After completion of the reaction, the reaction mixture was added with 10 mL of dichloromethane, and then washed successively with water (5 mL×2) and saturated sodium chloride solution, dried over anhydrous sodium sulfate, filtered and the filtrate was concentrated under reduced pressure. The residues were purified by silica gel column chromatography using eluent system B to give the title product of (2S,5S)-tert-butyl 2-((1R,2R)-3-((S)-1-tert-butoxy-1-oxo-3-phenylpropan-2-ylamino)-1-methoxy-2-methyl-3-oxopropyl)-5-methylpyrrolidine-1-carboxylate 3e (138 mg, colorless viscous liquid), yield 78%.

### Step 5

### (S)-tert-butyl 2-((2R,3R)-3-methoxy-2-methyl-3-((2S,5S)-5-methylpyrrolidin-2-yl)propanamido)-3-phenylpropanoate

(2S,5S)-tert-butyl 2-((1R,2R)-3-((S)-1-tert-butoxy-1-oxo-3-phenylpropan-2-ylamino)-1-methoxy-2-methyl-3-oxopropyl)-5-methylpyrrolidine-1-carboxylate 3e (150 mg, 0.267 mmol) was dissolved in 2.2 mL of dioxane, and then added with a 4 M solution of hydrogen chloride in dioxane (0.160 mL, 0.896 mmol). The reaction system was sealed and stirred for 7 hours at room temperature and then placed in a refrigerator for 16 hours at 4° C. The reaction solution was concentrated under reduced pressure, and the residues were added with 15 mL of dichloromethane, and then cooled to 0° C., and added with saturated sodium bicarbonate solution dropwise to adjust to pH 8. The aqueous phase was extracted with dichloromethane (8 mL×2) and the organic phases were combined. The organic phase was washed with saturated sodium chloride solution and dried over anhydrous sodium sulfate, filtered and the filtrate was concentrated under reduced pressure to give the title product of (S)-tert-butyl 2-((2R,3R)-3-methoxy-2-methyl-3-((2S,5S)-5-methylpyrrolidin-2-yl)propanamido)-3-phenylpropanoate 3f (80 mg, colorless viscous solid), yield 74%.

### Step 6

### (S)-tert-butyl 2-((2R,3R)-3-((2S,5S)-1-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-5-methylpyrrolidin-2-yl)-3-methoxy-2-methylpropanamido)-3-phenylpropanoate

(S)-tert-butyl 2-((2R,3R)-3-methoxy-2-methyl-3-2S,5S)-5-methylpyrrolidin-2-yl)propanamido)-3-phenylpropanoate 3f (80 mg, 0.198 mmol), (5S,8S,11S,12R)-11-((S)-sec-butyl)-1-(9H-fluoren-9-yl)-5,8-diisopropyl-12-methoxy-4,1 0-dimethyl-3,6,9-trioxo-2-oxa-4,7,10-triazatetradecan-14-oic acid 1i (136 mg, 0.213 mmol) was dissolved in 4.8 mL of a mixed solvent of dichloromethane and dimethylformamide (V/V=5:1), and then added with N,N-diisopropylethylamine (0.170 mL, 0.98 mmol) and 2-(7-azobenzotriazole)-N,N,N′,N′-tetramethyluronium hexafluorophosphate (100 mg, 0.263 mmol). The reaction mixture was stirred under argon atmosphere at room temperature for 1 hour. After completion of the reaction, the reaction mixture was added with 15 mL of dichloromethane and washed with water (6 mL×2). The aqueous phase was extracted with dichloromethane (5 mL), and the combined dichloromethane phases were washed with saturated sodium chloride solution and dried over anhydrous sodium sulfate, filtered and the filtrate was concentrated under reduced pressure. The residues were purified by silica gel column chromatography using eluent system B to give the title product of (S)-tert-butyl 2-((2R,3R)-3-2S,5S)-1-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-5-methylpyrrolidin-2-yl)-3-methoxy-2-methylpropanamido)-3-phenylpropanoate 3g (168 mg, white foam solid), yield 81%.

### Step 7

### (S)-tert-butyl 2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoate

(S)-tert-butyl 2-((2R,3R)-3-((2S,5S)-1-((5S,8S,11S,12R)-11-sec-butyl-1-(9H-fluoren-9-yl)-5,8-diisopropyl-12-methoxy-4,10-dimethyl-3,6,9-trioxo-2-oxa-4,7,10-triazatetradecane)-5-methylpyrrolidin-2-yl)-3-methoxy-2-methylpropanamido)-3-phenylpropanoate 3g (167 mg, 0.16 mmol) was dissolved in 2 mL of dichloromethane, and added with 2 mL of diethylamine. The reaction mixture was stirred under argon atmosphere at room temperature for 3 hours. After completion of the reaction, the reaction solution was concentrated under reduced pressure to give the crude title product of (S)-tert-butyl 2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoate 3h (180 mg, yellow liquid). The product was used in the next step without further purification.

MS m/z (ESI): 802.6 [M+1]

### Step 8

### (S)-2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoi c acid

The crude product of (S)-tert-butyl 2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoate 3h (130 mg, 0.16 mmol) was dissolved in 1 mL of dioxane, and added with 3 mL of a 5.6 M solution of hydrogen chloride in dioxane. The reaction system was sealed and stirred for 12 hours at room temperature. After completion of the reaction, the reaction solution was concentrated under reduced pressure. The residues were purified by high performance liquid chromatography to give the title product of (S)-2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoic acid 3 (28 mg, white solid), yield 23%.

MS m/z (ESI): 746.7 [M+1]

¹H NMR (400 MHz, CD₃OD): δ 7.31-7.14 (m, 5H), 4.79-4.67 (m, 2H), 4.25-4.13 (m, 1H), 4.10-3.97 (m, 2H), 3.77-3.66 (m, 1H), 3.60-3.52 (m, 1H), 3.51-3.42 (m, 1H), 3.41-3.12 (m, 7H), 2.97-2.85 (m, 1H), 2.67 (d, 3H), 2.48-2.40 (m, 2H), 2.30-2.02 (m, 4H), 1.93-1.73 (m, 2H), 1.70-1.55 (m, 1H), 1.53-1.28 (m, 5H), 1.26-1.11 (m, 7H), 1.10-0.99 (m, 14H), 0.98-0.83 (m, 4H).

### Example 4

### (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-phenylpropanoic acid

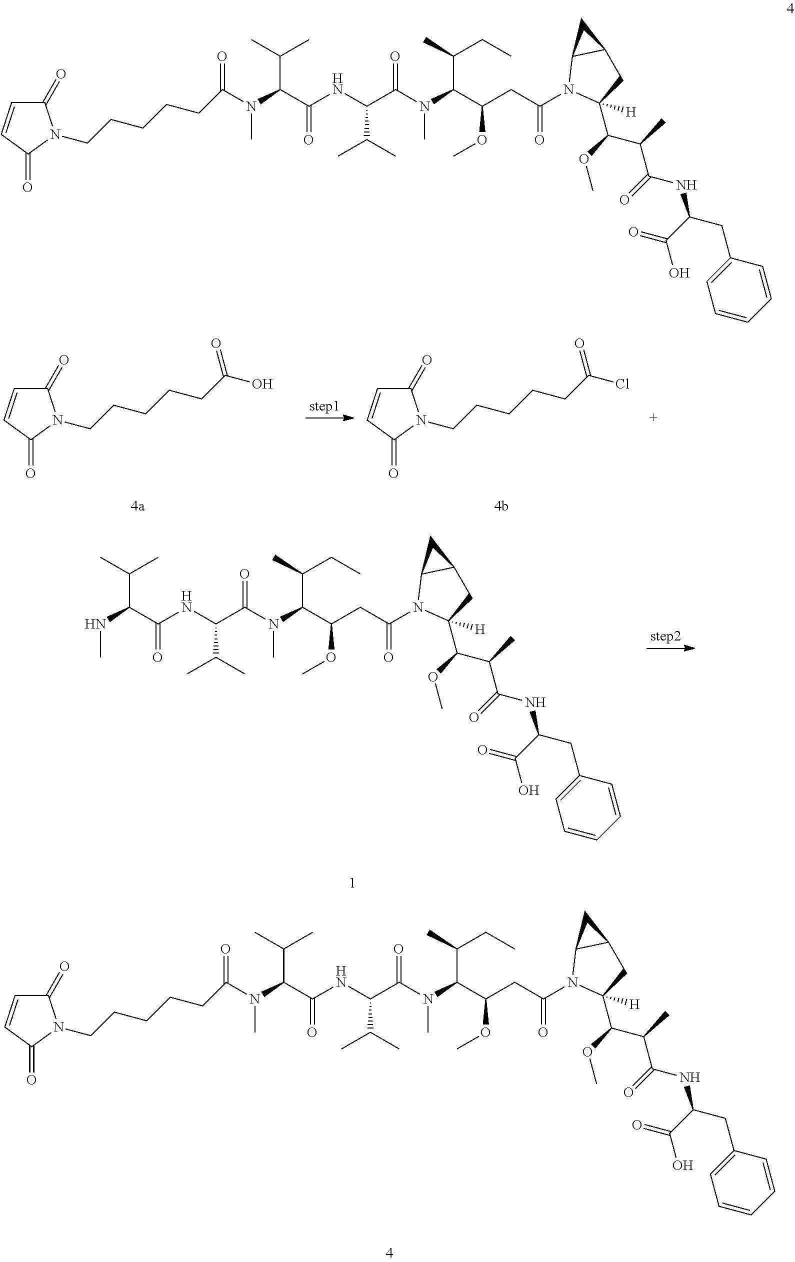

### Step 1

### 6-(2,5-di oxo-2,5-dihydro-1H-pyrrol-1-yl)hexanoyl chloride

6-(2,5-di oxo-2,5-dihydro-1H-pyrrol-1-y 1)hexanoic acid 4a (1.5 g, 7.10 mmol, prepared according to the known method of “Journal of Medicinal Chemistry, 2013, 56(24), 9955-9968”) was added with a drop of N,N-dimethylformamide, and then added with 15 mL of oxalyl chloride dropwise with vigorous stirring under an argon atmosphere after cooling in a dry ice bath. Then the reaction mixture was stirred at room temperature for 1 hour. After completion of the reaction, the reaction mixture was concentrated under reduced pressure, and the residues were dissolved in methylene chloride and concentrated under reduced pressure to give the crude title product of 6-(2,5-dioxo-2,5-dihydro-1H-pyrrol-1-yl)hexanoyl chloride 4b. The product was used in the next step without further purification.

### Step 2

### (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3 0.1.0]hexan-3-yl)-3-methoxy-2-methylpropanamido)-3-phenylpropanoic acid

(S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-phenylpropanoic acid 1 (17 mg, 0.023 mmol) was dissolved in 1 mL of dichloromethane and added with N,N-diisopropylethylamine (20 μL, 0.115 mmol). Then the mixture was added dropwise with a preformed solution of 6-(2,5-dioxo-2,5-dihydro-1H-pyrrol-1-yl) hexanoyl chloride 4b (7.9 mg, 0.034 mmol) ire dichloromethane under an argon atmosphere in an ice bath, and then stirred at room temperature for 5 hours. After completion of the reaction, 10 mL of methanol was added and the reaction mixture was concentrated under reduced pressure. The residues were purified by high performance liquid chromatography to give the title product of (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3 0.1.0]hexan-3-yl)-3-methoxy-2-methylpropanamido)-3-phenylpropanoic acid 4 (9 mg, white solid), yield 42%.

MS m/z (ESI): 937.4 [M+1]

¹H NMR (400 MHz, CD₃OD): δ 7.37-7.17 (m, 5H), 6.84-6.79 (m, 2H), 4.80-4.71 (m, 2H), 4.69-4.56 (m, 2H), 4.26-4.19 (m, 1H), 4.14-4.07 (m, 1H), 3.92-3.86 (m, 1H), 3.84-3.78 (m, 1H), 3.77-3.60 (m, 1H), 3.55-3.47 (m, 2H), 3.42-3.23 (m, 5H), 3.18-3.12 (m, 2H), 3.07-3.03 (m, 2H), 3.02-2.82 (m, 2H), 2.66-2.58 (m, 2H), 2.54-2.46 (m, 1H), 2.46-2.38 (m, 2H), 2.30-2.14 (m, 2H), 2.09-1.99 (m, 1H), 1.90-1.78 (m, 1H), 1.75-1.56 (m, 6H), 1.48-1.28 (m, 6H), 1.20-0.79 (m, 22H), 0.77-0.69 (m, 1H).

### Example 5

### (S)-3-(2-chlorophenyl)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)propanoic acid

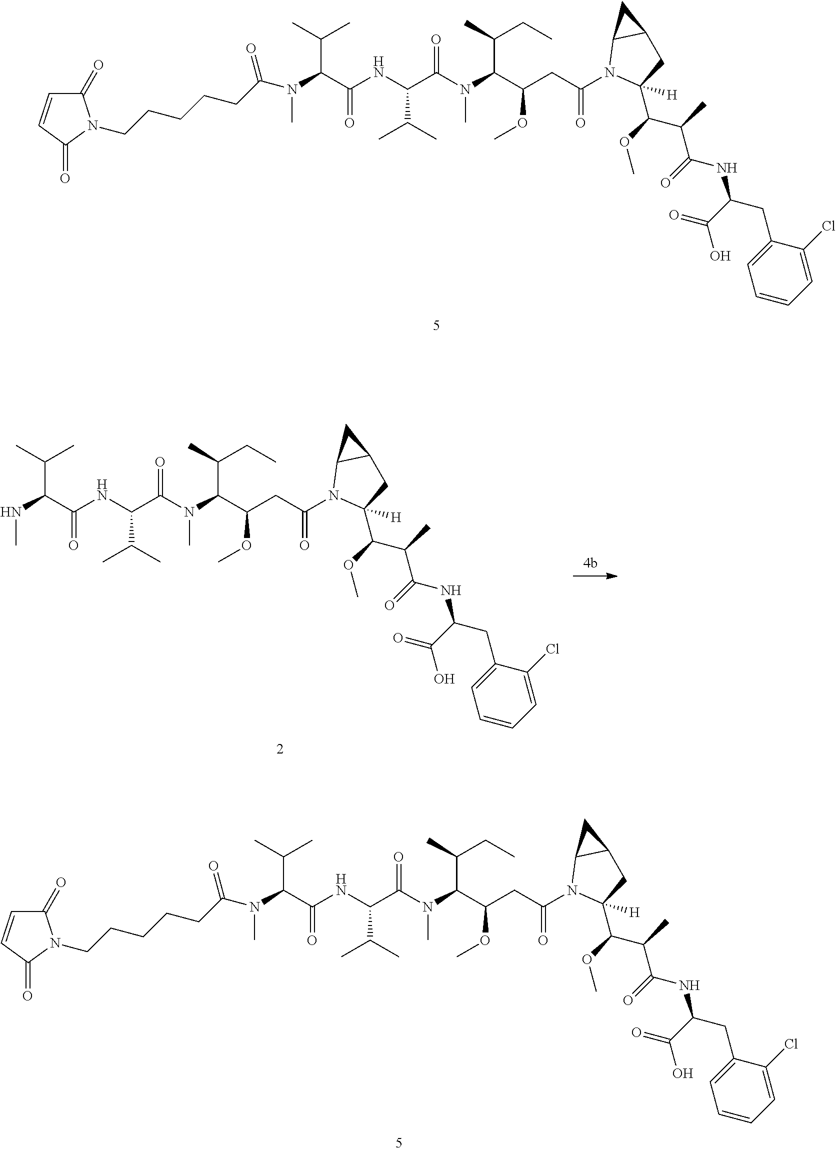 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1Cl)C(=O)O)OC)N(C)C(=O)[C@@H](NC(=O)[C@H](C(C)C)N(C)C(=O)CCCCCN1C(=O)C=CC1=O)C(C)C]

(S)-3-(2-chlorophenyl)-2-((2R,3R)-3-((1S,3S,5S)-2-((3R,4S,5S)-4-((S)-N,3-dimethyl-2-4S)-3-methyl-2-(methylamino)butanamido)butanamido)-3-methoxy-5-methylheptanoyl)-2-azabicyclo[3.1.0]hexan-3-yl)-3-methoxy-2-methylpropanamido)propanoic acid 2 (18 mg, 0.023 mmol) was dissolved in 1 mL of dichloromethane and added with N,N-diisopropylethylamine (0.02 mL, 0.115 mmol). The mixture was added dropwise with a preformed solution of 6-(2,5-dioxo-25-dihydro-1H-pyrrol-1-yl) hexanoyl chloride 4b (7.9 mg, 0.034 mmol) in dichloromethane under an argon atmosphere in an ice bath, and then stirred at room temperature for 4 hours. The reaction was quenched with 5 mL of methanol and then concentrated under reduced pressure. The residues were purified by high performance liquid chromatography to give the title product of (S)-3-(2-chlorophenyl)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)propanoic acid 5 (6 mg, white solid), yield 26.6%.

MS m/z (ESI): 971.5 [M+1]

¹H NMR (400 MHz, CD₃OD): δ 7.42-7.34 (m, 2H), 7.33-7.19 (m, 2H), 6.83-6.78 (m, 2H), 4.83-4.70 (m, 2H), 4.68-4.56 (m, 2H), 4.24-4.16 (m, 1H), 4.13-4.05 (m, 1H), 4.03-3.95 (m, 1H), 3.91-3.84 (m, 1H), 3.76-3.65 (m, 1H), 3.54-3.48 (m, 2H), 3.47-3.18 (m, 5H), 3.17-2.96 (m, 6H), 2.67-2.58 (m, 2H), 2.53-2.38 (m, 3H), 2.28-2.18 (m, 2H), 2.09-2.00 (m, 1H), 1.92-1.57 (m, 7H), 1.51-1.28 (m, 6H), 1.21-0.82 (m, 22H), 0.78-0.69 (m, 1H).

### Example 6

### (S)-2-((2R,3R)-3-((2S,5S)-1-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-5-methylpyrrolidin-2-yl)-3-methoxy-2-methylpropanamido)-3-phenylpropanoic acid

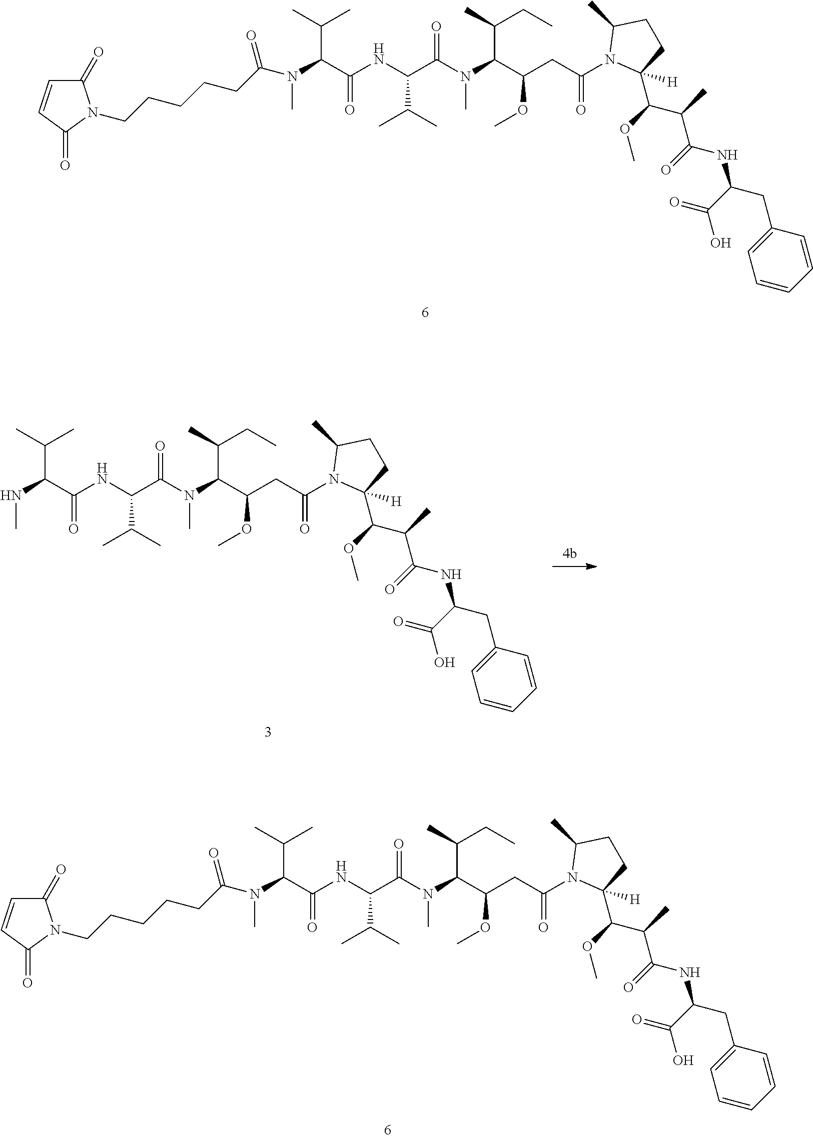 [SMILES: CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C ; CC[C@H](C)[C@@H]([C@@H](CC(=O)N1[C@@H](C)CC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((2S,5S)-1-((3R,4S,5S)-4-((S)—N,3-dimethyl-2-((S)-3-methyl-2-(methylamino)butanamido)butanamido)-3-methoxy-5-methylheptanoyl)-5-methylpyrrolidin-2-yl)-3-methoxy-2-methylpropanamido)-3-phenylpropanoic acid 3 (18 mg, 0.024 mmol) was dissolved in 1 mL of dichloromethane and added with N,N-diisopropylethylamine (0.02 mL, 0.12 mmol). The mixture was added dropwise with a preformed solution of 6-(2,5-dioxo-2,5-dihydro-1H-pyrrol-1-yl)hexanoyl chloride 4b (8.3 mg, 0.036 mol) in dichloromethane under an argon atmosphere in an ice bath. The above reaction mixture was stirred at room temperature for 4 hours. The reaction was quenched with 5 mL of methanol and then concentrated under reduced pressure. The residues were purified by high performance liquid chromatography to give the title product of (S)-2-((2R,3R)-3-((2S,5S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-methylpyrrolidin-2-yl)-3-methoxy-2-methylpropanamido)-3-phenylpropanoic acid 6 (7 mg, white solid), yield 30.9%.

MS m/z (ESI): 939.5 [M+1]

¹H NMR (400 MHz, CD₃OD): δ 7.30-7.15 (m, 5H), 6.83-6.78 (m, 2H), 4.78-4.69 (m, 2H), 4.69-4.56 (m, 2H), 4.24-4.12 (m, 1H), 4.10-3.96 (m, 2H), 3.60-3.44 (m, 3H), 3.41-3.22 (m, 4H), 3.16-3.10 (m, 2H), 3.07-3.02 (m, 2H), 3.01-2.86 (m, 2H), 2.52-2.38 (m, 4H), 2.31-2.15 (m, 3H), 2.09-1.99 (m, 1H), 1.91-1.77 (m, 2H), 1.71-1.56 (m, 5H), 1.52-1.28 (m, 9H), 1.26-1.14 (m, 6H), 1.11-0.77 (m, 18H).

### Example 7

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid

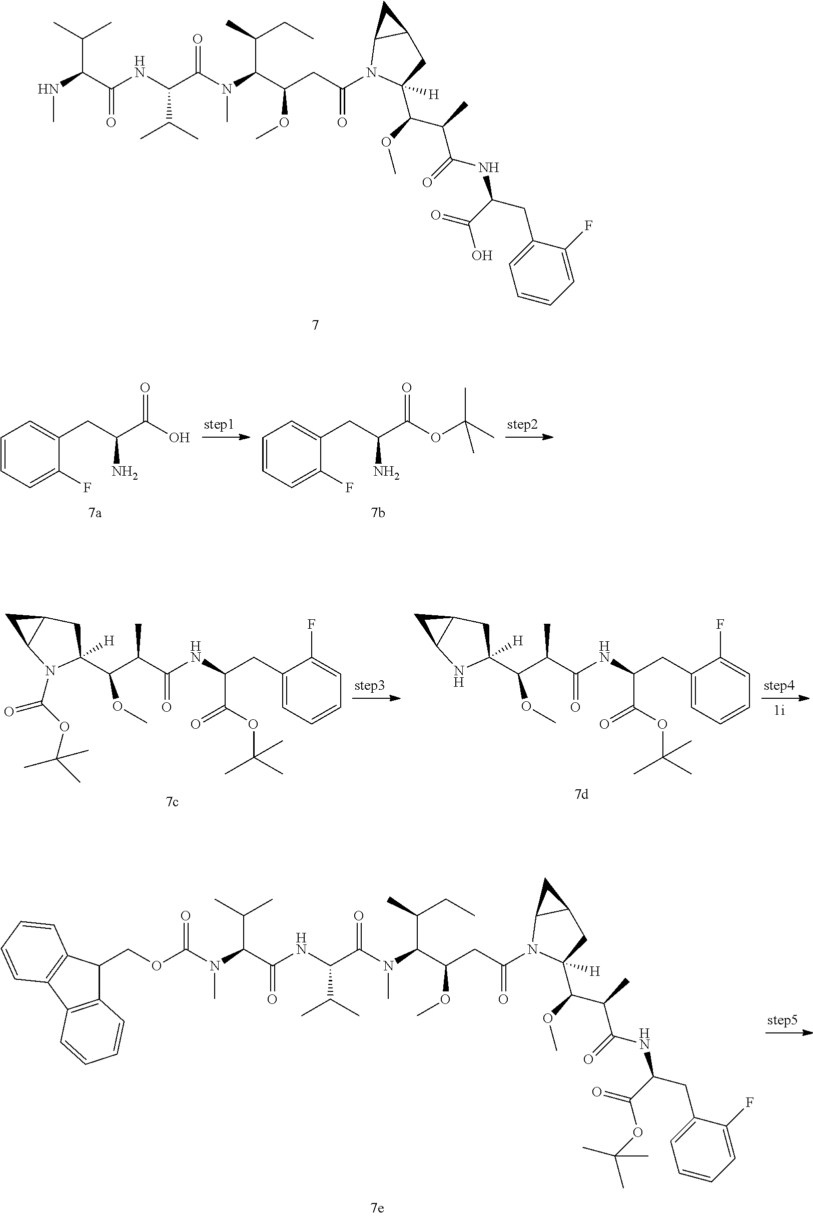

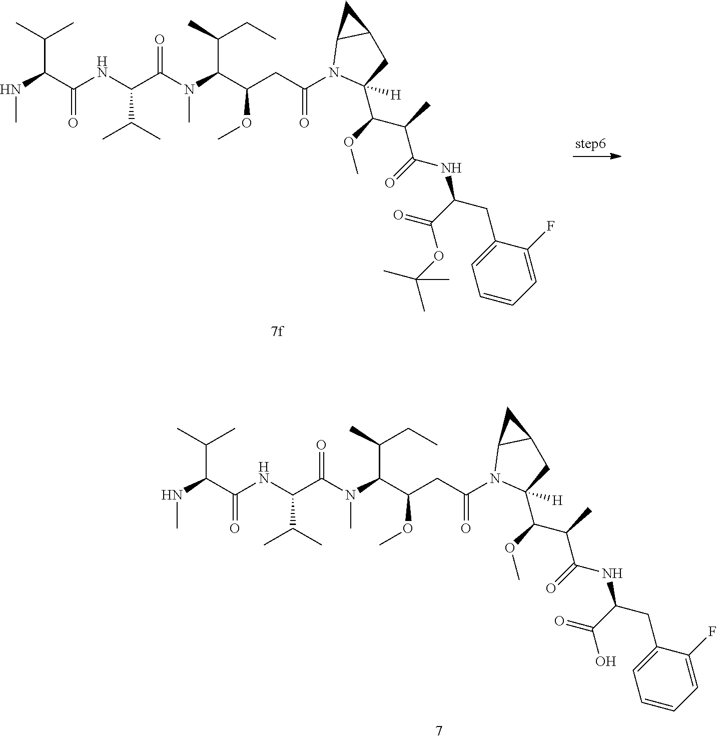 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C]

### Step 1

### (S)-tert-butyl 2-amino-3-(2-fluorophenyl)propanoate

(S)-butyl 2-amino-3-(2-fluorophenyl)propanoate 7a (400 mg, 2.18 mmol, prepared according to the known method of “Advanced Synthesis & Catalysis, 2012, 354(17), 3327-3332”) was dissolved in 10 mL of tert-butyl acetate, and added with perchloric acid (300 mg (70%), 3.3 mmol). The mixture was stirred at room temperature for 16 hours. After the reaction, 6 mL of water were added and the organic phase was washed with saturated sodium bicarbonate solution (3 mL). The aqueous phase was adjusted to pH=8 with saturated sodium bicarbonate solution and extracted with dichloromethane (5 mL×3). The organic phases were combined, washed successively with water (3 mL) and saturated sodium chloride solution (5 mL), dried over anhydrous sodium sulfate, filtered and the filtrate was concentrated under reduced pressure to obtain the crude title product of (S)-tert-butyl 2-amino-3-(2-fluorophenyl)propanoate 7b (390 mg, yellow oil). The product was used in the next step without further purification.

### Step 2

### (1S,3S,5S)-tert-butyl 3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexane-2-carboxylate

(2R,3R)-3-((1S,3S,5S)-2-(tert-butoxy carbonyl)-2-azabicyclo[3.1.0]hexan-3-yl)-3-methoxy-2-methylpropanoic acid 1e (100 mg, 0.334 mmol) was dissolved in 6 mL of a mixed solvent of dichloromethane and dimethylformamide (V/V=5:1), and added with the crude product of (S)-tert-butyl 2-amino-3-(2-fluorophenyl)propanoate 7b (80 mg, 0.334 mmol), N,N-diisopropylethylamine (0.29 mL, 1.67 mmol) and 2-(7-azobenzotriazole)-N,N,N′,N′-tetramethyluronium hexafluorophosphate (152.3 mg, 0.40 mmol). The reaction mixture was stirred under argon atmosphere at room temperature for 1 hour. After completion of the reaction, the reaction mixture was added with 10 mL of water under stirring, and the dichloromethane phase was washed with saturated sodium chloride solution (10 mL), dried over anhydrous sodium sulfate, filtered and the filtrate was concentrated under reduced pressure. The residues were purified by silica gel column chromatography using eluent system B to give the title product of (1S,3S,5S)-tert-butyl 3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexane-2-carboxylate 7c (173 mg, colorless liquid), yield 99.5%.

MS m/z (ESI): 521.2 [M+1]

### Step 3

### (S)-tert-butyl 2-((2R,3R)-3-((1S,3S,5S)-2-azabicyclo[3.1.0]hexan-3-yl)-3-methoxy-2-methylpropanamido)-3-(2-fluorophenyl)propanoate

(1S,3S,5S)-tert-butyl 3-((1R,2R)-3-(((S)-1-(tert-butoxy)-3-(2-fluorophenyl)-1-oxopropan-2-yl)amino)-1-methoxy-2-methyl-3-oxopropyl)-2-azabicyclo[3.1.0]hexane-2-carboxylate 7c (173 mg, 0.33 mmol) was dissolved in 2 mL of dioxane, and added with a 5.6 M solution of hydrogen chloride in dioxane (0.21 mL, 1.16 mmol). The mixture was stirred under argon atmosphere for 1 hour at room temperature and then placed in a refrigerator for 12 hours at 0° C. The reaction solution was then concentrated under reduced pressure, and added with 5 mL of dichloromethane and 10 mL of saturated sodium bicarbonate solution, and stirred for 10 minutes. The aqueous phase was extracted with dichloromethane (5 mL×3). The combined dichloromethane phase was washed with saturated sodium chloride solution (10 mL), dried over anhydrous sodium sulfate, filtered and the filtrate was concentrated under reduced pressure to give the crude title product of (S)-tert-butyl 2-((2R,3R)-3-((1S,3S,5S)-2-azabicyclo[3 0.1.0]hexan-3-yl)-3-methoxy-2-methylpropanamido)-3-(2-fluorophenyl)propanoate 7d (77 mg, yellow liquid). The product was used in the next step without further purification.

MS m/z (ESI): 421.2 [M+1]

### Step 4

### (S)-tert-butyl 2-((2R,3R)-3-((1S,3S,5S)-2-((5S,8S,11S,12R)-11-((5)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate

The crude product of (S)-tert-butyl 2-((2R,3R)-3-((1S,3S,5S)-2-azabicyclo[3.1.0]hexan-3-yl)-3-methoxy-2-methylpropanamido)-3-(2-fluorophenyl)propanoate 7d (77 mg, 0.183 mmol), (5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid 1i (116.8 mg, 0.183 mmol) was dissolved in 6 mL of a mixed solvent of dichloromethane and dimethylformamide (V/V=5:1), and then added with N,N-diisopropylethylamine (0.16 mL, 0.915 mmol) and 2-(7-azobenzotriazole)-N,N,N′,N′-tetramethyluronium hexafluorophosphate (84 mg, 0.22 mmol). The reaction mixture was stirred under argon atmosphere at room temperature for 1 hour. The reaction mixture was then added with 6 mL of water and the dichloromethane phase was washed with saturated sodium chloride solution (10 mL) and dried over anhydrous sodium sulfate, filtrated and the filtrate was concentrated under reduced pressure. The residues were purified by silica gel column chromatography using eluent system B to give the title product of (S)-tert-butyl 2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate 7e (190.5 mg, yellow sticky material), yield 100%.

MS m/z (ESI): 1040.6 [M+1]

### Step 5

### (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate

(S)-tert-butyl 2-((2R,3R)-3-((1S,3S,5S)-2-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-2-azabicyclo[3.1.0]hexan-3-yl)-3-methoxy-2-methylpropanamido)-3-(2-fluorophenyl)propanoate 7e (190.5 mg, 0.183 mmol) was dissolved in 1.5 mL of dichloromethane and added with 2 mL of diethylamine. The reaction mixture was stirred under argon atmosphere at room temperature for 3 hours. After completion of the reaction, the reaction solution was concentrated under reduced pressure to give the crude title product of (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate 7f (150 mg, yellow sticky material). The product was used in the next step without further purification.

MS m/z (ESI): 818.5 [M+1]

### Step 6

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid

The crude product of (S)-tert-butyl 2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoate 7f (150 mg, 0.183 mmol) was dissolved in 1 mL of dioxane, and added with 3 mL of a 5.6 M solution of hydrogen in chloride dioxane. The resulting mixture was stirred under argon atmosphere for 12 hours at room temperature. After completion of the reaction, the reaction solution was concentrated under reduced pressure, and the residual solvent was spin-coated with ethylether. The residues were purified by high performance liquid chromatography to give the title product of (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid 7 (28 mg, white solid), yield 20%.

MS m/z (ESI): 762.7 [M+1]

¹H NMR (400 MHz, CD₃OD): δ 7.38-7.18 (m, 2H), 7.13-7.01 (m, 2H), 4.80-4.67 (m, 2H), 4.30-4.15 (m, 1H), 4.13-4.01 (m, 1H), 3.96-3.83 (m, 2H), 3.75-3.60 (m, 2H), 3.42-3.11 (m, 9H), 3.06-2.95 (m, 1H), 2.70-2.58 (m, 4H), 2.28-2.01 (m, 4H), 1.88-1.70 (m, 3H), 1.57-1.25 (m, 4H), 1.22-0.95 (m, 18H), 0.92-0.80 (m, 4H), 0.78-0.65 (m, 1H).

### Example 8

### (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3 0.1.0]hexan-3-yl)-3-methoxy-2-methylpropanamido)-3-(2-fluorophenyl)propanoic acid

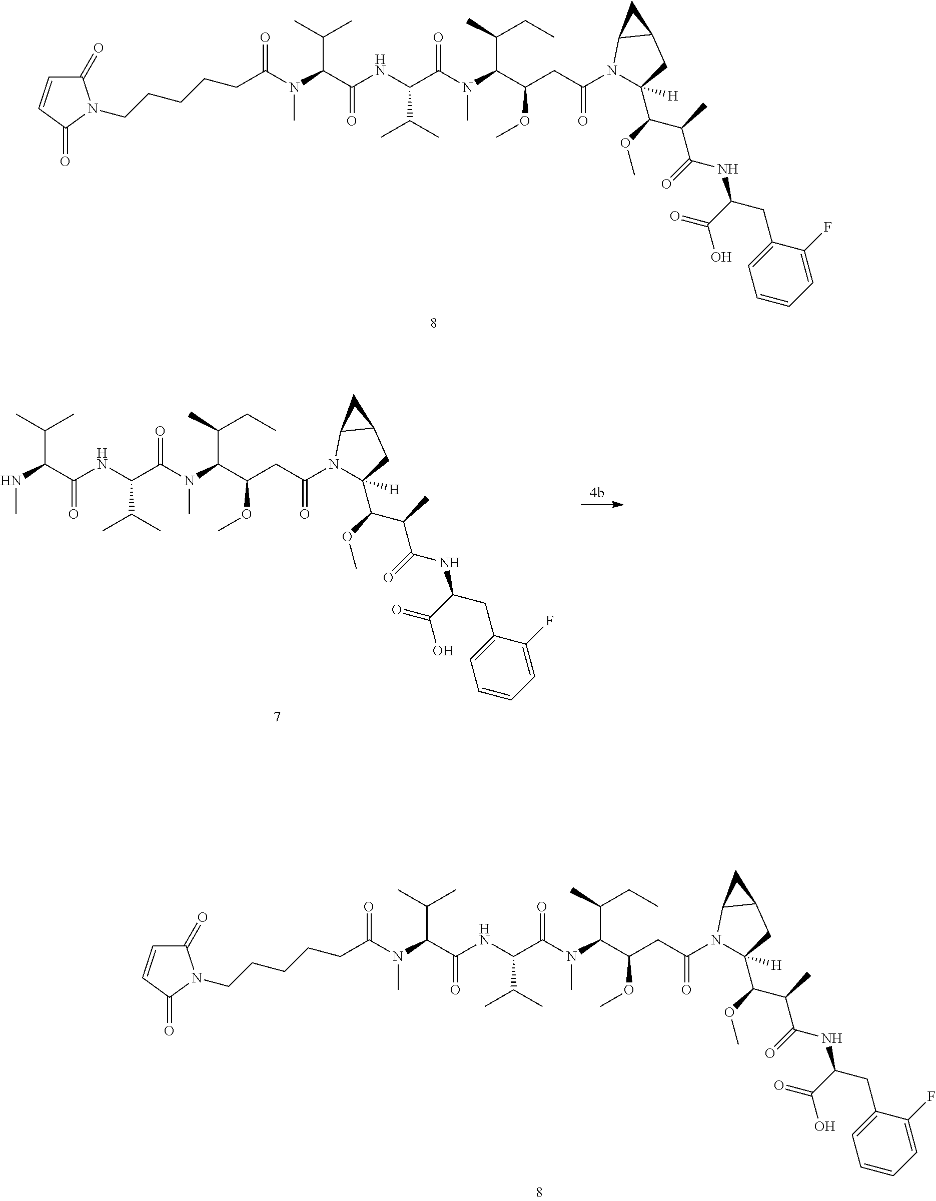 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(2-fluorophenyl)propanoic acid 7 (25 mg, 0.033 mmol) was dissolved in 3 mL of dichloromethane and added with N, N-diisopropylethylamine (0.029 mL, 0.164 mmol) The mixture was added dropwise with a preformed solution of 6-(2,5-dioxo-2,5-dihydro-1H-pyrrol-1-yl) hexanoyl chloride 4b (11.3 mg, 0.049 mmol) in dichloromethane under an argon atmosphere in an ice bath, and then stirred at room temperature for 3 hours. The reaction mixture was then added with 5 mL of water and stirred for 20 minutes. The organic phase was dried over anhydrous sodium sulfate, filtered, and the filtrate was concentrated under reduced pressure. The residues were purified by high performance liquid chromatography to give the title product of (S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-(2-fluorophenyl)propanoic acid 8 (7 mg, yellow sticky material), yield 22.4%.

MS m/z (ESI): 955.4 [M+1]

¹H NMR (400 MHz, CD₃OD): δ 7.36-7.30 (m, 1H), 7.29-7.21 (m, 1H), 7.17-7.02 (m, 2H), 6.83-6.79 (m, 2H), 4.81-4.71 (m, 2H), 4.69-4.55 (m, 2H), 4.25-4.15 (m, 1H), 4.13-4.04 (m, 1H), 3.96-3.85 (m, 2H), 3.70-3.61 (m, 1H), 3.55-3.46 (m, 3H), 3.40-3.21 (m, 4H), 3.18-3.10 (m, 2H), 3.07-2.96 (m, 4H), 2.67-2.56 (m, 2H), 2.54-2.34 (m, 3H), 2.29-2.17 (m, 2H), 2.10-1.99 (m, 1H), 1.89-1.57 (m, 7H), 1.52-1.28 (m, 6H), 1.21-1.11 (m, 4H), 1.07-0.96 (m, 6H), 0.95-0.81 (m, 12H), 0.80-0.69 (m, 1H).

### Example 9

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoic acid

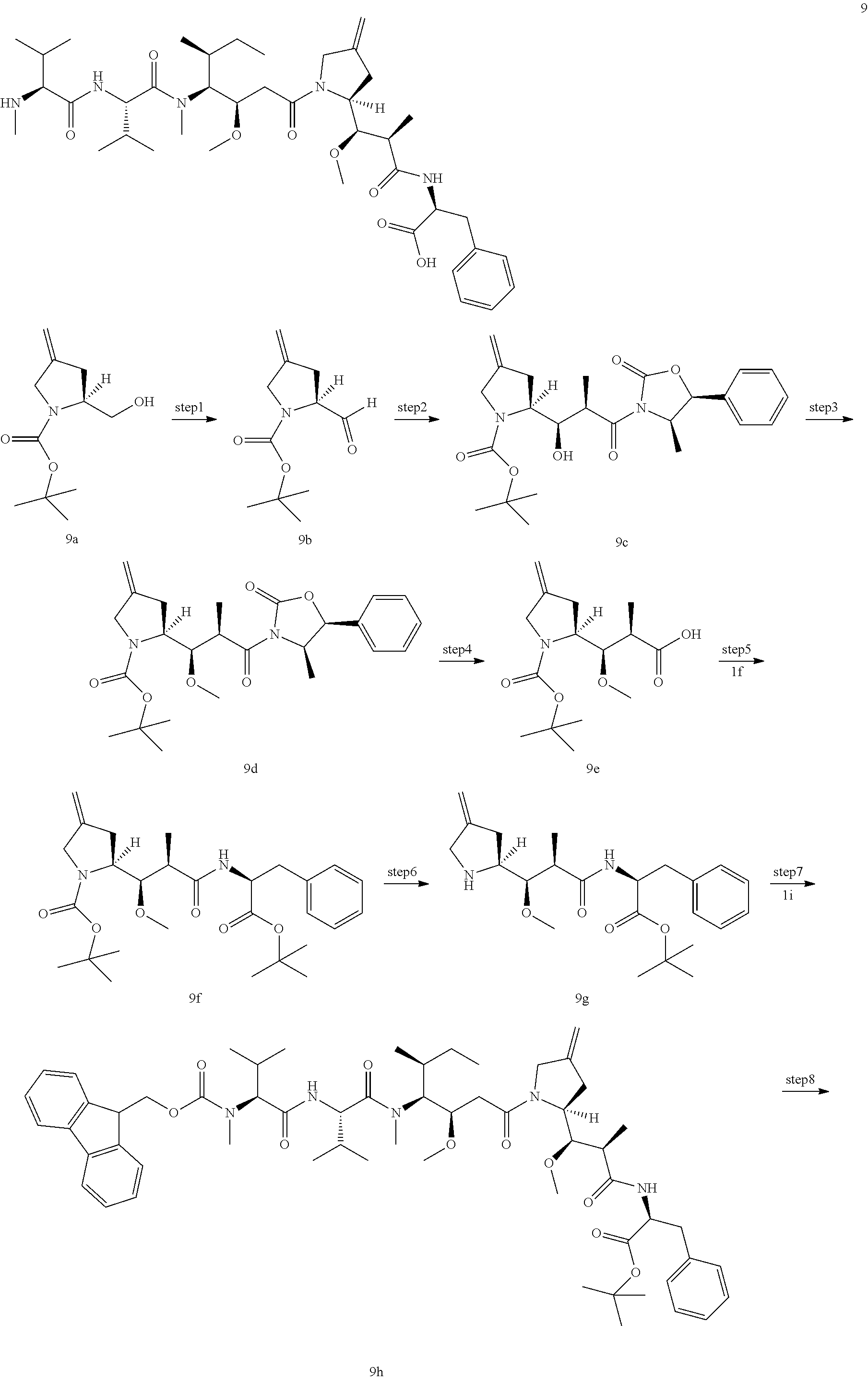

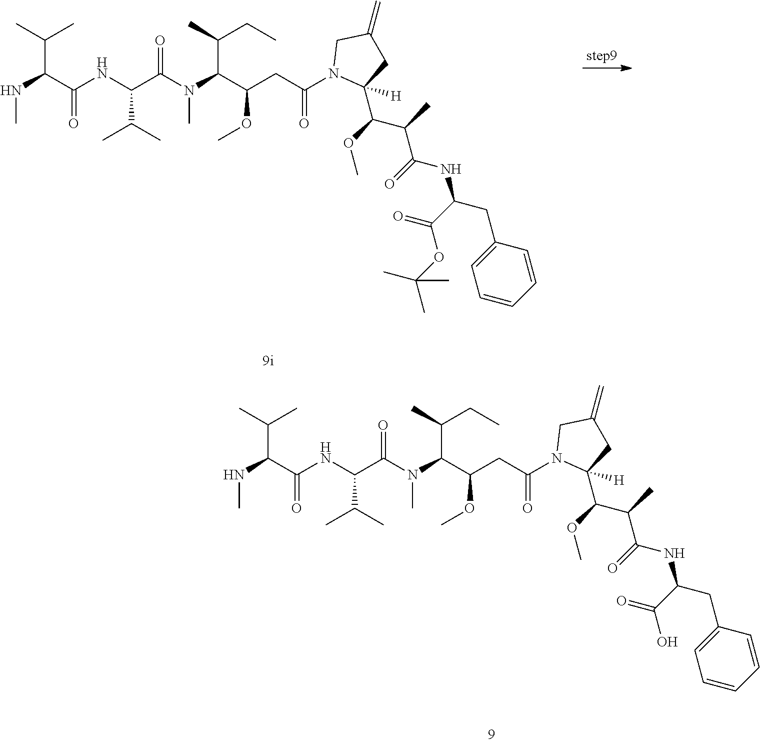 [SMILES: C=C1C[C@@H]([C@H](OC)[C@@H](C)C(=O)N[C@@H](Cc2ccccc2)C(=O)OC(C)(C)C)N(C(=O)C[C@@H](OC)[C@H]([C@@H](C)CC)N(C)C(=O)[C@@H](NC(=O)[C@@H](NC)C(C)C)C(C)C)C1 ; C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@@H](NC)C(C)C)C(C)C)C1]

### Step 1

### (S)-tert-butyl 2-formyl-4-methylenepyrrolidine-1-carboxylate

(S)-tert-butyl 2-(hydroxymethyl)-4-methylenepyrrolidine-1-carboxylate 9a (1.32 g, 6.19 mmol, prepared according to the known method of “From Journal of Organic Chemistry, 2003, 68(10), 3923-3931”) was dissolved in 15 mL of dichloromethane and cooled to 0° C. The mixture was added with N,N-diisopropylethylamine (5.38 mL, 30.9 mmol), dimethyl sulfoxide (7.26 g, 92.9 mmol) and sulfur trioxide-pyridine complex (7.26 g, 92.9 mmol) under an argon atmosphere, and stirred at 0° C. for 3 hours. The reaction was then quenched by phosphate buffer (pH=7), and washed successively with water and saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-formyl-4-methylenepyrrolidine-1-carboxylate 9b (1.1 g, light yellow liquid), yield 84.2%.

### Step 2

### (S)-tert-butyl 2-((1R,2R)-1-hydroxy-2-methyl-3-((4R,5S)-4-methyl-2-oxo-5-phenyloxazolidin-3-yl)-3-oxopropyl)-4-methylenepyrrolidine-1-carboxylate

(4R,5S)-4-methyl-5-phenyl-3-propionyloxazolidin-2-one 1b (1.43 g, 6.15 mmol) was dissolved in 30 mL of dichloromethane, and cooled to 0° C. under an argon atmosphere. The solution was added dropwise with triethylamine (0.98 mL, 7.08 mmol) and dibutylboron trifluoromethanesulfonate (6.65 mL, 6.65 mmol), and then stirred at 0° C. for 50 min. The reaction mixture was cooled to −78° C. and added with a preformed solution of (S)-tert-butyl 2-formyl-4-methylenepyrrolidine-1-carboxylate 9b (1.43 g, 6.15 mmol) in dichloromethane, then the mixture was stirred at −78° C. for 2 hours, at 0° C. for 1 hour, and at room temperature for 1 hour. The reaction mixture was quenched by a mixture of 36 mL of phosphate buffer (pH=7) and methanol (V/V=3:1), then added with a mixture of methanol and hydrogen peroxide (V/V=1:2) at 0° C., and stirred at room temperature for 1 hour. After completion of the reaction, the methanol and the organic phase were removed. The residual aqueous phase was extracted with methylene chloride, the organic phases were combined, washed successively with water and saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((1R,2R)-1-hydroxy-2-methyl-3-((4R,5S)-4-methyl-2-oxo-5-phenyloxazolidin-3-yl)-3-oxopropyl)-4-methylenepyrrolidine-1-carboxylate 9c (810 mg, white foam solid), yield 30%.

MS m/z (ESI): 354.2 [M-100+1]

### Step 3

### (S)-tert-butyl 2-((1R,2R)-1-methoxy-2-methyl-3-((4R,5S)-4-methyl-2-oxo-5-phenyloxazolidin-3-yl)-3-oxopropyl)-4-methylenepyrrolidine-1-carboxylate

(S)-tert-butyl 2-((1R,2R)-1-hydroxy-2-methyl-3-((4R,5S)-4-methyl-2-oxo-5-phenyloxazolidin-3-yl)-3-oxopropyl)-4-methylenepyrrolidine-1-carboxylate 9c (810 mg, 1.82 mmol) was dissolved in 15 mL of dichloromethane, and added with crushed molecular sieves, potassium carbonate (1.25 g, 9.11 mmol), and methyl trifluoromethanesulfonate (897.7 mg, 5.47 mmol). Then the mixture was stirred at room temperature for 12 hours. After completion of the reaction, the reaction solution was filtered, and the filter cake was washed with dichloromethane. The organic phases were combined and concentrated under reduced pressure. The residues were purified by silica gel column chromatography using eluent system B to give the title product of (S)-tert-butyl 2-((1R,2R)-1-methoxy-2-methyl-3-((4R,5S)-4-methyl-2-oxo-5-phenyloxazolidin-3-yl)-3-oxopropyl)-4-methylenepyrrolidine-1-carboxylate 9d (280 mg, white foam solid), yield 33.5%.

MS m/z (ESI): 359.2 [M-100+1]

### Step 4

### (2R,3R)-3-45)-1-(tert-butoxy carbonyl)-4-methylenepyrrolidin-2-yl)-3-methoxy-2-methylpropanoic acid

(S)-tert-butyl 2-((1R,2R)-1-methoxy-2-methyl-3-((4R,5S)-4-methyl-2-oxo-5-phenyloxazolidin-3-yl)-3-oxopropyl)-4-methylenepyrrolidine-1-carboxylate 9d (400 mg, 0.87 mmol) was dissolved in 20 mL of tetrahydrofuran, and added with lithium hydroxide monohydrate (62.2 mg, 1.484 mmol). The reaction mixture was cooled to 0° C. under an argon atmosphere, and then added with 30% hydrogen peroxide (112.7 mg, 3.31 mmol) dropwise. The mixture was allowed to react at room temperature for 12 hours. After completion of the reaction, sodium sulfite (416 mg, 3.3 mmol) was added to the reaction solution, and the mixture was stirred at room temperature for 1 hour. The tetrahydrofuran was concentrated under reduced pressure and the residues were dissolved in water and extracted with dichloromethane. The aqueous phase was diluted with dilute hydrochloric acid to adjust the pH to 3 to 4, and then extracted with dichloromethane (30 mL×3). The combined organic phases were washed successively with water and saturated sodium chloride solution, dried over anhydrous sodium sulfate, filtered and the filtrate was concentrated under reduced pressure to give the crude title product of (2R,3R)-3-((S)-1-(tert-butoxy carbonyl)-4-methylenepyrrolidin-2-yl)-3-methoxy-2-methylpropanoic acid 9e (230 mg, colorless liquid). The product was used in the next step without further purification.

MS m/z (ESI): 298.2 [M-1]

### Step 5

### (S)-tert-butyl 2-((1R,2R)-3-(((S)-1-(tert-butoxy)-1-oxo-3-phenylpropan-2-yl)amino)-1-methoxy-2-methyl-3-oxopropyl)-4-methylenepyrrolidine-1-carboxylate

(2R,3R)-3-((S)-1-(tert-butoxy carbonyl)-4-methylenepyrrolidin-2-yl)-3-methoxy-2-methylpropanoic acid 9e (220 mg, 0.735 mmol) was dissolved in 6 mL of dichloromethane, and added with (S)-tert-butyl 2-amino-3-phenylpropanoate if (178.8 mg, 0.809 mmol), N,N-diisopropylethylamine (0.51 mL, 2.94 mmol) and 2-(7-azobenzotriazole)-N,N,N′,N′-tetramethyluronium hexafluorophosphate (363 mg, 0.956 mmol). The reaction mixture was stirred under an argon atmosphere at room temperature for 3 hours. After completion of the reaction, the reaction solution was washed successively with water and a saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((1R,2R)-3-(((S)-1-(tert-butoxy)-1-oxo-3-phenylpropan-2-yl)amino)-1-methoxy-2-methyl-3-oxopropyl)-4-methylenepyrrolidine-1-carboxylate 9f (271 mg, white solid), yield 66%.

MS m/z (ESI): 503.3 [M+1]

### Step 6

### (S)-tert-butyl 2-((2R,3R)-3-methoxy-2-methyl-3-((S)-4-methylenepyrrolidin-2-yl)propanamido)-3-phenylpropanoate

(S)-tert-butyl 2-((1R,2R)-3-(((S)-1-(tert-butoxy)-1-oxo-3-phenylpropan-2-yl)amino)-1-methoxy-2-methyl-3-oxopropyl)-4-methylenepyrrolidine-1-carboxylate 9f (270 mg, 0.537 mmol) was dissolved in 4 mL of 1,4-dioxane, and added with a 4 M solution of hydrogen in chloride dioxane (0.335 mL, 1.881 mmol). The mixture was stirred for 1 hour at room temperature and placed in a refrigerator for 12 hours at 0 to 4° C. The reaction mixture was concentrated under reduced pressure, and the residues were dissolved in methylene chloride and added with saturated sodium bicarbonate solution to adjust the pH to 8 to 9. The aqueous phase was extracted with methylene chloride. The organic phases were combined, washed successively with water and saturated sodium chloride solution, dried over anhydrous sodium sulfate, filtered and the filtrate was concentrated under reduced pressure to give the crude title product of (S)-tert-butyl 2-((2R,3R)-3-methoxy-2-methyl-3-((S)-4-methylenepyrrolidin-2-yl)propanamido)-3-phenylpropanoate 9g (210 mg, light yellow oil). The product was used in the next step without further purification.

MS m/z (ESI): 403.4 [M+1]

### Step 7

### (S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-4-methylenepyrrolidin-2-yl)-3-methoxy-2-methylpropanamido)-3-phenylpropanoate

The crude product of (S)-tert-butyl 2-((2R,3R)-3-methoxy-2-methyl-3-((S)-4-methylenepyrrolidin-2-yl)propanamido)-3-phenylpropanoate 9g (210 mg, 0.521 mmol), (5S,8S,11S,12R)-11-((S)-sec-butyl)-1-(9H fluoren-9-yl)-5,8-diisopropyl-12-methoxy-4,10-dimethyl-3,6,9-trioxo-2-oxa-4,7,10-triazatetradecan-14-oic acid 1i (365.9 mg, 0.547 mmol) was dissolved in 6 mL of dichloromethane, and added with N,N-diisopropylethylamine (0.4537 mL, 2.609 mmol) and 2-(7-azobenzotriazole)-N,N,N′,N′-tetramethyluronium hexafluorophosphate (257.8 mg, 0.678 mmol). The reaction mixture was stirred under an argon atmosphere at room temperature for 2 hours. After completion of the reaction, the reaction solution was washed successively with water and a saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-4-methylenepyrrolidin-2-yl)-3-methoxy-2-methylpropanamido)-3-phenylpropanoate 9h (345 mg, white foam solid), yield 64.7%.

MS m/z (ESI): 1022.5 [M+1]

### Step 8

### (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoate

(S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-4-methylenepyrrolidin-2-yl)-3-methoxy-2-methylpropanamido)-3-phenylpropanoate 9h (345 mg, 0.337 mmol) was dissolved in 2 mL of dichloromethane, and added with 3 mL of diethylamine. The reaction mixture was stirred under an argon atmosphere at room temperature for 2 hours. After completion of the reaction, the reaction solution was concentrated under reduced pressure to give the crude title product of (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoate 9i (375 mg, yellow oil). The product was used in the next step without further purification.

MS m/z (ESI): 800.5 [M+1]

### Step 9

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoic acid

The crude product of (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoate 9i (370 mg, 0.462 mmol) was added with 7 mL of a 4M solution of hydrogen chloride in dioxane solution. The reaction system was sealed and stirred at room temperature for 12 hours. After completion of the reaction, the reaction solution was concentrated under reduced pressure. The residues were purified by high performance liquid chromatography to obtain the product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoic acid 9 (30 mg, white solid), yield 11.9%.

MS m/z (ESI): 744.7 [M+1]

¹H NMR (400 MHz, CDCl₃) δ 10.57 (s, 1H), 10.00 (s, 1H), 8.41-8.26 (m, 2H), 7.52-6.99 (m, 5H), 5.34 (s, 1H), 5.02-4.95 (m, 4H), 4.38-4.33 (m, 2H), 4.16 (m, 2H), 3.93-3.75 (m, 3H), 3.49-3.07 (m, 6H), 2.94-2.32 (m, 16H), 2.30-2.01 (m, 4H), 1.75-1.65 (m, 2H), 1.32-0.83 (m, 16H).

### Example 10

(S)-2-((2R,3R)-3-((S)-1-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-4-methylenepyrrolidin-2-yl)-3-methoxy-2-methylpropanamido)-acid

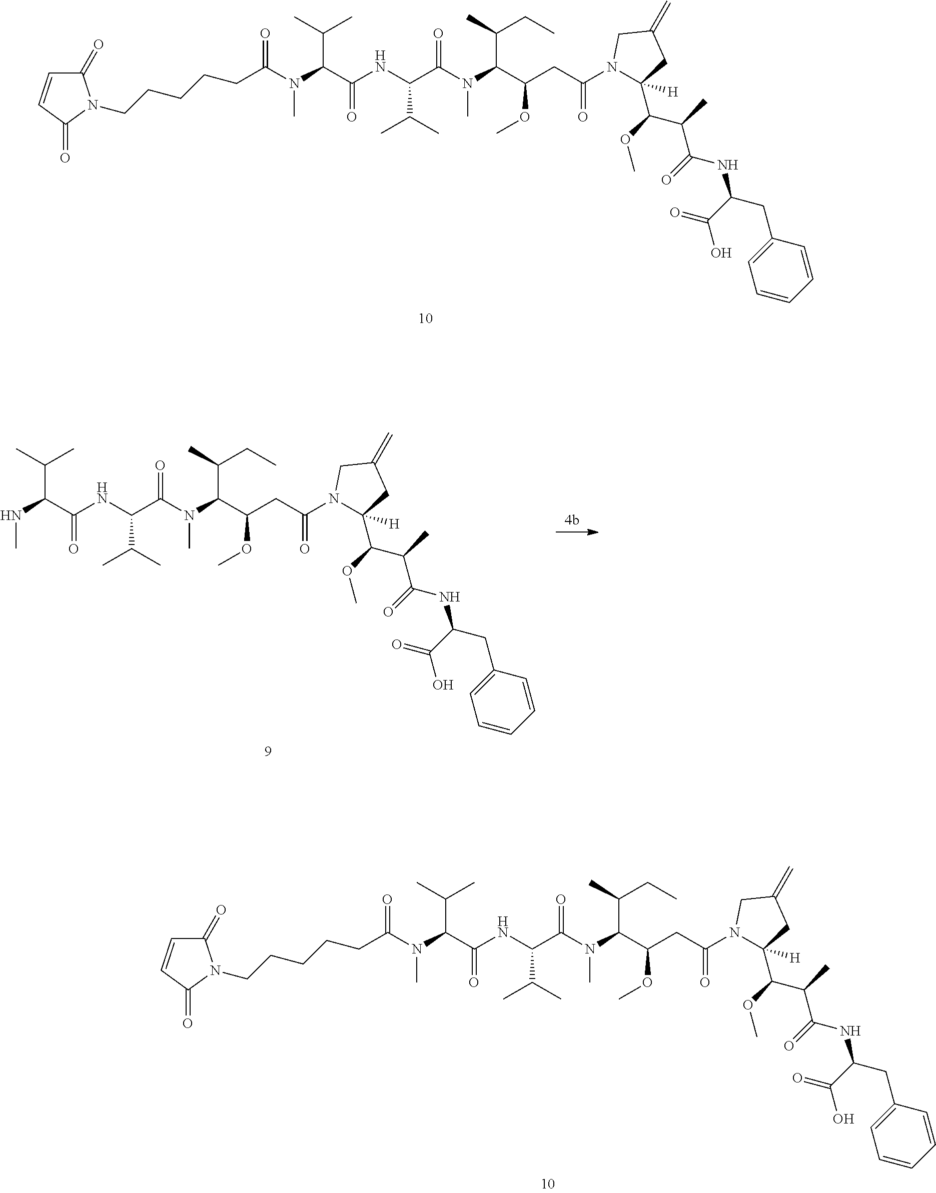 [SMILES: C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@H](C(C)C)N(C)C(=O)CCCCCN2C(=O)C=CC2=O)C(C)C)C1 ; C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@@H](NC)C(C)C)C(C)C)C1 ; C=C1C[C@@H]([C@H](OC)[C@@H](C)C(=O)N[C@@H](Cc2ccccc2)C(=O)O)N(C(=O)C[C@@H](OC)[C@H]([C@@H](C)CC)N(C)C(=O)[C@@H](NC(=O)[C@H](C(C)C)N(C)C(=O)CCCCCN2C(=O)C=CC2=O)C(C)C)C1]

(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-4-methylenepyrrolidin-2-yl)-3-methoxy-2-methylpropanamido)-3-phenylpropanoic acid 9 (22 mg, 0.0295 mmol) was dissolved in 3 mL of dichloromethane, and added with N,N-diisopropylethylamine (1 9 mg, 0.1479 mmol). The mixture was cooled to 0° C., and added dropwise with a preformed solution of 6-(2,5-dioxo-2,5-dihydro-1H-pyrrol-1-yl) hexanoyl chloride 4b (10.1 mg, 0.0443 mmol) in dichloromethane. The reaction mixture was stirred under an argon atmosphere at room temperature for 2.5 hours. The reaction was then quenched with methanol, and then concentrated under reduced pressure. The residues were purified by high performance liquid chromatography to obtain the title product of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-methylenepyrrolidin-2-yl)-3-methoxy-2-methylpropanamido)-3-phenylpropanoic acid 10 (1.3 mg, white sticky material), yield 4.6%.

MS m/z (ESI): 937.9 [M+1]

### Example 11

### (S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoic acid

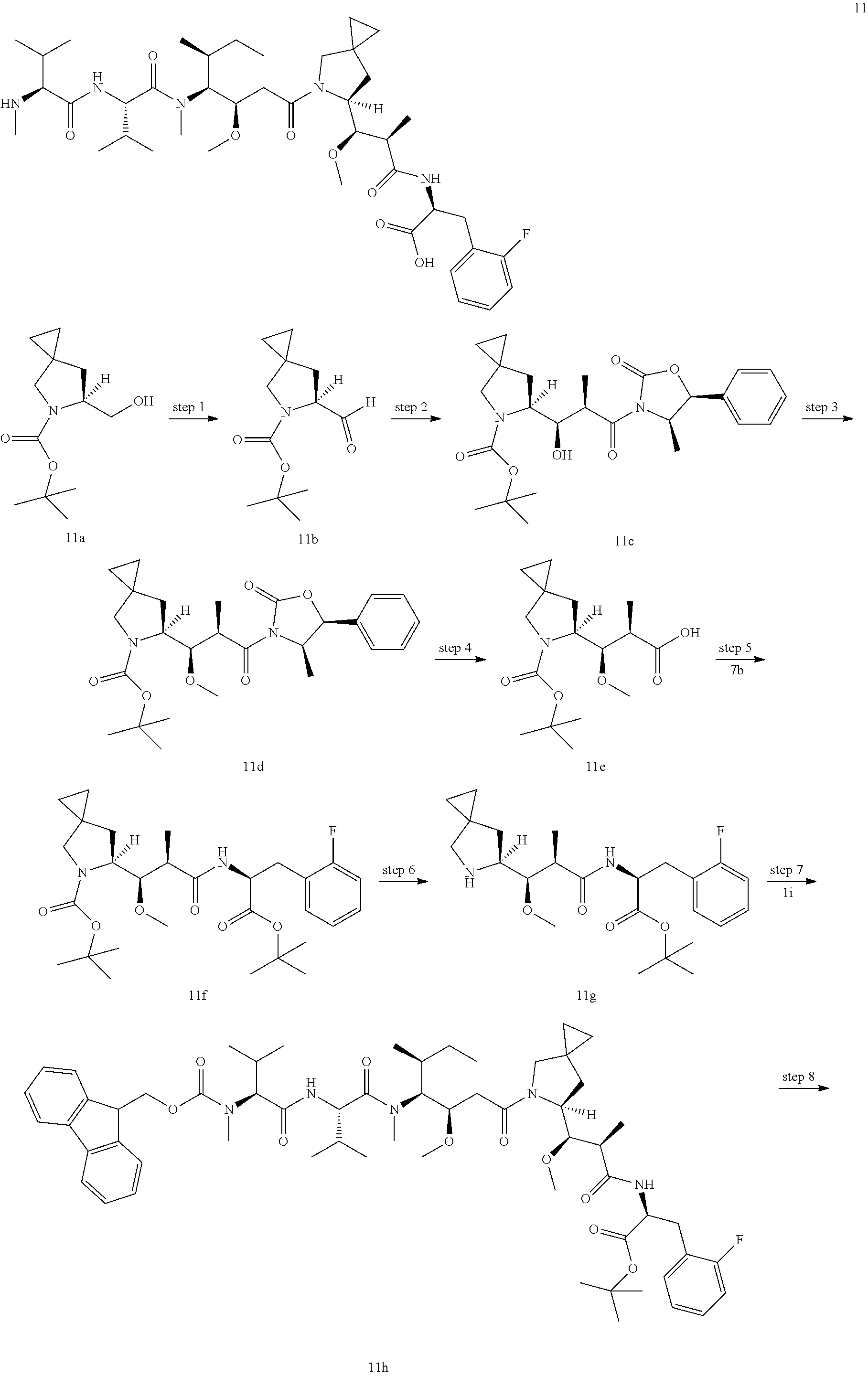

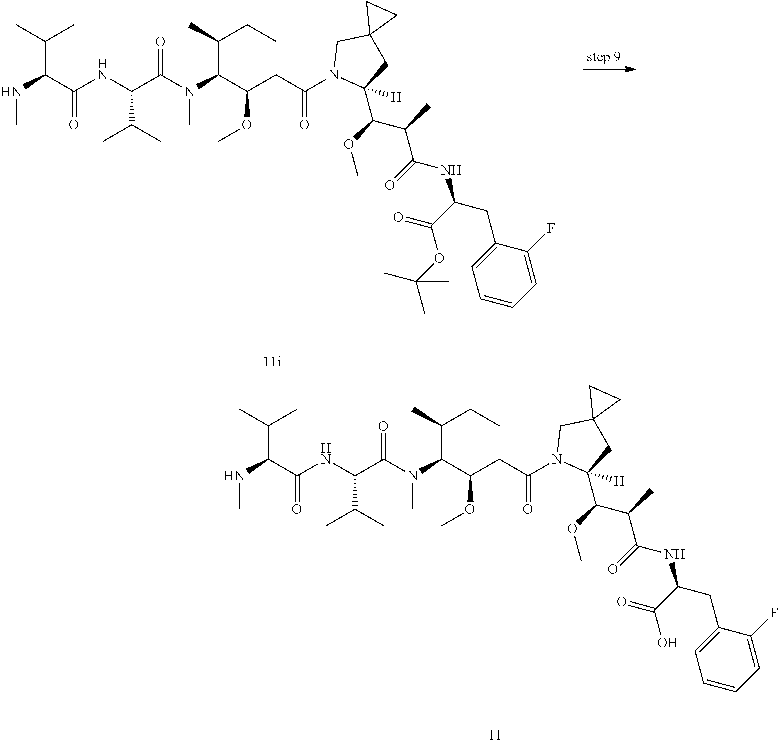 [SMILES: CC[C@H](C)[C@@H]([C@@H](CC(=O)N1CC2(CC2)C[C@H]1[C@H](OC)[C@@H](C)C(=O)N[C@@H](Cc1ccccc1F)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C]

### Step 1

### (S)-tert-butyl 6-formyl-5-azaspiro[2.4]heptane-5-carboxylate

(S)-tert-butyl 6-(hydroxymethyl)-5-azaspiro[2.4]heptane-5-carboxylate 11a (2.37 g, 10.4 mmol, prepared according to the known method of “Bioorganic & Medicinal Chemistry Letters, 2013, 23(9), 2653-2658”) was dissolved in 40 mL of dichloromethane, and cooled to 0° C. The solution was added with N,N-diisopropylethylamine (10.8 mL, 62.5 mmol), dimethylsulfoxide (12.2 g, 156.4 mmol) and a sulfur trioxide-pyridine complex (6.63 g, 41.7 mmol) under an argon atmosphere. The reaction mixture was stirred at 0° C. for 3 hours. After completion of the reaction, the reaction mixture was quenched by addition of phosphate buffer (pH=7), and washed successively with water and saturated sodium chloride solution, dried over anhydrous sodium sulfate, filtered and the filtrate was concentrated under reduced pressure. The residues were purified by silica gel column chromatography using eluent system B to give the title product of (S)-tert-butyl 6-formyl-5-azaspiro[2.4]heptane-5-carboxylate 11b (2 g, yellow liquid), yield 88%.

### Step 2

### (S)-tert-butyl 6-((1R,2R)-1-hydroxy-2-methyl-3-((4R,5S)-4-methyl-2-oxo-5-phenyloxazolidin-3-yl)-3-oxopropyl)-5-azaspiro[2.4]heptane-5-carboxylate

(4R,5S)-4-methyl-5-phenyl-3-propionyloxazolidin-2-one 1b (2 g, 8.88 mmol) was dissolved in 35 mL of dichloromethane and added with triethylamine (1.42 mL, 10.2 mmol) dropwise under an argon atmosphere. The mixture was cooled to 0° C., and added with dibutylboron trifluoromethanesulfonate (9.59 mL, 9.59 mmol) dropwise, and then stirred at 0° C. for 50 minutes. A preformed solution of (S)-tert-butyl 6-formyl-5-azaspiro[2.4]heptane-5-carboxylate 11b (2 g, 8.88 mmol) in dichloromethane was added at 78° C. and stirred at −78° C. for 2 hours, then stirred at 0° C. for 1 hour and at room temperature for 1 hour. The reaction mixture was quenched by the addition of a mixture of phosphate buffer (pH=7.0) and methanol (V/V=3:1). A mixture of methanol and hydrogen peroxide (V/V=1:2) was added at 0° C., and the mixture was stirred at room temperature for 1 hour. After concentration under reduced pressure, the residues were dissolved in water and extracted with methylene chloride. The organic phases were combined, washed successively with water and saturated sodium chloride solution, dried over anhydrous sodium sulfate, filtered and the filtrate was concentrated under reduced pressure. The resulting residues were purified by silica gel column chromatography using eluent system B to give the title product of (S)-tert-butyl 6-((1R,2R)-1-hydroxy-2-methyl-3-((4R,5S)-4-methyl-2-oxo-5-phenyloxazolidin-3-yl)-3-oxopropyl)-5-azaspiro[2.4]heptane-5-carboxylate 11c (1.8 g, white foam solid), yield 44.2%.

MS m/z (ESI): 459.4 [M+1]

### Step 3

### (S)-tert-butyl 6-((1R,2R)-1-methoxy-2-methyl-3-((4R,5S)-4-methyl-2-oxo-5-phenyloxazolidin-3-yl)-3-oxopropyl)-5-azaspiro[2.4]heptane-5-carboxylate

(S)-tert-butyl 6-((1R,2R)-1-hydroxy-2-methyl-3-((4R,5S)-4-methyl-2-oxo-5-phenyloxazolidin-3-yl)-3-oxopropyl)-5-azaspiro[2.4]heptane-5-carboxylate 11c (1.8 g, 3.92 mmol) was dissolved in 30 mL of dichloromethane, and added with crushed molecular sieves. Potassium carbonate (3.78 g, 27.48 mmol) and methyl trifluoromethanesulfonate (3.22 g, 19.64 mmol) was added under an argon atmosphere and the reaction mixture was stirred at room temperature for 12 hours. After completion of the reaction, the reaction solution was filtered and the filter cake was washed with dichloromethane. The organic phases were combined and the organic phase was concentrated under reduced pressure. The residues were purified by silica gel column chromatography using eluent system A to give the title product of (S)-tert-butyl 6-((1R,2R)-1-methoxy-2-methyl-3-((4R,5S)-4-methyl-2-oxo-5-phenyloxazolidin-3-yl)-3-oxopropyl)-5-azaspiro[2.4]heptane-5-carboxylate 11d (930 mg, colorless oil), yield 50.2%.

MS m/z (ESI): 473.4 [M+1]

### Step 4

### (2R,3R)-3-((S)-5-(tert-butoxycarbonyl)-5-azaspiro[2.4]heptan-6-yl)-3-methoxy-2-methylpropanoic acid

(S)-tert-butyl 6-((1R,2R)-1-methoxy-2-methyl-3-((4R,5S)-4-methyl-2-oxo-5-phenyloxazolidin-3-yl)-3-oxopropyl)-5-azaspiro[2.4]heptane-5-carboxylate 11d (1.03 g, 2.8 mmol) was dissolved in 20 mL of tetrahydrofuran, and added with lithium hydroxide monohydrate (155 mg, 3.7 mmol). 30% hydrogen peroxide (939 mg, 8.28 mmol) was added dropwise under an argon atmosphere. The reaction mixture was stirred at room temperature for 12 hours. After completion of the reaction, sodium sulfite (1.04 g, 8.28 mmol) was added to the reaction solution, and the mixture was stirred at room temperature for 1 hour. The tetrahydrofuran was concentrated under reduced pressure and the residues were dissolved in water and extracted with dichloromethane. The aqueous phase was diluted with dilute hydrochloric acid to adjust the pH to 3 to 4, and then extracted with dichloromethane (30 mL×5). The combined organic phases were washed successively with water and saturated sodium chloride solution, dried over anhydrous sodium sulfate, filtered and the filtrate was concentrated under reduced pressure to give the crude title product of (2R,3R)-3-((S)-5-(tert-butoxy carbonyl)-5-azaspiro[2.4]heptan-6-yl)-3-methoxy-2-methylpropanoic acid 11e (700 mg, colorless viscous liquid). The product was used in the next step without purification.

MS m/z (ESI): 314.4 [M+1]

### Step 5

### (S)-tert-butyl 6-((1R,2R)-3-(((S)-1-(tert-butoxy)-3-(2-fluorophenyl)-1-oxopropan-2-yl)amino)-1-methoxy-2-methyl-3-oxopropyl)-5-azaspiro[2.4]heptane-5-carboxylate

The crude product of (2R,3R)-3-((S)-5-(tert-butoxy carbonyl)-5-azaspiro[2.4]heptan-6-yl)-3-methoxy-2-methylpropanoic acid 11e (350 mg, 1.117 mmol) was dissolved in 6 mL of dichloromethane, and added with (S)-tert-butyl 2-amino-3-(2-fluorophenyl)propanoate 7b (267 mg, 1.117 mmol). The mixture was added with N,N-diisopropylethylamine (720 mg, 5.587 mmol) and 2-(7-azobenzotriazole)-N,N,N′,N′-tetramethyluronium hexafluorophosphate (509.8 mg, 1.341 mmol) under an argon atmosphere, and then stirred at room temperature for 2 hours. After completion of the reaction, the reaction solution was washed successively with water and saturated sodium chloride solution, dried over anhydrous sodium sulfate, filtered and the filtrate was concentrated under reduced pressure. The residues were purified by silica gel column chromatography using eluent system B to give the title product of (S)-tert-butyl 6-((1R,2R)-3-(((S)-1-(tert-butoxy)-3-(2-fluorophenyl)-1-oxopropan-2-yl)amino)-1-methoxy-2-methyl-3-oxopropyl)-5-azaspiro[2.4]heptane-5-carboxylate 11f (570 mg, colorless oil), yield 95.3%.

MS m/z (ESI): 535.3 [M+1]

### Step 6

### (S)-tert-butyl 3-(2-fluorophenyl)-2-((2R,3R)-3-methoxy-2-methyl-3-((S)-5-azaspiro[2.4]heptan-6-yl)propanamido)propanoate

(S)-tert-butyl 6-((1R,2R)-3-(((S)-1-(tert-butoxy)-3-(2-fluorophenyl)-1-oxopropan-2-yl)amino)-1-methoxy-2-methyl-3-oxopropyl)-5-azaspiro[2.4]heptane-5-carboxylate 11f (570 mg, 1.049 mmol) was dissolved in 8 mL of 1,4-dioxane, and added with a 4 M solution of hydrogen chloride in dioxane (0.749 mL, 4.196 mmol). The mixture was stirred for 1 hour at room temperature and placed in a refrigerator for 12 hours at 0-4° C. The reaction mixture was concentrated under reduced pressure, and the residues were dissolved in methylene chloride. The saturated sodium bicarbonate solution was added dropwise to adjust the pH to 8 to 9, and the aqueous phase was extracted with methylene chloride. The organic phases were combined, washed successively with water and saturated sodium chloride solution, dried over anhydrous sodium sulfate, filtered and the filtrate was concentrated under reduced pressure to give the crude title product of (S)-tert-butyl 3-(2-fluorophenyl)-2-((2R,3R)-3-methoxy-2-methyl-3-((S)-5-azaspiro[2.4]heptan-6-yl)propanamido)propanoate 11g (440 mg, light yellow oil). The product was used in the next step without further purification.

MS m/z (ESI): 435.4 [M+1]

### Step 7

### (S)-tert-butyl 2-((2R,3R)-3-((S)-5-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-5-azaspiro[2.4]heptan-6-yl)-3-methoxy-2-methylpropanamido)-3-(2-fluorophenyl)propanoate

The crude product of (S)-tert-butyl 3-(2-fluorophenyl)-2-((2R,3R)-3-methoxy-2-methyl-3-((S)-5-azaspiro[2.4]heptan-6-yl)propanamido)propanoate 11g (440 mg, 1.013 mmol), (5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid 1i (645.8 mg, 1.013 mmol) was dissolved in 10 mL of dichloromethane, and added with N, N-diisopropylethylamine (0.88 mL, 5.06 mmol) and 2-(7-azobenzotriazole)-N,N,N′,N′-tetramethyluronium hexafluorophosphate (500.5 mg, 1.317 mmol). The reaction mixture was stirred under an argon atmosphere at room temperature for 2 hours. After completion of the reaction, the reaction solution was successively washed with water and a saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((2R,3R)-3-((S)-5-((5S,8S,11S,12R)-11-((5)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-5-azaspiro[2.4]heptan-6-yl)-3-methoxy-2-methylpropanamido)-3-(2-fluorophenyl)propanoate 11h (570 mg, white solid), yield 53.4%.

MS m/z (ESI): 1054.9 [M+1]

### Step 8

### (S)-tert-butyl 2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoate

(S)-tert-butyl 2-((2R,3R)-3-((S)-5-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)-5-azaspiro[2.4]heptan-6-yl)-3-methoxy-2-methylpropanamido)-3-(2-fluorophenyl)propanoate 11h (560 mg, 0.531 mmol) was dissolved in 2 mL of dichloromethane, and added with 6 mL of diethylamine. The mixture was stirred at room temperature for 2.5 hours. After completion of the reaction, the reaction solution was concentrated under reduced pressure to give the crude title product of (S)-tert-butyl 2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoate 11i (550 mg, white sticky material). The product was used in the next step without further purification.

MS m/z (ESI): 832.5 [M+1]

### Step 9

### (S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoic acid

The crude product of (S)-tert-butyl 2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoate 11i (450 mg, 0.541 mmol) was added with 7 mL of a 4M solution of hydrogen chloride in dioxane. The reaction system was sealed and stirred at room temperature for 12 hours. After completion of the reaction, the reaction solution was concentrated under reduced pressure. The residues were purified by high performance liquid chromatography to obtain the title product (S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoic acid 11 (403 mg, white solid), yield 96%.

MS m/z (ESI): 776.7 [M+1]

¹H NMR (400 MHz, CD₃OD) δ 7.30-7.22 (m, 2H), 7.12-7.04 (m, 2H), 4.72-4.68 (m, 2H), 4.13-4.07 (m, 2H), 3.96-3.94 (m, 1H), 3.70-3.66 (m, 2H), 3.50-3.47 (m, 2H), 3.40-3.37 (m, 3H), 3.34-3.28 (m, 4H), 3.26-3.22 (m, 2H), 3.11 (s, 1H), 3.05-2.91 (m, 2H), 2.67-2.65 (m, 3H), 2.57-2.43 (m, 2H), 2.39-2.28 (m, 2H), 2.25-2.16 (m, 3H), 1.93-1.88 (m, 2H), 1.55-1.43 (m, 2H), 1.23-1.21 (d, 2H), 1.16-1.08 (m, 3H), 1.08-0.97 (m, 10H), 0.89-0.83 (m, 3H), 0.66-0.53 (m, 3H), 0.46-0.43 (m, 2H).

### Example 12

### (S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-(2-fluorophenyl)propanoic acid

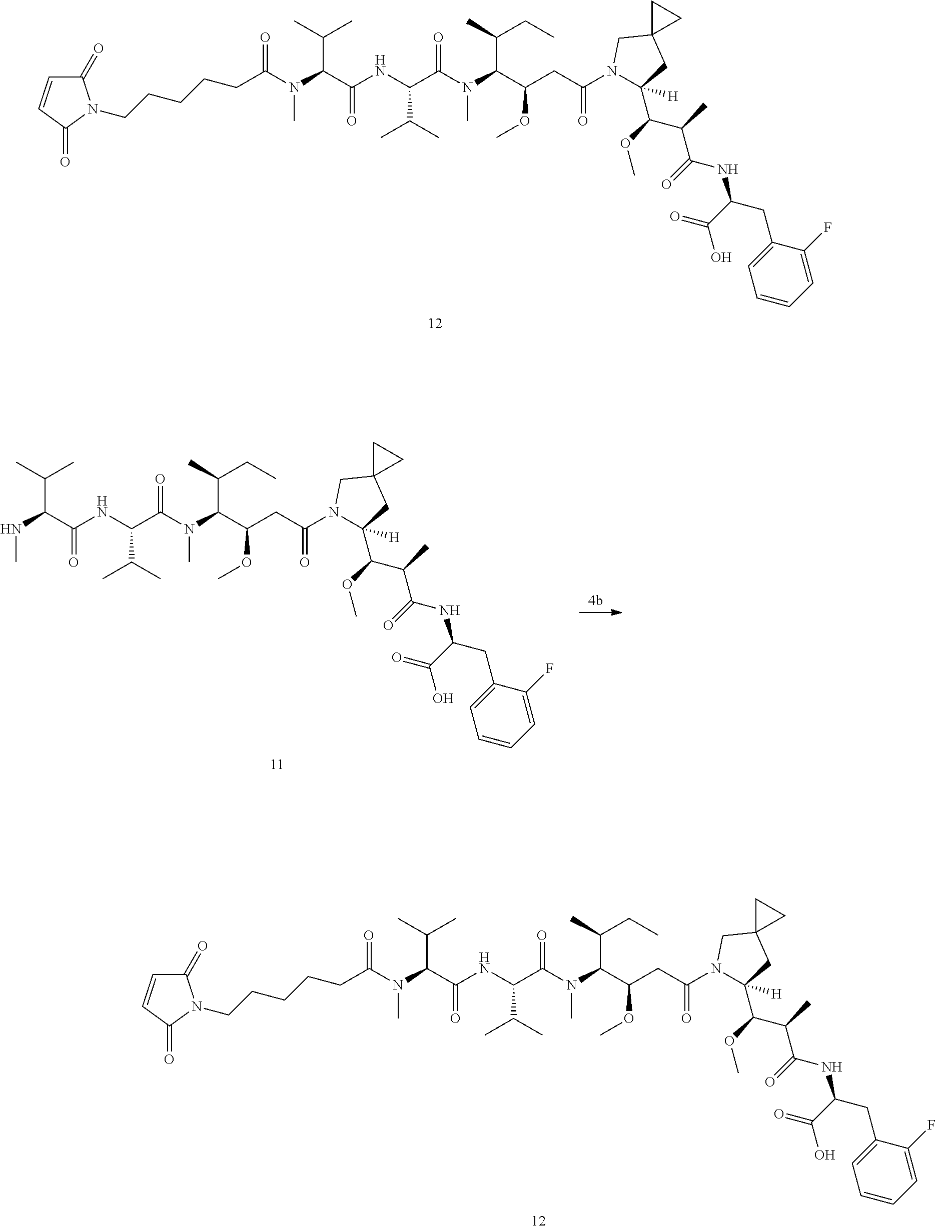 [SMILES: CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C ; CC[C@H](C)[C@@H]([C@@H](CC(=O)N1CC2(CC2)C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-(2-fluorophenyl)propanoic acid 11 (150 mg, 0.193 mmol) was dissolved in 7 mL of dichloromethane, and added with N,N-diisopropylethylamine (87.3 nag, 0.677 mmol). The reaction mixture was cooled to 0° C. under an argon atmosphere, and added dropwise with a preformed solution of 6-(2,5-dioxo-2,5-dihydro-1H-pyrrol-1-yl)hexanoyl chloride 4b (57.6 mg, 0.251 mmol) in dichloromethane, and then stirred at room temperature for 2 hours. The reaction was quenched with methanol and concentrated under reduced pressure. The residues were purified by high performance liquid chromatography to obtain the title product of (S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-(2-fluorophenyl)propanoic acid 12 (14.7 mg, white solid, yield 7.8%.

MS m/z (ESI): 969.9 [M+1]

¹H NMR (400 MHz, CD₃OD) δ 7.29-7.23 (m, 2H), 7.10-7.04 (m, 2H), 6.79-6.78 (m, 2H), 4.69-4.54 (m, 3H), 4.18-4.07 (m, 3H), 3.98-3.92 (m, 1H), 3.75-3.71 (m, 2H), 3.50-3.47 (m, 3H), 3.42-3.39 (m, 2H), 3.34-3.32 (m, 5H), 3.27-3.19 (m, 4H), 3.09-2.95 (m, 5H), 2.49-2.47 (m, 2H), 2.41-2.36 (m, 2H), 2.29-2.18 (m, 3H), 2.09-2.02 (m, 2H), 1.90-1.87 (m, 2H), 1.63-1.59 (m, 4H), 1.49 (s, 2H), 1.32-1.28 (m, 3H), 1.21-1.12 (m, 3H), 1.00-0.81 (m, 12H), 0.62-0.55 (m, 3H), 0.46-0.40 (m, 2H).

### Example 13

### (S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-phenylpropanoic acid

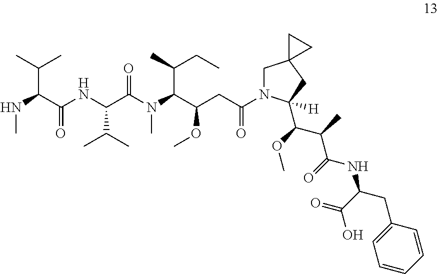 [SMILES: CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

The preparation method was similar to Example 11, except that the material of Step 5 was replaced with (2R,3R)-3-((S)-5-(tert-butoxy carbonyl)-5-azaspiro[2.4]heptan-6-yl)-3-methoxy-2-methylpropanoic acid 11e and (S)-tert-butyl 2-amino-3-phenylpropanoate if to give the title product of (S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-phenylpropanoic acid 13 (219 mg, white solid).

MS m/z (ESI): 758.7 [M+1]

¹H NMR (400 MHz, CD₃OD) δ 7.31-7.19 (m, 5H), 4.70-4.68 (m, 2H), 4.14-4.03 (m, 2H), 3.95-3.93 (m, 1H), 3.69-3.66 (m, 2H), 3.48-3.45 (m, 2H), 3.43-3.38 (m, 3H), 3.34-3.29 (m, 4H), 3.23-3.21 (m, 2H), 3.11 (s, 1H), 2.97-2.89 (m, 2H), 2.67-2.65 (m, 3H), 2.51-2.43 (m, 2H), 2.39-2.27 (m, 2H), 2.21-2.06 (m, 3H), 1.88-1.82 (m, 2H), 1.41-1.39 (m, 2H), 1.23-1.21 (d, 2H), 1.16-1.10 (m, 3H), 1.08-0.98 (m, 10H), 0.89-0.84 (m, 3H), 0.63-0.52 (m, 3H), 0.46-0.43 (m, 2H).

### Example 14

### (S)-2-((2R,3R)-3-((S)-5-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-phenylpropanoic acid

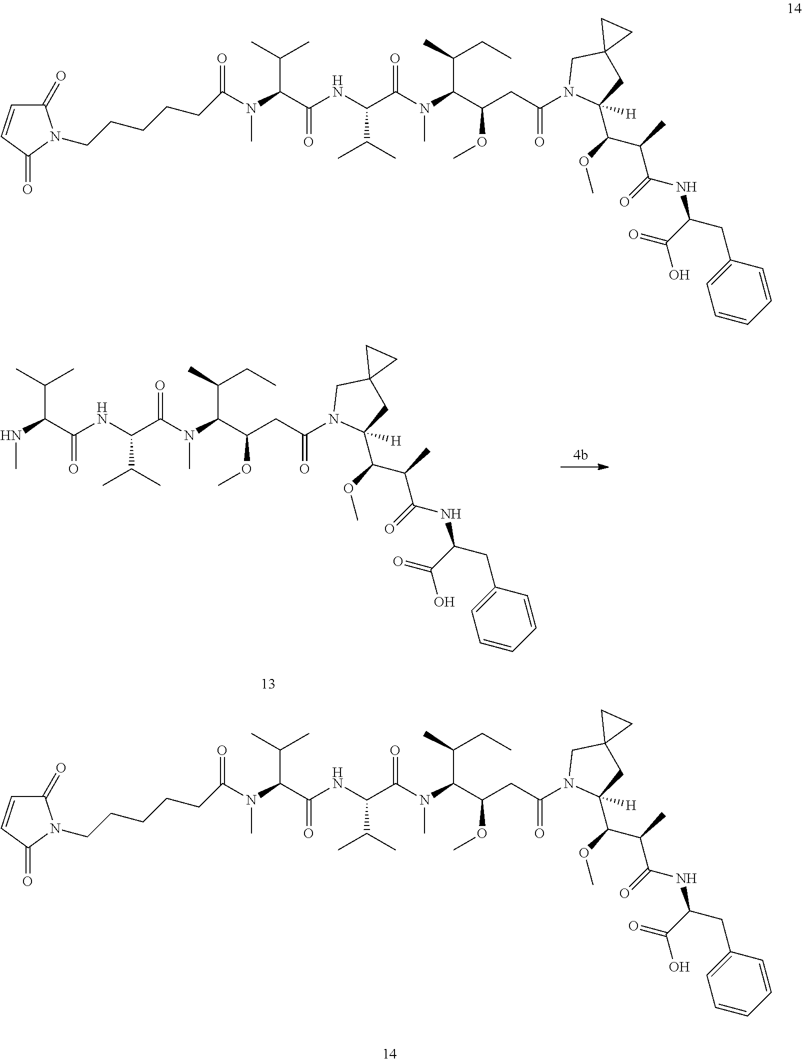 [SMILES: CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C ; CC[C@H](C)[C@@H]([C@@H](CC(=O)N1CC2(CC2)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((S)-5-((3R,4S,5S)-4-((S)—N,3-dimethyl-2-((S)-3-methyl-2-(methylamino)butanamido)butanamido)-3-methoxy-5-methylheptanoyl)-5-azaspiro[2.4]heptan-6-yl)-3-methoxy-2-methylpropanamido)-3-phenylpropanoic acid 13 (120 mg, 0.158 mmol) was dissolved in 5 mL of dichloromethane, and added with N,N-diisopropylethylamine (71.5 mg, 0.554 mmol) The mixture was cooled to 0° C. under an argon atmosphere, and added dropwise with a preformed solution of 6-(2,5-di oxo-2,5-dihydro-1H-pyrrol-1-yl)hexanoyl chloride 4b (47.1 mg, 0.2059 mmol) in dichloromethane, and then stirred at room temperature for 2 hours. After completion of the reaction, 1 mL of methanol was added, and stirred for 10 minutes. The mixture was concentrated under reduced pressure. The residues were purified by high performance liquid chromatography to give the title product of (S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-phenylpropanoic acid 14 (5.1 mg, white solid), yield 3.39%.

MS m/z (ESI): 951.9 [M+1]

¹H NMR (400 MHz, CD₃OD) δ 7.29-7.18 (m, 5H), 6.80-6.79 (m, 2H), 4.75-4.61 (m, 3H), 4.21-3.99 (m, 3H), 3.94-3.91 (m, 1H), 3.74-3.70 (m, 2H), 3.51-3.44 (m, 3H), 3.42-3.37 (m, 2H), 3.34-3.28 (m, 5H), 3.23-3.21 (m, 4H), 3.10-2.86 (m, 5H), 2.49-2.36 (m, 4H), 2.32-2.17 (m, 3H), 2.12-2.03 (m, 2H), 1.88-1.80 (m, 2H), 1.67-1.58 (m, 4H), 1.49 (s, 2H), 1.37-1.26 (m, 3H), 1.22-1.13 (m, 3H), 1.00-0.83 (m, 12H), 0.63-0.51 (m, 3H), 0.43-0.40 (m, 2H).

### Example 15

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(p-tolyl)propanoic acid

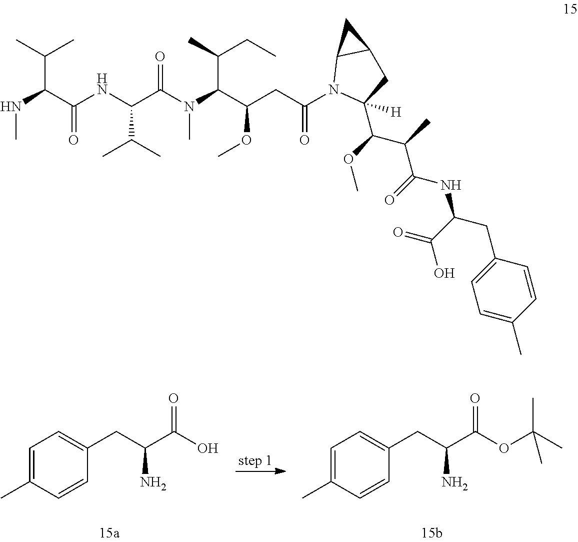

(S)-tert-butyl 2-amino-3-(p-tolyl)propanoate (S)-2-amino-3-(p-tolyl)propanoic acid 15a (400 mg, 2.23 mmol, prepared according to the known method of “Organic Biomolecular Chemistry, 2004, 2(18), 2684-2691”) was dissolved in 10 mL of tert-butyl acetate. The mixture was added with perchloric acid (336.3 mg (70%), 3.34 mmol) under an argon atmosphere, and stirred at room temperature for 16 hours. After the reaction, 10 mL of water was added. The aqueous phase was adjusted to pH=8 with saturated sodium bicarbonate solution and extracted with dichloromethane (5 mL×3). The organic phases were combined, washed with saturated sodium chloride solution (5 mL), dried over anhydrous sodium sulfate, filtered and the filtrate was concentrated under reduced pressure to obtain the title product (S)-tert-butyl 2-amino-3-(p-tolyl)propanoate 15b (370 mg, white solid), yield 70%.

The preparation method was similar to Example 1, except that the raw material in step 4 was replaced with (2R,3R)-3-((1S,3S,5S)-2-(tert-butoxycarbonyl)-2-azabicyclo[3.1.0]hexan-3-yl)-3-methoxy-2-methylpropanoic acid 1e and (S)-tert-butyl 2-amino-3-(p-tolyl)propanoate 15b, to obtain the title product (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(p-tolyl)propanoic acid 15 (30 mg, white solid).

MS m/z (ESI): 758.8 [M+1]

¹H NMR (400 MHz, CD₃OD): δ 7.19-7.07 (m, 4H), 4.82-4.68 (m, 2H), 4.30-4.18 (m, 1H), 4.15-4.05 (m, 1H), 3.89-3.84 (m, 1H), 3.83-3.76 (m, 1H), 3.74-3.62 (m, 2H), 3.47-3.12 (m, 9H), 2.89-2.79 (m, 1H), 2.70-2.59 (m, 4H), 2.34-2.03 (m, 7H), 1.91-1.75 (m, 1H), 1.73-1.53 (m, 2H), 1.50-1.24 (m, 4H), 1.22-0.92 (m, 18H), 0.90-0.79 (m, 4H), 0.75-0.64 (m, 1H).

### Example 16

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(thiophen-2-yl)propanoic acid

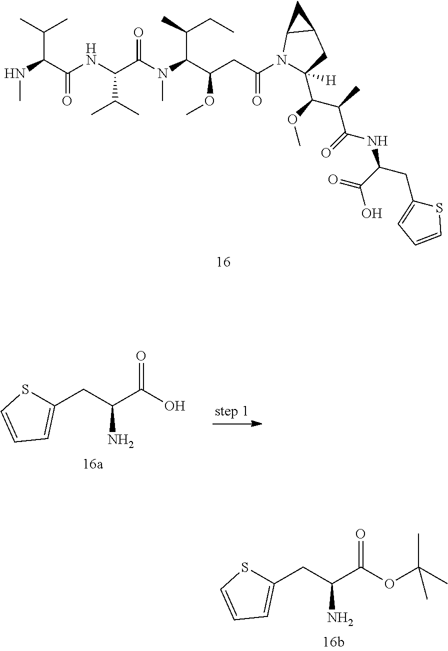 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccs1)C(=O)O)OC)N(C)C(=O)[C@@H](NC(=O)[C@@H](NC)C(C)C)C(C)C ; N[C@@H](Cc1cccs1)C(=O)O ; CC(C)(C)OC(=O)[C@@H](N)Cc1cccs1]

### Step 1

### (S)-tert-butyl 2-amino-3-(thiophen-2-yl)propanoate

(S)-2-amino-3-(thiophen-2-yl)propanoic acid 16a (400 mg, 2.33 mmol, prepared according to the known method of “European Journal of Organic Chemistry, 2006, (5), 1113-1116”) was dissolved in 10 mL of tert-butyl acetate. The mixture was cooled to 0° C. under an argon atmosphere, and added dropwise with perchloric acid (352 mg (70%), 3.5 mmol). The reaction mixture was stirred at room temperature for 16 hours and then added with water, and adjusted to pH=8 to 9 with saturated sodium bicarbonate solution and extracted with dichloromethane. The organic phases were combined, washed successively with water and saturated sodium chloride solution, dried over anhydrous sodium sulfate, filtered and the filtrate was concentrated under reduced pressure to obtain the crude title product of (S)-tert-butyl 2-amino-3-(thiophen-2-yl)propanoate 16b (370 mg, light yellow oil). The product was used in the next step without further purification.

MS m/z (ESI): 228.3 [M+1].

The preparation method was similar to Example 1, except that the raw material in step 4 was replaced with (2R,3R)-3-((1S,3S,5S)-2-(tert-butoxycarbonyl)-2-azabicyclo[3.1.0]hexan-3-yl)-3-methoxy-2-methylpropanoic acid 1e and (S)-tert-butyl 2-amino-3-(thiophen-2-yl)propanoate 16b, to obtain the title product of (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(thiophen-2-yl)propanoic acid 16 (18 mg, white solid).

MS m/z (ESI): 527.6 [M+1]

¹H NMR (400 MHz, CDCl₃) δ 11.00 (s, 1H), 8.44 (s, 1H), 7.17-7.15 (d, 1H), 6.94-6.88 (d, 2H), 4.97-4.95 (m, 2H), 4.10-4.08 (m, 1H), 3.85-3.83 (m, 2H), 3.71-3.69 (m, 2H), 3.52-3.50 (m, 2H), 3.37-3.34 (m, 12H), 3.06-3.04 (m, 2H), 2.80-2.78 (m, 2H), 2.3-2.26 (m, 4H), 1.64-1.59 (m, 3H), 1.48-1.46 (m, 2H), 1.31-1.259 (m, 12H), 1.08-0.98 (m, 8H), 0.89-0.82 (m, 4H).

### Example 17

### (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(3-fluorophenyl)propanoic acid

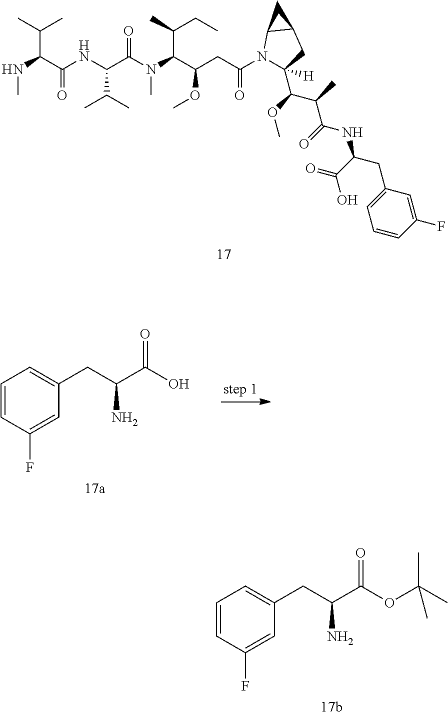 [SMILES: CC[C@H](C)[C@@H]([C@@H](CC(=O)N1[C@H]2C[C@H]2C[C@H]1[C@H](OC)[C@@H](C)C(=O)N[C@@H](Cc1cccc(F)c1)C(=O)O)OC)N(C)C(=O)[C@@H](NC(=O)[C@@H](NC)C(C)C)C(C)C ; N[C@@H](Cc1cccc(F)c1)C(=O)O ; CC(C)(C)OC(=O)[C@@H](N)Cc1cccc(F)c1]

### Step 1

### (S)-tert-butyl 2-amino-3-(3-fluorophenyl)propanoate

(S)-2-amino-3-(3-fluorophenyl)propanoic acid 17a (549 mg, 3 mmol, prepared according to the known method of “Advanced Synthesis & Catalysis, 2012, 354(17), 3327-3332”) was dissolved in 15 mL of tert-butyl acetate. The mixture was added with perchloric acid (450 mg (70%), 4.5 mmol) under an argon atmosphere at 0° C., then stirred at room temperature for 12 hours. After the reaction, 30 mL of water was added, and the organic phase was washed successively with 20 mL of 1N hydrochloric acid and saturated sodium bicarbonate solution. The aqueous phase was combined, adjusted to pH=8 to 9 with saturated sodium bicarbonate solution and extracted with dichloromethane (100 mL×2). The organic phases were combined, dried over anhydrous sodium sulfate, filtered and the filtrate was concentrated under reduced pressure to obtain the crude title product of (S)-tert-butyl 2-amino-3-(3-fluorophenyl)propanoate 17b (600 mg, oil). The product was used in the next step without further purification.

The preparation method was similar to Example 1, except that the raw material in step 4 was replaced with (2R,3R)-3-((1S,3S,5S)-2-(tert-butoxycarbonyl)-2-azabicyclo[3.1.0]hexan-3-yl)-3-methoxy-2-methylpropanoic acid 1e and(S)-tert-butyl 2-amino-3-(3-fluorophenyl)propanoate 17b, to obtain the title product of (S)-2-((2R,3R)-3-((1S,3S,5S)-2-((3R,4S,5S)-4-((S)—N,3-dimethyl-2-((S)-3-methyl-2-(methylamino)butanamido)butanamido)-3-methoxy-5-methylheptanoyl)-2-azabicyclo[3.1.0]hexan-3-yl)-3-methoxy-2-methylpropanamido)-3-(3-fluorophenyl)propanoic acid 17 (25 mg, white solid).

MS m/z (ESI): 762.8 [M+1]

¹H NMR (400 MHz, CD₃OD): δ 7.29-7.21 (m, 1H), 7.08-6.87 (m, 3H), 4.81-4.73 (m, 1H), 4.72-4.67 (m, 1H), 4.61-4.53 (m, 1H), 4.29-4.22 (m, 1H), 4.15-4.06 (m, 1H), 3.99-3.93 (m, 1H), 3.75-3.58 (m, 2H), 3.43-3.12 (m, 9H), 2.99-2.90 (m, 1H), 2.69-2.60 (m, 4H), 2.30-1.97 (m, 4H), 1.87-1.77 (m, 1H), 1.63-1.53 (m, 1H), 1.49-1.36 (m, 1H), 1.18-0.92 (m, 22H), 0.91-0.82 (m, 4H), 0.81-0.71 (m, 1H).

### Example 18

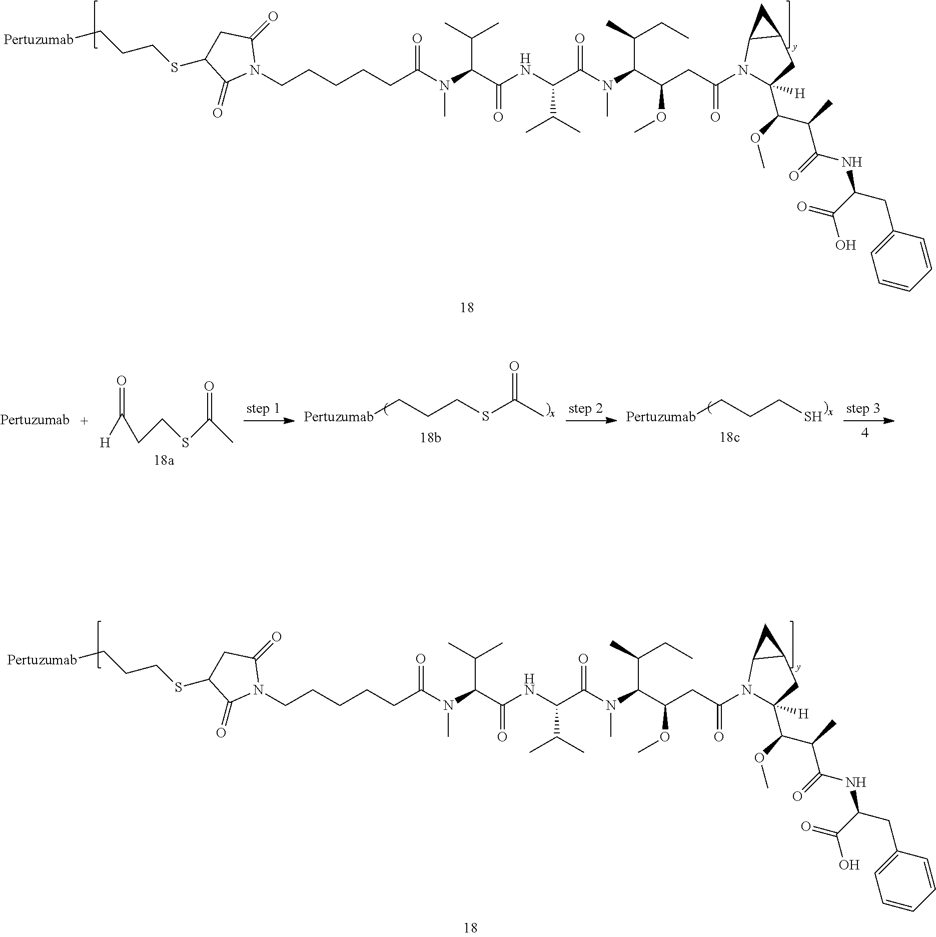

### Step 1

S-(3-oxopropyl) ethanethioate 18a (0.35 mg, 2.65 μmol) was dissolved in 0.45 mL of acetonitrile. The Pertuzumab in acetic acid/sodium acetate buffer (10.85 mg/ml, 4.5 mL, 0.488 mmol) with pH 4.5 was added with the solution of S-(3-oxopropyl) ethanethioate 18a in acetonitrile and then added with 1.0 mL of an aqueous solution of sodium cyanoborohydride (7.06 mg, 112 μmol) dropwise. The reaction mixture was stirred at 25° C. for 2 hours. After completion of the reaction, the residues were desalted and purified with a Sephadex G25 gel column (elution phase: 0.05 M PBS solution at pH 6.5) to give the title product 18b solution, which was used directly in the next step.

### Step 2

The solution of 18b (15.0 mL) was added with 0.45 mL of a 2.0 M solution of hydroxylamine hydrochloride and stirred at 25° C. for 30 minutes. The reaction solution was desalted with Sephadex G25 gel column (Elution phase: 0.05 M PBS solution at pH 6.5) to give the solution of Pertuzumab-propanethiol 18c (concentration 1.65 mg/ml, 22.6 mL).

### Step 3

(S)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido) 4 (1.09 mg, 1.16 μmop was dissolved in 1.1 mL of acetonitrile and added with Pertuzumab-propanethiol solution 18c (1.65 mg/mL, 11.3 mL). After stirring at 25° C. for 4 hours, the reaction mixture was desalted with Sephadex G25 gel column (Elution phase: 0.05M PBS solution at pH 6.5), and filtered under sterile conditions through a 0.2 μm filter to give the title product 18 in PBS buffer (0.75 mg/mL, 19.5 mL), which was then stored at 4° C.

Q-TOF LC/MS: characteristic peaks: 148119.54(M_{Ab}+0D), 149331.45(M_{Ab}+1D), 150407.02 (M_{Ab}+2D, 151297.79(M_{Ab}+3D), 152448.85(M_{Ab}+4D), 153782.23(M_{Ab}+5D). Average value: y=2.0.

### Example 19

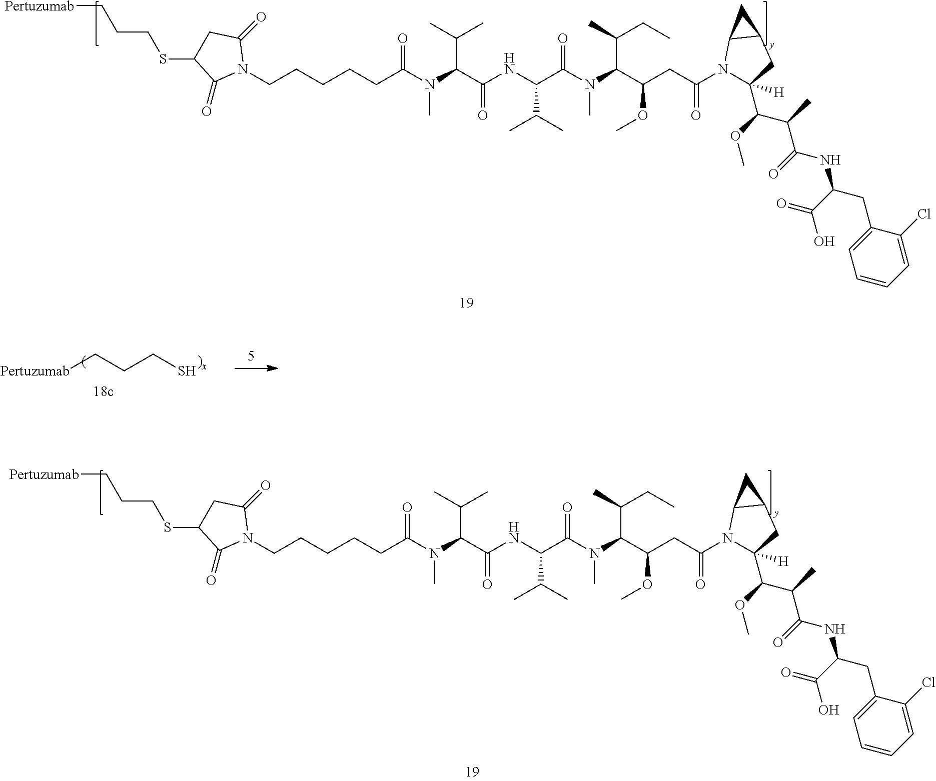

(S)-3-(2-chlorophenyl)-2-((2R,3R)-3-((1S,3S,5S)-2-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)propanoic acid 5 (1.39 mg, 1.43 μmol) was dissolved in 1.1 mL of acetonitrile and added with pertuzumab-propanethiol solution 18c (1.65 mg/mL, 11.3 mL), and stirred at 25° C. for 4 hours. The reaction mixture was desalted and purified by Sephadex G25 gel column (elution phase: 0.05 M PBS solution, pH 6.5), filtrated under a sterile condition through a 0.2 μm filter to obtain the title product of 19 in PBS buffer (0.78 mg/mL, 20.0 mL), and then stored at 4° C.

Q-TOF LC/MS: characteristic peaks: 148119.68(M_{Ab}+0D), 149308.79(M_{Ab}+1D), 150194.76 (M_{Ab}+2D), 151354.52(M_{Ab}+3D), 152410.57(M_{Ab}+4D), 153375.3 1 (M_{Ab}+5D).

Average value: y=1.9.

### Example 20

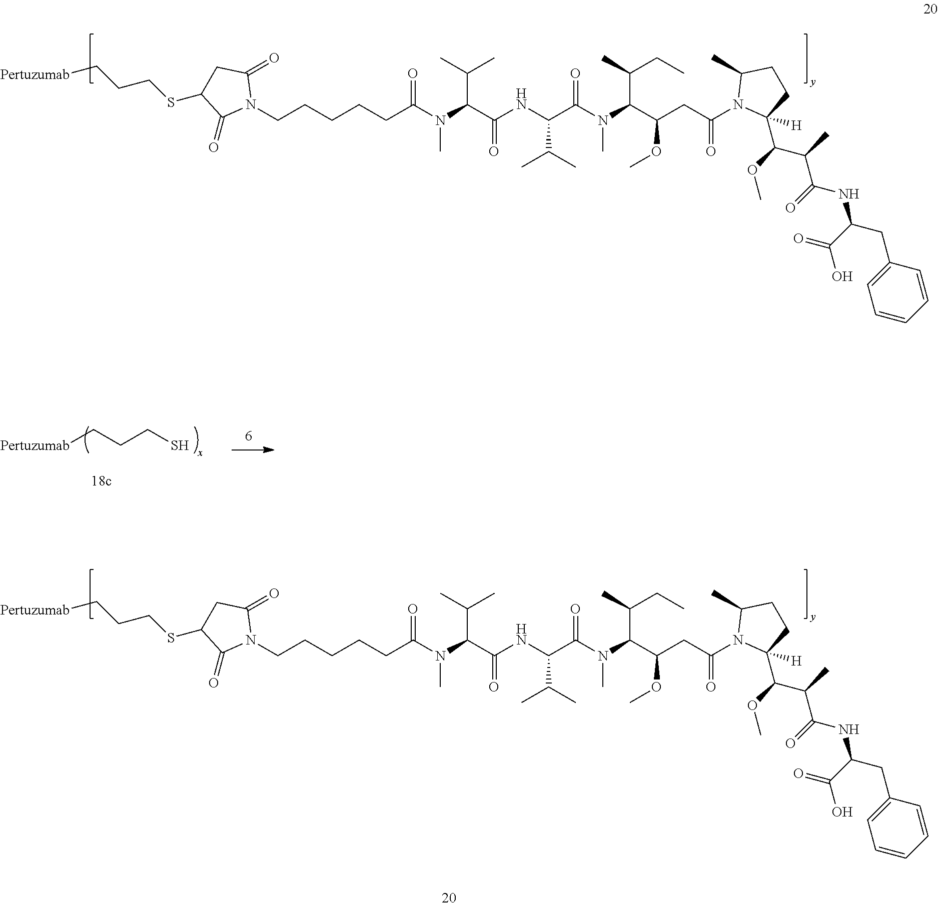

(S)-2-((2R,3R)-3-((2S,5S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-methylpyrrolidin-2-yl)-3-methoxy-2-methylpropanamido)-3-phenylpropanoic acid 6 (1.08 mg, 1.15 μmol) was dissolved in 1.25 mL of acetonitrile and added with Pertuzumab-propanethiol solution 18c (1.50 mg/mL, 12.5 mL), and stirred at 25° C. for 4 hours. The reaction mixture was desalted and purified by Sephadex G25 gel column (elution phase: 0.05 M PBS solution, pH 6.5), filtered under a sterile condition through a 0.2 μm filter to obtain the title product of 20 in PBS buffer (0.74 mg/mL, 19.0 mL), and then stored at 4° C.

Q-TOF LC/MS: characteristic peaks: Q-TOF LC/MS: 148253.27(M_{Ab}+0D), 149263.59 (M_{Ab}+1D), 150315.25(M_{Ab}+2D), 151334.45(M_{Ab}+3D), 152383 0.92(M_{Ab}+4D), 153446.37 (M_{Ab}+5D).

Average value: y=2.2.

### Example 21

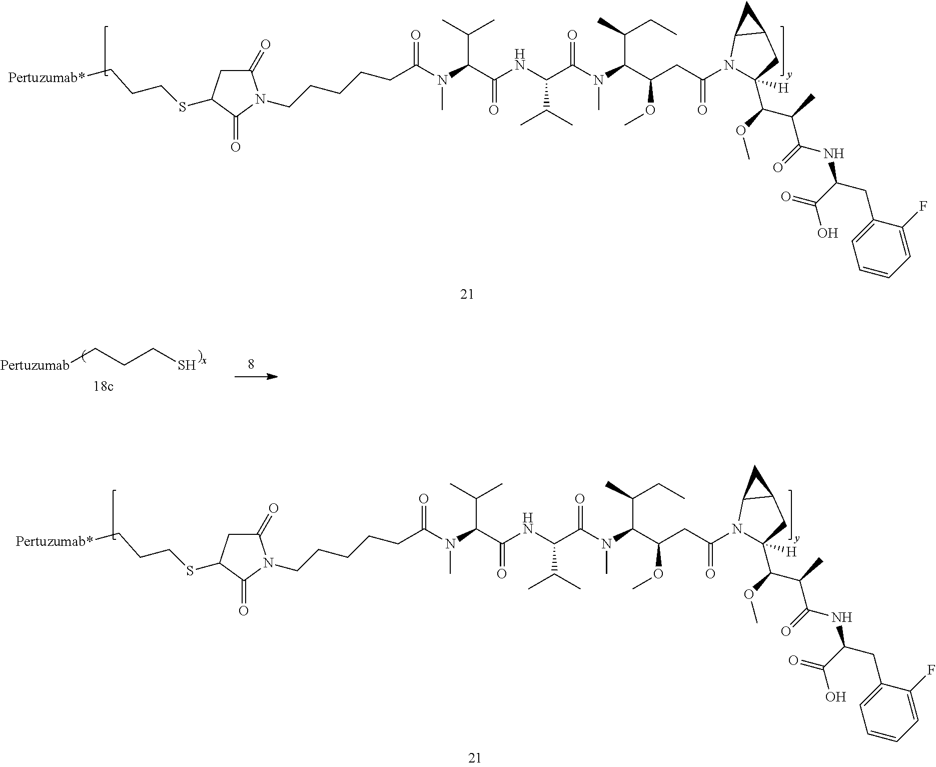

(S)-2-((2R,3R)-3-((1S,3S,5S)-2-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-(2-fluorophenyl)propanoic acid 8 (3.0 mg, 3.0 μmol) was dissolved in 1.0 mL of acetonitrile and added with pertuzumab-propanethiol solution 18c (2.11 mg/mL, 10.0 mL), and stirred at 25° C. for 4 hours. Then the above reaction mixture was desalted and purified by Sephadex G25 gel column (eluting phase: 0.05 M PBS solution, pH 6.5), filtrated under a sterile condition through a 0.2 μm filter to obtain the title product of 21 in PBS buffer (1.31 mg/mL, 12.5 mL), and then stored at 4° C. frozen storage.

Q-TOF LC/MS: characteristic peaks: 148312.73(M_{Ab}+0D), 149515.61(M_{Ab}+1D), 150459.55 (M_{Ab}+2D), 151521.47(M_{Ab}+3D), 152580.02(M_{Ab}+4D).

Average value: y=1.7.

### Example 22

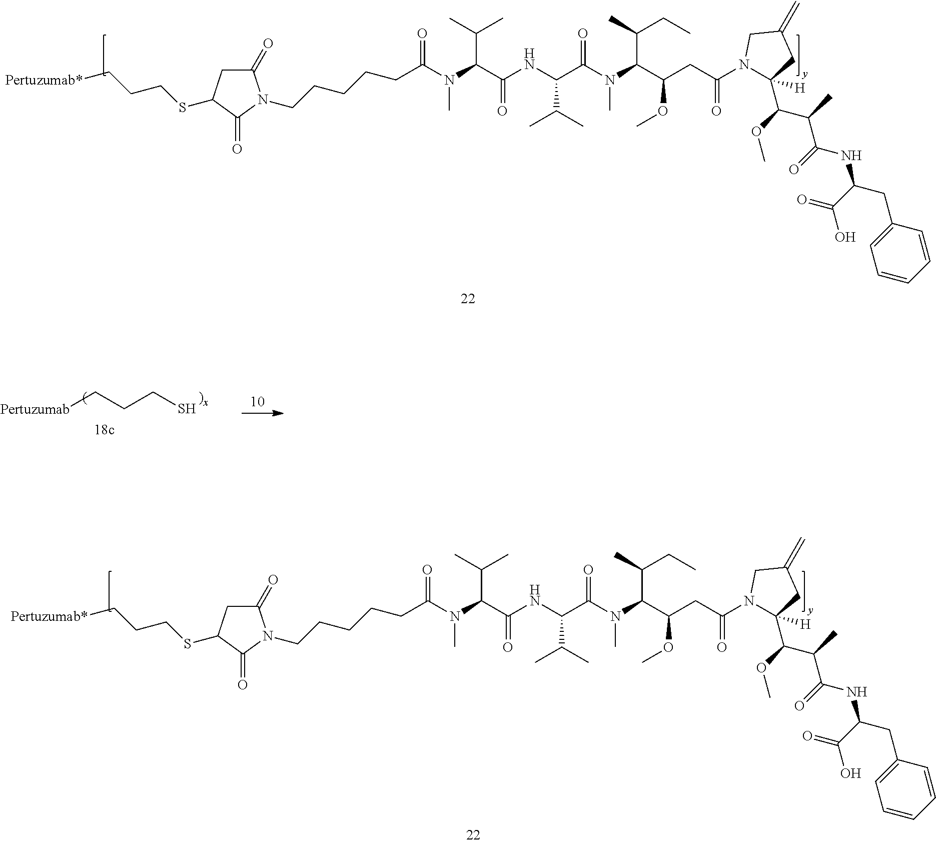

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-methylenepyrrolidin-2-yl)-3-methoxy-2-methylpropanamido)-3-phenylpropanoic acid 10 (1.50 mg, 1.60 μmol) was dissolved in 1.0 mL of acetonitrile and added with pertuzumab-propanethiol solution 18c (2.11 mg/mL, 10.0 mL), and stirred at 25° C. for 4 hours. The reaction mixture was desalted and purified by Sephadex G25 gel column (eluting phase: 0.05 M PBS solution, pH 6.5), filtrated under a sterile condition through a 0.2 μm filter to obtain the title product of 22 in PBS buffer (1.28 mg/mL, 13.0 mL), and then stored at 4° C.

Q-TOF LC/MS: characteristic peaks: 148411.82(M_{Ab}+0D), 149412.97(M_{Ab}+1D), 150468.08 (M_{Ab}+2D), 151496.41(M_{Ab}+3D), 152580.37(M_{Ab}+4D).

Average value: y=2.1.

### Example 23

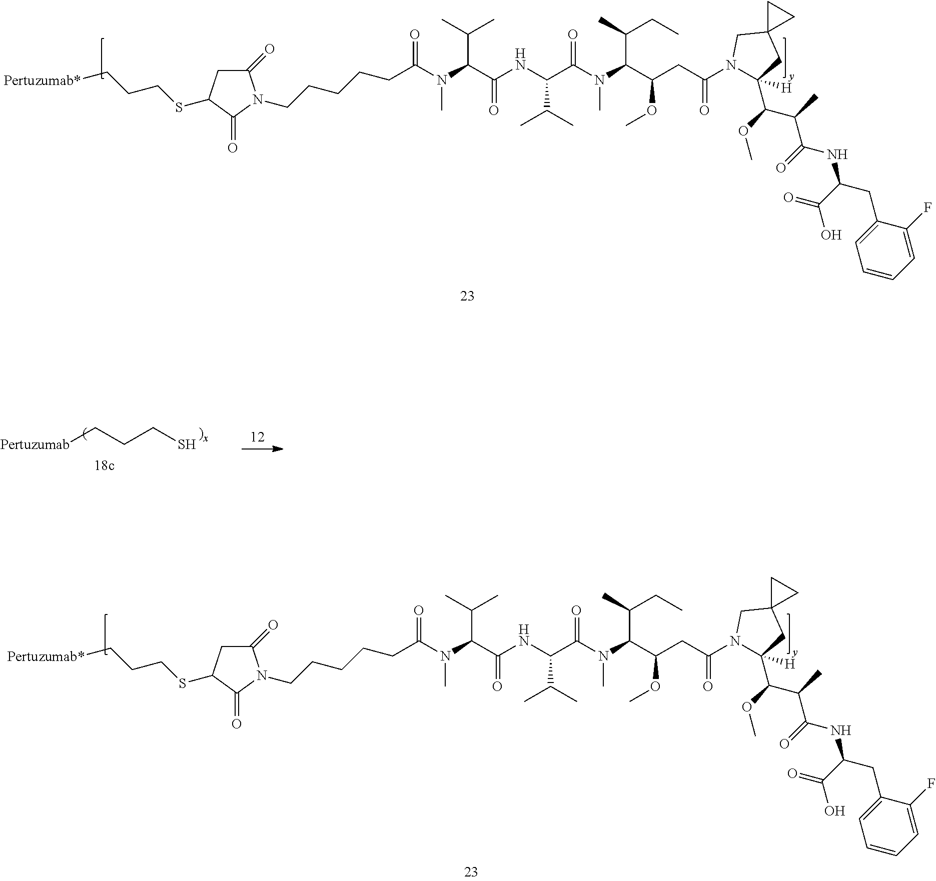

(S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-(2-fluorophenyl)propanoic acid 12 (0.86 mg, 0.89 μmol) was dissolved in 0.6 mL of acetonitrile and added with Pertuzumab-propanethiol solution 18c (2.06 mg/mL, 6.0 mL), and stirred at 25° C. for 4 hours. The reaction mixture was desalted and purified by Sephadex G25 gel column (elution phase: 0.05 M PBS solution, pH 6.5), filtrated under a sterile condition through a 0.2 μm filter to obtain the title product of 23 in PBS buffer (0.70 mg/mL, 15 mL), and then stored at 4° C.

Q-TOF LC/MS: characteristic peaks: 148092.94(M_{Ab}+0D), 149296.82(M_{Ab}+1D), 150339.86 (M_{Ab}+2D), 151416.51(M_{Ab}+3D), 152516.25(M_{Ab}+4D), 153422.64(M_{Ab}+5D).

Average value: y=1.7.

### Example 24

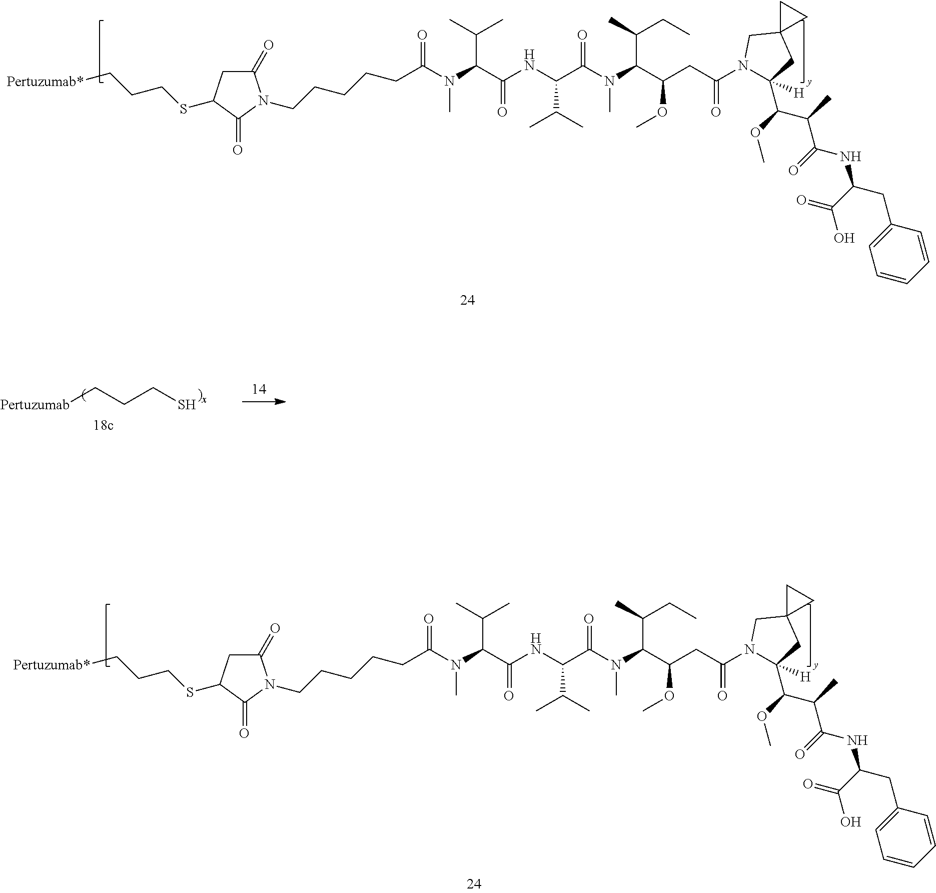

(S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-phenylpropanoic acid 14 (0.73 mg, 0.78 μmol) was dissolved in 0.6 mL of acetonitrile and added with Pertuzumab-propanethiol solution 18c (2.06 mg/mL, 6.0 mL), and stirred at 25° C. for 4 hours. The reaction mixture was desalted and purified by Sephadex G25 gel column (elution phase: 0.05 M PBS solution, pH 6.5), filtrated under a sterile condition through a 0.2 μm filter to obtain the title product of 24 in PBS buffer (0.68 mg/mL, 15.5 mL), and then stored at 4° C.

Q-TOF LC/MS: characteristic peaks: 148094.99(M_{Ab}+0D), 149277.83(M_{Ab}+1D), 150343.15 (M_{Ab}+2D), 151359.29(M_{Ab}+3D), 152478.14(M_{Ab}+4D), 153449.92(M_{Ab}+5D).

Average value: y=1.6.

### Example 25

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoic acid

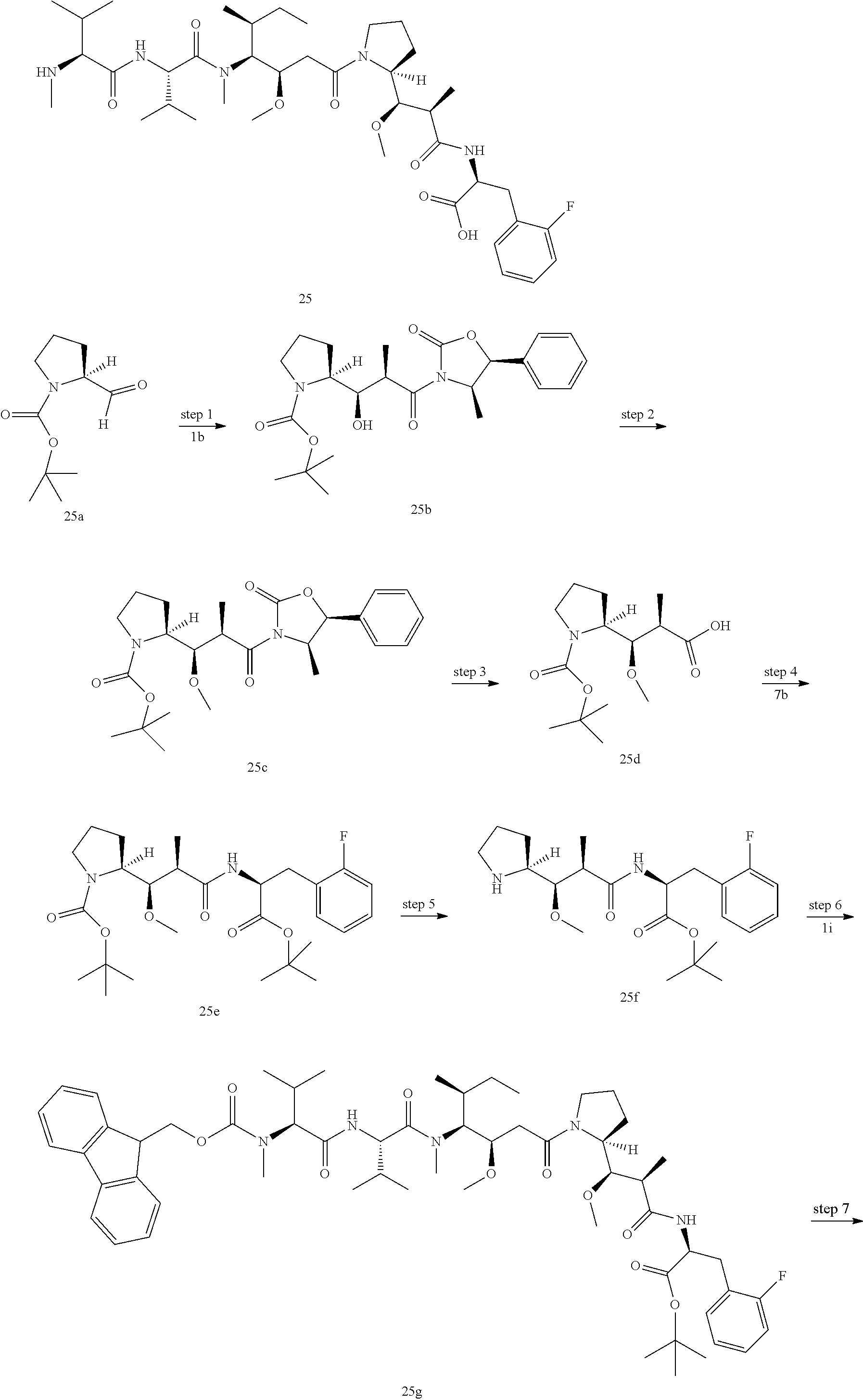

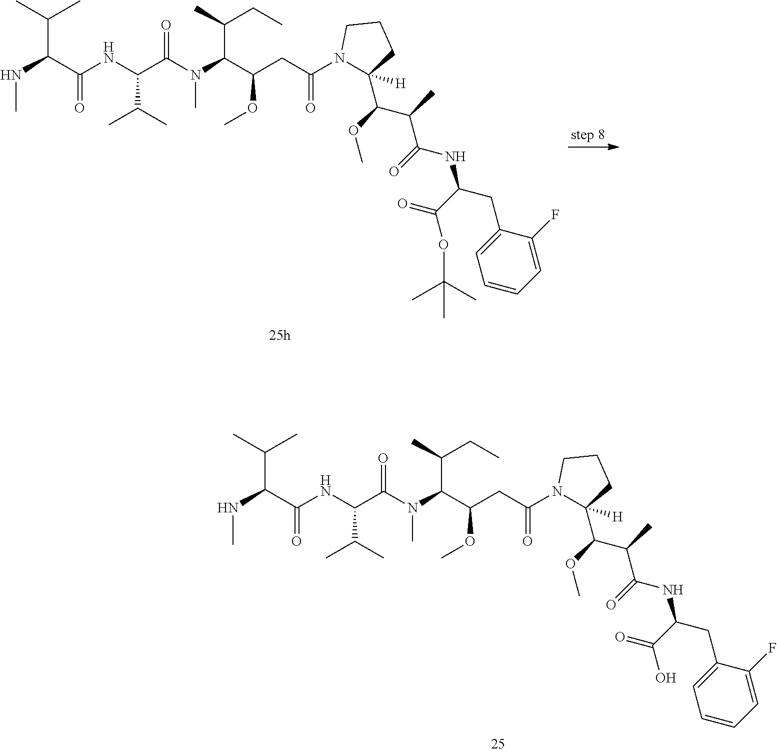 [SMILES: CC[C@H](C)[C@@H]([C@@H](CC(=O)N1CCC[C@H]1[C@H](OC)[C@@H](C)C(=O)N[C@@H](Cc1ccccc1F)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C]

### Step 1

### (S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate

(4R,5S)-4-methyl-5-phenyl-3-propionyloxazolidin-2-one 1b (4.6 g, 20.1 mmol) was dissolved in 80 mL of dichloromethane, and cooled to 0° C. under argon atmosphere. The reaction solution was added dropwise with triethylamine (3.2 mL, 23.1 mmol) and dibutylboron trifluoromethanesulfonate (20 mL, 20.7 mmol) at 0° C., and stirred at 0° C. for 50 minutes, then added dropwise with 5 mL preformed solution of (S)-tert-butyl 2-formylpyrrolidine-1-carboxylate 25a (4 g, 20.1 mmol, prepared according to the known method of “Journal of the American Chemical Society, 2011, 133(42), 16901-16910”) in dichloromethane at −75° C., and stirred for 1 hour at −75° C., then 2 hours at 0° C. and 1 hour at room temperature. A mixture of 60 mL of a phosphate buffer (pH=7.0) and methanol (V/V=1:3) was added to the reaction solution. A mixture of 60 mL of methanol and hydrogen peroxide (30%) (V/V=2:1) was added at 0° C. and stirred at room temperature for 1 hour. After completion of the reaction, the organic phase was concentrated under reduced pressure and 15 mL of water was added. The aqueous phase was extracted with ether (30 mL×3) and the ether phases were combined, washed successively with 5% sodium bicarbonate solution, water, and saturated sodium chloride solution, and dried over anhydrous sodium sulfate, filtered, the filtrate was concentrated under reduced pressure. The residues were purified by silica gel column chromatography using eluent system B to give the title product of (S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate 25b (2.26 g, white foam solid), yield 24.9%.

MS m/z (ESI): 333.3 [M-100+1]

### Step 2

### (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate

(S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate 25b (2 g, 4.62 mmol) was dissolved in 20 mL of dichloromethane and added with 2 g of crushed molecular sieves. The mixture was added with 1,8-bisdimethylaminonaphthalene (2.57 g, 12 mmol), trimethyloxonium tetrafluoroborate (1.71 g, 11.5 mmol) at 0° C. under an argon atmosphere and stirred at room temperature for 17 hours. The resulting mixture was filtered and the filter cake was washed with dichloromethane. The filtrate was combined and the organic phase was washed with saturated ammonium chloride solution (20 mL×3) and saturated sodium chloride solution, dried over anhydrous sodium sulfate, filtered, and the filtrate was concentrated under reduced pressure. The residues were purified by silica gel column chromatography using eluent system B to give the title product of (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate 25c (1.3 g, white foam solid), yield 63%.

MS m/z (ESI): 447.3 [M+1]

### Step 3

### (2R,3R)-3-((S)-1-(tert-butoxycarbonyl)pyrrolidin-2-yl)-3-methoxy-2-methylpropanoic acid

(S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate 25c (1.3 g, 2.9 mmol) was dissolved in 80 mL of tetrahydrofuran. The solution was cooled to 0° C. under argon atmosphere, and added with 30% hydrogen peroxide (1.25 g, 11 mmol) dropwise and lithium hydroxide monohydrate (207 mg, 4.95 mmol). The reaction mixture was stirred at room temperature for 12 hours. After completion of the reaction, sodium sulfite solid (1.47 g, 11.6 mmol) was added to the reaction solution, and the mixture was stirred at room temperature for 1 hour. A small amount of water was added and the organic phase was concentrated under reduced pressure. A small amount of water was added to dissolve the residues and then extracted with dichloromethane (50 mL×2). The aqueous phase was added with hydrochloric acid until pH=3 and extracted with dichloromethane (40 mL 3). The organic phase was washed with water and saturated sodium chloride solution, dried over anhydrous sodium sulfate, filtered and the filtrate was concentrated under reduced pressure to give the crude title product of (2R,3R)-3-((S)-1-(tert-butoxycarbonyl)pyrrolidin-2-yl)-3-methoxy-2-methylpropanoic acid 25d (870 mg, colorless oil). The product was used in the next step without further purification.

### Step 4

### (S)-tert-butyl 2-((1R,2R)-3-(((S)-1-(tert-butoxy)-3-(2-fluorophenyl)-1-oxopropan-2-yl)amino)-1-methoxy-2-methyl-3-oxopropyl)pyrrolidine-1-carboxylate

(2R,3R)-3-((S)-1-(tert-butoxycarbonyl)pyrrolidin-2-yl)-3-methoxy-2-methylpropanoic acid 25d (100 mg, 0.368 mmol) was dissolved in 6 mL of dichloromethane and 1.8 mL of dimethylformamide, and added with (S)-tert-butyl 2-amino-3-(2-fluorophenyl)propanoate 7b (97 mg, 0.405 mmol). The mixture was added with N,N-diisopropylethylamine (237.8 mg, 2.844 mmol) and 2-(7-azobenzotriazole)-N,N,N′,N′-tetramethyluronium hexafluorophosphate (168.2 mg, 0.442 mmol) under an argon atmosphere, and stirred at room temperature for 2 hours. The reaction mixture was then added with 10 mL of dichloromethane, and washed successively with water, saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((1R,2R)-3-(((S)-1-(tert-butoxy)-3-(2-fluorophenyl)-1-oxopropan-2-yl)amino)-1-methoxy-2-methyl-3-oxopropyl)pyrrolidine-1-carboxylate 25e (157 mg, colorless oil), yield 83.9%.

MS m/z (ESI): 509.3 [M+1]

### Step 5

### (S)-tert-butyl 3-(2-fluorophenyl)-2-((2R,3R)-3-methoxy-2-methyl-3-((S)-pyrrolidin-2-yl)propanamido) propanoate

(S)-tert-butyl 2-((1R,2R)-3-(((S)-1-(tert-butoxy)-3-(2-fluorophenyl)-1-oxopropan-2-yl)amino)-1-methoxy-2-methyl-3-oxopropyl)pyrrolidine-1-carboxylate 25e (157 mg, 0.308 mmol) was dissolved in 2 mL of dioxane, and added with a 4 M solution of hydrogen chloride in dioxane (0.193 mL, 1.08 mmol). The reaction system was sealed and stirred for 1 hour at room temperature, and then placed in a refrigerator for 12 hours at 4° C. The reaction solution was concentrated under reduced pressure, and the residues were dissolved in dichloromethane and washed with saturated sodium bicarbonate solution. The aqueous phase was extracted with dichloromethane. The organic phases were combined, washed with saturated sodium chloride solution, dried over anhydrous sodium sulfate, filtered and the filtrate was concentrated under reduced pressure to give the crude title product of (S)-tert-butyl 3-(2-fluorophenyl)-2-((2R,3R)-3-methoxy-2-methyl-3-((S)-pyrrolidin-2-yl)propanamido) propanoate 25f (100 mg, pale yellow oil). The product was used in the next step without further purification.

MS m/z (ESI): 407.2 [M-1]

### Step 6

### (S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-3-(2-fluorophenyl)propanoate

(S)-tert-butyl 3-(2-fluorophenyl)-2-((2R,3R)-3-methoxy-2-methyl-3-((S)-pyrrolidin-2-yl)propanamido) propanoate 25f (100 mg, 0.244 mmol), (5S,8S,11S,12R)-11-((S)-sec-butyl)-1-(9H-fluoren-9-yl)-5,8-diisopropyl-12-methoxy-4,1 0-dimethyl-3,6,9-trioxo-2-oxa-4,7,10-triazatetradecan-14-oic acid 1i (156.1 mg, 0.244 mmol) was dissolved in 7.5 mL of a mixed solvent of dichloromethane and dimethylformamide (V/V=4:1), and added with N,N-diisopropylethylamine (158 mg, 1.224 mmol). The mixture was added with 2-(7-azobenzotriazole)-N, N, N N′-tetramethyluronium hexafluorophosphate (121 mg, 0.318 mmol) under an argon atmosphere, and stirred at room temperature for 1 hour. The reaction mixture was then diluted with dichloromethane, washed successively with water and saturated sodium chloride solution, dried over anhydrous sodium sulfate, filtered and the filtrate was concentrated under reduced pressure. The residues were purified by silica gel column chromatography using eluent system B to give the title product of (S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-3-(2-fluorophenyl)propanoate 25g (242 mg, pale yellow oil), yield 96.5%.

MS m/z (ESI): 1028.4 [M+1]

### Step 7

### (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoate

(S)-tert-butyl 2-((2R,3R)-3-((S)-1-((5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-3-(2-fluorophenyl)propanoate 25g (242 mg, 0.235 mmol) was dissolved in 3 mL of dichloromethane, and added with 3 mL of diethylamine. The mixture was stirred at room temperature for 3 hours and then concentrated under reduced pressure to give the crude title product of (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoate 25h (250 mg, yellow oil). The product was used in the next step without further purification.

MS m/z (ESI): 806.7[M+1]

### Step 8

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoic acid

The crude product of (S)-tert-butyl 2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoate 25h (262 mg, 0.325 mmol) was placed in the reaction flask and added with 7 mL of 4 M solution of hydrogen chloride in dioxane. The reaction system was sealed and stirred at room temperature for 12 hours. The reaction solution was then concentrated under reduced pressure, and the residues were purified by high performance liquid chromatography to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoic acid 25 (50 mg, white solid), yield 28.4%.

MS m/z (ESI): 750.7 [M+1]

¹H NMR (400 MHz, CD₃OD) δ 7.35-7.30 (m, 1H), 7.20-7.15 (m, 1H), 7.09-6.09 (m, 2H), 4.81-4.75 (m, 1H), 4.70-4.64 (m, 1H), 4.15-4.13 (m, 1H), 4.05-4.03 (m, 1H), 3.84-3.81 (m, 1H), 3.69-3.64 (m, 1H), 3.55-3.51 (m, 1H), 3.48-3.13 (m, 13H), 3.03-2.91 (m, 2H), 2.66-2.53 (m, 3H), 2.47-2.45 (m, 1H), 2.40-2.33 (m, 2H), 2.27-2.23 (m, 1H), 2.17-2.11 (m, 2H), 1.96-1.85 (m, 3H), 1.63-1.40 (m, 4H), 1.21-1.14 (m, 3H), 1.09-0.94 (m, 12H), 0.87-0.84 (m, 3H).

### Example 26

(S)-2-((2R,3R)-3-45)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-fluorophenyl)propanoic acid

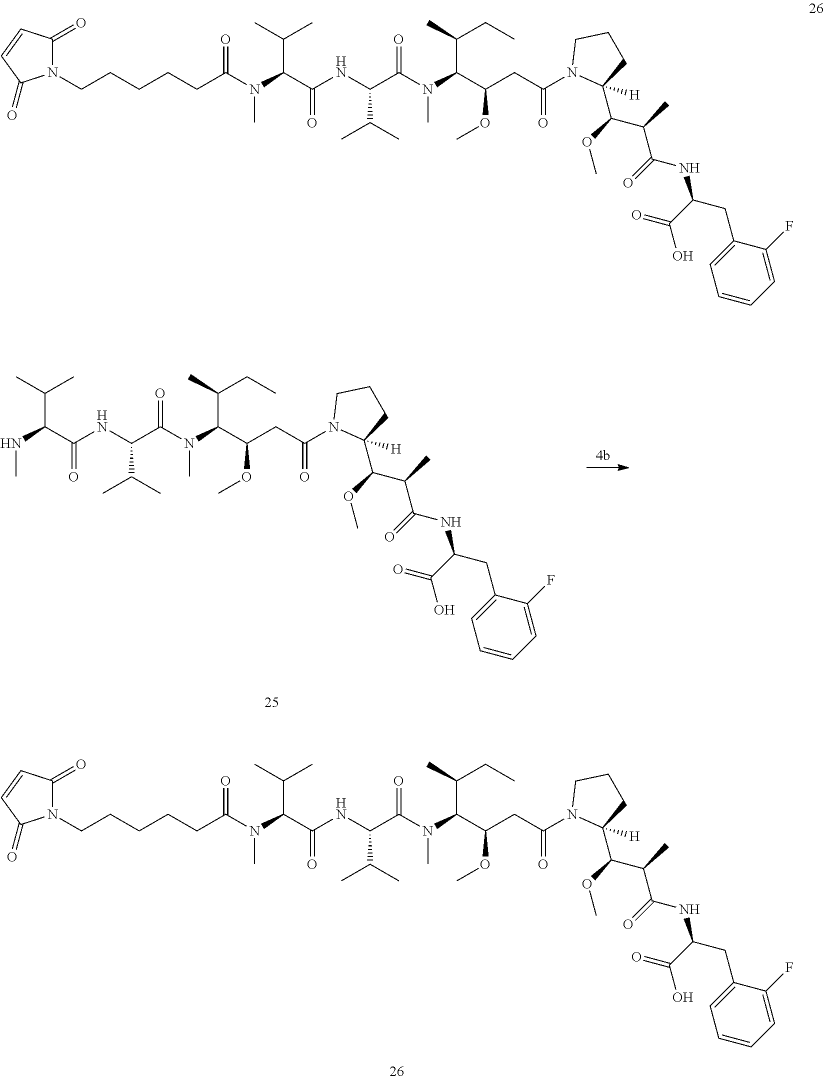 [SMILES: CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-fluorophenyl)propanoic acid 25 (20 mg, 0.026 mmol) was dissolved in 3 mL of dichloromethane and then added with N,N-diisopropylethylamine (13.7 mg, 0.106 mmol). The mixture was cooled to 0° C. under an argon atmosphere, and then added dropwise with a preformed solution of 6-(2,5-dioxo-2,5-dihydro-1H-pyrrol-1-yl) hexanoyl chloride 4b (10.1 mg, 0.0443 mmol) in dichloromethane, and stirred at room temperature for 2 hours. The reaction was quenched with methanol, and then concentrated under reduced pressure. The residues were purified by high performance liquid chromatography to obtain the title product of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-fluorophenyl)propanoic acid 26 (7 mg, white solid), yield 28.5%.

MS m/z (ESI): 941.6 [M-1]

¹H NMR (400 MHz, CD₃OD) δ 7.32-7.21 (m, 2H), 7.10-7.00 (m, 2H), 6.80-6.78 (m, 2H), 4.75-4.55 (m, 3H), 4.12-4.06 (m, 2H), 3.93-3.89 (m, 1H), 3.87-3.78 (m, 2H), 3.69-3.63 (m, 1H), 3.52-3.47 (m, 3H), 3.44-3.28 (m, 3H), 3.21-3.10 (m, 4H), 3.04-2.96 (m, 4H), 2.53-2.40 (m, 4H), 2.35-2.18 (m, 2H), 2.13-2.00 (m, 2H), 1.94-1.75 (m, 4H), 1.68-1.56 (m, 5H), 1.37-1.27 (m, 4H), 1.20-1.13 (m, 3H), 1.05-0.81 (m, 18H).

### Example 27

(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-methoxyphenyl)propanoic acid

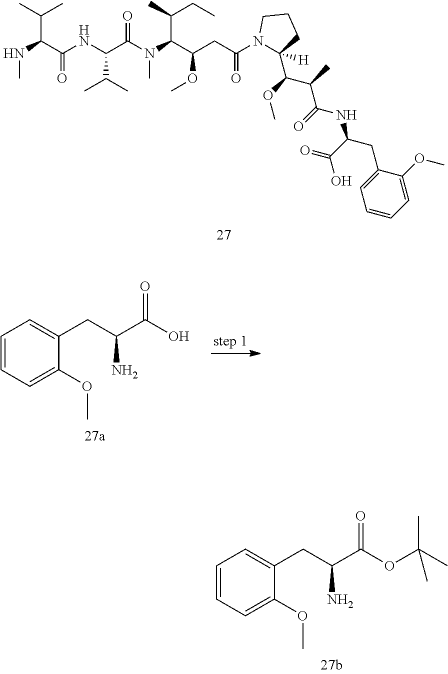 [SMILES: CC[C@H](C)[C@@H]([C@@H](CC(=O)N1CCC[C@H]1[C@H](OC)[C@@H](C)C(=O)N[C@@H](Cc1ccccc1OC)C(=O)O)OC)N(C)C(=O)[C@@H](NC(=O)[C@@H](NC)C(C)C)C(C)C ; COc1ccccc1C[C@H](N)C(=O)O ; COc1ccccc1C[C@H](N)C(=O)OC(C)(C)C]

### Step 1

### (S)-tert-butyl 2-amino-3-(2-methoxyphenyl)propanoate

(S)-2-amino-3-(2-methoxyphenyl)propanoic acid 27a (250 mg, 1.28 mmol, prepared according to the known method of “Chemical Communications (Cambridge, United Kingdom), 2013, 49(70), 7744-766”) was dissolved in 7 mL of tert-butyl acetate. The solution was added with perchloric acid (270 mg (70%), 1.88 mmol) under an argon atmosphere, and stirred at room temperature for 16 hours. The reaction mixture was then added with 10 mL of dichloromethane, and adjusted to pH=8 with saturated sodium bicarbonate solution. The aqueous phase was separated and extracted with dichloromethane (10 mL×3). The organic phases were combined, washed with saturated sodium chloride solution (10 mL), dried over anhydrous sodium sulfate, filtered and the filtrate was concentrated under reduced pressure to obtain the title product of (S)-tert-butyl 2-amino-3-(2-methoxyphenyl)propanoate 27b (280 mg, light yellow oil), yield 87%.

The preparation method was similar to Example 1, except that the raw material in step 4 was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(2-methoxyphenyl)propanoate 27b, to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-methoxyphenyl)propanoic acid 27 (22 mg, off white solid).

MS m/z (ESI): 760.7[M-1].

### Example 28

### (S)-2-((2R,3R)-3-((S)-1-((3R,4S,5S)-4-((S)-2-(S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-methoxyphenyl)propanoic acid

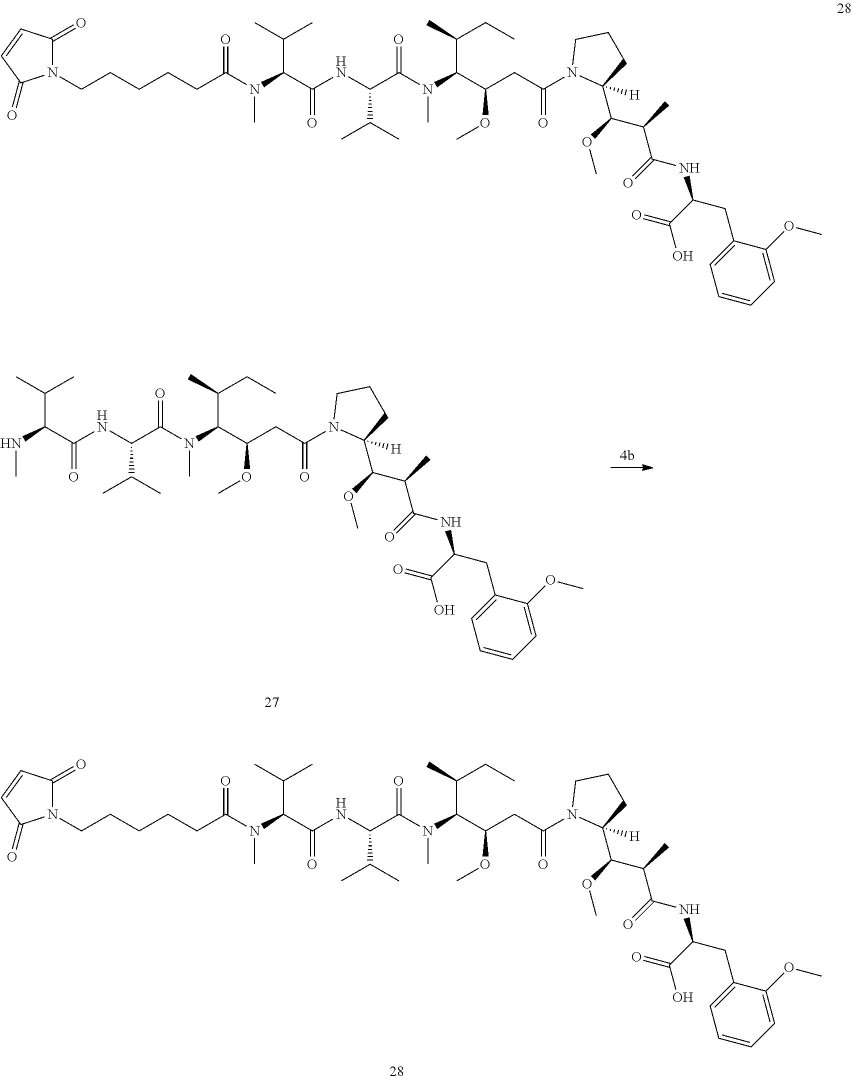

(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(2-methoxyphenyl)propanoic acid 27 (18 mg, 0.023 mmol) was dissolved in 5 mL of dichloromethane. The solution was added with N, N-diisopropylethylamine (12.19 mg, 0.29 mmol) under an argon atmosphere, and then added dropwise with a preformed solution of 6-(2,5-dioxo-2,5-dihydro-1H-pyrrol-1-yl) hexanoyl chloride 4b (6.5 mg, 0.028 mmol) in dichloromethane at 0° C., and stirred at room temperature for 2 hours. The reaction was quenched with methanol, and then concentrated under reduced pressure. The residues were purified by high performance liquid chromatography to give the title product of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-methoxyphenyl)propanoic acid 28 (4 mg, white solid), yield 18.2%.

MS m/z (ESI): 955.5 [M+1]

### Example 29

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(p-tolyl)propanoic acid

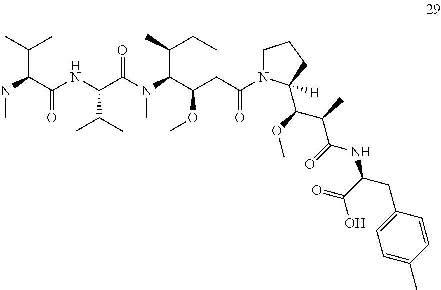 [SMILES: CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@@H](NC)C(C)C)C(C)C]

The preparation method was similar to Example 25, except that the raw material was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(p-tolyl)propanoate 15b, to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(p-tolyl)propanoic acid 29 (20 mg, white solid).

MS m/z (ESI): 746.6 [M+1]

¹H NMR (400 MHz, CD₃OD): δ 7.17-7.05 (m, 4H), 4.84-4.67 (m, 2H), 4.27-4.15 (m, 1H), 4.12-4.05 (m, 1H), 3.89-3.82 (m, 1H), 3.78-3.63 (m, 2H), 3.57-3.47 (m, 1H), 3.43-3.11 (m, 7H), 2.92-2.79 (m, 1H), 2.67 (d, 3H), 2.52-2.44 (m, 1H), 2.40-2.15 (m, 7H), 2.12-2.01 (m, 1H), 1.95-1.69 (m, 3H), 1.67-1.49 (m, 2H), 1.48-1.27 (m, 4H), 1.23-1.12 (m, 5H), 1.11-0.95 (m, 14H), 0.94-0.84 (m, 3H).

### Example 30

### (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(p-tolyl)propanoic acid

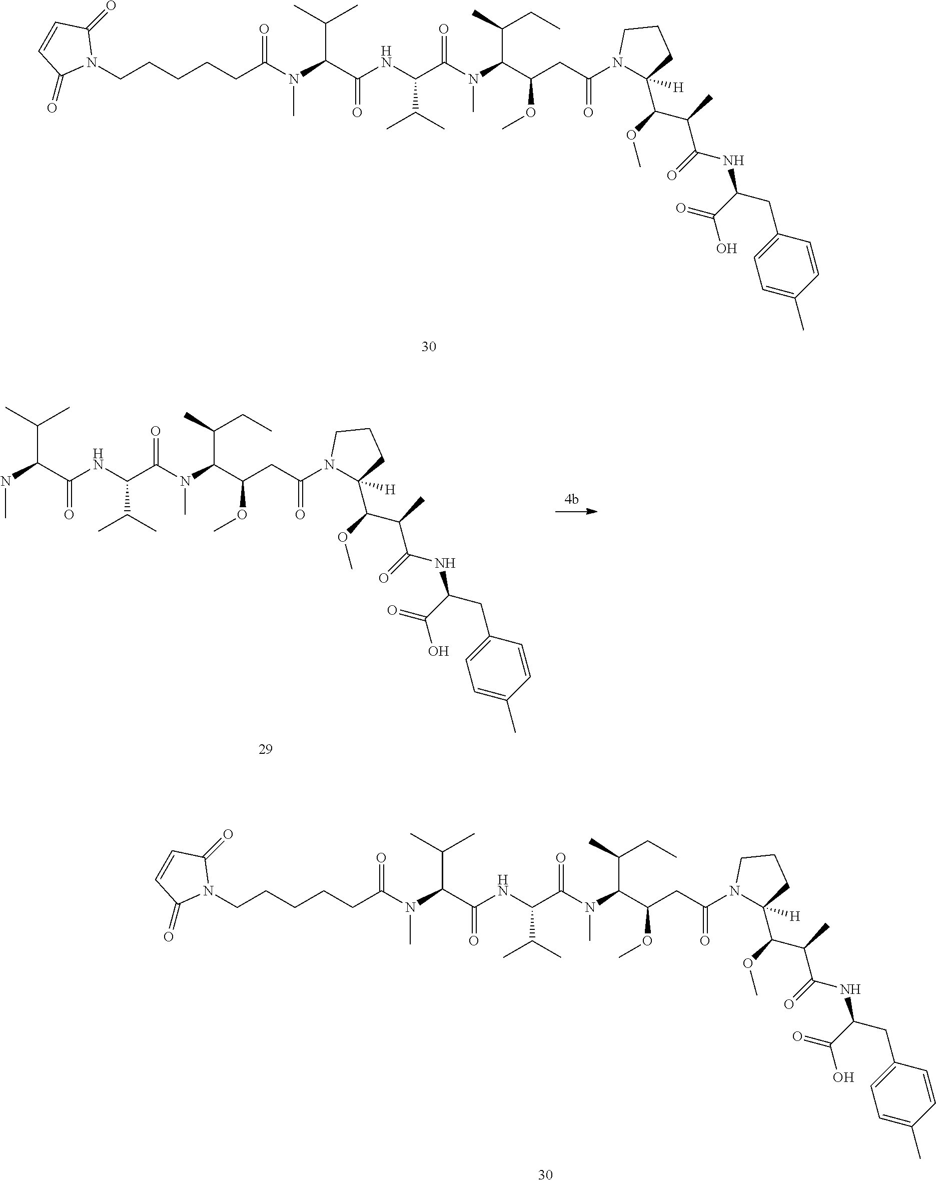 [SMILES: CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(p-tolyl)propanoic acid 29 (12 mg, 0.016 mmol) was dissolved in 1 mL of dichloromethane. The solution was added with N, N-diisopropylethylamine (0.014 0.08 mmol) under an argon atmosphere, and then added dropwise with a preformed solution of 6-(2,5-dioxo-2,5-dihydro-1H-pyrrol-1-yl) hexanoyl chloride 4b (5.54 mg, 0.024 mmol) in dichloromethane at 0° C. and stirred at room temperature for 4 hours. The reaction was quenched with methanol and concentrated under reduced pressure. The residues were purified by high performance liquid chromatography to give the title product of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(p-tolyl)propanoic acid 30 (4 mg, white solid), yield 26.5%.

MS m/z (ESI): 939.4 [M+1]

¹H NMR (400 MHz, CD₃OD): δ 7.22-7.03 (m, 4H), 6.85-6.78 (m, 2H), 4.83-4.55 (m, 3H), 4.27-3.97 (m, 3H), 3.90-3.62 (m, 3H), 3.59-2.95 (m, 14H), 2.94-2.79 (m, 1H), 2.54-2.35 (m, 3H), 2.34-2.12 (m, 5H), 2.08-1.99 (m, 1H), 1.93-1.72 (m, 3H), 1.71-1.51 (m, 5H),1.48-1.24 (m, 8H), 1.23-1.12 (m, 3H), 1.11-0.78 (m, 17H).

### Example 31

### (S)-3-(3-chlorophenyl)-2-((2R,3R)-3-((S)-1-((3R,4S,5S)-4-((S)—N,3-dimethyl-2-((S)-3-m ethyl-2-(methylamino)butanamido)butanamido)-3-methoxy-5-methylheptanoyl)pyrrolidin-2-yl)-3-methoxy-2-methylpropanamido)propanoic acid

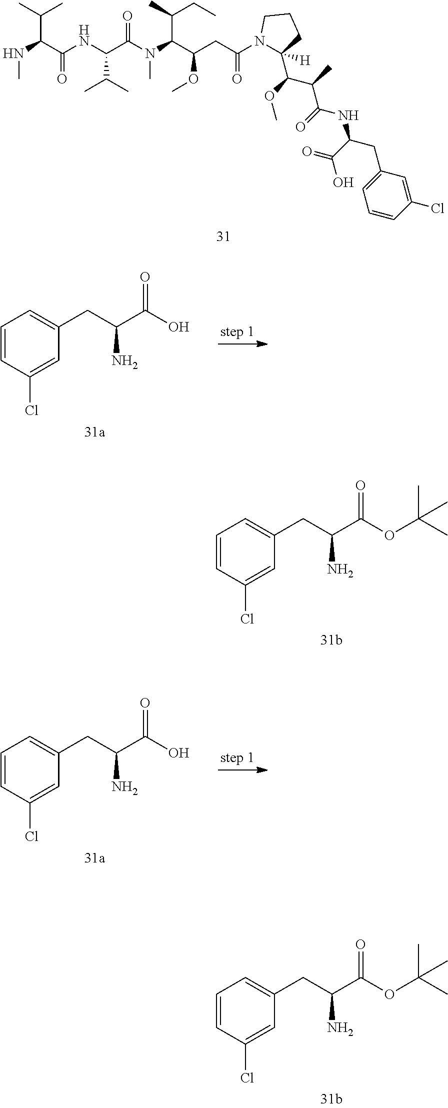 [SMILES: CC[C@H](C)[C@@H]([C@@H](CC(=O)N1CCC[C@H]1[C@H](OC)[C@@H](C)C(=O)N[C@@H](Cc1cccc(Cl)c1)C(=O)O)OC)N(C)C(=O)[C@@H](NC(=O)[C@@H](NC)C(C)C)C(C)C ; N[C@@H](Cc1cccc(Cl)c1)C(=O)O ; CC(C)(C)OC(=O)[C@@H](N)Cc1cccc(Cl)c1 ; N[C@@H](Cc1cccc(Cl)c1)C(=O)O ; CC(C)(C)OC(=O)[C@@H](N)Cc1cccc(Cl)c1]

### Step 1

### (S)-tert-butyl2-amino-3-(3-chlorophenyl)propanoate

(S)-2-amino-3-(3-chlorophenyl)propanoic acid 31a (600 mg, 3 mmol) was dissolved in 15 mL of tert-butyl acetate. The solution was added dropwise with perchloric acid (450 mg (70%), 4.5 mmol) under an argon atmosphere at 0° C., and stirred at room temperature for 12 hours. The reaction mixture was then added with 10 mL of water and the organic phase was washed successively with 20 mL of 1N hydrochloric acid and saturated sodium bicarbonate solution. The aqueous phase was combined and adjusted to pH=8 to 9 by adding dropwise with saturated sodium bicarbonate solution and extracted with dichloromethane (100 mL×2). The organic phases were combined, dried over anhydrous sodium sulfate, filtered and the filtrate was concentrated under reduced pressure to give the crude title product of (S)-tert-butyl 2-amino-3-(3-chlorophenyl)propanoate 31b (500 mg, oil). The product was used in the next step without further purification.

The preparation method was similar to Example 25, except that the raw material in step was replaced with (2R,3R)-3-45)-1-(tert-butoxycarbonyl)pyrrolidin-2-yl)-3-methoxy-2-methylpropanoic acid 25d and (S)-tert-butyl 2-amino-3-(3-chlorophenyl)propanoate 31b, to give the title product of (S)-3-(3-chlorophenyl)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)propanoic acid 31 (4 mg, white solid).

MS m/z (ESI): 766.6 [M+1]

¹H NMR (400 MHz, CD₃OD): δ 7.31-7.12 (m, 4H), 4.73-4.51 (m, 2H), 4.22-4.05 (m, 1H), 3.92-3.83 (m, 1H), 3.76-3.65 (m, 1H), 3.62-3.51 (m, 1H), 3.50-3.12 (m, 9H), 3.09-2.98 (m, 1H), 2.59-2.39 (m, 4H), 2.38-2.24 (m, 1H), 2.23-2.17 (m, 1H), 2.16-2.01 (m, 3H), 2.00-1.85 (m, 2H), 1.84-1.76 (m, 1H), 1.73-1.57 (m, 2H), 1.43-1.27 (m, 5H), 1.25-1.14 (m, 3H), 1.10-0.95 (m, 13H), 0.94-0.83 (m, 5H).

### Example 32

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(3-fluorophenyl)propanoic acid

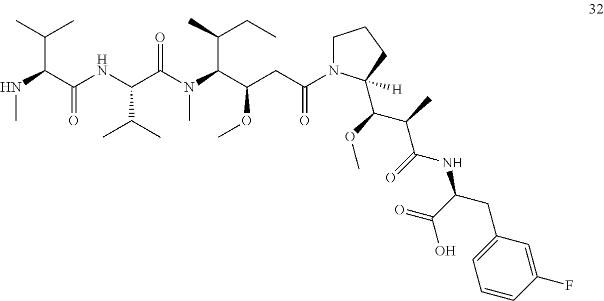 [SMILES: CC[C@H](C)[C@@H]([C@@H](CC(=O)N1CCC[C@H]1[C@H](OC)[C@@H](C)C(=O)N[C@@H](Cc1cccc(F)c1)C(=O)O)OC)N(C)C(=O)[C@@H](NC(=O)[C@@H](NC)C(C)C)C(C)C]

The preparation method was similar to Example 25, except that the raw material in step was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(3-fluorophenyl)propanoate 17b, to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(3-fluorophenyl)propanoic acid 32 (33.5 mg, white solid).

MS m/z (ESI): 750.7 [M+1]

¹H NMR (400 MHz, CD₃OD) δ 7.31-7.28 (m, 1H), 7.08-6.95 (m, 3H), 4.77-4.69 (m, 2H), 4.09-3.94 (m, 3H), 3.69-3.66 (m, 2H), 3.48-3.44 (m, 2H), 3.38-3.36 (m, 3H), 3.34-3.28 (m, 6H), 3.26-3.19 (m, 2H), 3.13 (m, 1H), 3.01-2.91 (m, 2H), 2.67-2.65 (m, 2H), 2.54-2.47 (m, 2H), 2.34-2.28 (m, 2H), 2.18-2.00 (m, 2H), 1.98-1.77 (m, 2H), 1.55-1.42 (m, 2H), 1.08-0.98 (m, 18H), 0.88-0.83 (m, 3H).

### Example 33

### (S)-3-(2,4-dichlorophenyl)-2-((2R,3R)-3-45)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)propanoic acid

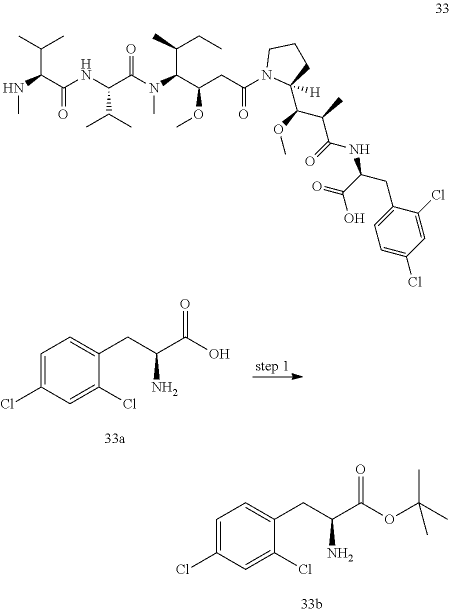 [SMILES: CC[C@H](C)[C@@H]([C@@H](CC(=O)N1CCC[C@H]1[C@H](OC)[C@@H](C)C(=O)N[C@@H](Cc1ccc(Cl)cc1Cl)C(=O)O)OC)N(C)C(=O)[C@@H](NC(=O)[C@@H](NC)C(C)C)C(C)C ; N[C@@H](Cc1ccc(Cl)cc1Cl)C(=O)O ; CC(C)(C)OC(=O)[C@@H](N)Cc1ccc(Cl)cc1Cl]

### Step 1

### (S)-tert-butyl 2-amino-3-(2,4-dichlorophenyl)propanoate

(S)-2-amino-3-(2,4-dichlorophenyl)propanoic acid 33a (1.3 g, 5.57 mmol, prepared according to the known method of “International Journal of Peptide & Protein Research, 1987, 30(1), 13-21”) was dissolved in 10 mL of tert-butyl acetate. The solution was added dropwise with perchloric acid (1.2 g (70%), 8.36 mmol) at 0° C. under an argon atmosphere, and stirred at room temperature for 12 hours. The reaction mixture was diluted with methylene chloride and added with saturated sodium bicarbonate solution dropwise until pH reached 8 to 9. The aqueous phase was extracted with methylene chloride. The organic phases were combined, washed successively with water and saturated sodium chloride solution, dried over anhydrous sodium sulfate, filtered and the filtrate was concentrated under reduced pressure to give the crude title product of (S)-tert-butyl 2-amino-3-(2,4-dichlorophenyl)propanoate 33b (3.4 g, light yellow oil). The product was used in the next step without further purification.

The preparation method was similar to Example 25, except that the raw material in step was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(2,4-dichlorophenyl)propanoate 33b, to give the title product of (S)-3-(2,4-di chlorophenyl)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)propanoic acid 33 (23 mg, white solid).

MS m/z (ESI): 800.6 [M+1]

¹H NMR (400 MHz, CD₃OD) δ 7.43-7.42 (m, 1H), 7.26-7.23 (m, 2H), 4.75-4.67 (m, 2H), 4.14-4.04 (m, 2H), 4.00-3.98 (m, 1H), 3.91-3.89 (m, 1H), 3.68-3.67 (m, 3H), 3.39-3.26 (m, 12H), 3.21-3.12 (m, 3H), 2.67-2.64 (m, 3H), 2.50-2.46 (m, 3H), 2.31-2.28 (m, 2H), 2.17-2.15 (m, 2H), 2.02-2.00 (m, 4H), 1.90-1.88 (m, 2H), 1.74-1.72 (m, 2H), 1.39-1.37 (m, 2H), 1.06-0.98 (m, 12H).

### Example 34

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(o-tolyl)propanoic acid

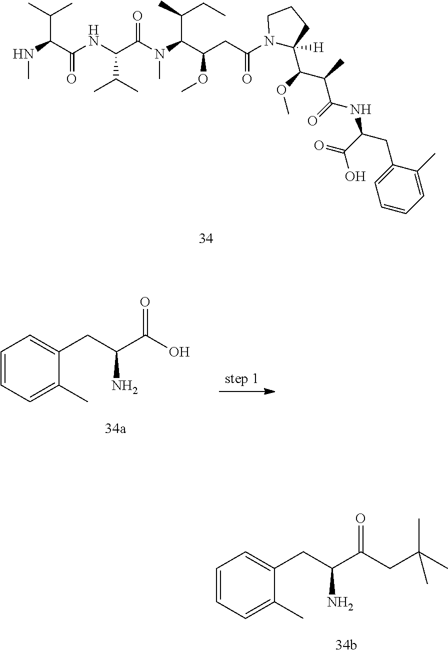 [SMILES: CC[C@H](C)[C@@H]([C@@H](CC(=O)N1CCC[C@H]1[C@H](OC)[C@@H](C)C(=O)N[C@@H](Cc1ccccc1C)C(=O)O)OC)N(C)C(=O)[C@@H](NC(=O)[C@@H](NC)C(C)C)C(C)C ; Cc1ccccc1C[C@H](N)C(=O)O ; Cc1ccccc1C[C@H](N)C(=O)CC(C)(C)C]

### Step 1

### (S)-tert-butyl 2-amino-3-(o-tolyl)propanoate

(S)-2-amino-3-(o-tolyl)propanoic acid 34a (100 mg, 0.55 mmol, prepared according to the known method of “International Journal of Peptide & Protein Research, 1987, 30(1), 13-21”) was dissolved in 2.5 mL of tert-butyl acetate, and added dropwise with perchloric acid (107 mg (70%), 0.83 mmol) under an argon atmosphere. The mixture was stirred at room temperature for 16 hours and then diluted with 5 mL of dichloromethane, and saturated sodium bicarbonate solution was added dropwise to adjust to pH=8. The aqueous phase was extracted with dichloromethane (5 mL×3). The organic phases were combined, washed successively with water and saturated sodium chloride solution, dried over anhydrous sodium sulfate, filtered and the filtrate was concentrated under reduced pressure to give the crude title product of (S)-tert-butyl 2-amino-3-(o-tolyl)propanoate 34b (140 mg, colorless oil). The product was used in the next step without further purification.

The preparation method was similar to Example 25, except that the raw material in step 4 was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(o-tolyl)propanoate 34b, to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(o-tolyl)propanoic acid 34 (40 mg, white solid).

MS m/z (ESI): 746.8 [M+1]

¹H NMR (400 MHz, CD₃OD) δ 8.41-8.39 (d, 1H), 8.19-8.17 (d, 1H), 7.18-7.05 (m, 4H), 4.80-4.78 (d, 1H), 4.71-4.69 (d, 1H), 3.86-3.84 (m, 1H), 3.76-3.72 (m, 1H), 3.69-3.63 (m, 2H), 3.48-3.42 (d, 2H), 3.38-3.23 (m, 5H), 3.20-3.12 (m, 4H), 2.98-2.87 (m, 2H), 2.66-2.65 (d, 3H), 2.53-2.50 (d, 1H), 2.46-2.44 (d, 1H), 2.41-2.26 (m, 4H), 2.21-2.14 (m, 2H), 2.10-2.04 (m, 1H), 1.87-1.85 (m, 2H), 1.77-1.74 (m, 1H), 1.62-1.53 (m, 2H), 1.33-1.28 (m, 3H), 1.23-1.21 (d, 2H), 1.16-1.14 (d, 2H), 1.08-0.96 (m, 14H), 0.90-0.84 (m, 3H).

### Example 35

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(thiophen-2-yl)propanoic acid

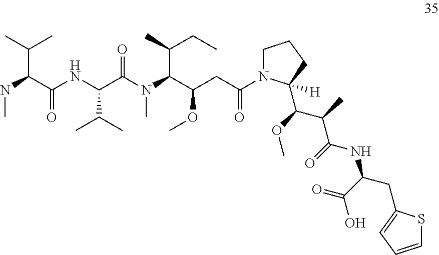 [SMILES: CC[C@H](C)[C@@H]([C@@H](CC(=O)N1CCC[C@H]1[C@H](OC)[C@@H](C)C(=O)N[C@@H](Cc1cccs1)C(=O)O)OC)N(C)C(=O)[C@@H](NC(=O)[C@@H](NC)C(C)C)C(C)C]

The preparation method was similar to Example 25, except that the raw material in step 4 was replaced with (2R,3R)-3-((S)-1-(tert-butoxycarbonyl)pyrrolidin-2-yl)-3-methoxy-2-methylpropanoic acid 25d and (S)-tert-butyl 2-amino-3-(thiophen-2-yl)propanoate 16b, to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(thiophen-2-yl)propanoic acid 35 (2.6 mg, white solid).

¹H NMR (400 MHz, CD₃OD) δ 7.21-7.19 (m, 1H), 6.91-6.90 (m, 2H), 4.80-4.65 (m, 2H), 4.16-4.08 (m, 2H), 3.93-3.90 (m, 1H), 3.70-3.66 (m, 2H), 3.55-3.52 (m, 1H), 3.48-3.45 (m, 2H), 3.40-3.26 (m, 6H), 3.25-3.13 (m, 4H), 2.96-2.82 (t, 1H), 2.69-2.65 (d, 3H), 2.52-2.47 (m, 2H), 2.39-2.29 (m, 1H), 2.21-2.14 (m, 2H), 1.92- (s, 3H), 1.63-1.61 (m, 2H), 1.40-1.29 (m, 3H), 1.23-1.14 (m, 3H), 1.10-0.98 (m, 13H), 0.88-0.85 (t, 3H).

### Example 36

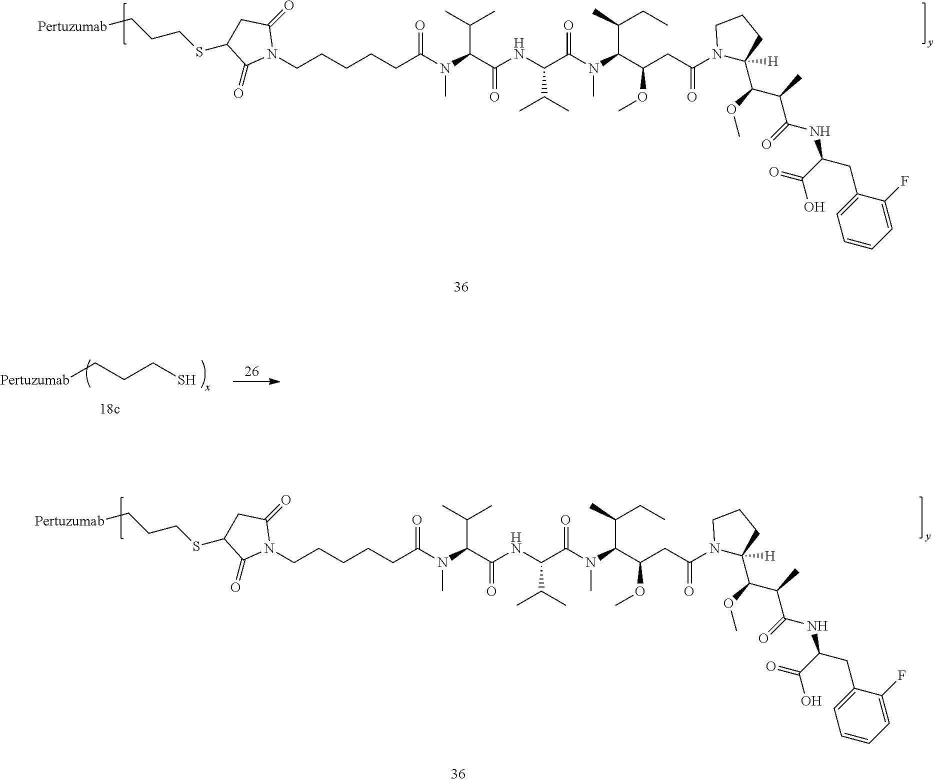

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-fluorophenyl)propanoic acid 26 (5.45 mg, 5.78 μmol) was dissolved in 1.2 mL of acetonitrile and added with Pertuzumab-propanethiol solution 18c (7.56 mg/mL, 12 mL), and stirred at 25° C. for 4 hours. Then the reaction mixture was desalted and purified by Sephadex G25 gel column (elution phase: 0.05 M PBS solution, pH 6.5), filtrated under a sterile condition through a 0.2 μm filter to obtain the title product of 36 in PBS buffer (3.51 mg/mL, 27.5 mL), and then stored at 4° C.

Q-TOF LC/MS: characteristic peaks: 148094.39(M_{Ab}+0D), 149111.06(M_{Ab}+1D), 150167.12 (M_{Ab}+2D), 151188.93(M_{Ab}+3D), 152243.46(M_{Ab}+4D), 153272.96(M_{Ab}+5D).

Average value: y=1.9

### Example 37

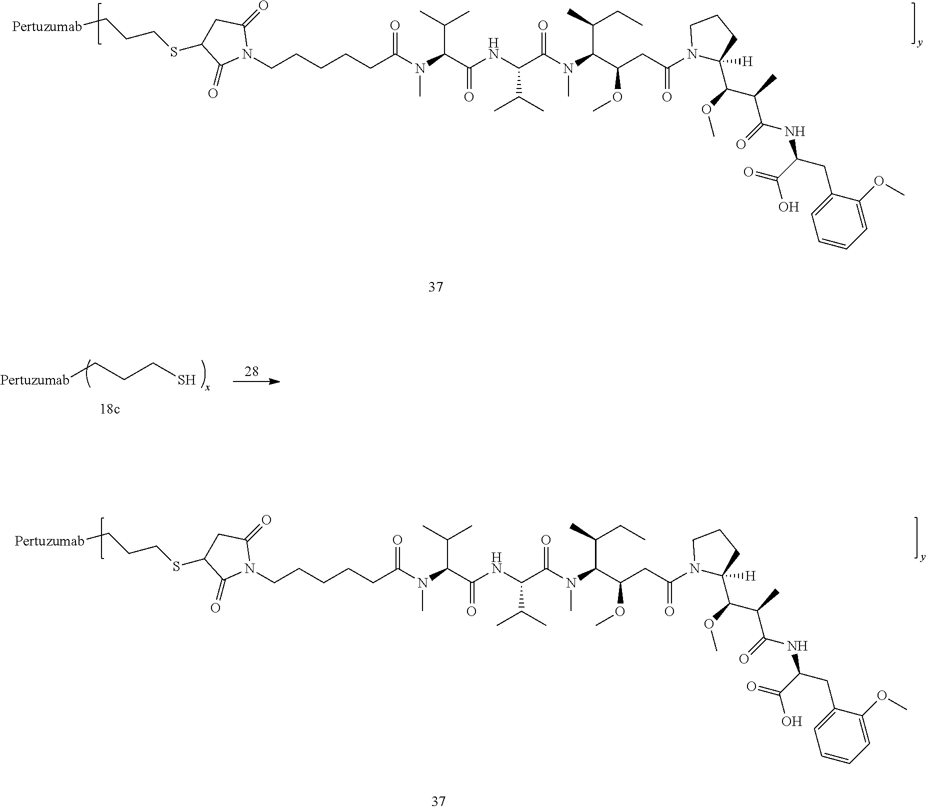

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-methoxyphenyl)propanoic acid 28 (1.75 mg, 1.83 μmol) was dissolved in 0.9 mL of acetonitrile and added with Pertuzumab-propanethiol solution 18c (2.31 mg/mL, 9.0 mL), and stirred at 25° C. for 6 hours. Then the reaction mixture was desalted and purified by Sephadex G25 gel column (eluting phase: 0.05 M PBS solution, pH 6.5), filtrated under a sterile condition through a 0.2 μm filter to obtain the title product of 37 in PBS buffer (1.35 mg/mL, 13.0 mL), and then stored at 4° C.

Q-TOF LC/MS: characteristic peaks: 148093.25(M_{Ab}+0D), 149281.78(M_{Ab}+1D), 150474.33 (M_{Ab}+2D), 151372.72(M_{Ab}+3D), 152373.24(M_{Ab}+4D), 153469.40(M_{Ab}+5D).

Average value: y=2.3.

### Example 38

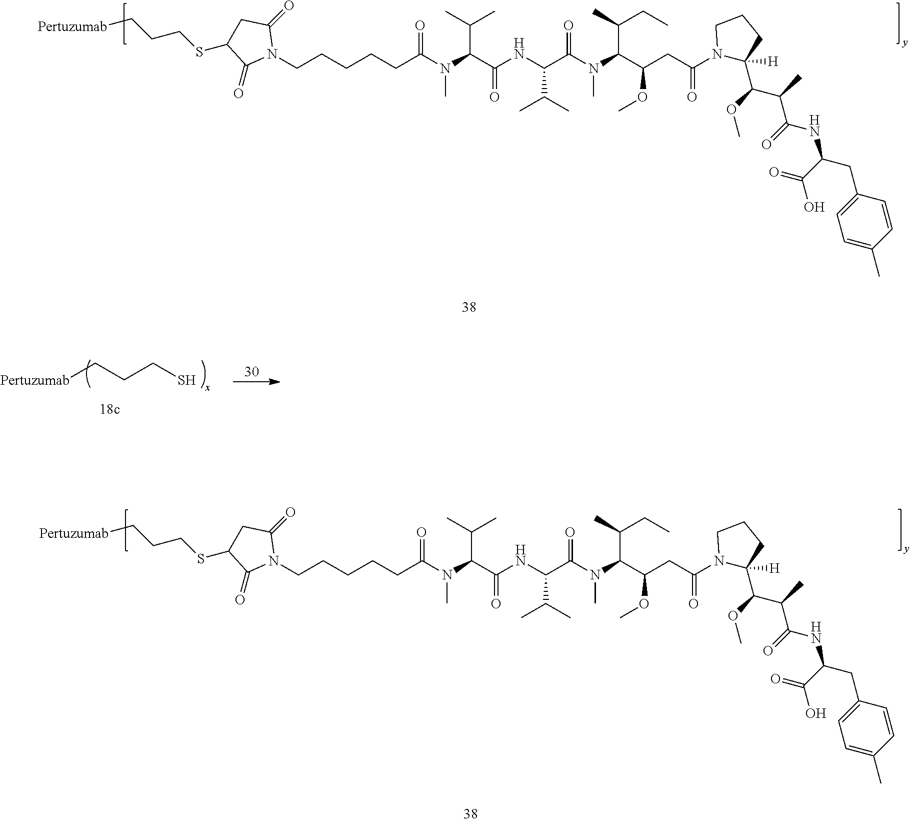

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(p-tolyl)propanoic acid 30 (1.07 mg, 1.14 μmol) was dissolved in 1.25 mL of acetonitrile and added with Pertuzumab-propanethiol solution 18c (1.5 mg/mL, 12.5 mL), and stirred at 25° C. for 4.5 hours. The reaction mixture was desalted and purified by Sephadex G25 gel column (elution phase: 0.05 M PBS solution, pH 6.5), filtered under a sterile condition through a 0.2 μm filter to obtain the title product of 38 in PBS buffer (0.74 mg/mL, 17.5 mL), and then stored at 4° C.

Q-TOF LC/MS: characteristic peaks: 148247.08(M_{Ab}+0D), 149265.32(M_{Ab}+1D), 150276.10 (M_{Ab}+2D), 151334.50(M_{Ab}+3D), 152392.21(M_{Ab}+4D), 153388.72(M_{Ab}+5D).

Average value: y=2.4.

### Example 39

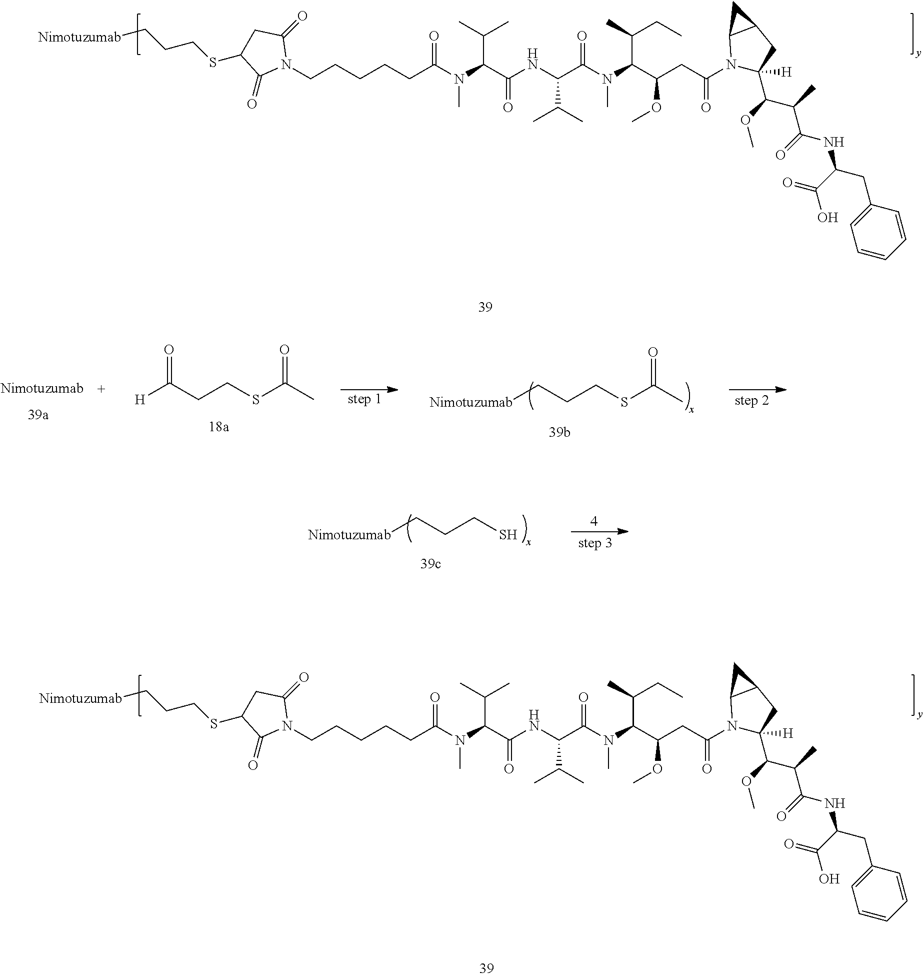

### Step 1

S-(3-oxopropyl) ethanethioate 18a (2.44 mg, 18.5 μmop was dissolved in 3.0 mL of acetonitrile. The Nimotuzumab in acetic acid/sodium acetate buffer (10.22 mg/ml, 30 mL, 0.204 mmol) with pH 4.3 was added with the solution of S-(3-oxopropyl) ethanethioate 18a in acetonitrile, and then added with 1.2 mL of an aqueous solution of sodium cyanoborohydride (49.86 mg, 793 μmop dropwise. The reaction mixture was stirred at 25° C. for 2 hours. The reaction solution was first purified with PBS buffer (250 mL) containing 10% acetonitrile by 30 KDa ultrafiltration, then purified by a 30 KDa ultrafiltration pack using PBS buffer (200 mL) with pH=6.5 to remove the unreacted S-(3-oxopropyl) ethanethioate 18a and sodium cyanoborohydride to give the title product of 39b in PBS buffer (about 75 mL), which was used directly in the next step.

### Step 2

39b in PBS buffer solution (75.0 mL) was added with 2.0 mL of a 2.0 M solution of hydroxylamine hydrochloride, and then placed in a shaking in a water bath at 25° C. for 30 minutes. The reaction solution was purified by 30 KDa ultrafiltration with PBS buffer with pH=6.5 to give the title product of Nimotuzumab-propanethiol 39c in PBS buffer solution (concentration 5.38 mg/ml, 55 mL).

### Step 3

(S)-2-((2R,3R)-3-((1S,3S,5S)-2-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido) 4 (4.48 mg, 4.78 μmop was dissolved in 1.1 mL of acetonitrile and added with Nimotuzumab-propanethiol in PBS buffer 39c (5.38 mg/mL, 11 mL). After shaking on a shaker in a water bath at 25° C. for 4 hours, the reaction was stopped. The resulting mixture was desalted and purified with a Sephadex G25 gel column (Elution phase: 0.05 M PBS solution with pH 6.5) to give the crude title product of 39 in PBS buffer (2.96 mg/mL, 20.5 mL), which was further concentrated to about 6 mL by centrifugation, and desalted and purified again with a Sephadex G25 gel column (Elution phase: 0.05 M PBS solution at pH 6.5) to give the title product of 39 in PBS buffer (4.25 mg/mL, 11.8 mL), and then stored at 4° C.

Q-TOF LC/MS: characteristic peaks: 150188.68(M_{Ab}+0D), 151234.76(M_{Ab}+1D), 152248.46 (M_{Ab}+2D), 153419.10(M_{Ab}+3D), 154312.17(M_{Ab}+4D), 155358.48 (M_{Ab}+5D).

Average value: y=2.2.

### Example 40

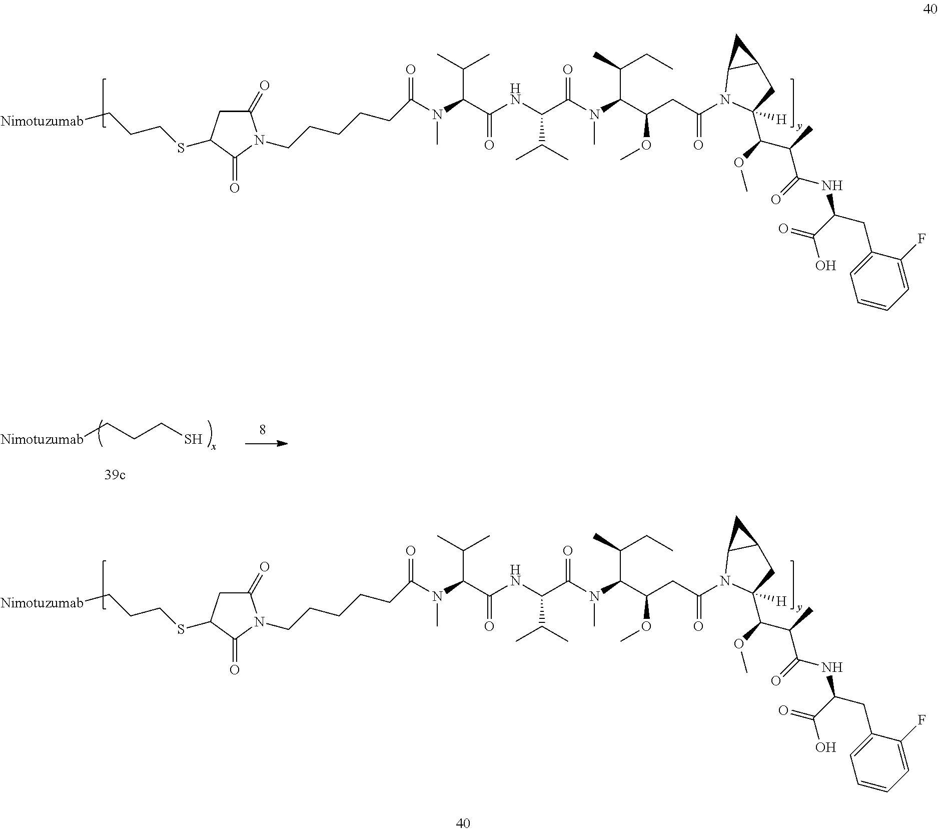

(S)-2-((2R,3R)-3-((1S,3S,5S)-2-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-2-azabicyclo[3.1.0]hexan-3-yl)-3-methoxy-2-methylpropanamido)-3-(2-fluorophenyl)propanoic acid 8 (4.32 mg, 4.52 μmol) was dissolved in 1.1 mL of acetonitrile and added with Nimotuzumab-propanethiol 39c in PBS buffer (5.38 mg/mL, 11 mL). After shaking on a shaker in a water bath at 25° C. for 4 hours, the reaction was stopped.

The resulting mixture was desalted with Sephadex G25 gel column (Elution phase: 0.05 M PBS solution with pH 6.5) to give the crude title product of 40 in PBS buffer (2.92 mg/mL, 20 mL), which was further concentrated to about 5.5 mL, and desalted again with a Sephadex G25 gel column (Elution phase: 0.05 M PBS solution with pH 6.5) to give the title product of 40 in PBS buffer (4.25 mg/mL, 11.6 mL), then stored at 4° C. Q-TOF LC/MS:characteristic peaks: 150186.98(M_{Ab}+0D), 151374.09(M_{Ab}+1D), 152287.22 (M_{Ab}+2D), 153353.26(M_{Ab}+3D), 154501.80(M_{Ab}+4D), 155575.57(M_{Ab}+5D).

Average value: y=2.2.

### Example 41

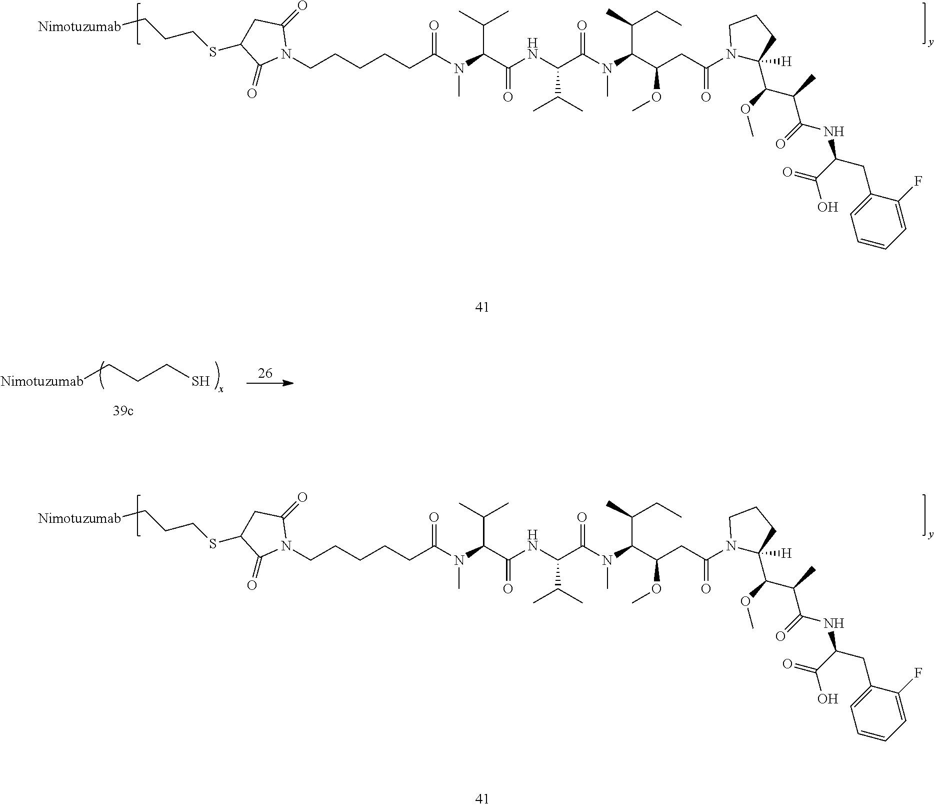

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(2-fluorophenyl)propanoic acid 26 (4.45 mg, 4.72 μmol) was dissolved in 1.1 mL of acetonitrile and added with Nimotuzumab-propanethiol 39c in PBS buffer (5.38 mg/mL, 11 mL). After shaking on a shaker in a water bath at 25° C. for 4 hours, the reaction was stopped. The resulting mixture was desalted with a Sephadex G25 gel column (Elution phase: 0.05 M PBS solution at pH 6.5) to give the crude title product of 41 in PBS buffer (2.96 mg/mL, 20 mL), which was further centrifuged and concentrated to about 5.5 mL and desalted again with a Sephadex G25 gel column (Elution phase: 0.05 M PBS solution with pH 6.5) to give the title product of 41 in PBS buffer (4.33 mg/mL, 11 mL), then stored at 4° C.

Q-TOF LC/MS: characteristic peaks: 150186.05(M_{Ab}+0D), 151362.44(M_{Ab}+1D), 152261.90 (M_{Ab}+2D), 153438.29(M_{Ab}+3D), 154339.30(M_{Ab}+4D), 155511.23(M_{Ab}+5D).

Average value: y=2.3.

### Example 42

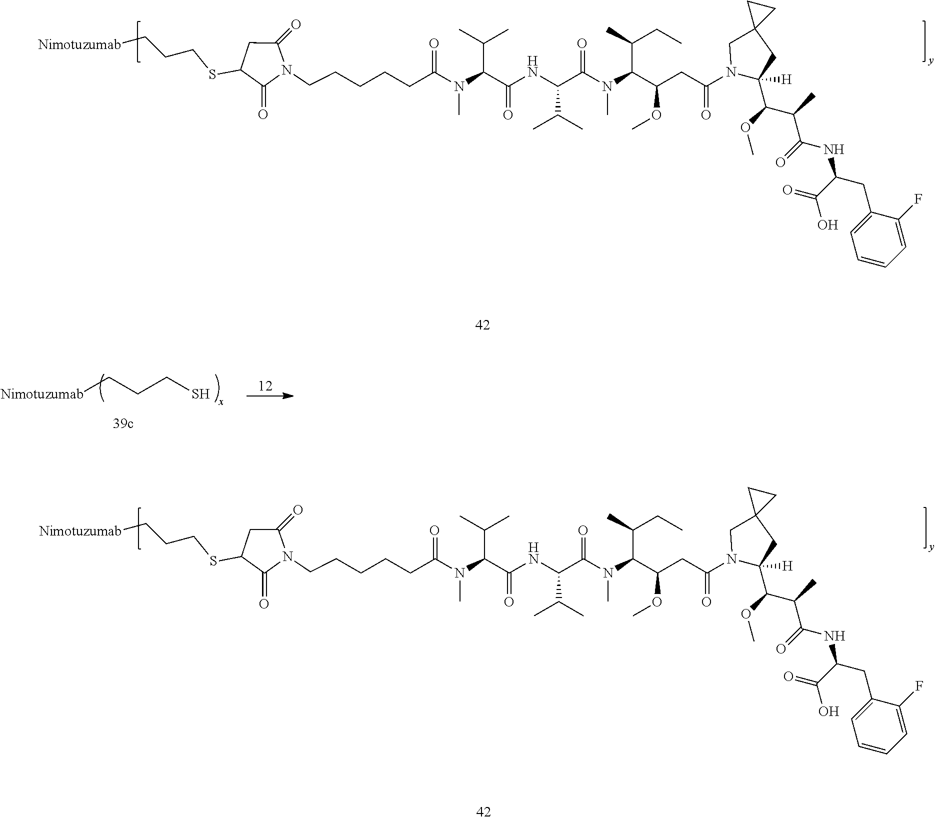

(S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-(2-fluorophenyl)propanoic acid 12 (0.56 mg, 0.58 μmol) was dissolved in 0.42 mL of acetonitrile and added with Nimotuzumab-propanethiol solution 39c (2.06 mg/mL, 4.2 mL). After shaking on a shaker in a water bath at 25° C. for 5 hours, the reaction was stopped.

The reaction mixture was desalted with a Sephadex G25 gel column (Elution phase: 0.05 M PBS solution at pH 6.5) to give the crude title product of 42 in PBS buffer (0.74 mg/mL, 10 mL), which was further centrifuged to about 5.5 mL and desalted again with a Sephadex G25 gel column (Elution phase: 0.05 M PBS solution with pH 6.5) to give the title product of 42 in PBS buffer (1.15 mg/mL, 6 mL), then stored at 4° C.

Q-TOF LC/MS: characteristic peaks: 150188.42(M_{Ab}+0D), 151387.82(M_{Ab}+1D), 152472.27 (M_{Ab}+2D), 153528.33(M_{Ab}+3D).

Average value: y=1.0.

### Example 43

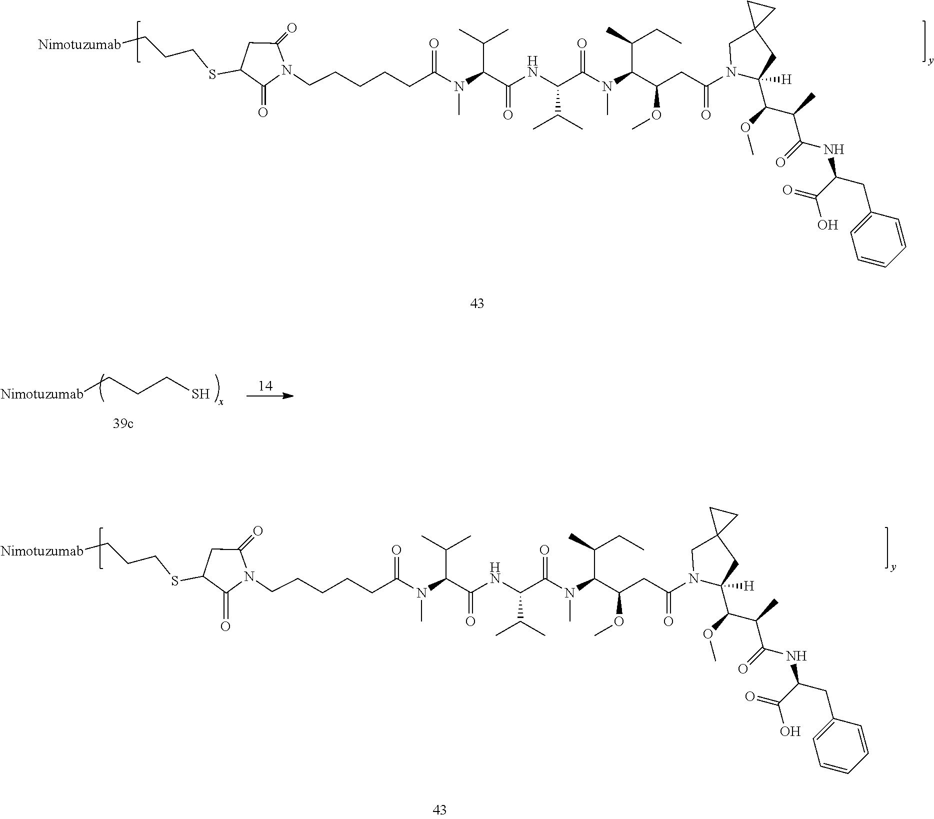

(S)-2-((2R,3R)-3-((S)-5-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-5-azaspiro[2.4]heptan-6-yl)-3-methoxy-2-methylpropanamido)-3-phenylpropanoic acid 14 (0.60 mg, 0.63 μmol) was dissolved in 0.42 mL of acetonitrile and added with Nimotuzumab-propanethiol solution 39c (2.06 mg/mL, 4.2 mL). After shaking on a shaker in a water bath at 25° C. for 5 hours, the reaction was stopped.

The reaction mixture was desalted with a Sephadex G25 gel column (Elution phase: 0.05 M PBS solution at pH 6.5) to give the crude title product of 43 in PBS buffer (0.78 mg/mL, 9.5 mL), which was further centrifuged and concentrated to about 5.5 mL and desalted again with a Sephadex G25 gel column (Elution phase: 0.05 M PBS solution with pH 6.5) to give the title product of 43 in PBS buffer (1.16 mg/mL, 6 mL), then stored at 4° C.

Q-TOF LC/MS: characteristic peaks: 150188.39(M_{Ab}+0D), 151251.46(M_{Ab}+1D), 152442.90 (M_{Ab}+2D), 153507.73(M_{Ab}+3D).

Average value: y=1.0.

### Example 44

### (S)-2-((2R,3R)-3-((2S,4S)-1-((3R,4S,5S)-4-((S)—N,3-dimethyl-2-((S)-3-methyl-2-(methylamino)butanamido)butanamido)-3-methoxy-5-methylheptanoyl)-4-fluoropyrrolidin-2-yl)-3-methoxy-2-methylpropanamido)-3-phenylpropanoic acid

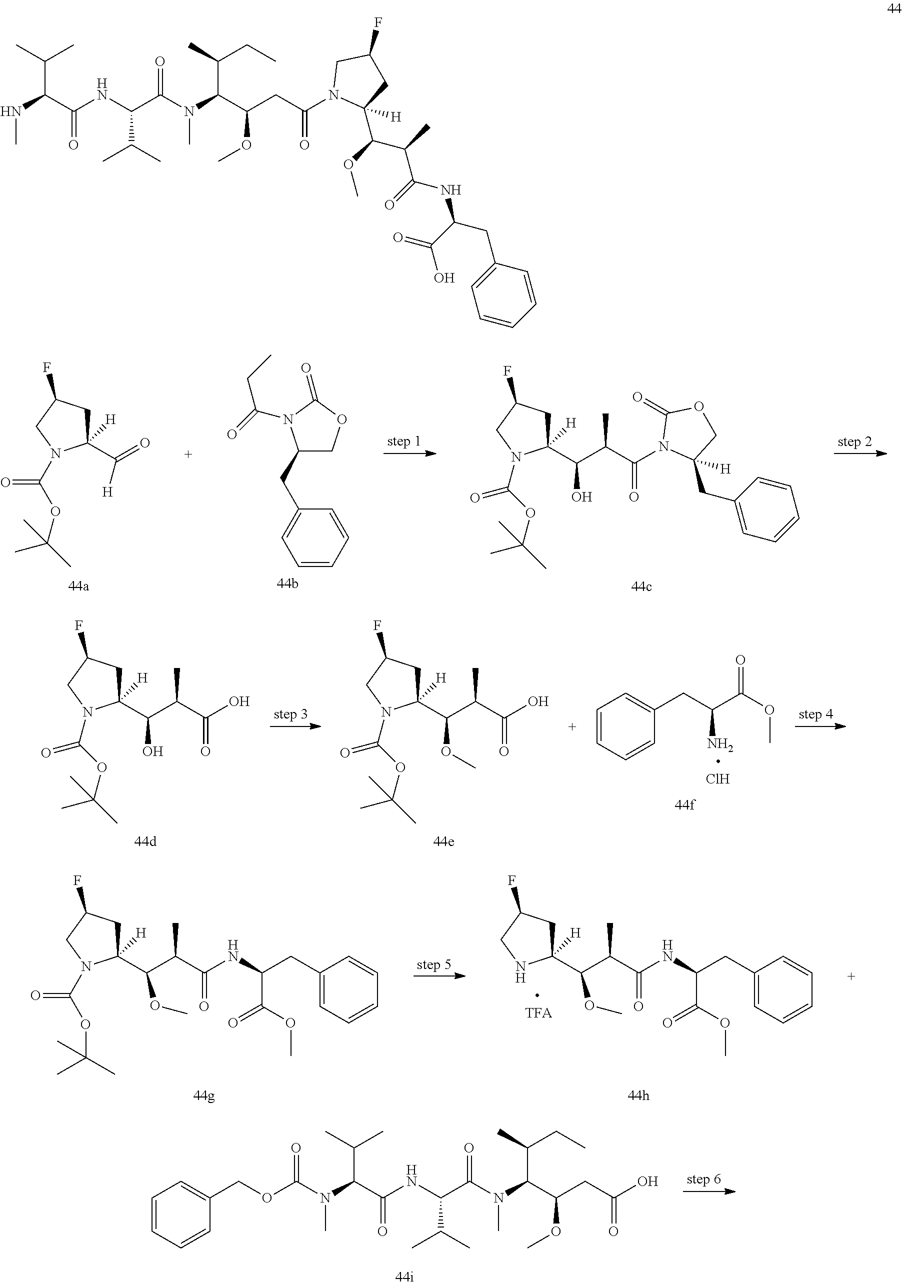

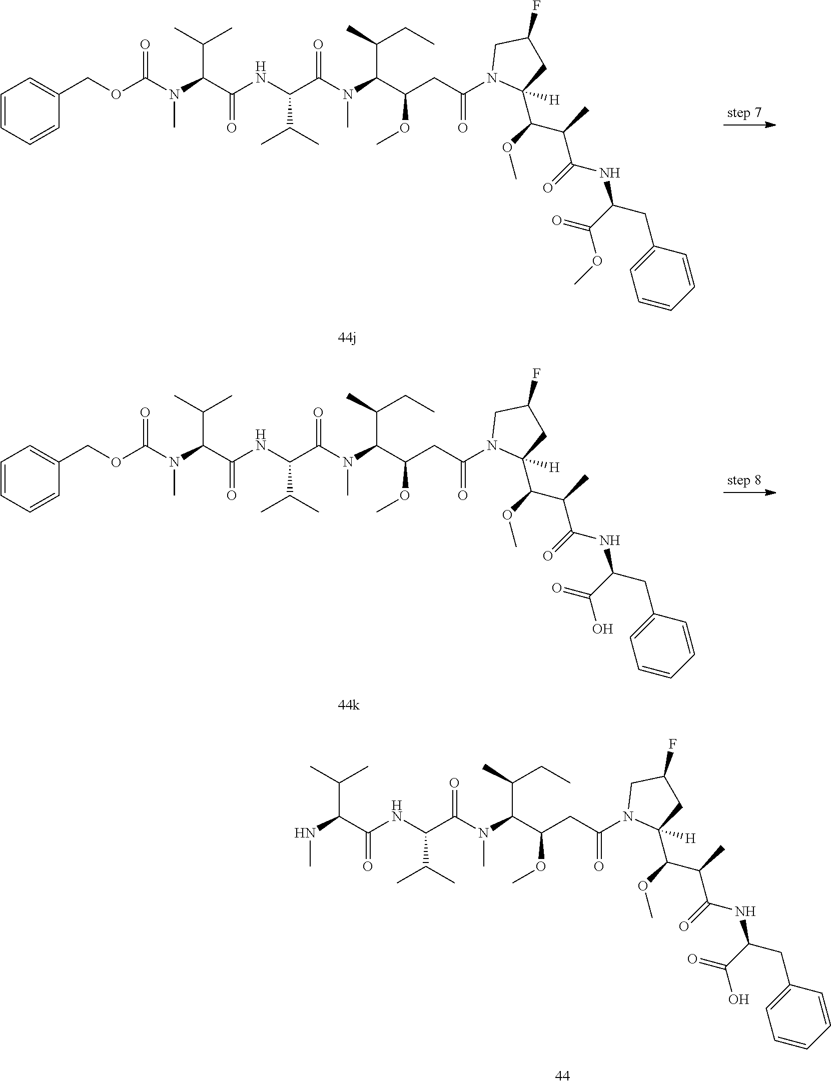 [SMILES: CC[C@H](C)[C@@H]([C@@H](CC(=O)N1C[C@@H](F)C[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)OCc1ccccc1)C(C)C ; CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccccc1)C(C)C ; CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

### Step 1

### (2S,4S)-tert-butyl 2-((1R,2R)-3-((R)-4-benzyl-2-oxooxazolidin-3-yl)-1-hydroxy-2-methyl-3-oxopropyl)-4-fluoropyrrolidine-1-carboxylate

(R)-4-benzyl-3-propionyloxazolidin-2-one 44b (21 g, 90 mmol, prepared according to the known method of “Tetrahedron Letters, 1999, 40(36), 6545-6547”) was dissolved in 300 mL of dichloromethane, and cooled to 0° C. under an argon atmosphere. The reaction solution was added dropwise with titanium tetrachloride (9.8 mL, 1.1 mmol) at 0° C., and the solution gradually changed from colorless to yellow to form a yellow solid. N,N-diisopropylethylamine (40 mL, 225 mmol) was slowly added dropwise with a formation of white smoke, and the solution changed from yellow to reddish brown. The mixture was stirred at 0° C. for 1 hour, and then cooled to −78° C. and added with 50 mL of (2S,4S)-tert-butyl 4-fluoro-2-formylpyrrolidine-1-carboxylate 44a (21.27 g, 98 mmol, prepared according to the known method of “Tetrahedron: Asymmetry, 2014, 25(3), 212-218”) in dichloromethane, and stirred for another 1.5 hours at −78° C. The completion of the reaction was monitored by TLC. The reaction solution was added with 200 mL of sodium bicarbonate solution (5%) and the aqueous phase was extracted with dichloromethane (300 mL×2). The organic phases were combined, washed successively with water (200 mL) and saturated sodium chloride solution (200 mL), dried over anhydrous magnesium sulfate, filtered, and the filtrate was concentrated under reduced pressure. The residues were purified by silica gel column chromatography using eluent system B to give the title product of (2S,4S)-tert-butyl 2-((1R,2R)-3-((R)-4-benzyl-2-oxooxazolidin-3-yl)-1-hydroxy-2-methyl-3-oxopropyl)-4-fluoropyrrolidine-1-carboxylate 44c (19 g, light yellow solid), yield 43.2%. MS m/z (ESI): 351.06 [M-100+1]

### Step 2

### (2R,3R)-3-((2S,4S)-1-(tert-butoxycarbonyl)-4-fluoropyrrolidin-2-yl)-3-hydroxy-2-methylpropanoic acid

(2S,4S)-tert-butyl 2-((1R,2R)-3-((R)-4-benzyl-2-oxooxazolidin-3-yl)-1-hydroxy-2-methyl-3-oxopropyl)-4-fluoropyrrolidine-1-carboxylate 44c (19 g, 42 mmol) was dissolved in 200 mL of tetrahydrofuran and 50 mL of water, and cooled to 0° C. under argon atmosphere. 30% hydrogen peroxide (17.2 mL, 147 mmol) was slowly added dropwise, and 80 mL of lithium hydroxide monohydrate (2.86 g, 68 mmol) was added. The reaction mixture was stirred at 0° C. for 5 hours. A solution of 100 mL of sodium sulfite (21.2 g, 168 mmol) was added to the reaction solution and further stirred at 25° C. for 16 hours. After completion of the reaction, the organic phase was concentrated under reduced pressure and the residues were washed with dichloromethane (200 mL×3). The aqueous phase was adjusted to pH 3 with 1N hydrochloric acid and extracted with ethyl acetate (200 mL×4). The organic phases were combined and concentrated under reduced pressure. The residues were dissolved in 350 mL of sodium bicarbonate solution (5%), and washed with dichloromethane (200 mL×2). The aqueous phase was adjusted to pH 3 with 2N hydrochloric acid, extracted with ethyl acetate (200 mL×5), and the organic phases were combined, dried over anhydrous sodium sulfate, filtered, and the filtrate was concentrated under reduced pressure to give the crude title product of (2R,3R)-3-((2S,4S)-1-(tert-butoxy carbonyl)-4-fluoropyrrolidin-2-yl)-3-hydroxy-2-methylpropanoic acid 44d (11.6 g, light yellow viscous liquid). The product was used in the next step without further purification.

MS m/z (ESI): 191.47 [M-100+1]

### Step 3

### (2R,3R)-3-((2S,4S)-1-(tert-butoxycarbonyl)-4-fluoropyrrolidin-2-yl)-3-methoxy-2-methylpropanoic acid

(2R,3R)-3-((2S,4S)-1-(tert-butoxy carbonyl)-4-fluoropyrrolidin-2-yl)-3-hydroxy-2-methylpropanoic acid 44d (11.6 g, 39.8 mmol) was dissolved in 200 mL of tetrahydrofuran and added with methyl iodide (91 g, 640 mmol) under a nitrogen atmosphere at 0° C. Then, sodium hydride (7.32 g (60%), 183 mmol) was added in portions, and stirred at 0° C. for 48 hours. The reaction mixture was then quenched by the addition of 500 mL of ice water and then extracted with ethyl acetate (150 mL). The aqueous phase was washed with ethylether (150 mL×2), adjusted to pH=3 with 2N hydrochloric acid and extracted with ethyl acetate (250 mL×3). The organic phases were combined, dried over anhydrous sodium sulfate, filtered, and the filtrate was concentrated under reduced pressure. The residues were purified by silica gel column chromatography using eluent system B to give the title product of (2R,3R)-3-((2S,4S)-1-(tert-butoxycarbonyl)-4-fluoropyrrolidin-2-yl)-3-methoxy-2-meth ylpropanoic acid 44e (8.0 g, light yellow viscous liquid), yield 65.6%.

MS m/z (ESI): 205.68 [M-100+1]

### Step 4

### (2S,4S)-tert-butyl 4-fluoro-2-((1R,2R)-1-methoxy-3-(((S)-1-methoxy-1-oxo-3-phenylpropan-2-yl)amino)-2-methyl-3-oxopropyl)pyrrolidine-1-carboxylate

(2R,3R)-3-((2S,4S)-1-(tert-butoxycarbonyl)-4-fluoropyrrolidin-2-yl)-3-methoxy-2-methylpropanoic acid 44e (580 mg, 1.9 mmol) was dissolved in 15 mL of acetonitrile, and added with (S)-methyl 2-amino-3-phenylpropanoate hydrochloride 44f (480 mg, 2.2 mmol, prepared according to the known method of “Journal of Heterocyclic Chemistry, 2013, 50(2), 320-325”), N,N-diisopropylethylamine (1.42 mL, 8 mmol) and 2-(7-azobenzotriazole)-N,N,N′,N′-tetramethyluronium hexafluorophosphate (1.07 g, 2.8 mmol). The reaction mixture was stirred at room temperature for 12 hours. When the reaction was complete by TLC, the reaction mixture was diluted with 40 mL of ethyl acetate, washed successively with saturated ammonium chloride solution (20 mL) and saturated sodium chloride solution (20 mL), dried over anhydrous sodium sulfate, filtered, and the filtrate was concentrated under reduced pressure. The residues were purified by silica gel column chromatography using eluent system B to give the title product of (2S,4S)-tert-butyl 4-fluoro-2-((1R,2R)-1-methoxy-3-(((S)-1-methoxy-1-oxo-3-phenylpropan-2-yl)amino)-2-methyl-3-oxopropyl)pyrrolidine-1-carboxylate 44g (750 mg, white solid), yield 83.2%.

MS m/z (ESI): 222.62 [M-100+1]

### Step 5

### (S)-methyl 2-((2R,3R)-3-((2S,4S)-4-fluoropyrrolidin-2-yl)-3-methoxy-2-methylpropanamido)-3-phenylpropanoate trifluoroacetate

(2S,4S)-tert-butyl 4-fluoro-2-((1R,2R)-1-methoxy-3-(((S)-1-methoxy-1-oxo-3-phenylpropan-2-yl)amino)-2-methyl-3-oxopropyl)pyrrolidine-1-carboxylate 44g (738 mg, 1.58 mmol) was dissolved in 15 mL of dichloromethane, and added dropwise with trifluoroacetic acid (3 mL, 30 mmol), then stirred at room temperature for 12 hours. When the reaction was complete by TLC, the reaction solution was concentrated under reduced pressure to give the crude title product of (S)-methyl 2-((2R,3R)-3-((2S,4S)-4-fluoropyrrolidin-2-yl)-3-methoxy-2-methylpropanamido)-3-phenylpropanoate trifluoroacetate 44h (1.24 g, yellow viscous liquid). The product was used in the next step without further purification.

MS m/z (ESI): 236.39 [M+1]

### Step 6

### (S)-methyl 2-((2R,3R)-3-((2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoate

(S)-methyl 2-((2R,3R)-3-((2S,4S)-4-fluoropyrrolidin-2-yl)-3-methoxy-2-methylpropanamido)-3-phenylpropanoate trifluoroacetate 44h (572 mg, 1.56 mmol) and (5S,8S,11S,12R)-11-((S)-sec-butyl)-5,8-diisopropyl-12-methoxy-4,10-dimethyl-3,6,9-tri oxo-1-phenyl-2-oxa-4,7,10-triazatetradecan-14-oic acid 44i (868 mg, 1.56 mmol, prepared according to the known method of “WO2007008848”) was dissolved in 15 mL of acetonitrile, and added with N,N-diisopropylethylamine (2.0 mL, 12 mmol) and 2-(7-azobenzotriazole)-N,N,N′,N′-tetramethyluronium hexafluorophosphate (890 mg, 2.34 mmol). The reaction mixture was stirred at room temperature for 12 hours. When the reaction was complete by TLC, the reaction mixture was diluted with 50 mL of ethyl acetate, washed successively with saturated ammonium chloride solution (30 mL) and saturated sodium chloride solution (30 mL), dried over anhydrous sodium sulfate, filtered, and the filtrate was concentrated under reduced pressure. The residues were purified by silica gel column chromatography using eluent system B and system A to give the title product of (S)-methyl 2-((2R,3R)-3-((2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoate 44j (1.4 g, white foam solid), yield 99.9%.

MS m/z (ESI): 898.99 [M+1]

### Step 7

### (S)-2-((2R,3R)-3-((2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoic acid

(S)-methyl 2-((2R,3R)-3-((2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoate 44j (180 mg, 0.2 mmol) was dissolved in 0.5 mL of methanol, 0.5 mL of tetrahydrofuran and 0.5 mL of water, and then cooled to 0° C., and added with lithium hydroxide monohydrate (34 mg, 0.8 mmol). The mixture was stirred at 23° C. for 2 hours. When the reaction was complete by TLC and MS monitoring, the reaction mixture was diluted with 2 mL of water, added with 1N hydrochloric acid to adjust the pH about 4.5, and extracted with ethyl acetate (5 mL×3). The combined organic phases were dried over anhydrous magnesium sulfate, filtered and the filtrate was concentrated under reduced pressure to give the crude title product of (S)-2-((2R,3R)-3-(2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoic acid 44k (160 mg, light yellow foamy solid). The product was used in the next step without further purification.

MS m/z (ESI): 883.73 [M-1].

### Step 8

### (S)-2-((2R,3R)-3-((2S,4S)-1-((3R,4S,5S)-4-((S)—N,3-dimethyl-2-((S)-3-methyl-2-(methylamino)butanamido)butanamido)-3-methoxy-5-methylheptanoyl)-4-fluoropyrrolidin-2-yl)-3-methoxy-2-methylpropanamido)-3-phenylpropanoic acid

The crude product of (S)-2-((2R,3R)-3-((2S,4S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)-4-fluoropyrrolidin-2-yl)-3-methoxy-2-methylpropanamido)-3-phenylpropanoic acid 44k (160 mg, 0.2 mmol) was dissolved in 10 mL of anhydrous ethanol and added with 30 mg of palladium on carbon (10%). The reaction system was purged with hydrogen three times and stirred at 23° C. for 15 hours. When the reaction was complete by TLC, the reaction mixture was filtered through celite to remove palladium on carbon, and the filtrate was concentrated under reduced pressure to give 120 mg of residue. The residue was purified by high performance liquid chromatography to give the title product of (S)-2-((2R,3R)-3-(2S,4S)-1-((3R,4S,5S)-4-((S)—N,3-dimethyl-2-((S)-3-methyl-2-(methylamino)butanamido)butanamido)-3-methoxy-5-methylheptanoyl)-4-fluoropyrrolidin-2-yl)-3-methoxy-2-methylpropanamido)-3-phenylpropanoic acid 44 (29 mg, white solid), yield 21.5%.

MS m/z (ESI): 749.94 [M+1].

¹H NMR (400 MHz, CD₃OD) δ 7.29-7.14 (m, 5H), 4.81-4.71 (m, 2H), 4.18-4.13 (m, 2H), 4.05-4.03 (m, 2H), 3.84-3.81 (m, 1H), 3.63-3.60 (m, 1H), 3.58-3.52 (m, 1H), 3.48-3.13 (m, 14H), 3.00-2.91 (m, 1H), 2.66-1.81 (m, 11H), 1.63-1.38 (m, 2H), 1.21-1.10 (m, 5H), 1.09-0.94 (m, 14H), 0.91-0.82 (m, 3H).

### Example 45

### (S)-2-((2R,3R)-3-((2S,4S)-1-((3R,4S,5S)-4-((S)-2-((S)-2-(6-(2,5-di oxo-2,5-dihydro-1H-pyrrol-1-yl)-N-methylhexanamido)-3-methylbutanamido)-N,3-dimethylbutanamido)-3-methoxy-5-methylheptanoyl)-4-fluoropyrrolidin-2-yl)-3-methoxy-2-methylpropanamido)-3-phenylpropanoic acid

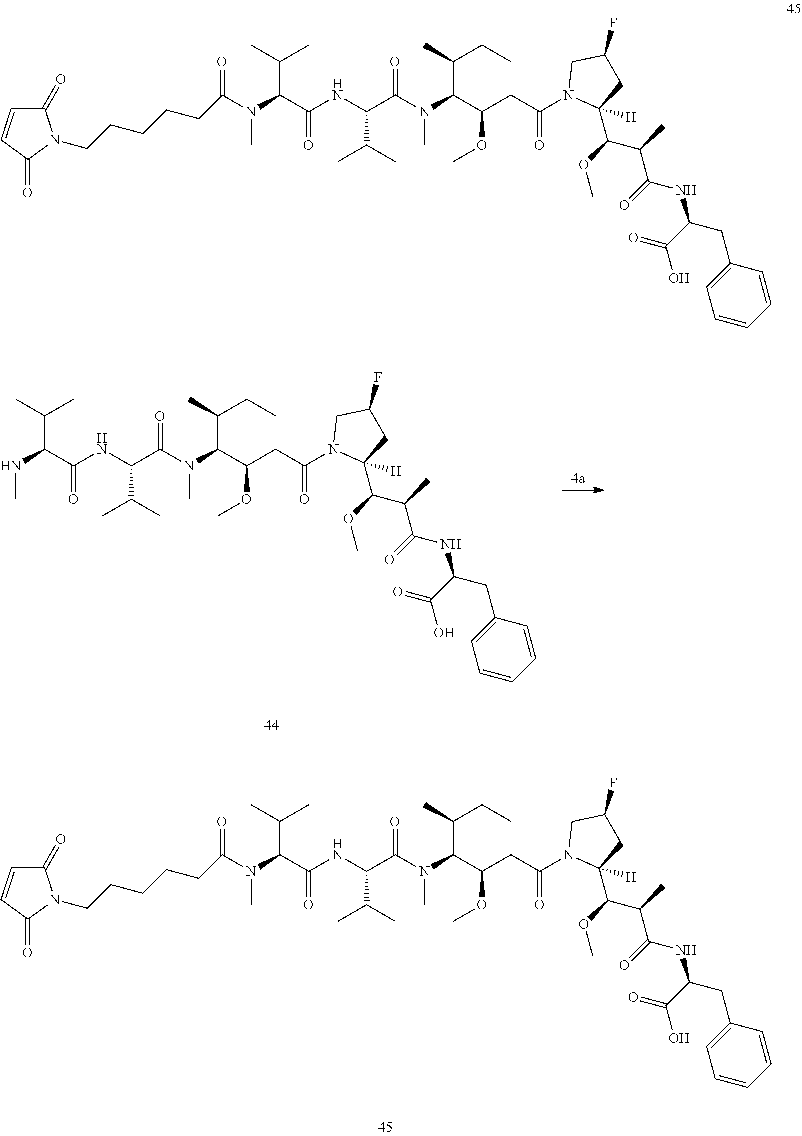 [SMILES: CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C ; CC[C@H](C)[C@@H]([C@@H](CC(=O)N1C[C@@H](F)C[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

(S)-2-((2R,3R)-3-((2S,4S)-1-((3R,4S,5S)-4-((S)—N,3-dimethyl-2-((S)-3-methyl-2-(methylamino)butanamido)butanamido)-3-methoxy-5-methylheptanoyl)-4-fluoropyrrolidin-2-yl)-3-methoxy-2-methylpropanamido)-3-phenylpropanoic acid 44 (1.0 g, 1.34 mmol) was dissolved in 20 mL of acetonitrile, and added with N,N-diisopropylethylamine 10.53 mL, 3.0 mmol) and 2-(7-azobenzotriazole)-N,N,N′,N′-tetramethyluronium hexafluorophosphate (560 mg, 1.48 mmol). The reaction mixture was stirred at 23° C. for 30 minutes and added with 6-(2,5-dioxo-2,5-dihydro-1H-pyrrol-1-yl)hexanoic acid 4a (300 mg, 1.41 mmol). When the reaction was complete by TLC, the reaction mixture was added with 50 mL of ethyl acetate and concentrated under reduced pressure. The residues were purified by flash column chromatography eluting with eluent systems B and A to give the crude title product of 1.0 g, which was further purified by high performance liquid chromatography to give the title product of (S)-2-((2R,3R)-3-((2S,4S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-fluoropyrrolidin-2-yl)-3-methoxy-2-methylpropanamido)-3-phenylpropanoic acid 45 (140 mg, white solid), yield 11%.

MS m/z (ESI): 942.67 [M+1]

¹H NMR (400 MHz, CD₃OD) δ 7.34-7.14 (m, 5H), 6.83-6.80 (m, 2H), 5.20-5.10 (m, 1H), 5.01-4.96 (m, 1H), 4.78-4.57 (m, 2H), 4.18-4.01 (m, 2H), 4.00-3.80 (m, 1H), 3.56-3.41 (m, 5H), 3.35-3.24 (m, 10H), 3.19-2.90 (m, 4H), 2.60-2.40 (m, 4H), 2.39-2.00 (m, 4H), 1.93-1.58 (m, 6H), 1.50-1.10 (m, 7H), 1.09-0.82 (m, 21H).

### Example 46

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(4-fluorophenyl)propanoic acid

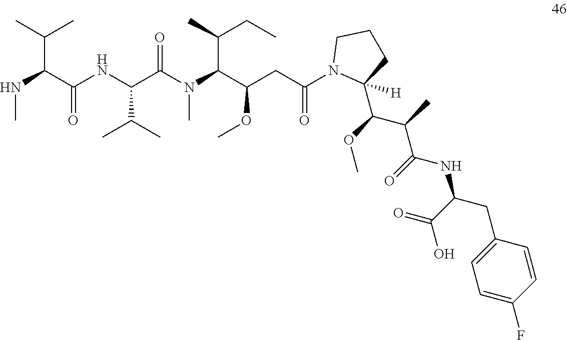 [SMILES: CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@@H](NC)C(C)C)C(C)C]

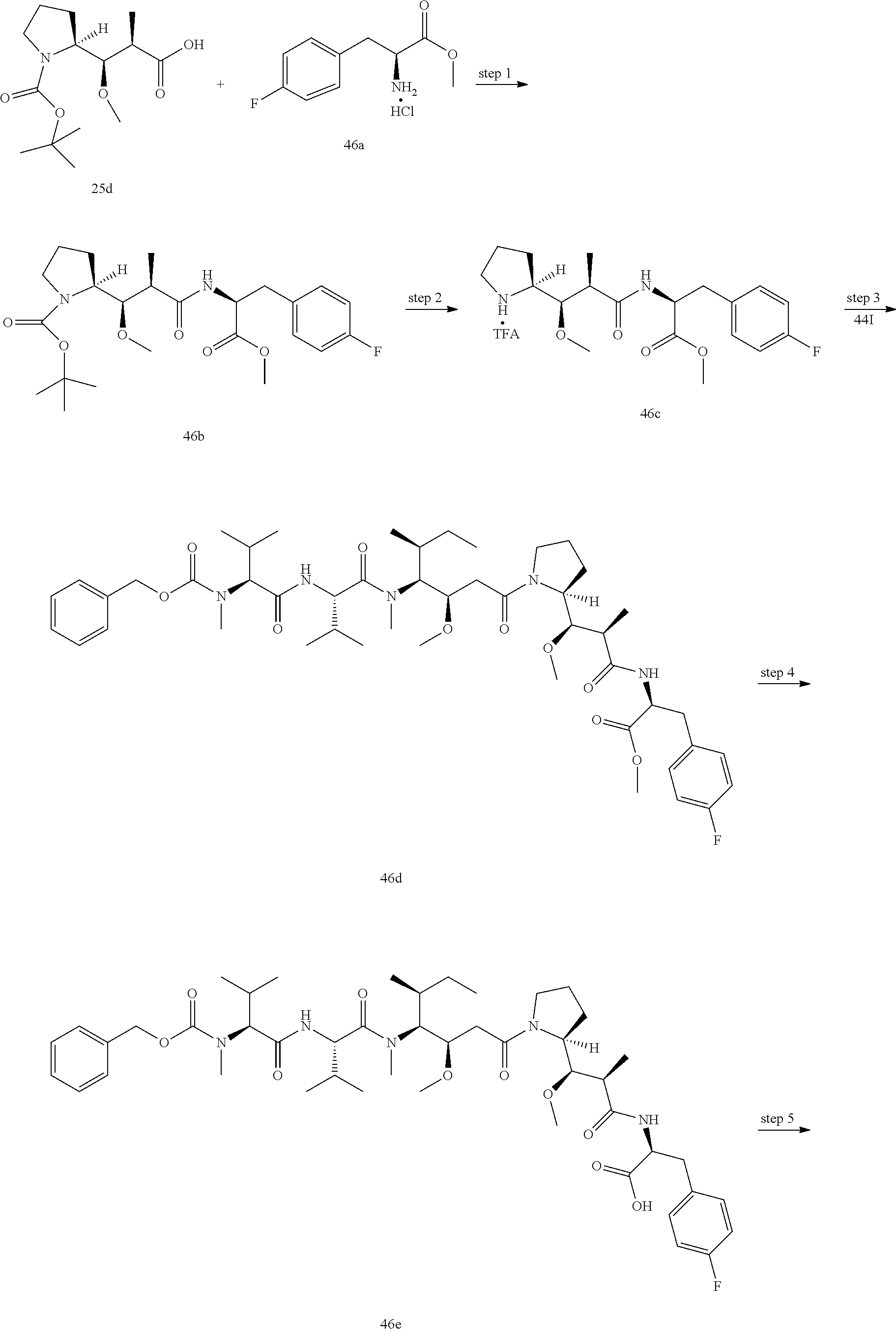

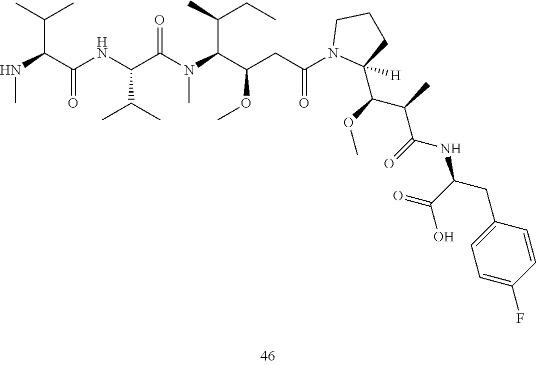 [SMILES: CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@@H](NC)C(C)C)C(C)C]

### Step 1

### (S)-tert-butyl 2-((1R,2R)-3-(((S)-3-(4-fluorophenyl)-1-methoxy-1-oxopropan-2-yl)amino)-1-methoxy-2-methyl-3-oxopropyl)pyrrolidine-1-carboxylate

(2R,3R)-3-((S)-1-(tert-butoxycarbonyl)pyrrolidin-2-yl)-3-methoxy-2-methylpropanoic acid 25d (1.0 g, 3.5 mmol) was dissolved in 15 mL of acetonitrile, and added with (S)-methyl 2-amino-3-(4-fluorophenyl)propanoate hydrochloride 46a (820 mg, 4 mmol, prepared according to the known method of “Tetrahedron, 2003, 59(21), 3719-3727”), and N,N-diisopropylethylamine (2.7 mL, 15 mmol) and 2-(7-azobenzotriazole)-N,N,N′,N′-tetramethyluronium hexafluorophosphate (1.9 g, 5.0 mmol). The reaction mixture was stirred at 23° C. for 12 hours. When the reaction was complete by TLC, the reaction mixture was diluted with 50 mL of ethyl acetate and washed successively with saturated ammonium chloride solution (30 mL) and saturated sodium chloride solution (30 mL), dried over anhydrous sodium sulfate, filtered, and the filtrate was concentrated under reduced pressure. The residues were purified by silica gel column chromatography using eluent system B to give the title product of (S)-tert-butyl 2-((1R,2R)-3-((S)-3-(4-fluorophenyl)-1-methoxy-1-oxopropan-2-yl)amino)-1-methoxy-2-methyl-3-oxopropyl)pyrrolidine-1-carboxylate 46b (1.35 g, white solid), yield 82.6%.

MS m/z (ESI): 366.46 [M-100-1].

### Step 2

### (S)-methyl 3-(4-fluorophenyl)-2-((2R,3R)-3-methoxy-2-methyl-3-((S)-pyrrolidin-2-yl)propanamido)propanoate trifluoroacetate

(S)-tert-butyl 2-((1R,2R)-3-(((S)-3-(4-fluorophenyl)-1-methoxy-1-oxopropan-2-yl)amino)-1-methoxy-2-methyl-3-oxopropyl)pyrrolidine-1-carboxylate 46b (1.34 g, 2.87 mmol) was dissolved in 20 mL of dichloromethane and added dropwise with trifluoroacetic acid (3 mL, 30 mmol), and stirred at 23° C. for 12 hours. When the reaction was complete by TLC, the reaction solution was concentrated under reduced pressure to give the crude title product of (S)-methyl 3-(4-fluorophenyl)-2-((2R,3R)-3-methoxy-2-methyl-3-((S)-pyrrolidin-2-yl)propanamido) propanoate trifluoroacetate 46c (1.92 g, yellow viscous liquid). The product was used in the next step without further purification.

MS m/z (ESI): 366.85 [M+1]

### Step 3

### (S)-methyl 2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoate

(S)-methyl 3-(4-fluorophenyl)-2-((2R,3R)-3-methoxy-2-methyl-3-((S)-pyrrolidin-2-yl)propanamido)propanoate trifluoroacetate 46c (367 mg, 1.0 mmol) and (5S,8S,11S,12R)-11-((S)-sec-butyl)-5,8-diisopropyl-12-methoxy-4,10-dimethyl-3,6,9-tri oxo-1-phenyl-2-oxa-4,7,10-triazatetradecan-14-oic acid 44i (549 mg, 1.0 mmol) were dissolved in 10 mL of acetonitrile, and added with N,N-diisopropylethylamine (1.24 mL, 7 mmol) and 2-(7-azobenzotriazole)-N,N,N′,N′-tetramethyluronium hexafluorophosphate (570 mg, 1.5 mmol). The reaction mixture was stirred at 23° C. for 12 hours. When the reaction was complete by TLC, the reaction mixture was diluted with 30 mL of ethyl acetate and washed successively with saturated ammonium chloride solution (20 mL) and saturated sodium chloride solution (20 mL), dried over anhydrous sodium sulfate, filtered, and the filtrate was concentrated under reduced pressure. The residues were purified by silica gel column chromatography eluting with eluent systems B and A to give the title product of (S)-methyl 2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoate 46d (760 mg, white foam solid), yield 84.6%.

MS m/z (ESI): 898.10 [M+1]

### Step 4

### (S)-2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoic acid

(S)-methyl 2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoate 46d (180 mg, 0.2 mmol) was dissolved in 0.5 mL of methanol, 0.5 mL of tetrahydrofuran and 0.5 mL of water, and cooled to 0° C., and then added with lithium hydroxide monohydrate (34 mg, 0.8 mmol) and stirred at 23° C. for 2 hours. When the reaction was complete by TLC and MS monitoring, the reaction mixture was diluted with 2 mL of water, and added with 1N hydrochloric acid to adjust the pH to about 4.5, and extracted with ethyl acetate (5 mL×3). The combined organic phases were dried over anhydrous magnesium sulfate, filtered and the filtrate was concentrated under reduced pressure to give the crude title product of (S)-2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoic acid 46e (160 mg, white solid). The product was used in the next step without further purification.

MS m/z (ESI): 883.78 [M-1]

### Step 5

### (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(4-fluorophenyl)propanoic acid

The crude product of (S)-2-((2R,3R)-3-((S)-1-((5S,8S,11S,12R)-11-((S)-sec-butyl)-5,8-diisopropyl-12-methoxy-4,10-dimethyl-3,6,9-trioxo-1-phenyl-2-oxa-4,7,10-triazatetradecan-14-oyl)pyrrolidin-2-yl)-3-methoxy-2-methylpropanamido)-3-(4-fluorophenyl)propanoic acid 46e (160 mg, 0.2 mmol) was dissolved in 10 mL of anhydrous ethanol and added with 30 mg of palladium on carbon (10%). The reaction mixture was replaced with hydrogen three times and stirred at room temperature for 15 hours. When the reaction was complete by TLC, the reaction mixture was filtered through celite to remove palladium on carbon, and the filtrate was concentrated under reduced pressure to give 130 mg of residue. The residue was purified by high performance liquid chromatography to give the title product of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(4-fluorophenyl)propanoic acid 46 (34 mg, white solid), yield 25.2%.

MS m/z (ESI): 749.94 [M+1]

¹H NMR (400 MHz, CD₃OD) δ 7.31-7.24 (m, 2H), 7.02-6.92 (m, 2H), 4.48-4.58 (m, 2H), 4.22-4.11 (m, 1H), 3.85-3.40 (m, 4H), 3.39-3.04 (m, 14H), 3.00-2.90 (m, 1H), 2.66-2.40 (m, 5H), 2.39-2.02 (m, 4H), 1.99-1.75 (m, 3H), 1.73-1.24 (m, 3H), 1.20-1.10 (m, 4H), 1.09-0.95 (m, 16H), 0.94-0.80 (m, 3H).

### Example 47

### (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(4-fluorophenyl)propanoic acid

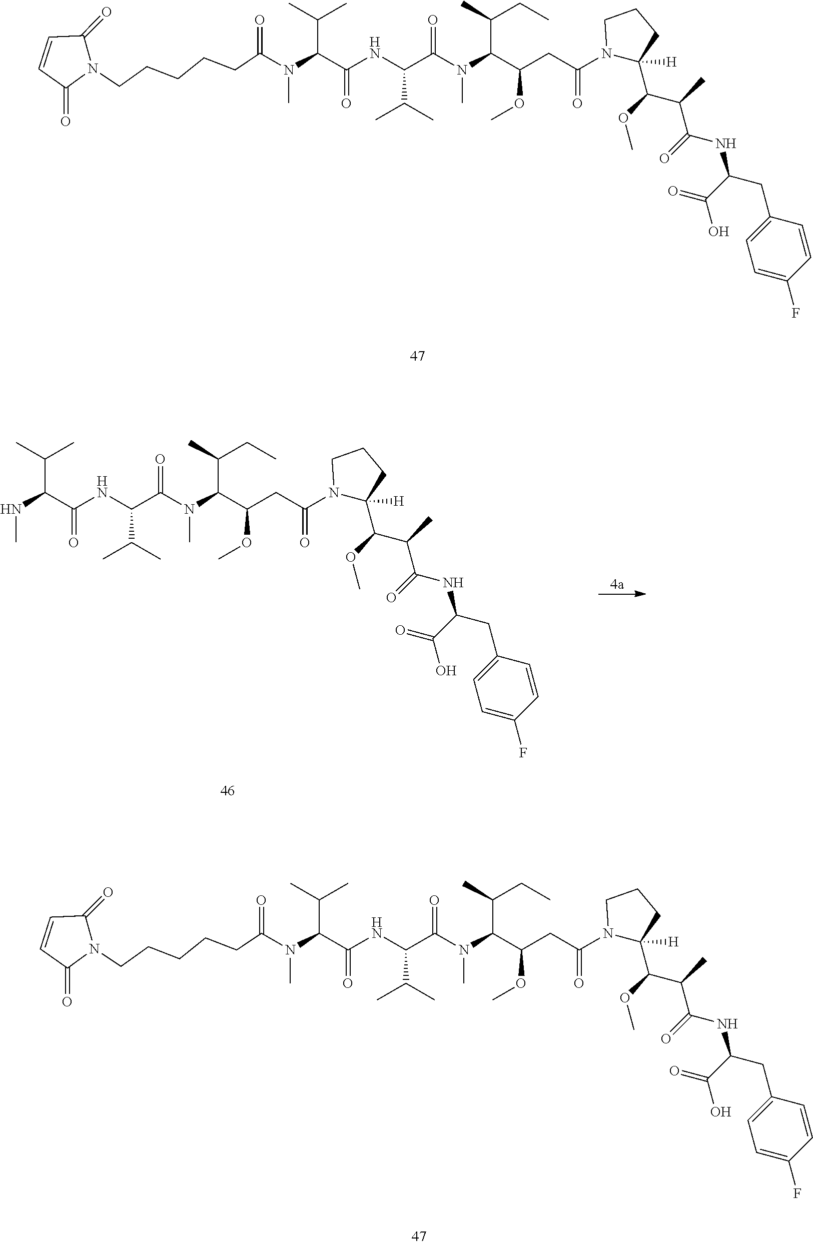 [SMILES: CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@H](C(C)C)N(C)C(=O)CCCCCN1C(=O)C=CC1=O)C(C)C]

6-(2,5-dioxo-2,5-dihydro-1H-pyrrol-1-yl)hexanoic acid 4a (434 mg, 2.06 mmol) was dissolved in 20 mL of acetonitrile, and added with N,N-diisopropylethylamine (844 mL, 6.55 mmol) and 2-(7-azobenzotriazole)-N,N,N′,N′-tetramethyluronium hexafluorophosphate (781 mg, 1.87 mmol). The reaction mixture was stirred at 23° C. for 30 minutes and added with (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-(4-fluorophenyl)propanoic acid 46 (1.4 g, 1.87 mmol). When the reaction was complete by TLC, the reaction mixture was diluted with 50 mL of ethyl acetate, and concentrated under reduced pressure. The residues were purified by silica gel column chromatography eluting with eluent systems B and A to give the crude title product (1.2 g), which was further purified by high performance liquid chromatography to give the title product of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(4-fluorophenyl)propanoic acid 47 (670 mg, white solid), yield 37.9%.

MS m/z (ESI): 944.54 [M+1]

¹H NMR (400 MHz, CD₃OD) δ 12.80 (br.s, 1H), 8.56-8.52 (m, 1H), 8.42-8.20 (m, 1H), 8.18-8.10 (m, 1H), 7.30-7.18 (m, 2H), 7.07-6.75 (m, 4H), 4.64-4.34 (m, 3H), 4.08-3.94 (m, 1H), 3.80-3.50 (m, 2H), 3.46-2.70 (m, 15H), 2.50-1.80 (m, 6H), 1.73-1.38 (m, 7H), 1.30-1.10 (m, 12H), 1.09-0.62 (m, 20H).

### Example 48

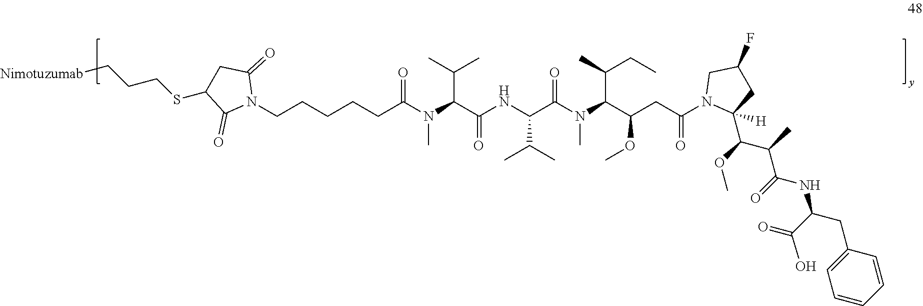

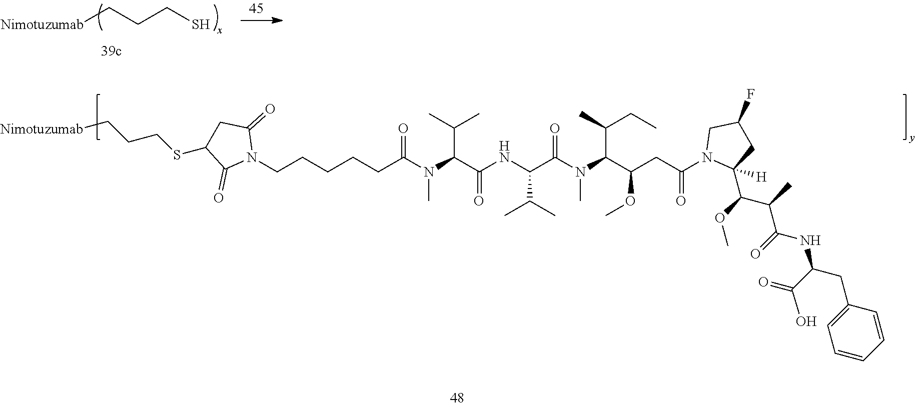

(S)-2-((2R,3R)-3-((2S,4S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-fluoropyrrolidin-2-yl)-3-methoxy-2-methylpropanamido)-3-phenylpropanoic acid 45 (1.1 mg, 1.16 μmol) was dissolved in 1.1 mL of acetonitrile and added with Nimotuzumab-propanethiol 39c in PBS buffer (1.63 mg/mL, 11.4 mL). After stirring on a shaker in a water bath at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 48 in PBS buffer (0.75 mg/mL, 19.8 mL), then stored at 4° C.

Q-TOF LC/MS: characteristic peaks: 150186.5 (M_{Ab}+0D), 151364.1 (M_{Ab}+1D), 152262.3 (M_{Ab}+2D), 153435.7(M_{Ab}+3D), 154499.6(M_{Ab}+4D), 155427.5(M_{Ab}+5D).

Average value: y=2.0.

### Example 49

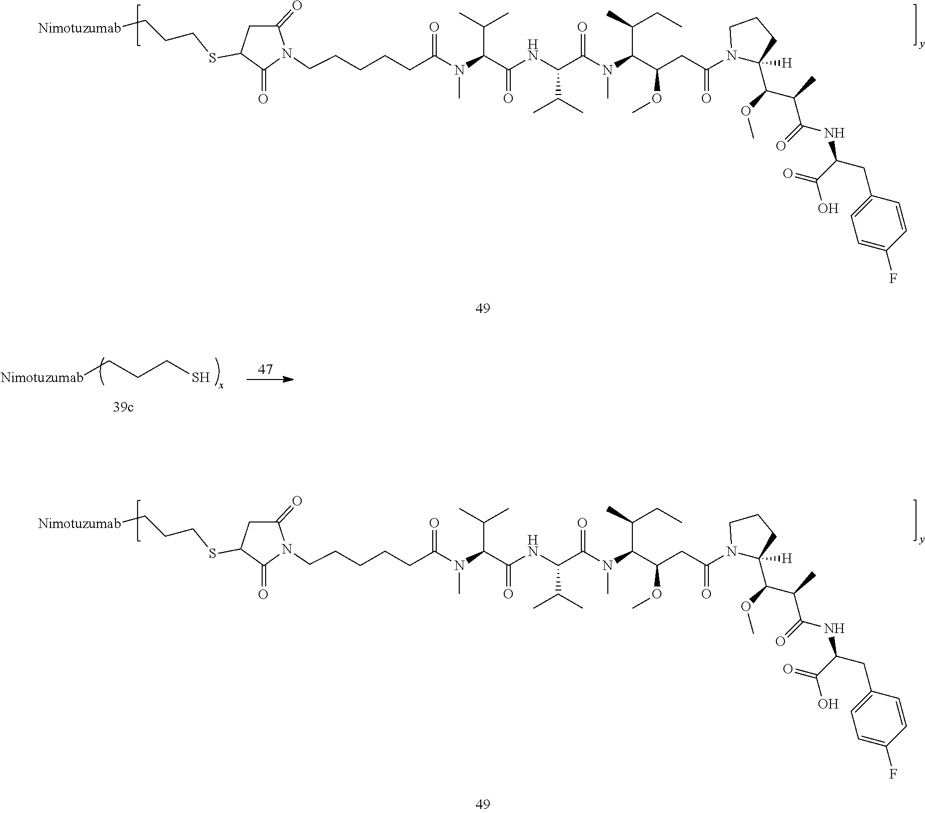

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(4-fluorophenyl)propanoic acid 47 (1.1 mg, 1.16 μmol) was dissolved in 1.1 mL of acetonitrile and added with Nimotuzumab-propanethiol 39c in PBS buffer (1.63 mg/mL, 11.4 mL). After stirring on a shaker in a water bath at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 49 in PBS buffer (0.75 mg/mL, 19.8 mL), then stored at 4° C.

Q-TOF LC/MS: characteristic peaks: 150185 0.5 (M_{Ab}+0D), 151364.7(M_{Ab}+1D), 152261.2 (M_{Ab}+2D), 153436.2(M_{Ab}+3D), 154499.9(M_{Ab}+4D), 155428.6(M_{Ab}+5D).

Average value: y=2.0.

### Example 50

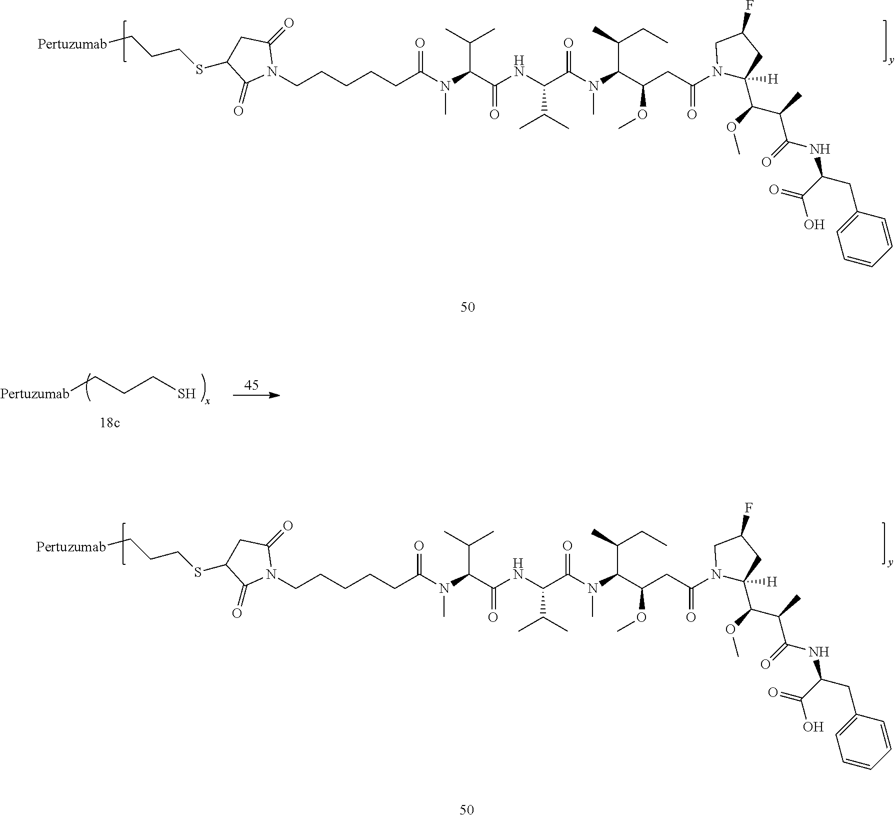

(S)-2-((2R,3R)-3-((2S,4S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-fluoropyrrolidin-2-yl)-3-methoxy-2-methylpropanamido)-3-phenylpropanoic acid 45 (1.09 mg, 1.16 μmol) was dissolved in 1.1 mL of acetonitrile and added with Pertuzumab-propanethiol 18c solution (1.65 mg/mL, 11.3 mL). After stirring on a shaker at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 50 in PBS buffer (0.75 mg/mL, 19.5 mL), then stored at 4° C.

Q-TOF LC/MS: characteristic peaks: 148095.6(M_{Ab}+0D), 149111.5(M_{Ab}+1D), 150165.3 (M_{Ab}+2D), 151184.7(M_{Ab}+3D), 152255.2M_{Ab}+4D), 153297.5(M_{Ab}+5D).

Average value: y=2.0.

### Example 51

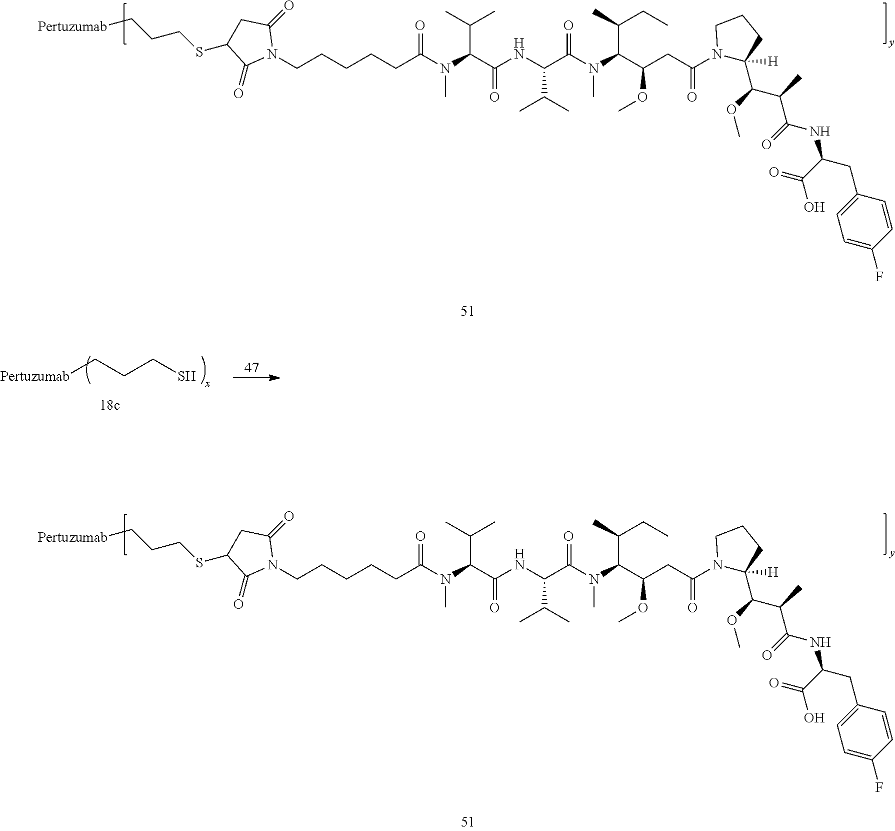

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(4-fluorophenyl)propanoic acid 47 (1.09 mg, 1.16 μmol) was dissolved in 1.1 mL of acetonitrile and added with Pertuzumab-propanethiol solution 47c (1.65 mg/mL, 11.3 mL). After stirring on a shaker at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 51 in PBS buffer (0.75 mg/mL, 19.5 mL), then stored at 4° C.

Q-TOF LC/MS: characteristic peaks: 148096.2(M_{Ab}+0D), 149112.2(M_{Ab}+1D), 150165.4 (M_{Ab}+2D), 151184.8(M_{Ab}+3D), 152255.1(M_{Ab}+4D), 153297.6(M_{Ab}+5D).

Average value: y=2.0.

### Example 52

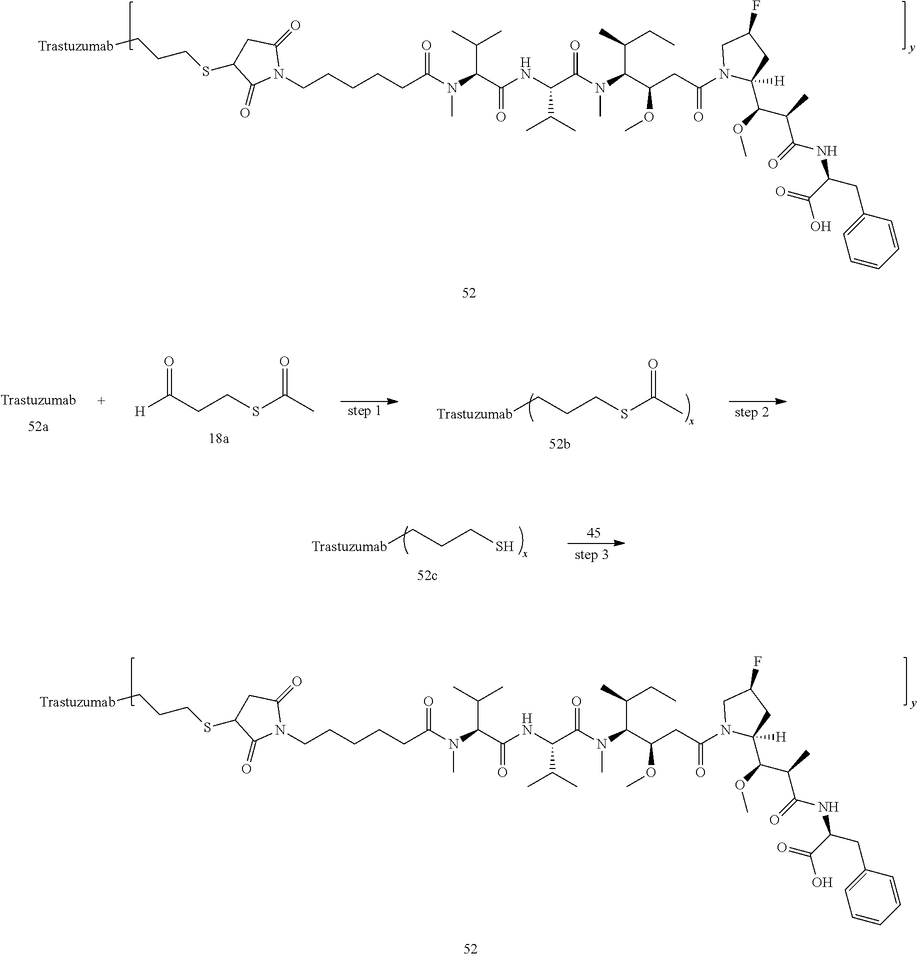

### Step 1

S-(3-oxopropyl) ethanethioate 18a (0.35 mg, 2.65 μmol) was dissolved in 0.45 mL of acetonitrile. Trastuzumab in acetic acid/sodium acetate buffer (10.0 mg/ml, 4.5 mL, 0.304 μmol) with pH 4.5 was added with a solution of S-(3-oxopropyl) ethanethioate 18a in acetonitrile, and then added with 1.0 mL of an aqueous solution of sodium cyanoborohydride (7.06 mg, 112 μmol) dropwise. The reaction mixture was stirred at 25° C. for 2 hours. After completion of the reaction, the reaction mixture was purified by desalting with a Sephadex G25 gel column (elution phase: 0.05 M PBS solution at pH 6.5) to give the title product 52b solution, which was used directly in the next step.

### Step 2

The solution of 52b (about 15.0 mL) was added with 0.45 mL of a 2.0 M solution of hydroxylamine hydrochloride and the reaction mixture was shaken in a shaker at 25° C. for 30 minutes. The reaction solution was desalted with a Sephadex G25 gel column (elution phase: 0.05 M of PBS solution) to give the title product of Trastuzumab-propanethiol 52c solution (concentration 1.65 mg/ml, 22.6 mL).

### Step 3

(S)-2-((2R,3R)-3-((2S,4S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)-4-fluoropyrrolidin-2-yl)-3-methoxy-2-methylpropanamido)-3-phenylpropanoic acid 45 (1.1 mg, 1.2 μmol) was dissolved in 1.1 mL of acetonitrile and added with Trastuzumab-propanethiol solution 52c (1.65 mg/mL, 11.3 mL). After being placed on a shaker at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 52 in PBS buffer (0.72 mg/mL, 20 mL), then stored at 4° C.

Q-TOF LC/MS: characteristic peaks: 148062.9(M_{Ab}+0D), 149235.2(M_{Ab}+1D), 150259.8 (M_{Ab}+2D), 151268.2(M_{Ab}+3D), 152341.9(M_{Ab}+4D), 153356.4(M_{Ab}+5D).

Average value: y=2.0.

### Example 53

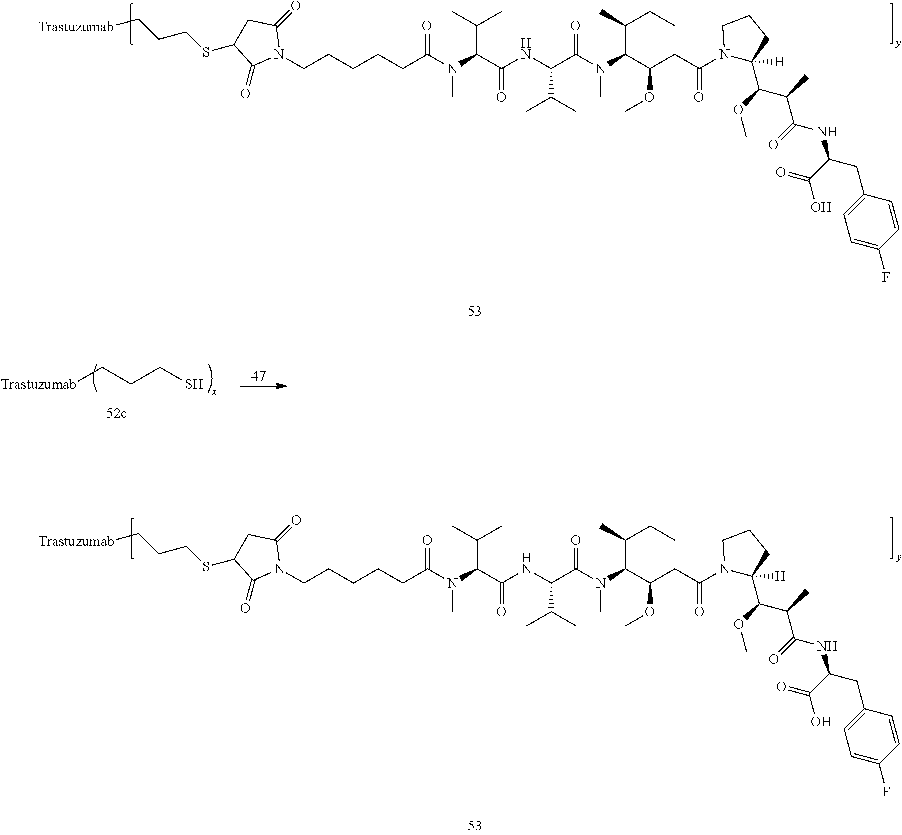

(S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-(4-fluorophenyl)propanoic acid 47 (1.12 mg, 1.2 μmop was dissolved in 1.1 mL of acetonitrile and added with Trastuzumab-propanethiol solution 52c (1.65 mg/mL, 11.3 mL). After being placed on a shaker at 25° C. for 4 hours, the reaction mixture was desalted with a Sephadex G25 gel column (Elution phase: 0.05 M PBS solution at pH 6.5), and filtered under sterile conditions through a 0.2 μm filter to give the title product 53 in PBS buffer (0.70 mg/mL, 20.5 mL), then stored at 4° C.

Q-TOF LC/MS: characteristic peaks: 148065.2(M_{Ab}+0D), 149244.6(M_{Ab}+1D), 150254.9 (M_{Ab}+2D), 151280.9(M_{Ab}+3D), 152301.6(M_{Ab}+4D), 153457.9(M_{Ab}+5D).

Average value: y=2.0.

### BIOLOGICAL ASSAY

### Test Example 1: In Vitro Inhibition Test of Compound of Formula (D) on Tumor Cell Proliferation

### 1. Experimental Objective

The objective of this experiment is to test the in vitro inhibition effect of the formula (D) according to the present invention on the proliferation of HepG2 tumor cells (human hepatocellular carcinoma cells, Chinese Academy of Sciences cell bank, No. #TCHu 72) and A549 tumor cells (human lung adenocarcinoma, Chinese Academy of Sciences cell bank, No. #TCHu150). The cells were treated with different concentrations of the compound in vitro. After 76 hours incubation, the cell proliferation was detected by CCK-8 reagent (Cell Counting Kit-8, Dojindo, No. CK04), and the activity of the compound in vitro was evaluated according to the IC₅₀ value.

### 2. Experimental Protocol

The following is an example of a method for in vitro proliferation inhibition test of HepG2 cells for the purpose of exemplifying the test of the compounds according to the present invention for the proliferation inhibitory activity on tumor cells in vitro. This method is also applicable to, but not limited to, the in vitro proliferation inhibition tests on other tumor cells.

### 2.1 Cell Preparation

HepG2 cells in logarithmic growth phase were washed with PBS (phosphate buffer, ThermoFisher) once, and added with 2-3 ml trypsin (0.25% trypsin-EDTA (1×), Gibico, Life Technologies) to digest for 2-3 min. Then, 10-15 ml of cell culture medium (DMEM/F12 medium, Invitrogen; 10% (v/v)) was added after the cells were completely digested. The digested HepG2 cells were eluted, and centrifuged at 1000 rpm for 3 min. The supernatant was discarded, and then 10-20 ml of cell culture medium were added to resuspend the cells to prepare a single cell suspension.

### 2.2 Cell Plating

The HepG2 single cell suspension was mixed and the cell density was adjusted to 6×10⁴ cells/ml with cell culture medium. The density-adjusted cell suspension was mixed uniformly and added to a 96-well cell culture plate at 100 μl/well. The plates were incubated in a 5% CO₂ incubator at 37° C. for 18-20 hours.

### 2.3 Compound Preparation

The compound was dissolved with DMSO (dimethylsulfoxide, Shanghai Titan Technology Co., Ltd.) to prepare a storage solution having an initial concentration of 10 mM.

10 μl of 10 mM compound was added to the wells of the first column of a U-shaped bottom 96-well plate (sample plate 1), and 90 μl of DMSO was added to each compound sample well of the first column, i.e., the stock solution was diluted 10 times as the starting point. Then, 3-fold gradient dilutions were performed, with each compound concentration diluted 10 times. The diluent was DMSO solution. 60 μl of 100% DMSO was added to 12^{th} column and 20 μl of 1 mM positive drug control was added to 11^{th} column.

In a new U-shaped bottom 96-well plate (sample plate 2), each sample from the sample plate 1 was diluted 20 times with the complete medium. Then, in another new U-shaped bottom 96-well plate (sample plate 3), each well sample from the sample plate 2 was subjected to a final 10-fold dilution.

### 2.4 Sample Loading

After the cell pre-culture was completed, the 96-well cell culture plate was removed and the supernatant was discarded. Each sample dilution in the 96-well sample plate 3 was successively added to a 96-well cell culture plate at 100 μl/well. Each compound sample was tested in duplicate at each concentration. The loading operation did not exceed 30 min. After completion of the loading operation, the 96-well cell culture plate was incubated at 37° C. for about 76 hours in a 5% CO₂ incubator.

### 2.5 Coloring Operation

The 96-well cell culture plate was taken and CCK-8 was added to each well at 100 μl/well. The cell culture plate was gently pat and mixed for more than 10 times and incubated at 37° C. for 2 hours in a 5% CO₂ incubator.

### 2.6 Reading Plate

The 96-well cell culture plate was taken and placed in a microplate reader (PerkinElmer, VICTOR 3) and the absorbance at 450 nm was measured using the microplate reader.

In accordance with the above procedure, the inhibition activity of the compound according to the present invention on the proliferation of A549 tumor cells in vitro was tested. The cell culture medium was also DMEM/F12.

### 3. Data Analysis

Data was analyzed with Microsoft Excel and Graphpad Prism 5. The results are shown in Table 1.

TABLE 1

IC₅₀/nM

| Compound No. | HepG2 | A549 |
|---|---|---|
| 1 | 8.60 | 12.75 |
| 2 | 4.12 | 2.33 |
| 3 | 9.33 | 16.17 |
| 7 | 283 | 776.4 |
| 9 | 55 | 114.4 |
| 11 | 6.6 | 27.23 |
| 13 | 11.7 | 30.24 |
| 15 | 47.45 | 75.45 |
| 16 | 13.92 | 52.99 |
| 44 | | 179 |
| 25 | 174.7 | 200.5 |
| 29 | 58.64 | 1.8 |
| 31 | 100.5 | 130.1 |
| 32 | 17.40 | 13.51 |
| 33 | 4.66 | 5.00 |
| 34 | 18.33 | 32.32 |
| 35 | 30.56 | 17.98 |
| 46 | | 106 |

### Test Example 2: In Vitro Tumor Cell Proliferation Inhibition Test of Antibody-Drug Conjugate of the Present Invention Against HER2 Target

The objective of this experiment is to test the in vitro proliferation inhibition effect of the antibody-drug conjugate against HER2 target according to the present invention on SK-BR-3 tumor cells (human breast cancer cells, ATCC, HTB-30). The cells were treated with different concentrations of the compound in vitro. After 76 hours of incubation, cell proliferation was detected by CCK-8 reagent (Cell Counting Kit-8, Dojindo, No. CK04), and the activity of the compound in vitro was evaluated according to the IC₅₀ value.

According to the test method of test example 1, the test cell was SK-BR-3, and the cell culture medium was McCoy's 5A medium (Gibco, NO. 16600-108) containing 10% FBS. The relevant compounds were tested and the results are shown in Table 2.

TABLE 2

| | IC₅₀/nM |
|---|---|
| Compound No. | SK-BR-3 |
| 18 | 0.026 |
| 19 | 0.147 |
| 20 | 0.466 |
| 21 | 0.031 |
| 22 | 0.28 |
| 23 | 0.201 |
| 24 | 0.174 |
| 36 | 0.052 |
| 37 | 0.215 |
| 38 | 0.101 |
| 50 | 0.1 |
| 51 | 0.373 |

Conclusion: The antibody-drug conjugates against HER2 target according to the present invention have significant proliferation inhibition effects on SK-BR-3 tumor cells.

### Test Example 3: In Vitro Tumor Cell Proliferation Inhibition Test of Antibody-Drug Conjugate of the Present Invention Against EGFR Target

The objective of this experiment is to test the in vitro proliferation inhibition effect of the antibody-drug conjugate against EGFR target according to the present invention on HCC827 tumor cells (non-small cell lung cancer cells, Chinese Academy of Sciences cell bank, No. #TCHu153). The cells were treated with different concentrations of the compound in vitro. After 76 hours of incubation, cell proliferation was detected by CCK-8 reagent (Cell Counting Kit-8, Dojindo, No. CK04), and the activity of the compound in vitro was evaluated according to the IC₅₀ value.

According to the test method of test example 1, the test cells were HCC827, and the cell culture medium was RPMI1640 medium (Invitrogen) containing 10% FBS (v/v). The relevant compounds were tested and the results are shown in Table 3.

TABLE 3

| | IC₅₀/nM |
|---|---|
| Compound No. | HCC827 |
| 39 | 0.353 |
| 40 | 0.255 |
| 41 | 0.451 |
| 48 | 0.993 |

Conclusion: The antibody-drug conjugates against EGFR target according to the present invention have significant proliferation inhibition effects on HCC827 tumor cells.

### Test Example 4: Tumor Inhibition Rate Test of NCI-N87

### 1. Experimental Objective

To evaluate and compare the effect of antibody-cytotoxin conjugates according to the present invention on transplanted tumors of NCI-N87 cells (HER2 overexpressing human gastric cancer cells, ATCC, CRL-5822) in nude mice.

### 2. Test Drugs

Samples: Compound 18, Compound 21, Compound 36.

Positive controls: Pertuzumab, Trastuzumab.

Preparation method: all prepared with physiological saline.

3. Test Animals

BALB/cA-nude mice, 6-7 weeks, female, purchased from Shanghai Slack Experimental Animal Co., Ltd. Certificate number: SCXK (Shanghai) 2012-0002. Feeding environment: SPF level.

### 4. Test Procedures

Nude mice were inoculated subcutaneously with human gastric cancer NCI-N87 cells, and the animals were randomly divided into groups (D0) after tumor growth to 100-200 mm³. Administration dosages and regimens are shown in Table 4. Mice were measured for tumor volume 2-3 times a week, the body weight was measured and the data was recorded.

The formula for calculating tumor volume (V) is as follows:

V=1/2×a×b²

wherein: a and b represent length and width, respectively.

T/C (%)=(T−T₀)/(C−C₀)×100%, wherein T and C represent the tumor volume at the end of the experiment; T₀ and C₀ represent the tumor volume at the beginning of the experiment.

TABLE 4
Effects of compounds (18, 21, 36) on transplanted tumor
of human gastric carcinoma NCI-N87 in nude mouse

| | | | Mean | | Mean | | | | | |
|---|---|---|---|---|---|---|---|---|---|---|
| | | | tumor | | tumor | | | % tumor | | |
| | | | volume | | volume | | | inhibition | P | Number of |
| | | | (mm³) | | (mm³) | | % T/C | rate | Value | animals/ |
| Groups | Administration | Route | D0 | SEM | D21 | SEM | D21 | D21 | D21 | group |
| Solvent | D0, 7 | IV | 107.8 | ±3.0 | 742.8 | ±92.6 | — | — | — | 12 |
| Pertuzumab 60 mg/kg | D0, 7 | IV | 115.5 | ±2.9 | 380.1 | ±76.3 | 42 | 58 | 0.018 | 6 |
| Trastuzumab 7.5 mg/kg | D0, 7 | IV | 109.7 | ±4.4 | 366.5 | ±90.9 | 40 | 60 | 0.019 | 6 |
| 18 (3 mg/kg) | D0, 7 | IV | 113.1 | ±2.4 | 87.6 | ±6.7 | −22 | 122 | 0 | 6 |
| 21 (3 mg/kg) | D0, 7 | IV | 122.1 | ±4.0 | 20 | ±20.0 | −84 | 184 | 0 | 6 |
| 36 (3 mg/kg) | D0, 7 | IV | 119.1 | ±6.1 | 95.8 | ±22.3 | −20 | 120 | 0 | 6 |

D0: The time of the first administration; Pertuzumab and Trastuzumab: First dose doubling; The P value was compared with the solvent control group; Student's t test was adopted. The number of mice at the beginning of the experiment was as follows: Control group n=12, Treatment group n=6.

### 5. Result

The compounds of the present invention can significantly inhibit the growth of HER2 overexpressing NCI-N87 nude mice subcutaneous transplanted tumor, and the tumor-bearing mice are better tolerant to the above drugs.

### Test Example 5. Efficacy Test of EGFRV3 Antibody and its ADC on HCC827 Transplanted Tumor in Nude Mice

### 1. Experimental Objective

In this test, Nude mice were used as the test animals to evaluate the efficacy of the ADC compounds 39 and 40 of the present invention on transplanted tumors of human non-small cell lung cancer HCC827 in nude mice after multi-dose intraperitoneal injection.

The tumor inhibition effect was observed once after multi-dose intraperitoneal injection of ADC compounds 39 and 40 of the present invention, and the experiment was finished on day 38 after administration. The test results show that the tumor inhibition rate of ADC compound 39 (0.05 mg/mouse) is 62.78%, and the difference is statistically significant compared with the control group (p<0.05). The tumor inhibition rate of ADC 40 (0.025 mg/mouse) is 49.20%, which is not significantly different from that of blank control group. The inhibition rate of ADC compound 40 (0.05 mg/mouse) is 86.8%, which is significantly different from that of blank control group (p<0.01). The tumor inhibition rate of ADC 40 (0.1 mg/mouse) is 93.41%, which is significantly different from that of blank control group (p<0.05).

### 2. Test Drugs and Materials

### 2.1 Test Drugs

The ADC compounds of the present invention: ADC compound 39, ADC compound 40.

### 2.2 Formulation Method

All prepared with physiological saline.

### 2.3 Test Animals

Nude mice, SPF, 16-20 g, female, purchased from Shanghai Xi Puer Bikai Experimental

Animal Co., Ltd. Certificate number: SCXK (Shanghai) 2008-0016.

### 3. Test Protocol

Tumor cell transplantation was performed after the nude mice were adapted to the laboratory environment for three days. HCC827 cells were inoculated subcutaneously in the right rib of nude mice (4×10⁶+50% matrigel/mouse). On day 21 after inoculation, the drug was administered when the tumor grew to 209.41+25.93 mm³ (dl). The specific administration dose and method are shown in Table 5.

Mice were measured for tumor volume twice a week, the body weight of the nude mice was weighed and the data were recorded.

Excel statistical software: mean value is calculated as average; SD is calculated as STDEV; SEM is calculated as STDEV/SQRT; P value between different groups is calculated as TTEST.

Tumor volume (V) is calculated as: V=1/2×L_{length}×Lₛₕₒᵣₜ²
Relative volume (RTV)=V_{T}/V₀
Tumor Inhibition Rate (%)=(C_{RTV}−T_{RTV})/C_{RTV} (%)
wherein V₀ and V_{T} represent the tumor volume at the beginning of the experiment and at the end of the experiment, respectively. C_{RTV} and T_{RTV} represent the relative tumor volume of blank control group (Blank) and test group at the end of the experiment, respectively.

TABLE 5
Effect of test antibody on HCC827 xenografts in nude mice

| | | | Mean | | Mean | | | | |
|---|---|---|---|---|---|---|---|---|---|
| | | | tumor | | tumor | | % tumor | | Number |
| | | | volume | | volum | | inhibition | PValue | of |
| | | | (mm³) | | (mm³) | | rate | (vs | animal/ |
| Groups | Administration | Route | D0 | SEM | D38 | SEM | D38 | blank) | group |
| Solvent | d1-14/once | ip | 210.6 | 23.1 | 1710.5 | 281.7 | — | — | 5 |
| 39 (0.05 mg/kg) | d1-14/once | ip | 208.6 | 26.5 | 598.4 | 115.1 | 62.78 | 0.020266 | 5 |
| 40 (0.025 mg/kg) | d1-14/once | ip | 209.6 | 25.5 | 868.8 | 167.4 | 49.20 | 0.058313 | 5 |
| 40 (0.05 mg/kg) | d1-14/once | ip | 209.6 | 28.1 | 273.6 | 129.6 | 86.8 | 0.003073 | 5 |
| 40 (0.1 mg/kg) | d1-14/once | ip | 210.5 | 30.1 | 130.4 | 53.5 | 93.41 | 0.001662 | 5 |

### 4. Result

The experimental results show that the tumor inhibition rate of ADC compound 39 (0.5 mg/mouse) is 62.78% at day 38, and the difference was statistically significant (p<0.05) compared with the blank control group. The tumor inhibition rate of ADC compound 40 (0.025 mg/mouse) is 49.20%, which is not significantly different from that of blank control group (P>0.05). The tumor inhibition rate of ADC compound 40 (0.05 mg/mouse) and ADC compound 40 (0.1 mg/mouse) are 86.8% and 93.41%, respectively, which is significantly different from the blank control group (P<0.01). In addition, the anti-tumor effect of ADC compound 40 in three different dose groups is dose dependent.

1. A compound of formula (PC-L-Dr) or a pharmaceutically acceptable salt or solvate thereof: 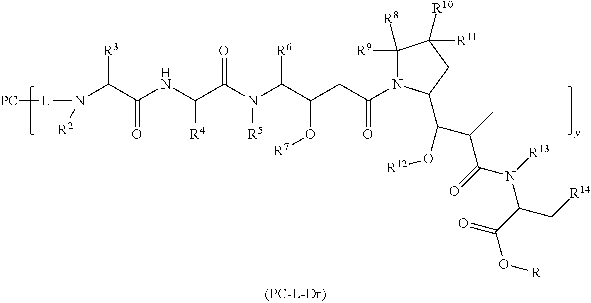
wherein:
each of R and R²-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are each hydrogen;
or any two of R⁸-R¹¹ are attached to form a cycloalkyl, and the other two are each selected from the group consisting of hydrogen, alkyl and cycloalkyl;
each of R¹²-R¹³ is selected from the group consisting of hydrogen, alkyl and halogen;
R¹⁴ is selected from the group consisting of aryl and heteroaryl, wherein the aryl or heteroaryl is optionally substituted with one or more groups selected from the group consisting of hydrogen, halogen, hydroxy, alkyl, alkoxy and cycloalkyl;
y is 1-8;
PC is a ligand; and
L is a linker.

2. The compound of (PC-L-Dr) according to claim 1, which is a compound of (PC-L-D), or a pharmaceutically acceptable salt or solvate thereof: 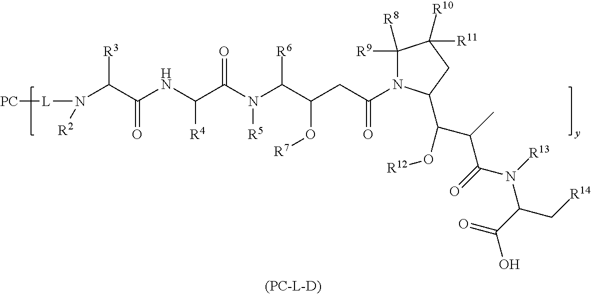
wherein R²-R¹⁴ are as defined in claim 1.

3. The compound of (PC-L-Dr) according to claim 1, which is a compound of (PC-L′-Dr), or a pharmaceutically acceptable salt or solvate thereof: 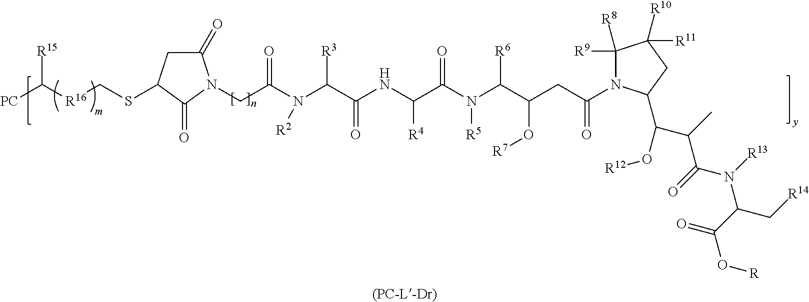
wherein:
R¹⁵ is selected from the group consisting of hydrogen, halogen, hydroxyl, cyano, alkyl, alkoxy and cycloalkyl;
R¹⁶ is selected from the group consisting of alkyl, cycloalkyl, alkoxy and heterocyclyl;
n is an integer of 2-6;
m is an integer of 0-5; and
PC, y, n, R, and R²-R¹⁴ are as defined in claim 1.

4. The compound of (PC-L-Dr) according to claim 1, which is a compound of (PC-L′-D), or a pharmaceutically acceptable salt or solvate thereof: 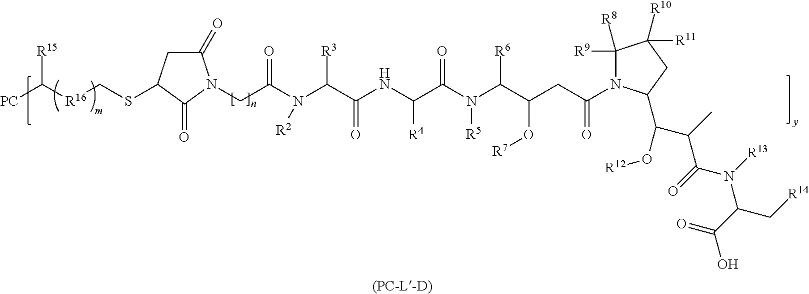
wherein:
R¹⁵ is selected from the group consisting of hydrogen, halogen, hydroxyl, cyano, alkyl, alkoxy and cycloalkyl;
R¹⁶ is selected from the group consisting of alkyl, cycloalkyl, alkoxy and heterocyclyl;
m is an integer of 0-5; and
PC, y, n, and R²-R¹⁴ are as defined in claim 1.

5. The compound of (PC-L′-D) according to claim 2, which is a compound of (PC-L′-D1), or a pharmaceutically acceptable salt or solvate thereof: 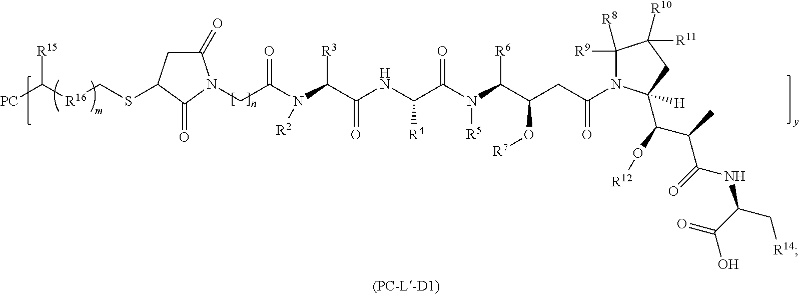
wherein PC, y, n, m, and R²-R¹⁶ are as defined in claim 2.

6. The compound of (PC-L-Dr), according to claim 1, wherein the compound is selected from the group consisting of: 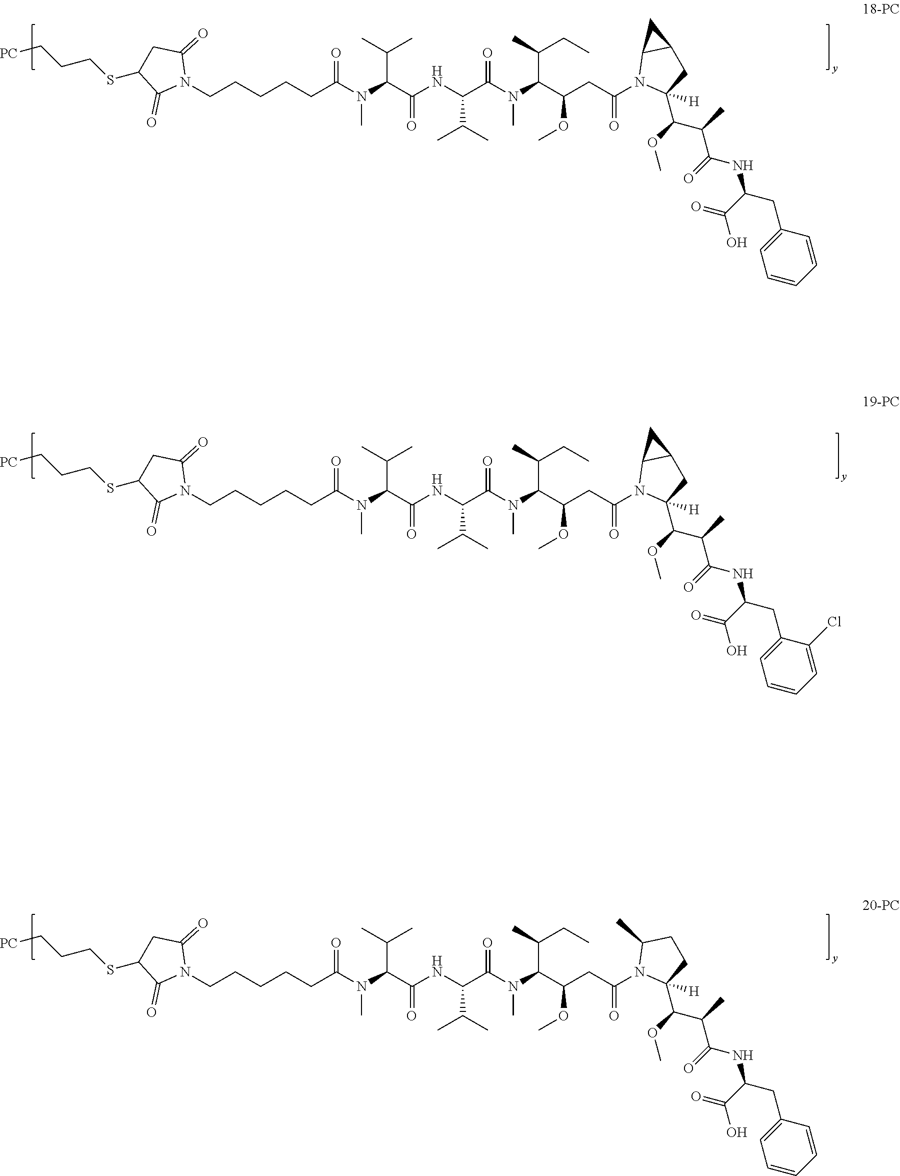 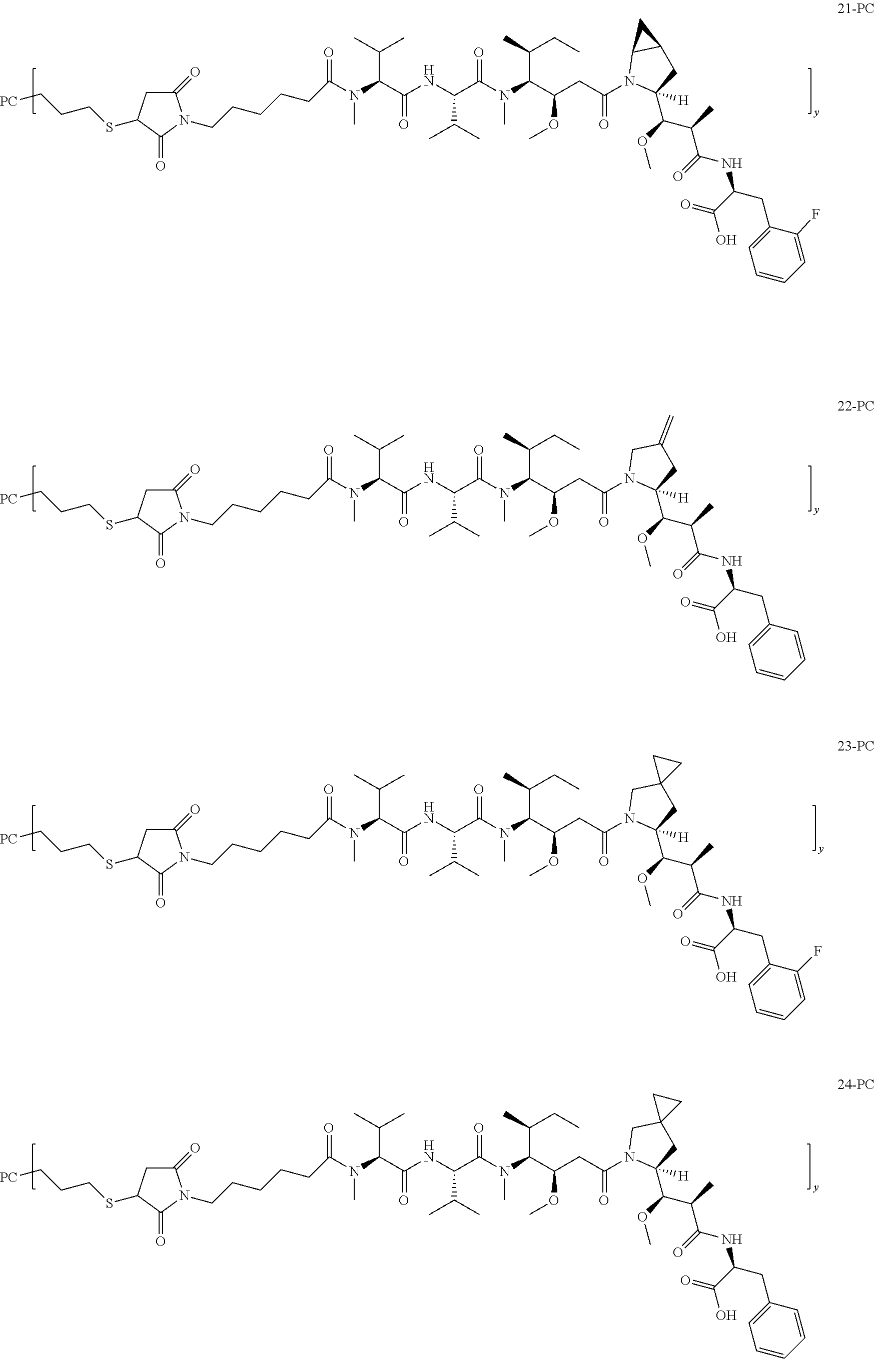 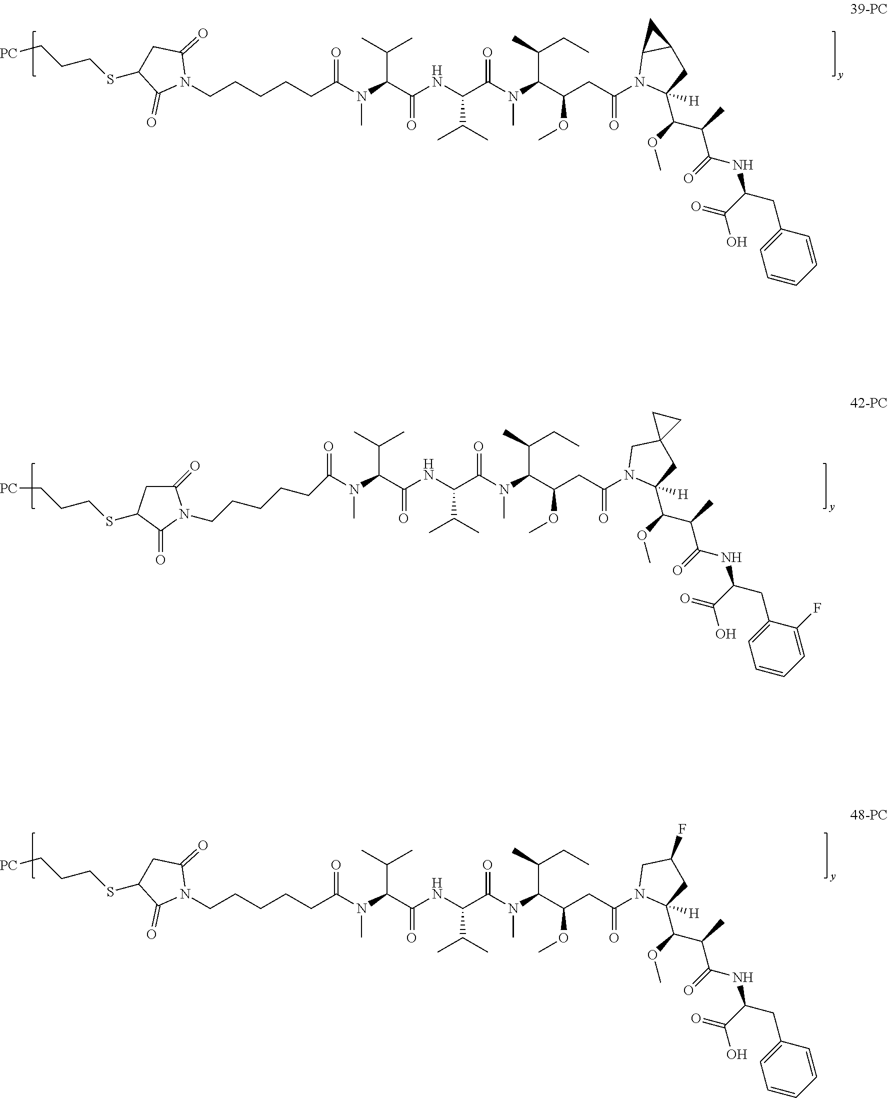 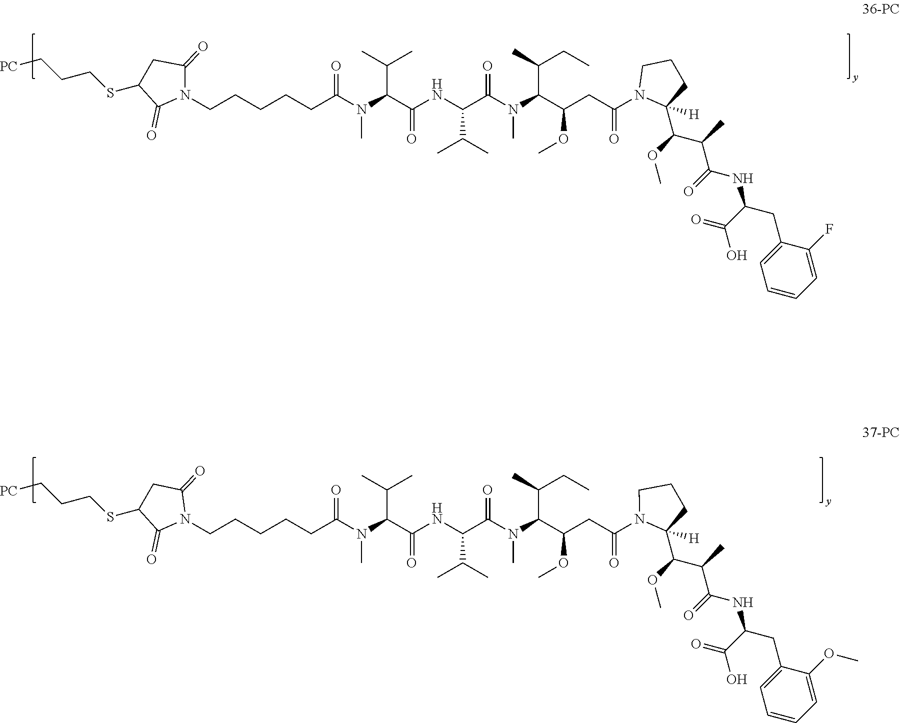 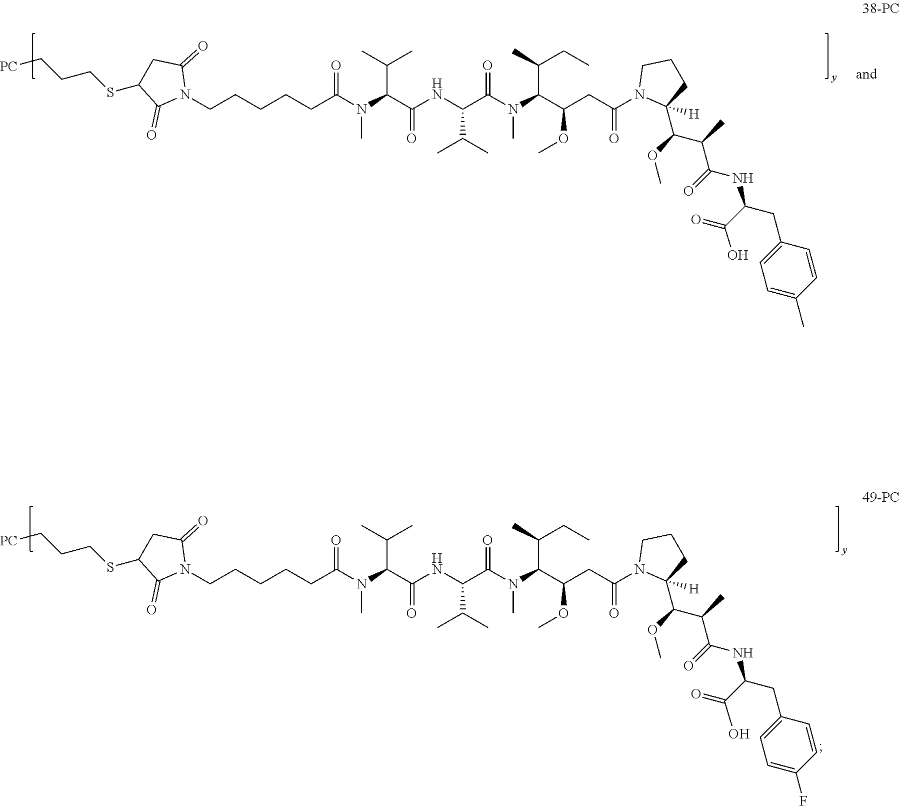
wherein PC and y are as defined in claim 1,
or a pharmaceutically acceptable salt or solvate thereof.

7. (canceled)

8. The compound of (PC-L-Dr) or a pharmaceutically acceptable salt or according to claim 1, wherein PC is an antibody.

9. The compound of (PC-L-Dr), according to claim 8, wherein the compound is selected from the group consisting of: 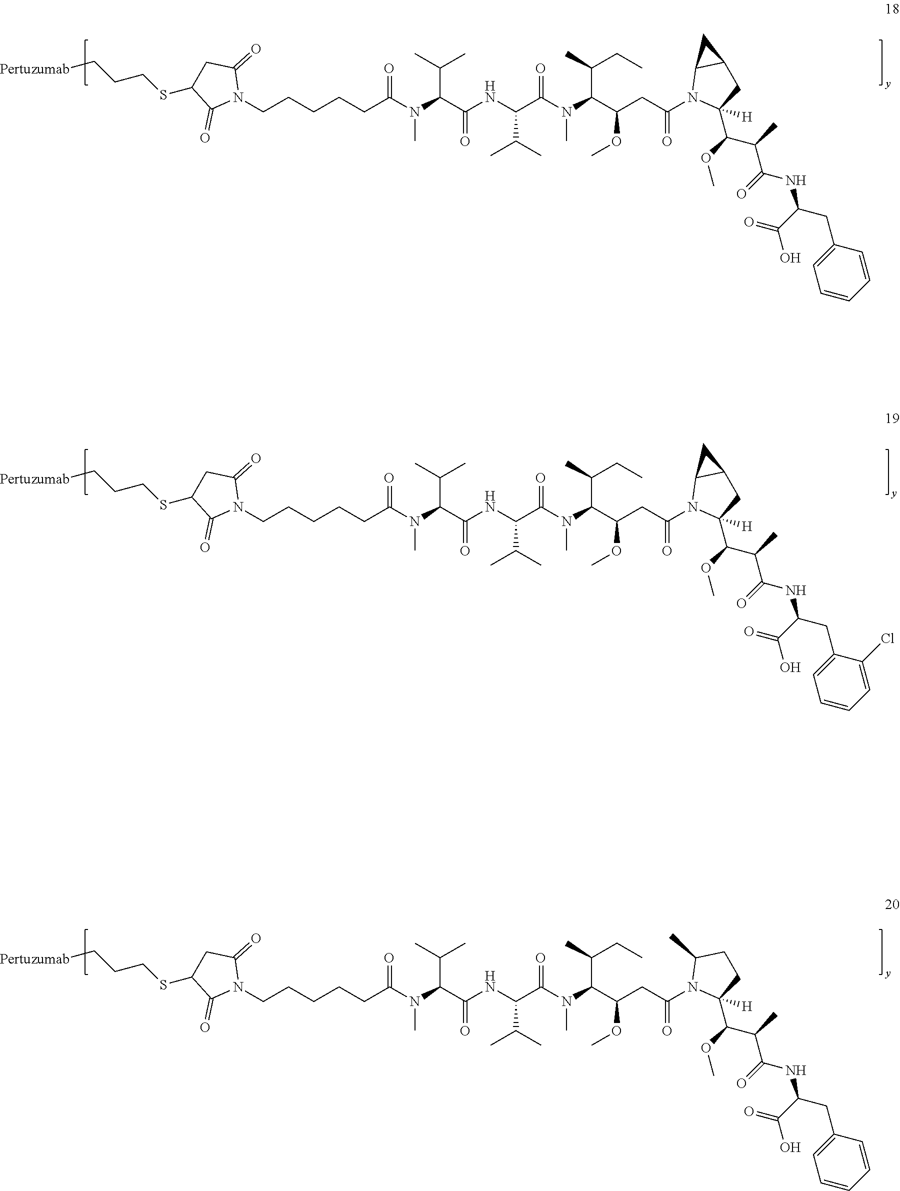 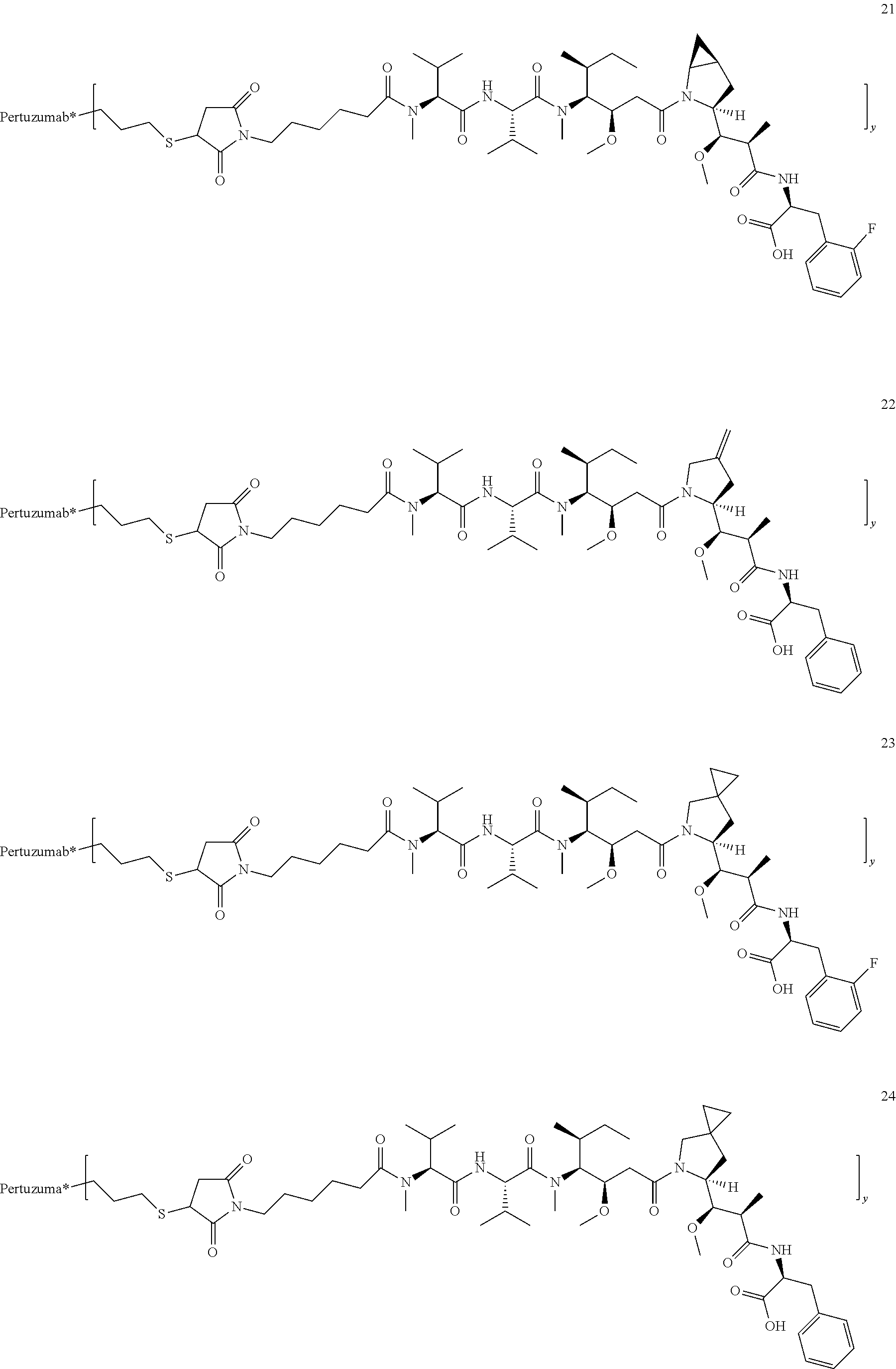 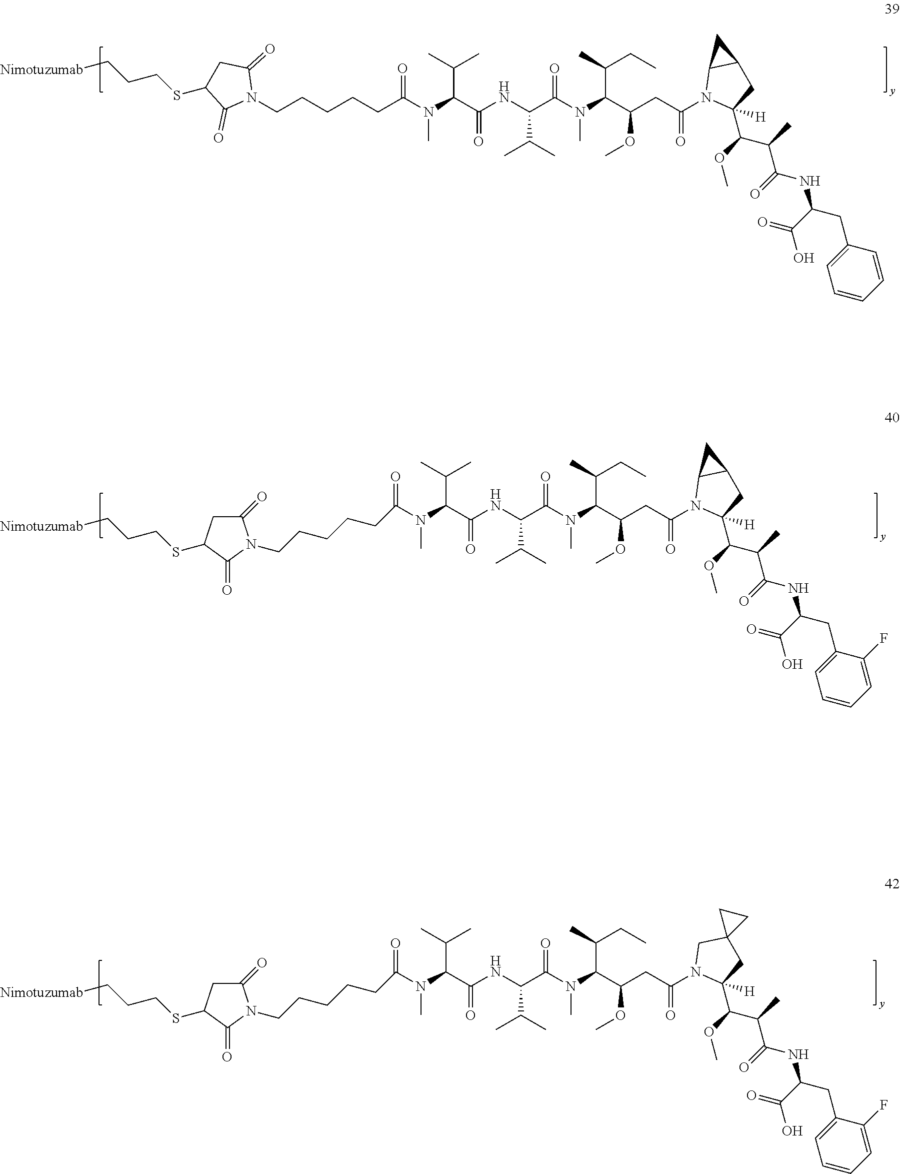 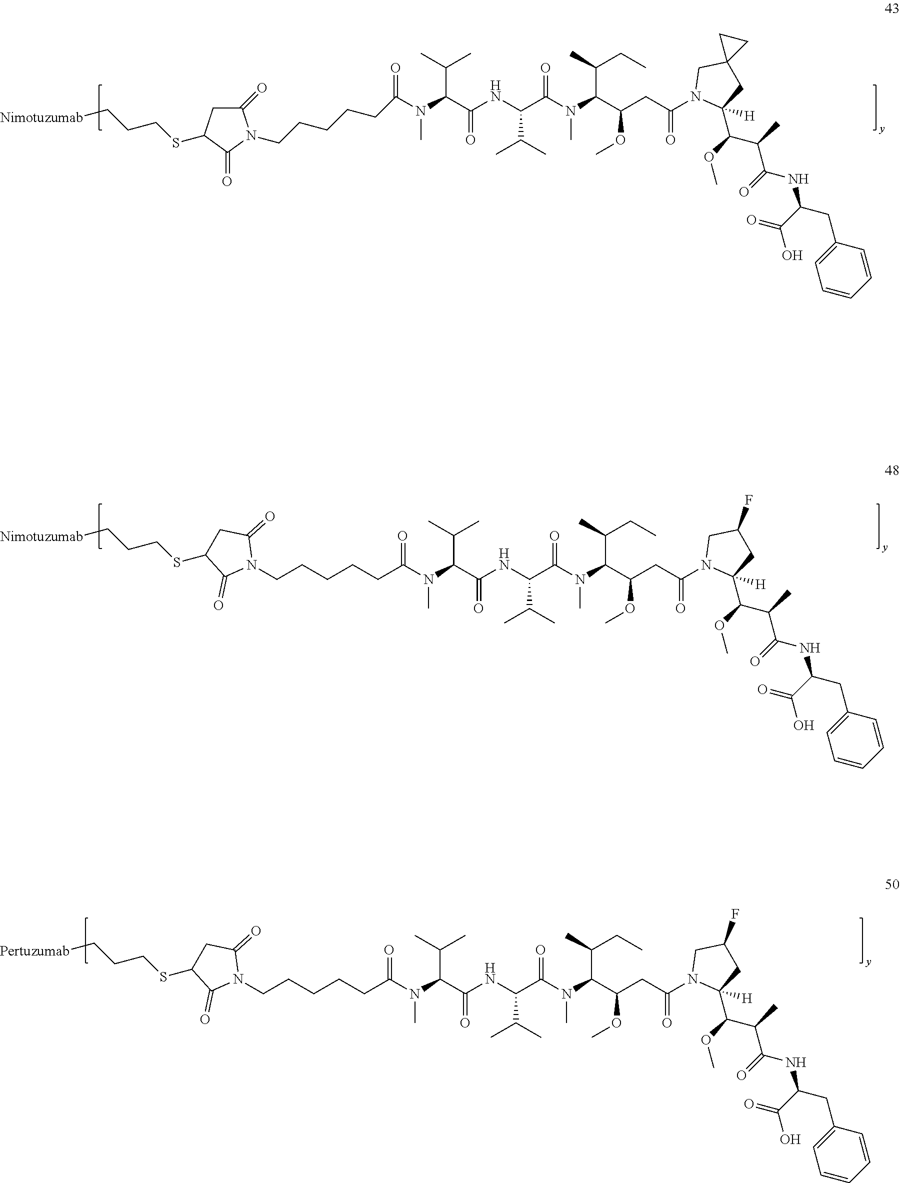 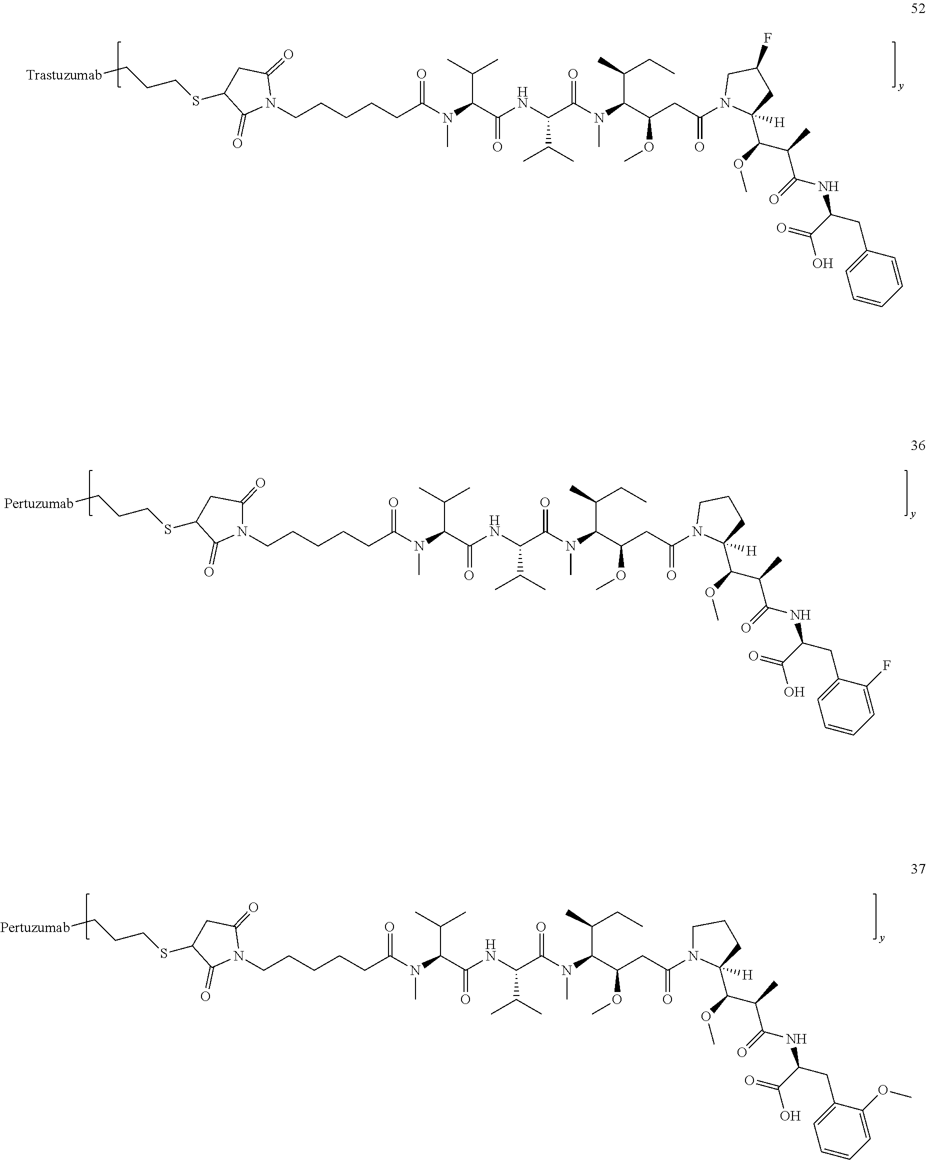 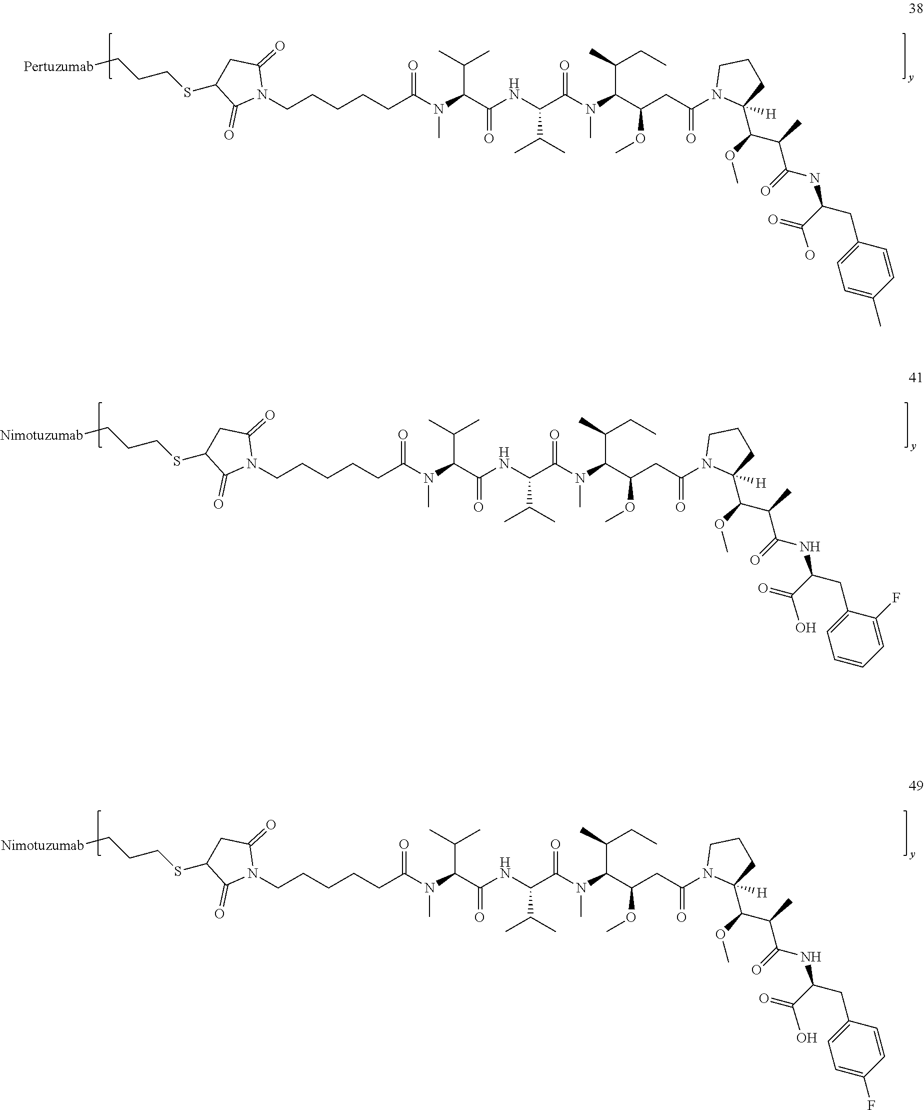 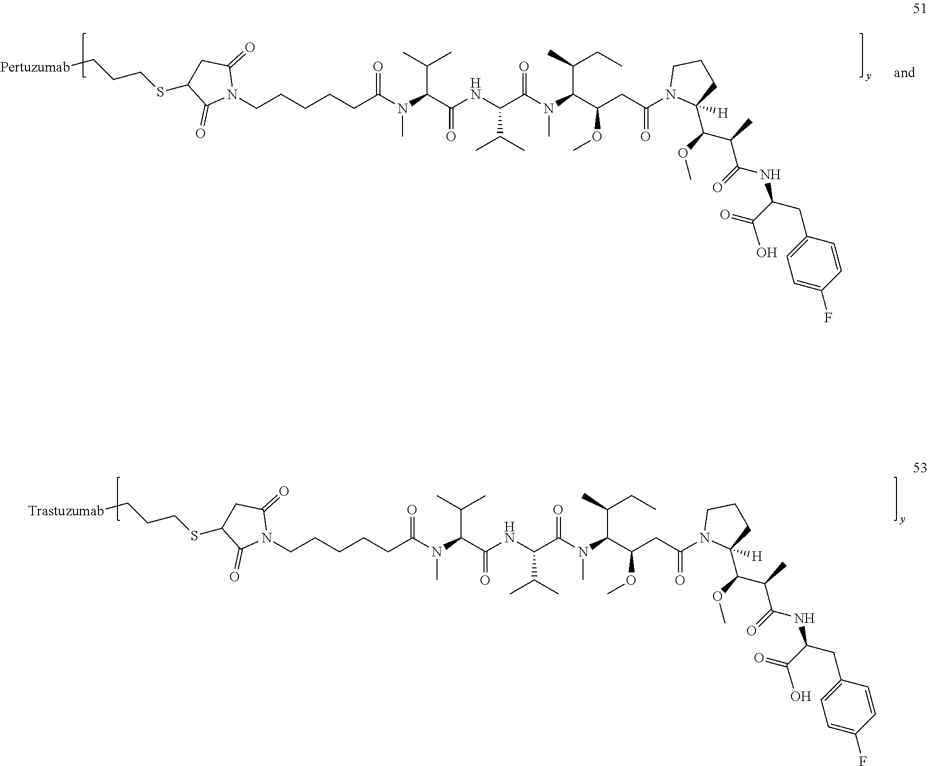
wherein y is 1-8,
or a pharmaceutically acceptable salt or solvate thereof.

10. (canceled)

11. A compound of formula (Dr): 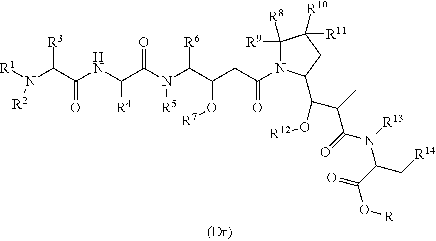
or a tautomer, mesomer, racemate, enantiomer, diastereomer, or mixture thereof, or pharmaceutically acceptable salt thereof,
wherein:
each of R and R¹-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are hydrogen;
or any two of R⁸-R¹¹ are attached to form a cycloalkyl, and the other two are each selected from the group consisting of hydrogen, alkyl and cycloalkyl;
R¹⁴ is selected from the group consisting of aryl and heteroaryl, wherein the aryl or heteroaryl is optionally substituted with one or more groups selected from the group consisting of hydrogen, halogen, hydroxy, alkyl, alkoxy and cycloalkyl; and
each of R¹²-R¹³ is selected from the group consisting of hydrogen, alkyl and halogen.

12. The compound of formula (Dr) according to claim 11, which is a compound of formula (D): 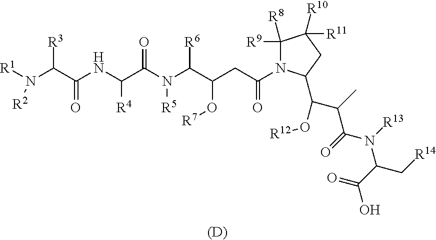
or a tautomer, mesomer, racemate, enantiomer, diastereomer, or mixture thereof, or pharmaceutically acceptable salt thereof, wherein R¹-R¹⁴ are as defined in claim 11.

13. The compound of formula (Dr) according to claim 11, which is a compound of formula (D1): 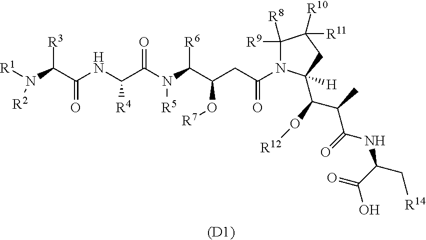
or a tautomer, mesomer, racemate, enantiomer, diastereomer, or mixture thereof, or pharmaceutically acceptable salt thereof, wherein R¹⁻-R¹⁴ are as defined in claim 11.

14. The compound of formula (Dr) according to claim 11, wherein the compound is selected from the group consisting of: 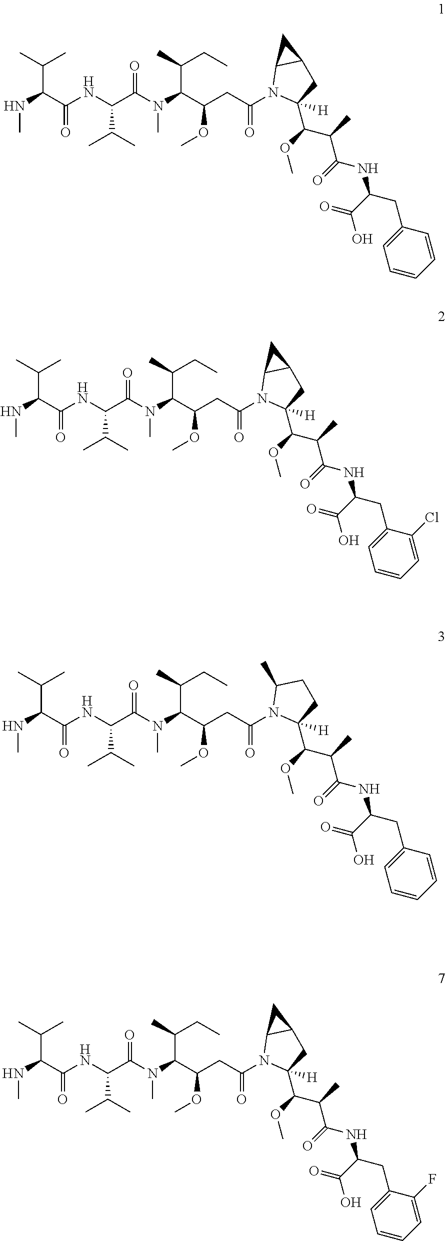 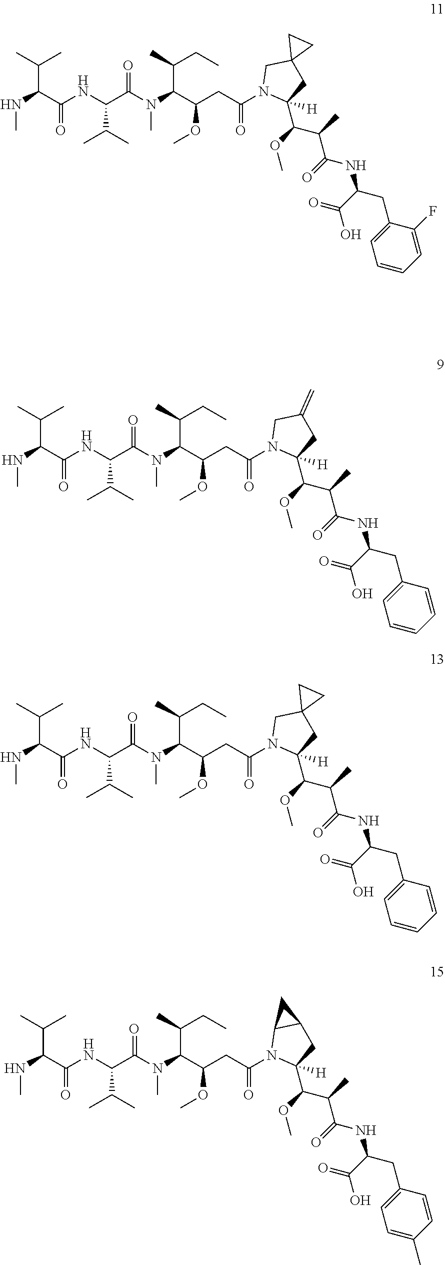 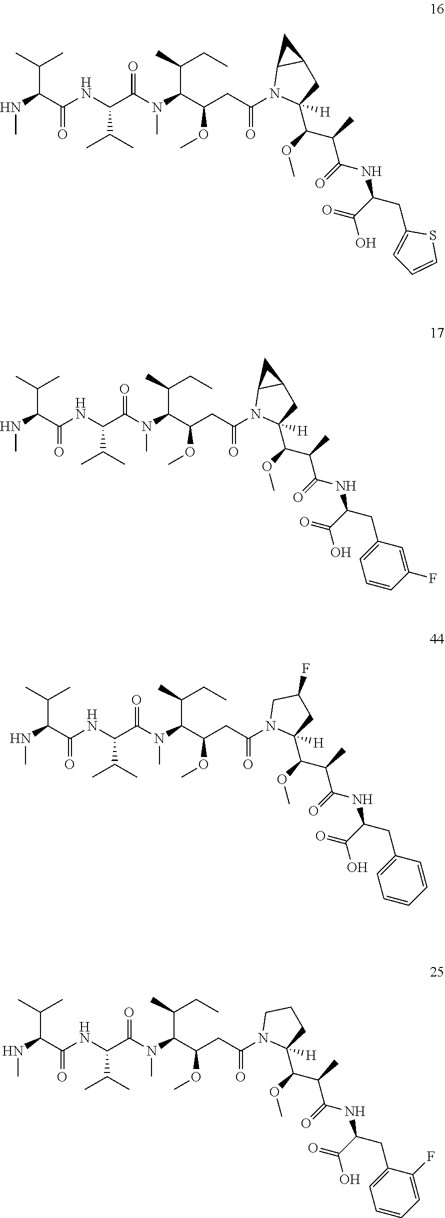 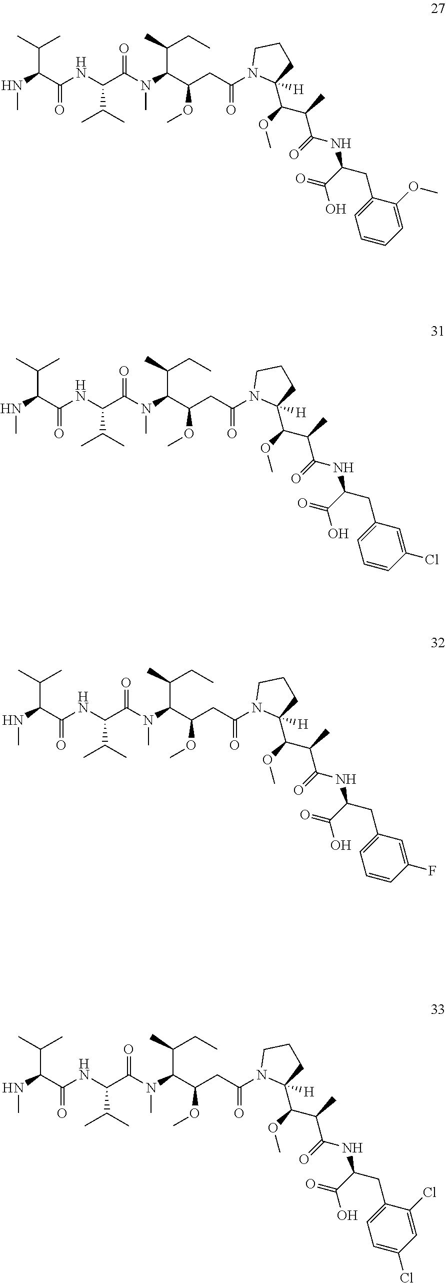 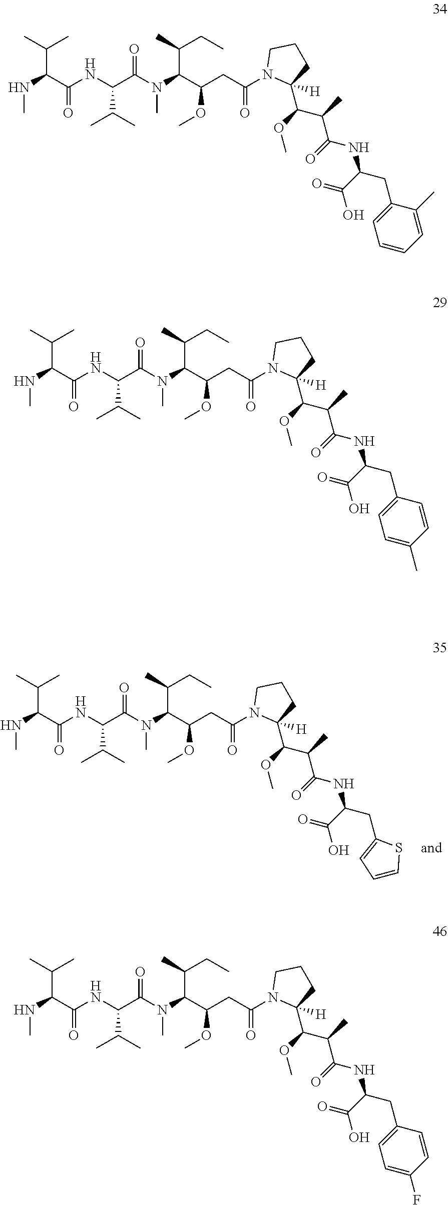
or a tautomer, mesomer, racemate, enantiomer, diastereomer, or mixture thereof, or pharmaceutically acceptable salt thereof.

15. (canceled)

16. A compound of formula (L₁-Dr): 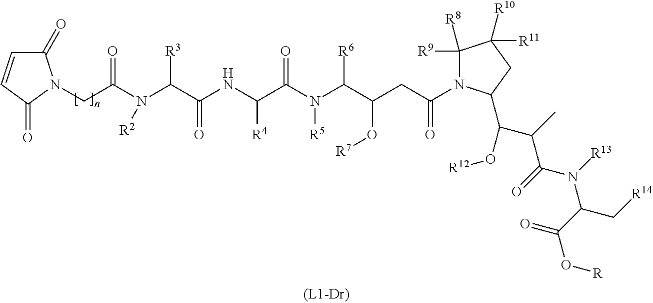
or a pharmaceutically acceptable salt or solvate thereof,
wherein:
n is an integer of 2-6;
each of R and R¹-R⁷ is selected from the group consisting of hydrogen, halogen, hydroxyl, cyano, alkyl, alkoxy and cycloalkyl;
at least one of R⁸-R¹¹ is selected from the group consisting of halogen, alkenyl, alkyl and cycloalkyl, and the rest of R⁸-R¹¹ are hydrogen;
or any two of R⁸-R¹¹ are attached to form a cycloalkyl, and the other two are each selected from the group consisting of hydrogen, alkyl and cycloalkyl;
each of R¹²-R¹³ is selected from the group consisting of hydrogen, alkyl and halogen;
R¹⁴ is selected from the group consisting of aryl and heteroaryl, wherein the aryl or heteroaryl is optionally substituted with one or more groups selected from the group consisting of hydrogen, halogen, hydroxy, alkyl, alkoxy and cycloalkyl.

17. The compound of (L1-Dr) according to claim 16, which is a compound of (L1-D): 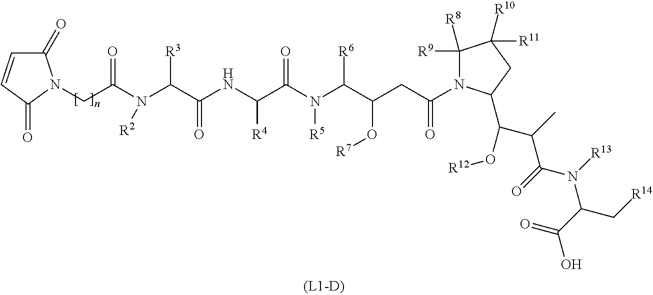
wherein n, and R²-R¹⁴ are as defined in claim 16, or
a pharmaceutically acceptable salt or solvate thereof.

18. The compound of formula (L₁-D) according to claim 16, wherein the compound is selected from the group consisting of: 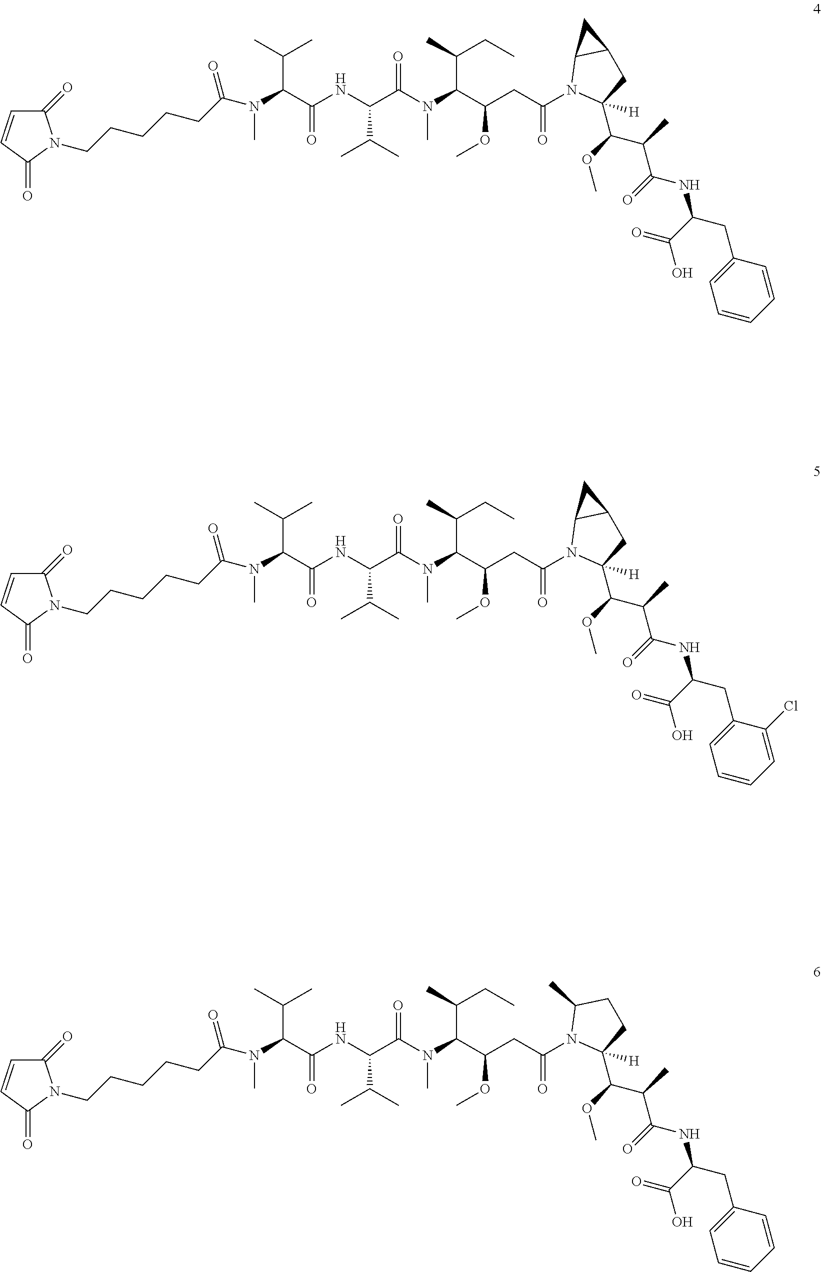 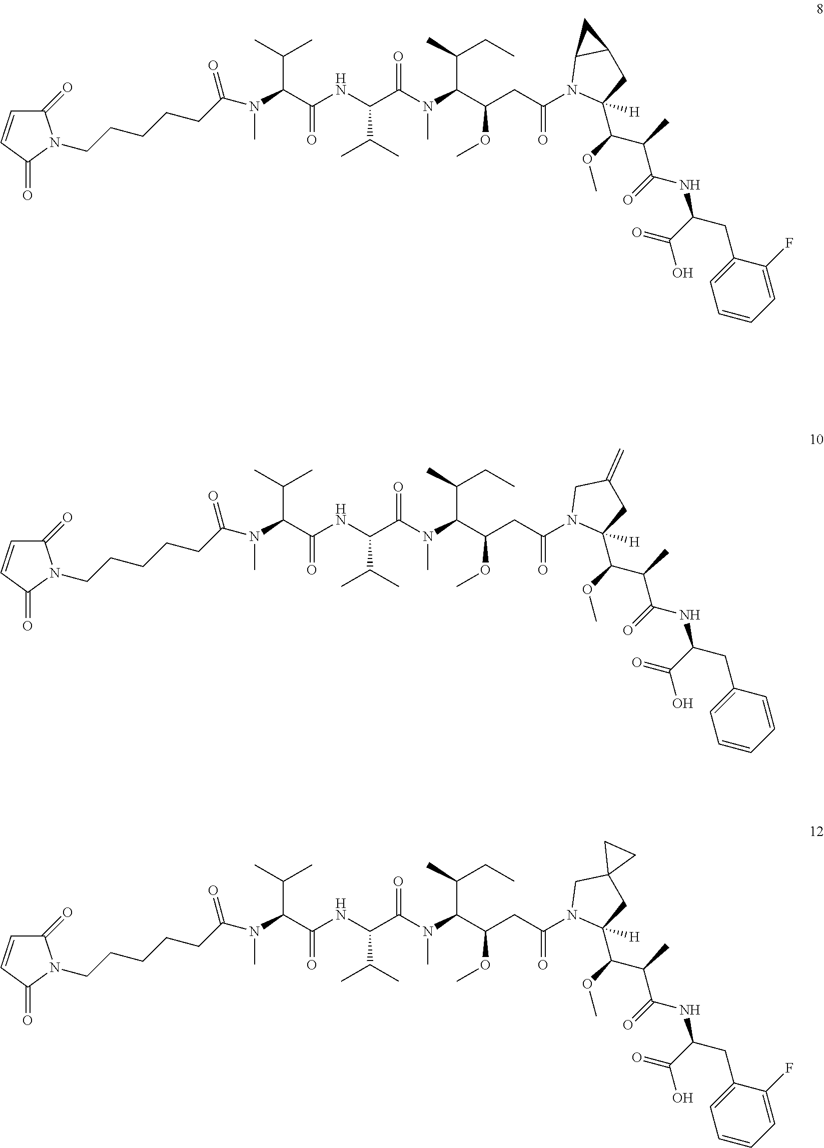 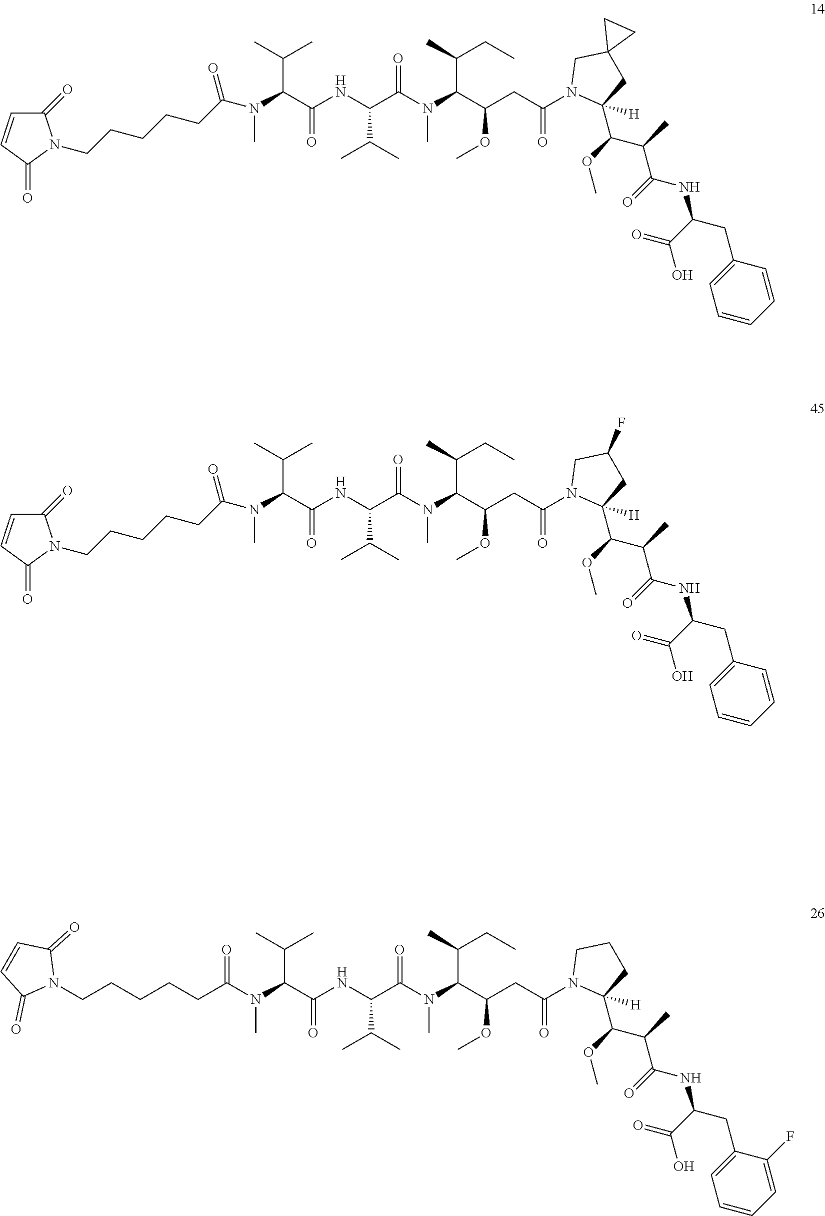 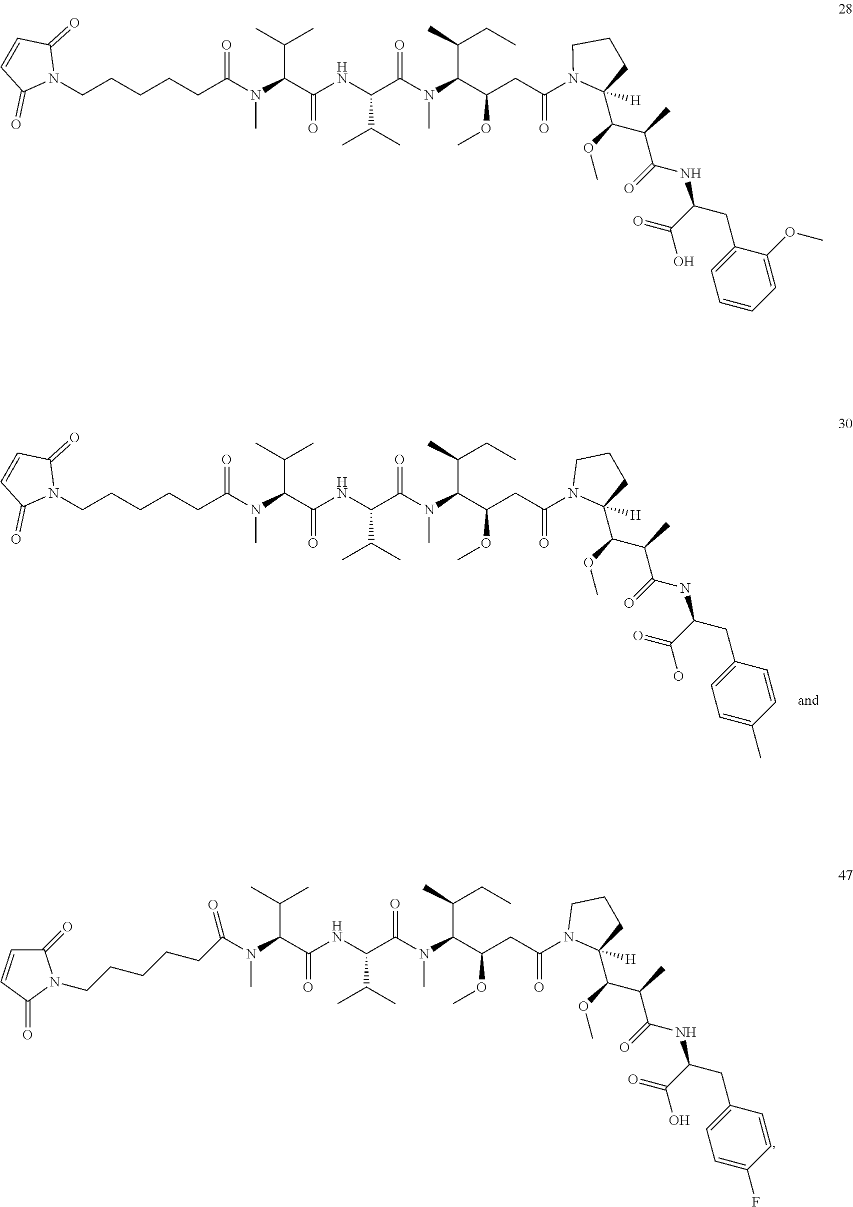 [SMILES: CC[C@H](C)[C@@H]([C@@H](CC(=O)N1CCC[C@H]1[C@H](OC)[C@@H](C)C(=O)N[C@@H](Cc1ccccc1OC)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CCC[C@H]1[C@H](OC)[C@@H](C)C(=O)N[C@@H](Cc1ccc(C)cc1)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1CCC[C@H]1[C@H](OC)[C@@H](C)C(=O)N[C@@H](Cc1ccc(F)cc1)C(=O)O)OC)N(C)C(=O)[C@@H](NC(=O)[C@H](C(C)C)N(C)C(=O)CCCCCN1C(=O)C=CC1=O)C(C)C]
or a pharmaceutically acceptable salt or solvate thereof.

19.-22. (canceled)

23. A process for preparing a compound of formula (PC-L′-D) according to claim 4, which comprises the steps of: 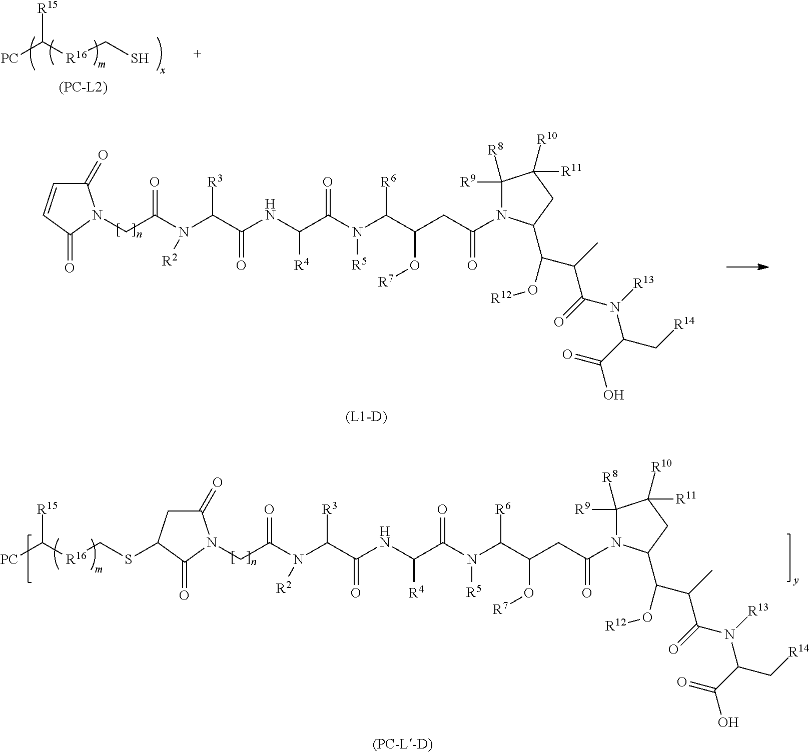
reacting a compound of formula (PC-L2) with a compound of formula (L1-D1) to obtain a compound of formula (PC-L′-D);
wherein R¹⁵ is selected from the group consisting of hydrogen, halogen, hydroxyl, cyano, alkyl, alkoxy and cycloalkyl;
R¹⁶ is selected from the group consisting of alkyl, cycloalkyl, alkoxy and heterocyclyl;
m is an integer of 0-5; and
PC, n, y, and R²-R¹⁴ are as defined in claim 4.

24.-26. (canceled)

27. A pharmaceutical composition comprising a therapeutically effective amount of the compound of formula (PC-L-Dr) according to claim 1 or a pharmaceutically acceptable salt or solvate thereof, and at least one pharmaceutically acceptable carrier, diluent or excipient.

28.-29. (canceled)

30. A pharmaceutical composition comprising a therapeutically effective amount of the compound of formula (Dr) according to claim 11, and at least one pharmaceutically acceptable carrier, diluent or excipient.

31. A method of inhibiting HER2, HER3, or EGFR in a subject in need thereof, the method comprising administering to the subject the pharmaceutical composition according to claim 27.

32. A method of inhibiting HER2, HER3, or EGFR in a subject in need thereof, the method comprising administering to the subject the pharmaceutical composition according to claim 30.

33. A method of treating cancer in a subject in need thereof, the method comprising administering to the subject the pharmaceutical composition according to claim 27, wherein the cancer is selected from the group consisting of breast cancer, ovarian cancer, stomach cancer, endometrial cancer, salivary gland cancer, lung cancer, colon cancer, renal cancer, colorectal cancer, thyroid cancer, pancreatic cancer, prostate cancer, bladder cancer, acute lymphocytic leukemia, acute myeloid leukemia, acute promyelocytic leukemia, chronic myelogenous leukemia, chronic lymphocytic leukemia, Hodgkin's lymphoma, non-Hodgkin's lymphoma and relapsed anaplastic large cell lymphoma.

34. A method of treating cancer in a subject in need thereof, the method comprising administering to the subject the pharmaceutical composition according to claim 30, wherein the cancer is selected from the group consisting of breast cancer, ovarian cancer, stomach cancer, endometrial cancer, salivary gland cancer, lung cancer, colon cancer, renal cancer, colorectal cancer, thyroid cancer, pancreatic cancer, prostate cancer, bladder cancer, acute lymphocytic leukemia, acute myeloid leukemia, acute promyelocytic leukemia, chronic myelogenous leukemia, chronic lymphocytic leukemia, Hodgkin's lymphoma, non-Hodgkin's lymphoma and relapsed anaplastic large cell lymphoma.
